(19) United States
(12) Patent Application Publication
(10) Pub. No.: US 2012/0003244 A1
(43) Pub. Date: Jan. 5, 2012

(54) METHODS FOR APCDD1 MEDIATED REGULATION OF HAIR GROWTH AND PIGMENTATION AND MUTANTS THEREOF

(76) Inventor: Angela M. Christiano, Saddle River, NJ (US)

(21) Appl. No.: 13/099,044

(22) Filed: May 2, 2011

Related U.S. Application Data
(63) Continuation-in-part of application No. PCT/US09/62970, filed on Nov. 2, 2009.
(60) Provisional application No. 61/110,029, filed on Oct. 31, 2008.

(51) Int. Cl. A61K 39/395; A61K 38/02; C07H 21/02; C07H 21/00; C40B 20/00; A61P 17/00; G01N 33/566; C12Q 1/68; G01N 33/573; G01N 21/64; A61K 31/7088; A61P 17/14

(52) U.S. Cl. 424/172.1; 514/44 A; 514/20.7; 514/44 R; 536/24.5; 536/24.31; 435/7.1; 435/6.13; 435/7.4; 435/6.11; 506/2

(57) ABSTRACT

The invention provides for methods for controlling hair growth by administering an APCDD1 modulating compound to a subject. The invention further provides for a method for screening compounds that bind to and modulate APCDD1.

This application is a continuation-in-part of International Application No. PCT/US09/62970 filed on Nov. 2, 2009, which claims the benefit of priority of U.S. Ser. No. 61/110,029, filed on Oct. 31, 2008, the contents of each which are hereby incorporated by reference in their entireties.

### GOVERNMENT INTERESTS

This invention was made with government support under RO1 AR44924 awarded by the National Institutes of Health/National Institute of Arthritis and Musculoskeletal and Skin Diseases. The United States Government has certain rights to the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted via EFS-Web and is hereby incorporated by reference in its entirety. Said ASCII copy, created on Nov. 25, 2009, is named 19240785.txt, and is 2,404,261 bytes in size.

### BACKGROUND OF THE INVENTION

Hereditary hypotrichosis simplex (HHS; OMIM 146520/605389) is an isolated form of hair loss. HHS is a rare autosomal dominant form of hereditary hair loss characterized by hair follicle (HF) miniaturization. APCDD1 (adenomatosis polyposis coli down-regulated 1) is a gene assigned at chromosomal band 18p11.2. It is a direct target of the WNT/β-catenin signaling pathway and has been identified to be over-expressed in certain cancers.

### SUMMARY OF THE INVENTION

The invention provides for an isolated mutant human APCDD1 polypeptide, methods for controlling hair growth by administering an APCDD1 modulating compound to a subject, and methods for screening compounds that bind to and modulate APCDD1. The invention also provides for diagnostic kits that can detect the presence of an aberrant APCDD1 protein.

One aspect of the invention provides for an isolated mutant human APCDD1 polypeptide comprising at least 1 amino acid mutation in SEQ ID NO: 1. In one embodiment, the mutation is a Leucine to Arginine mutation at amino acid position 9 of SEQ ID NO: 1, comprising the amino acid sequence of SEQ ID NO: 5.

One aspect of the invention also provides for an isolated mutant human APCDD1 polypeptide encoded by a nucleic acid sequence comprising at least about 70% identity, at least about 75% identity, at least about 80% identity, at least about 85% identity, at least about 90% identity, at least about 95% identity, or at least about 99% identity of SEQ ID NO: 2. In one embodiment, the nucleic acid sequence comprises the nucleic acid sequence of SEQ ID NO: 6.

An aspect of the invention provides for a nucleic acid encoding the polypeptide of the isolated mutant human APCDD1 of SEQ ID NO: 5 as well as for a vector that encodes the nucleic acid described herein.

One aspect of the invention provides methods for controlling hair growth in a subject, where the method comprises administering to the subject an effective amount of an APCDD1 modulating compound, thereby controlling hair growth in the subject. In one embodiment, controlling hair growth comprises an induction of hair growth in the subject or a promotion of hair loss in the subject. In one embodiment, the compound comprises an antibody that specifically binds to an APCDD1 protein or a fragment thereof; an antisense RNA or antisense DNA that inhibits expression of an APCDD1 polypeptide; a siRNA that specifically targets an APCDD1 gene; or a combination of those described herein. In another embodiment, the compound comprises a peptide comprising at least about 10 amino acids of SEQ ID NO: 1 or a vector comprising a nucleic acid sequence encoding a polypeptide comprising SEQ ID NO: 1. In a further embodiment, the subject is a human, a primate, a feline, a canine, or an equine. In some embodiments, the subject is afflicted with hypotrichosis. In other embodiments, the subject is afflicted with a hair-loss disorder. Non-limiting examples of the hair-loss disorder include androgenetic alopecia, Telogen effluvium, Alopecia greata, telogen effluvium, Alopecia greata, Tinea capitis, alopecia totalis, hypotrichosis, hereditary hypotrichosis simplex, or alopecia universalis. In further embodiments, the subject is afflicted with hypertrichosis. In other embodiments, administering comprises dispersing the APCDD1 modulating compound to a subject via subcutaneous, intradermal, intramuscular, intra-peritoneal, or intravenous injection; infusion; oral, nasal, or topical delivery; or a combination thereof; while in some embodiments, administering comprises dispersing the APCDD1 modulating compound to an epithelial cell derived from a hair follicle or skin. In some embodiments, hair growth can be regulated by modulating an APCDD1 target gene or APCDD1 interacting partner gene. Non-limiting examples of APCDD1 target genes or interacting partner genes include angiotensin receptor-related protein 1b (apelin receptor, agrtl 1b), foxi1-ema/Xema/Foxi1/HNF-3, histone 3r, polo-like kinase 2 (plk2), cyclin G1 (ccng1), PARP3-poly (ADP-ribose) polymerase family-member 3, haeme peroxidase E3 ubiquitin-protein ligase-Ring finger, ras-like 11b (rasl11b), Histone 2B, 5′-nucleotidase, cytosolic III (cytosolic 5′-nucleotidase III), angiotensin receptor-related protein 1 (agtrl1; e.g., XAngio1 in Xenopus), RAB40B, Histone 2, LRP5, WNT3A, and FGF receptor 2.

An aspect of the invention also provides for methods for controlling loss of hair pigmentation in a subject. The method comprises administering to the subject an effective amount of an APCDD1 modulating compound, thereby controlling hair pigmentation in the subject. In one embodiment, the compound comprises an antibody that specifically binds to an APCDD1 protein or a fragment thereof; an antisense RNA or antisense DNA that inhibits expression of an APCDD1 polypeptide; a siRNA that specifically targets an APCDD1 gene; or a combination of those described herein. In another embodiment, the compound comprises a peptide comprising at least about 10 amino acids of SEQ ID NO: 1 or a vector comprising a nucleic acid sequence encoding a polypeptide comprising SEQ ID NO: 1. In a further embodiment, the subject is a human, a primate, a feline, a canine, or an equine. In some embodiments, the subject is afflicted with hypotrichosis. In other embodiments, the subject is afflicted with a hair-loss disorder. Non-limiting examples of the hair-loss disorder include androgenetic alopecia, Telogen effluvium, Alopecia greata, telogen effluvium, Alopecia greata, Tinea capitis, alopecia totalis, hypotrichosis, hereditary hypotrichosis simplex, or alopecia universalis. In further embodiments, the subject is afflicted with hypertrichosis. In other embodiments, administering comprises dispersing the APCDD1 modulating compound to a subject via subcutaneous, intradermal, intramuscular, intra-peritoneal, or intravenous injection; infusion; oral, nasal, or topical delivery; or a combination thereof; while in some embodiments, administering comprises dispersing the APCDD1 modulating compound to an epithelial cell derived from a hair follicle or skin. In some embodiments, hair loss can be controlled by modulating an APCDD1 target gene or APCDD1 interacting partner gene. Non-limiting examples of APCDD1 target genes or interacting partner genes include angiotensin receptor-related protein 1b (apelin receptor, agrtl 1b), foxi1-ema/Xema/Foxi1/HNF-3, histone 3r, polo-like kinase 2 (plk2), cyclin G1 (ccng 1), PARP3-poly (ADP-ribose) polymerase family-member 3, haeme peroxidase E3 ubiquitin-protein ligase-Ring finger, ras-like 11b (rasl11b), Histone 2B, 5′-nucleotidase, cytosolic III (cytosolic 5′-nucleotidase III), angiotensin receptor-related protein 1 (agtrl1; e.g., XAngio1 in Xenopus), RAB40B, Histone 2, LRP5, WNT3A, and FGF receptor 2.

One aspect of the invention also provides for a composition for modulating APCDD1 protein expression or activity in a subject in need thereof, wherein the composition comprises an siRNA that specifically targets an APCDD1 gene. In one embodiment, the siRNA comprises a nucleic acid sequence comprising any one sequence of SEQ ID NO: 112-3776. In another embodiment, APCDD1 protein expression is decreased by at least about 40%, at least about 45%, at least about 50%, at least about 55%, at least about 60%, at least about 65%, at least about 70%, at least about 75%, at least about 80%, at least about 90%, at least about 95%, at least about 99%, or 100%. In a further embodiment, APCDD1 protein expression is increased by at least about 40%, at least about 45%, at least about 50%, at least about 55%, at least about 60%, at least about 65%, at least about 70%, at least about 75%, at least about 80%, at least about 90%, at least about 95%, at least about 99%, or 100%. In other embodiments, the subject is a human, a primate, a feline, a canine, or an equine. In further embodiments, the subject is afflicted with hypotrichosis; while in some embodiments, the subject is afflicted with a hair-loss disorder. Non-limiting examples of the hair-loss disorder includes androgenetic alopecia, Alopecia greata, telogen effluvium, Alopecia greata, alopecia totalis, or alopecia universalis. Yet, in some embodiments, the subject is afflicted with hypertrichosis.

An aspect of the composition for controlling hair growth or loss of hair pigmentation in a subject, the composition in an admixture of a pharmaceutically acceptable carrier comprising an APCDD1 modulating compound. In one embodiment, the pharmaceutically acceptable carrier comprises water, a glycol, an ester, an alcohol, a lipid, or a combination of the carriers described herein. In another embodiment, hair growth comprises an induction of hair growth in the subject or a promotion of hair loss in the subject. In a further embodiment, the compound comprises an antibody that specifically binds to an APCDD1 protein or a fragment thereof; an antisense RNA or antisense DNA that inhibits expression of an APCDD1 polypeptide; a siRNA that specifically targets an APCDD1 gene; or a combination thereof. In some embodiments, the compound comprises a peptide comprising at least about 10 amino acids of SEQ ID NO: 1 or a vector comprising a nucleic acid sequence encoding a polypeptide comprising SEQ ID NO: 1. In other embodiments, the subject is a human, a primate, a feline, a canine, or an equine. In further embodiments, the subject is afflicted with hypotrichosis; while in some embodiments, the subject is afflicted with a hair-loss disorder. Non-limiting examples of the hair-loss disorder includes androgenetic alopecia, Alopecia greata, telogen effluvium, Alopecia greata, alopecia totalis, or alopecia universalis. Yet, in some embodiments, the subject is afflicted with hypertrichosis.

An aspect of the invention provides for a kit for controlling hair growth. The kit comprises a container having a composition described above disposed within the kit and instructions for use.

An aspect of the invention also provides a method for identifying a compound that modulates APCDD1 protein activity. The method comprises (1) expressing APCDD1 protein in a cell; (2) contacting a cell with a ligand source for an effective period of time; (3) measuring a secondary messenger response, wherein the response is indicative of a ligand binding to APCDD1 protein; (4) isolating the ligand from the ligand source; and (5) identifying the structure of the ligand that binds APCDD1 protein, thereby identifying which compound would modulate the activity of APCDD1 protein. In one embodiment, the method further comprises (i) obtaining or synthesizing the compound determined to bind to APCDD1 protein or to modulate APCDD1 protein activity; (ii) contacting APCDD1 protein with the compound under a condition suitable for binding; and (iii) determining whether the compound modulates APCDD1 protein activity using a diagnostic assay. In another embodiment, the compound is an APCDD1 agonist or an APCDD1 antagonist. In a further embodiment, the antagonist decreases APCDD1 protein or RNA expression or APCDD1 activity by at least about 10%, at least about 20%, at least about 30%, at least about 40%, at least about 50%, at least about 60%, at least about 70%, at least about 75%, at least about 80%, at least about 90%, at least about 95%, or at least about 99%. In some embodiments, the antagonist decreases APCDD1 protein or RNA expression or APCDD1 activity by 100%. In other embodiments, the agonist increases APCDD1 protein or RNA expression or APCDD1 activity by at least about 10%, at least about 20%; at least about 30%, at least about 40%, at least about 50%, at least about 60%, at least about 70%, at least about 75%, at least about 80%, at least about 90%, at least about 95%, or at least about 99%. In some embodiments, the agonist increases APCDD1 protein or RNA expression or APCDD1 activity by 100%. In further embodiments, the compound comprises an antibody that specifically binds to an APCDD1 protein or a fragment thereof; an antisense RNA or antisense DNA that inhibits expression of an APCDD1 polypeptide; a siRNA that specifically targets an APCDD1 gene, a peptide comprising at least 10 amino acids of SEQ ID NO: 1 wherein the peptide competes with endogenous APCDD1 for ligand binding; or a combination of such. In yet, some embodiments, the cell is a bacterium, a yeast, an insect cell, or a mammalian cell. In one embodiment, the ligand source is a compound library or a tissue extract. In other embodiments, measuring comprises detecting an increase or decease in a secondary messenger concentration; while in some embodiments, the assay determines the concentration of the secondary messenger within the cell. Non-limiting examples of the secondary messenger include glycogen synthase kinase 3β (GSK3β), β-catenin, adenomatous polyposis coli (APC), axin, or a combination thereof. In one embodiment, contacting comprises administering the compound to a mammal in vivo or a cell in vitro. In another embodiment, the mammal is a mouse. In a further embodiment, the compound increases or decreases downstream signaling of the APCDD1 protein. Yet in other embodiments, the assay measures an intracellular concentration of glycogen synthase kinase 3β (GSK3β), β-catenin, adenomatous polyposis coli (APC), or axin. In some embodiments, the assay measures LEF/TCF transcription, while in other embodiments the assay measures β-catenin phosphorylation or β-catenin nuclear translocation.

An aspect of the invention provides a method for detecting the presence of or a predisposition to a hair-loss disorder in a human subject. The method comprises (1) obtaining a biological sample from a human subject; and (2) detecting whether or not there is an alteration in the expression of APCDD1 protein in the subject as compared to a subject not afflicted with a hair-loss disorder. In one embodiment, the detecting comprises detecting whether there is an alteration in the APCDD1 gene locus. In another embodiment, the alteration comprises a missense mutation. In a further embodiment, the mutation is thymine to guanine substitution at position 26 of SEQ ID NO: 2. In some embodiments, the detecting comprises detecting whether a small nuclear polymorphism (SNP) is present in the APCDD1 gene locus, while in other embodiments, the SNP comprises a single nucleotide change, or a cluster of SNPs in and around the APCDD1 gene, or other SNPS that are in linkage disequilibrium (LD) with APCDD1. In further embodiments, the detecting comprises detecting whether at least a portion of the APCDD1 gene is deleted. In yet other embodiments, the detecting comprises detecting whether the signal peptide sequence of the APCDD1 protein is altered. In some embodiments, the detecting comprises detecting whether there is an alteration in the APCDD1 protein. In other embodiments, the alteration comprises a Leucine to Arginine substitution at amino acid position 9 of SEQ ID NO: 1. In some embodiments, the detecting comprises detecting whether expression of APCDD1 is reduced, while in other embodiments, the detecting comprises detecting in the sample whether there is a reduction in APCDD1 mRNA, APCDD1 protein, or a combination thereof. In some further embodiments, detecting comprises gene sequencing, selective hybridization, amplification, gene expression analysis, or a combination of the methods described. In one embodiment, amplification comprises using forward and reverse RT-PCR primers comprising nucleotide sequences of SEQ ID NOS: 9, 10, 13, 14, 57, or 103. In another embodiment, the subject is a human, a dog, or a mouse. In a further embodiment, the sample comprises blood, serum, sputum, lacrimal secretions, semen, vaginal secretions, fetal tissue, skin tissue, epithelial tissue, muscle tissue, amniotic fluid, or a combination of the samples described. In some embodiments, a reduction in APCDD1 expression of at least 20% indicates a predisposition to or presence of a hair-loss disorder in the subject. In further embodiments of the invention, the hair-loss disorder comprises androgenetic alopecia, Alopecia greata, telogen effluvium, Alopecia greata, alopecia totalis, hypotrichosis, hereditary hypotrichosis simplex, or alopecia universalis. In some embodiments, detecting the presence of or a predisposition to a hair-loss disorder in a human subject occurs by detecting the upregulation or downregulation of the expression of one or more proteins encoded by an APCDD1 target gene or APCDD1 interacting partner gene, either alone or in combination with detecting whether there is an alteration in the expression of the APCDD1 protein. Non-limiting examples of APCDD1 target genes or interacting partner genes include angiotensin receptor-related protein 1b (apelin receptor, agrtl 1b), foxi1-ema/Xema/Foxi1/HNF-3, histone 3r, polo-like kinase 2 (plk2), cyclin G1 (ccng1), PARP3-poly (ADP-ribose) polymerase family-member 3, haeme peroxidase E3 ubiquitin-protein ligase-Ring finger, ras-like 11b (rasl11b), Histone 2B, 5′-nucleotidase, cytosolic III (cytosolic 5′-nucleotidase III), angiotensin receptor-related protein 1 (agtrl1; e.g., XAngio1 in Xenopus), RAB40B, Histone 2, LRP5, WNT3A, and FGF receptor 2.

An aspect of the invention provides a diagnostic kit for determining whether a sample from a subject exhibits reduced APCDD1 expression or exhibits an APCDD1 gene mutation. The kit comprises nucleic acid primers that specifically hybridize to and can prime a polymerase reaction from APCDD1. In one embodiment, the primers comprise a nucleotide sequence of SEQ ID NOS: 9, 10, 13, 14, 21, 22, 23, 24, 25, 67, 68, 69, 70, or 71. In another embodiment, the mutation comprises a Leucine to Arginine substitution at amino acid position 9 of SEQ ID NO: 1.

### BRIEF DESCRIPTION OF THE FIGURES

To conform to the requirements for U.S. patent applications, many of the figures presented herein are black and white representations of images originally created in color, such as many of those figures based on immunofluorescence microscopy, e.g., DAPI (blue) staining and APCDD1 staining in green. In the below descriptions and the examples, this colored staining is described in terms of its appearance in black and white. For example, DAPI staining which appeared blue in the original appears as a dark grey stain when presented in black and white. The original color versions can be viewed in Shimomura et al., Nature 2010 (Apr. 15), 464(7291):1043-7 (including the accompanying Supplementary Information available in the on-line version of the manuscript available on the Nature Genetics web site). For the purposes of the U.S., the contents of Shimomura et al., Nature 2010 (Apr. 15), 464(7291):1043-7, including the accompanying “Supplementary Information,” are herein incorporated by reference.

FIGS. 1A-F are photographs showing the clinical appearance of hereditary hypotrichosis simplex (HHS). The age of each individual is 7 (FIG. 1A), 3 (FIG. 1B), 10 (FIG. 1C), 28 (FIG. 1D), 20 (FIG. 1E), and 16 (FIG. 1F) years old, respectively

FIG. 1G is a bar graph depicting the results of autozygosity, fine mapping of HHS phenotype on chromosome 18p11.2. The maximum LOD score was obtained for a region on chromosome 18.

FIG. 1H represents haplotype analysis of a Pakistani family HHS1. The linked haplotype is indicated in red, and critical recombination events are indicated by an arrowhead.

FIG. 2A is a schematic representation of the candidate region harboring the HHS gene. Arrows indicate the position and the direction of transcription of genes in the region.

FIG. 2B is a DNA chromatogram identifying a mutation in the APCDD1 gene. A heterozygous 26T>G (L9R) mutation in the APCDD1 gene of both families HHS1 and HHS2 was observed [left panel, SEQ ID NO: 9737, amino acid sequence disclosed as SEQ ID NO: 9771; right panel (control), SEQ ID NO: 9738, amino acid sequence disclosed as SEQ ID NO: 9772]. Screening assays with the restriction enzyme DdeI in HHS1 are shown below the chromatograms as a gel image. The 191 bp fragment only from the wild-type allele was digested into 149 bp and 42 bp fragments.

FIG. 2C is a photographic image of a western blot. Tagged vs. untagged SWAMP wt and mutant was compared. Animal caps were injected with 1 ng RNA of each of the indicated molecules, and 1 ng LacZ RNA as control for amount of injected RNA. Western blot with antibodies against SWAMP (1:10,000) and β-Galactosidase (1:1000, ProScience Inc.). The HA tag stabilizes the L9R mutant, but has no effect on the wt molecule.

FIG. 3A is a schematic representation of APCDD1 protein and position of the mutation L9R.

FIG. 3B is a multiple amino-acid sequence alignment of the signal peptide sequences of APCDD1 between different species. Residues that are conserved among at least five species are colored yellow. The Leu9 is denoted as “Leu 9”. The accession numbers of GenBank or Ensembl databases for the respective APCDD1 proteins are: Homo sapiens, NP_{—}694545 [SEQ ID NO: 9750]; Equus caballus, ENSECAP00000009668 [SEQ ID NO: 9751]; Canis familiaris, XP_{—}537333 [SEQ ID NO: 9752]; Mus musculus, NP_{—}573500 [SEQ ID NO: 9753]; Myotis lucifugus, ENSMLUP00000001735 [SEQ ID NO: 9754]; Gallus gallus, ENSGALP00000001313 [SEQ ID NO: 9755]; Pelodiscus sinensis, BAD74115 [SEQ ID NO: 9756]; Xenopus laevis, BAE02564 [SEQ ID NO: 9757].

FIG. 3C is a photograph of a western blot analysis of cell lysates and medium from HEK293T cells that APCDD1 expression constructs were transfected. Strong expression of the wild-type L9V mutant APCDD1 was detected in both cell lysate and medium (lanes 1 and 3), whereas that of the L9R mutant APCDD1 was weakly detected only in cell lysate (lane 2). When the L9R mutant APCDD1 expression construct was co-transfected with the wild-type construct, the expression of the wild-type protein was significantly decreased (lane 6). Beta-actin was used as a normalization control in cell lysate, and also as a control to deny the contamination of cell lysate in medium.

FIGS. 3D-G are photographs of indirect immunofluorescence analysis in HEK293T cells.

FIG. 4A is a photograph of a northern blot analysis showing APCDD1 expression in the human hair follicles. RT-PCR amplification of the APCDD1 mRNA is shown from plucked human hair follicles. MWM, molecular weight marker.

FIG. 4B is a photograph of semiquantitative RT-PCR showing that the APCDD1 expression immediately decreased upon explant culture.

FIGS. 4C-D are photographs of in situ hybridization. Antisense probe (AS) detected the strong signals in the dermal papilla, the matrix, and the precortex of the human hair follicles (FIG. 4C), while sense probe (S) did not show any positive signals (FIG. 4D).

FIGS. 4E-J are photographs of indirect immunofluorescence. APCDD1 protein is abundantly expressed in the dermal papilla (DP), the matrix (Mx), the hair shaft cortex (HSCx), hair shaft cuticle (HSCu), and weakly in the inner root sheath (IRS) of the human hair follicles (FIGS. 4E-F). Double immunostaining of APCDD1 with an inner root sheath (IRS)-specific marker K71 confirmed that APCDD1 is expressed in the IRS as well (FIGS. 4G-I). In the upper portion of the hair follicles, APCDD1-expression is detected in the outer root sheath (ORS) and the sebaceous gland (SG)(FIG. 4J). Scale bars: 100 μm.

FIGS. 5A-F are photographs of the clinical appearance of affected individuals in the Pakistani families HHS1 (FIGS. 5A and 5B) and HHS2 (FIGS. 5C-F). The age of each individual is 2 (FIG. 5A), 6 (FIG. 5B), 8 (FIG. 5C), 9 (FIG. 5D), 12 (FIG. 5E), and 20 (FIG. 5F) years old, respectively.

FIGS. 5G-I are photographs of plucked hair shafts of affected individuals. Scale bars: 100 μm. FIG. 5J is a photograph of the clinical appearance of an affected individual in the Pakistani family HHS1. The age of the individual is 28.

FIG. 6 represents haplotype analysis of the Pakistani family HHS2 for the mutation L9R in the APCDD1 gene (TOP). The linked haplotype is indicated in red, and critical recombination events are indicated by an arrowhead. The disease-related haplotype and affected individuals are colored in red. Screening assays with a restriction enzyme in HHS2 are shown below the pedigree as a gel image. PCR product from wild-type allele, 191 bp in size, was digested into 149 bp and 42 bp fragments, while that from the mutant allele was undigested. MWM, molecular weight markers; C, control individual.

FIGS. 7A-E shows an Italian family with HHS. FIG. 7A depicts a pedigree of an Italian family with HHS, while FIGS. 7B-E are photographic images of the clinical appearance of affected individuals. Scale bars: 100 μm

FIG. 7F is a schematic of the candidate region for the Italian family that was defined previously³. Candidate region that was defined in Pakistani families, as well as the position of APCDD1 gene, are also shown.

FIG. 7G is a DNA chromatogram showing the identification of a heterozygous 26T>G (L9R) mutation in the APCDD1 gene in the Italian family [SEQ ID NO: 9758, amino acid sequence disclosed as SEQ ID NO: 9773].

FIG. 8 is a comparison of haplotypes between three families with an identical point mutation in the APCDD1 gene. The marker APCDD1-MS is located within intron 1 of the APCDD1 gene, which is only 5 Kb distant from the position of the mutation. Note that the three families had a distinct disease-related haplotype, suggesting that the mutation arose independently in each family, and that nucleotide 26 of the SWAMP gene may be a mutational hotspot.

FIG. 9 is a multiple amino acid sequence alignment of APCDD1 protein between different species. N-terminal signal peptide and C-terminal transmembrane sequences are boxed in red and black, respectively. Conserved residues among at least 6 species are indicated by asterisks. The Leu9 is indicated in blue and a black circle. Highly conserved cysteine residues are indicated by black arrowheads and highlighted in yellow. The accession numbers of GenBank or Ensembl databases for the respective APCDD1 proteins are: Homo sapiens, NP_{—}694545 [SEQ ID NO. 9759]; Equus caballus, ENSECAP00000009668 [SEQ ID NO. 9760]; Canis familiaris, XP_{—}537333 [SEQ ID NO. 9761]; Mus musculus, NP_{—}573500 [SEQ ID NO. 9762]; Myotis lucifugus, ENSMLUP00000001735 [SEQ ID NO. 9763]; Gallus gallus, ENSGALP00000001313 [SEQ ID NO. 9764]; Pelodiscus sinensis, BAD74115 [SEQ ID NO. 9765]; Xenopus tropicalis, ENSXETP00000056413 [SEQ ID NO. 9766]; Danio rerio, ENSDARP00000081410 [SEQ ID NO. 9767]; Ciona intestinalis, ENSCINP00000022338 [SEQ ID NO. 9768].

FIG. 10 are graphs depicting the prediction of the signal peptide of APCDD1 protein. The N-terminal signal peptide sequences of the wild-type (FIG. 10A) and the L9R mutant (FIG. 10B) APCDD1 protein was analyzed using the SignalP-HMM program (version 3.0; www.cbs.dtu.dk/services/SignalP/). The predicted hydrophobic core sequences are boxed in FIG. 10A [SEQ ID NO. 9769] and FIG. 10B [SEQ ID NO. 9770]. The amino acid poison 9 is indicated by red arrowheads.

FIG. 11 are images of western blots carried out to analyze APCDD1 protein. FIG. 11A depicts wild-type APCDD1 protein that was digested with PNGase F. FIG. 11B represents an immunoprecipitation experiment. Total cell lysates were immunoprecipitated with anti-c-myc antibody, which was followed by western blot with anti-HA antibody. 55 KDa fragment corresponds to the heavy chain of IgG.

FIG. 12 are images of western blots carried out to analyze APCDD1 expression in three different cell lines. Expression of APCDD1 protein in cell lysates from HEK293T (Left Panel), CHO (Center Panel), and primary human dermal fibroblast (Right Panel) was analyzed by western blots with anti-HA antibody. When the L9R mutant APCDD1 expression construct was co-transfected with the wild-type construct, the expression of the wild-type protein was markedly decreased in HEK293T cells.

FIG. 13. is a bar graph depicting that APCDD1 expression significantly decreases in cultured dermal papilla (DP) cells. The expression levels of APCDD1-mRNA between fresh and cultured (passages 0, 1, 3 and 5) DP cells were analyzed by real-time PCR. Relative RNA levels are shown as compared with the expression level in P5 cells.

FIG. 14. are images of western blots with a mouse polyclonal anti-APCDD1 antibody. In total cell lysates from human scalp skin, two fragments around 58 and 130 KDa in size, were detected, which is similar patterns with the HA-tagged wild-type APCDD1 overexpressed in HEK293T cells. The anti-APCDD1 antibody also showed a fragment in medium of wild-type APCDD1 construct-transfected cells (bottom panel).

FIG. 15 are photomicrographs of human hair follicles (HFs). FIG. 15A shows In situ hybridization with SWAMP (APCDD1) antisense mRNA probe in human HFs. SWAMP is present in the dermal papilla (DP), the matrix (Mx), the hair shaft cortex (HSCx), and the hair shaft cuticle (HSCu) of the human hair follicles, while the sense probe did not show any signal. FIGS. 15B-E are images of Indirect immunofluorescence in human HFs using a mouse polyclonal anti-APCDD1 antibody (Abnova). The expression of SWAMP protein in the HSCx (boxed with dotted line in FIG. 15B overlaps with that of E- and P-cadherin proteins (FIGS. 15C-E). Counterstaining with DAPI is shown in blue (FIGS. 15B, 15E). Scale bars: 100 μm (FIGS. 15A-B), 20 μm (FIG. 15C).

FIG. 16 is a schematic of the mechanism of action of wild-type and L9R mutant SWAMP (APCDD1). Wild type (Wt) SWAMP is processed in the ER and localized at the cell membrane, which inhibits Wnt signaling through interacting with WNT and LRP proteins (FIG. 16A). By contrast, when Wt-SWAMP co-expresses with L9R-SWAMP, Wt-SWAMP is forced to be retained and degraded in the ER, which is predicted to result in upregulation of Wnt signaling (FIG. 16B).

FIG. 17 is photographic images of RNA blots showing that SWAMP (APCDD1) mRNA is expressed in human scalp skin. FIG. 17A shows RT-PCR amplification of SWAMP mRNA from human scalp skin. Note that SWAMP-mRNA was amplified, while its homologue APCDD1L-mRNA was not. FIG. 17B shows RT-PCR using total RNA from human plucked hairs shows the expression of LRP5 and WNT3A in human hair follicles. MWM, molecular weight markers (FIGS. 17A, 17B).

FIG. 18 is photographic images of western blots showing that SWAMP (APCDD1) is binds with LRP5 and WNT3A in vitro. FIG. 18A demonstrates co-immunoprecipitation assays in HEK293T cells. HA-tagged extracellular domain of SWAMP protein (SWAMP-ΔTM-HA) was co-immunoprecipitated with the Flag-tagged extracellular domain of LRP5 (LRP5-EC-Flag; left panel). Flag-tagged extracellular domain of SWAMP protein (SWAMP-ΔTM-Flag) was co-immunoprecipitated with the HA-tagged WNT3A (WNT3A-HA), but not with the HA-tagged extracellular domain of CD40 (CD40-EC-HA; right panel). FIG. 18B depicts GST-pulldown assays. N-terminal GST fusion protein for extracellular domain of SWAMP (GST-SWAMP-ΔTM) was generated in bacteria, and was purified with glutathione-Sepharose beads (left panel). The purified GST-SWAMP-ΔTM was incubated with lysates of HEK293T cells overexpressing LRP5-EC-Flag, WNT3A-HA, or CD40-EC-HA, and was analyzed by western blots with mouse monoclonal anti-Flag-M2 (1:1,000; Sigma) or rabbit polyclonal anti-HA (1:4,000; Abcam) antibodies. The GST-SWAMP-ΔTM showed an affinity with LRP5-EC-Flag and WNT3A-HA, but not with CD40-EC-HA (right panels). CD40 is a Wnt signaling-unrelated single-pass transmembrane protein, and was used as a negative control (FIGS. 18A-18B).

FIGS. 19A-C are photographs of western blots demonstrating the characterization of the SWAMP (APCDD1) protein. FIG. 19A is a western blot of cell lysates from HA-tagged wild-type SWAMP-expressing HEK293T cells were treated with N-glycosidase (PNGase F). The 68 KDa fragment was clearly digested into a 53 KDa fragment with PNGase F, suggesting that the SWAMP protein undergoes N-glycosylation. FIG. 19B is a western blot of equal amounts of cell lysate from HA-tagged wild-type SWAMP-expressing HEK293T cells were separated by 10% SDS PAGE under either non-reducing (−) or reducing (+) conditions. The intensity of the 130 KDa fragment markedly increased under non-reducing conditions. FIG. 19C is a western blot of co-immunoprecipitation (Co-IP) assays between Flag-tagged SWAMP (SWAMP-Flag) and HA-tagged SWAMP (SWAMP-HA) proteins. SWAMP-Flag protein is co-immunoprecipitated with SWAMP-HA protein (left panel), and SWAMP-HA protein is co-immunoprecipitated with SWAMP-Flag protein (right panel). These results demonstrate homodimerization of the SWAMP protein.

FIG. 19D is a photograph of a western blot. HEK293T cells were transfected with a full-length SWAMP (APCDD1) expression construct containing a Flag-tag just downstream of the signal peptide and an HA-tag at the C-terminus, and analyzed cell lysates and supernatants by western blotting. An expression construct for a truncated SWAMP lacking the trans-membrane domain (SWAMP-ΔTM) was also transfected as a positive control. S, signal peptide. TM, transmembrane domain. Western blots with anti-Flag, anti-SWAMP and anti-Flag antibodies detected a strong fragment, around 63 KDa in size, in the medium of SWAMP-ΔTM-expressing cells, while no fragments were detected in medium of full-length SWAMP-expressing cells. beta-actin was used as a normalization control, and also used to show that the cell lysate did not contaminate the medium.

FIGS. 20A-H are photomicrographs demonstrating that the mutation L9R affects the co-translational processing of the mutant SWAMP (APCDD1). FIGS. 20A-H show immunofluorescence for SWAMP on HEK293T cells (FIGS. 20A, 20B) or Bend3.0 cells (FIGS. 20C-H) transfected with Wt SWAMP (FIGS. 20A, 20C, 20F), L9R mutant SWAMP (FIGS. 20B, 20D, 20G), or L9V mutant SWAMP (FIGS. 20E, 20H). Cell membrane was labeled with an anti-pan-cadherin antibody (FIGS. 20A, 20B). Scale bar: 20 μm (FIG. 20A). Bend3.0 cells were either not permeabilized with TritonX-100 (FIGS. 20C-E) to determine surface expression of SWAMP or permeabilized (FIGS. 20E-H) to detect total protein. Note that WT or L9V SWAMP isoforms localize to the plasma membrane (FIGS. 20A, 20C, 20F, 20E, 20H), whereas the L9R SWAMP is not present in the membrane, but is retained in the secretory pathway FIGS. 20B, 20D, 20G). The bottom panels are merged images and counterstaining with DAPI is shown in blue (FIGS. 20A, 20B).

FIGS. 20I-K are photographs of a western blot and microscopy images. N-terminal GFP-tagged SWAMP proteins (GST-SWAMP) were overexpressed in HEK293T cells, which were analyzed by western blot (FIG. 20I) and immunocytostainings (FIGS. 20J, 20K) with the rabbit polyclonal anti-SWAMP antibody. The western blot clearly showed that the signal peptide sequence of wild type SWAMP (GFP-Wt) was cleaved, while that of the L9R mutant (GFP-L9R) was not (FIG. 20I). beta-actin was used as a normalization control (FIG. 20I) GFP-Wt-SWAMP protein is detected at the cell membrane (FIG. 20J), while the GFP-L9R-SWAMP is retained within the cytoplasm (FIG. 20K). The bottom panels are merged images and counterstaining with DAPI is shown in blue (FIGS. 20J, 20K).

### DETAILED DESCRIPTION OF THE INVENTION

The invention provides for a new therapeutic target, namely APCDD1, for modulation of hair color (pigmentation) and hair growth/density. Therapies utilizing this gene target are provided to treat loss of hair pigment (“graying”), loss of hair density, as well as too much hair. In one embodiment, APCDD1 can be used to treat hair loss disorders, such as androgenetic alopecia.

Hair follicle (HF) miniaturization, seen in androgenetic alopecia (AGA) or male/female pattern baldness, is a degenerative process that occurs in humans, and causes a reduction in the epithelial and mesenchymal compartments of the HF^{A4}. Despite molecular characterization of several genes that are differentially expressed among various HF cell populations, family-based linkage approaches in AGA^{A5}, or recent genome-wide association studies^{A6, A7} have failed to elucidate the genetic architecture of this polygenic disorder.

In one embodiment, to gain insight into the genetic underpinning of HF miniaturization in humans, the causative gene for the autosomal dominant hereditary hypotrichosis simplex (HHS; OMIM 146520)^{A2} was identified. The disease is characterized histologically by HF miniaturization^{A1} and a progressive hair loss that begins in early childhood, continues throughout life, and is independent of hormonal effects. Without being bound by theory, HHS is likely caused by mutations in a single autosomal gene given the simple Mendelian inheritance pattern, the rarity of affected families, and the absence of gender predilection (for example, see infra Example 2).

Overview of the Integument and Hair Cells

The integument (or skin) is the largest organ of the body and is a highly complex organ covering the external surface of the body. It merges, at various body openings, with the mucous membranes of the alimentary and other canals. The integument performs a number of essential functions such as maintaining a constant internal environment via regulating body temperature and water loss; excretion by the sweat glands; but predominantly acts as a protective barrier against the action of physical, chemical and biologic agents on deeper tissues. Skin is elastic and except for a few areas such as the soles, palms, and ears, it is loosely attached to the underlying tissue. It also varies in thickness from 0.5 mm (0.02 inches) on the eyelids (“thin skin”) to 4 mm (0.17 inches) or more on the palms and soles (“thick skin”) (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The skin is composed of two layers: a) the epidermis and b) the dermis. The epidermis is the outer layer, which is comparatively thin (0.1 mm). It is several cells thick and is composed of 5 layers: the stratum germinativum, stratum spinosum, stratum granulosum, stratum lucidum (which is limited to thick skin), and the stratum corneum. The outermost epidermal layer (the stratum corneum) consists of dead cells that are constantly shed from the surface and replaced from below by a single, basal layer of cells, called the stratum germinativum. The epidermis is composed predominantly of keratinocytes, which make up over 95% of the cell population. Keratinocytes of the basal layer (stratum germinativum) are constantly dividing, and daughter cells subsequently move upwards and outwards, where they undergo a period of differentiation, and are eventually sloughed off from the surface. The remaining cell population of the epidermis includes dendritic cells such as Langerhans cells and melanocytes. The epidermis is essentially cellular and non-vascular, containing little extracellular matrix except for the layer of collagen and other proteins beneath the basal layer of keratinocytes (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The dermis is the inner layer of the skin and is composed of a network of collagenous extracellular material, blood vessels, nerves, and elastic fibers. Within the dermis are hair follicles with their associated sebaceous glands (collectively known as the pilosebaceous unit) and sweat glands. The interface between the epidermis and the dermis is extremely irregular and uneven, except in thin skin. Beneath the basal epidermal cells along the epidermal-dermal interface, the specialized extracellular matrix is organized into a distinct structure called the basement membrane (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The mammalian hair fiber is composed of keratinized cells and develops from the hair follicle. The hair follicle is a peg of tissue derived from a downgrowth of the epidermis, which lies immediately underneath the skin's surface. The distal part of the hair follicle is in direct continuation with the external, cutaneous epidermis. Although a small structure, the hair follicle comprises a highly organized system of recognizably different layers arranged in concentric series. Active hair follicles extend down through the dermis, the hypodermis (which is a loose layer of connective tissue), and into the fat or adipose layer (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

At the base of an active hair follicle lies the hair bulb. The bulb consists of a body of dermal cells, known as the dermal papilla, contained in an inverted cup of epidermal cells known as the epidermal matrix. Irrespective of follicle type, the germinative epidermal cells at the very base of this epidermal matrix produce the hair fiber, together with several supportive epidermal layers. The lowermost dermal sheath is contiguous with the papilla basal stalk, from where the sheath curves externally around all of the hair matrix epidermal layers as a thin covering of tissue. The lowermost portion of the dermal sheath then continues as a sleeve or tube for the length of the follicle (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

Developing skin appendages, such as hair and feather follicles, rely on the interaction between the epidermis and the dermis, the two layers of the skin. In embryonic development, a sequential exchange of information between these two layers supports a complex series of morphogenetic processes, which results in the formation of adult follicle structures. However, in contrast to general skin dermal and epidermal cells, certain hair follicle cell populations, following maturity, retain their embryonic-type interactive, inductive, and biosynthetic behaviors. These properties can be derived from the very dynamic nature of the cyclical productive follicle, wherein repeated tissue remodeling necessitates a high level of dermal-epidermal interactive communication, which is vital for embryonic development and would be desirable in other forms of tissue reconstruction.

The hair fiber is produced at the base of an active follicle at a very rapid rate. For example, follicles produce hair fibers at a rate 0.4 mm per day in the human scalp and up to 1.5 mm per day in the rat vibrissa or whiskers, which means that cell proliferation in the follicle epidermis ranks amongst the fastest in adult tissues (Malkinson F D and J T Kearn, Int J Dermatol 1978, 17:536-551). Hair grows in cycles. The anagen phase is the growth phase, wherein up to 90% of the hair follicles said to be in anagen; catagen is the involuting or regressing phase which accounts for about 1-2% of the hair follicles; and telogen is the resting or quiescent phase of the cycle, which accounts for about 10-14% of the hair follicles. The cycle's length varies on different parts of the body.

Hair follicle formation and cycling is controlled by a balance of inhibitory and stimulatory signals. The signaling cues are potentiated by growth factors that are members of the TGFβ-BMP family. A prominent antagonist of the members of the TGFβ-BMP family is follistatin. Follistatin is a secreted protein that inhibits the action of various BMPs (such as BMP-2, -4, -7, and -11) and activins by binding to said proteins, and purportedly plays a role in the development of the hair follicle (Nakamura M, et al., FASEB J, 2003, 17(3):497-9; Patel K Intl J Biochem Cell Bio, 1998, 30:1087-93; Ueno N, et al., PNAS, 1987, 84:8282-86; Nakamura T, et al., Nature, 1990, 247:836-8; Iemura S, et al., PNAS, 1998, 77:649-52; Fainsod A, et al., Mech Dev, 1997, 63:39-50; Gamer L W, et al., Dev Biol, 1999, 208:222-32).

The deeply embedded end bulb, where local dermal-epidermal interactions drive active fiber growth, is the signaling center of the hair follicle comprising a cluster of mesenchymal cells, called the dermal papilla (DP). This same region is also central to the tissue remodeling and developmental changes involved in the hair fiber's or appendage's precise alternation between growth and regression phases. The DP, a key player in these activities, appears to orchestrate the complex program of differentiation that characterizes hair fiber formation from the primitive germinative epidermal cell source (Oliver R F, J Soc Cosmet Chem, 1971, 22:741-755; Oliver R F and C A Jahoda, Biology of Wool and Hair (eds Roger et al.), 1971, Cambridge University Press:51-67; Reynolds A J and C A Jahoda, Development, 1992, 115:587-593; Reynolds A J, et al., J Invest Dermatol, 1993, 101:634-38).

The lowermost dermal sheath (DS) arises below the basal stalk of the papilla, from where it curves outwards and upwards. This dermal sheath then externally encases the layers of the epidermal hair matrix as a thin layer of tissue and continues upward for the length of the follicle. The epidermally-derived outer root sheath (ORS) also continues for the length of the follicle, which lies immediately internal to the dermal sheath in between the two layers, and forms a specialized basement membrane termed the glassy membrane. The outer root sheath constitutes little more than an epidermal monolayer in the lower follicle, but becomes increasingly thickened as it approaches the surface. The inner root sheath (IRS) forms a mold for the developing hair shaft. It comprises three parts: the Henley layer, the Huxley layer, and the cuticle, with the cuticle being the innermost portion that touches the hair shaft. The IRS cuticle layer is a single cell thick and is located adjacent to the hair fiber. It closely interdigitates with the hair fiber cuticle layer. The Huxley layer can comprise up to four cell layers. The IRS Henley layer is the single cell layer that runs adjacent to the ORS layer (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

Wnt Signaling

Wnt proteins are secreted from cells, however rarely as a soluble form (Papkoff J and B Schryver, Mol Cell Biol, 1990, 10:2723-30; Burrus L W and McMahon A P, Exp Cell Res, 1995, 220:363-73; Willert K, et al., Nature, 2003 423:448-52). Wnt proteins are glycosylated (Mason J O, et al., Mol Biol Cell, 1992, 3:521-33) and palmitoylated (Willert K, et al., Nature, 2003 423:448-52). In the Wnt signaling pathway, Wnt binds to Frizzled (Frz), a cell surface receptor that is found on various cell types. In the presence of Dishevelled (Dsh), binding of Wnt to the Frz receptor purportedly results in inhibiting GSK3β mediated phosphorylation. Inhibition of this phosphorylation event allegedly would then subsequently halt phosphorylation-dependent degradation of β-catenin. Thus, Wnt binding stabilizes cellular β-catenin. β-catenin can then accumulate in the cytoplasm in the presence of Wnt binding and can subsequently bind to a transcription factor, such as Lef1. The β-catenin-Lef1 complex is then able to translocate to the nucleus, where the β-catenin-Lef1 complex can mediate transcriptional activation. Other effects and components of the Wnt signaling pathway are described in the following: Arias A M, et al., Curr Opin Genet Dev, 1999, 9: 447-454; Nusse R, Development, 2003, 130(22):5297-305; Nelson W J and R Nusse, Science, 2004, 303:1483-7; Logan C Y and R Nusse, Annu Rev Cell Dev Biol, 2004, 20:781-810; Moon R T, et al., Nat Rev Genet, 2004, 5(9):691-701; Brennan K R and A M Brown, J Mammary Gland Biol Neoplasia, 2004, 9(2):119-31; Johnson M L, et al., Bone Miner Res, 2004, 19(11):1749-57; Nusse R, Nature, 2005, 438:747-9; Reya T and H Clevers Nature, 2005, 434:843-50; Gregorieff A and H Clevers, Genes Dev, 2005, 19(8):877-90; Bejsovec A, Cell, 2005, 120(1):11-4; Brembeck F H, et al., Curr Opin Genet Dev, 2006, 16(1):51-9; and Attisano et al., (2004) Cancer and Metastasis Reviews 23: 53-61, which are all herein incorporated by reference in their entireties. In one embodiment, APCDD1 is an inhibitor of the Wnt signaling pathway.

Hereditary Hypotrichosis Simplex (HHS)

The hair follicle (HF) is a complex organ which periodically regenerates in the form of a hair cycle. Recent advances in molecular genetics have enabled the identification of numerous genes that are expressed in the HF^{S1}. Disruption of some of these genes underlies different types of hereditary hypotrichosis (HH). HH can be largely divided into syndromic and non-syndromic forms. In syndromic forms of HH, hypotrichosis appears as a part of the disease. For example, mutations in P-cadherin gene (CDH3) are known to cause not only hypotrichosis, but also weak eyesight due to macular dystrophy of the retina (Hypotrichosis with Juvenile Macular Dystrophy; HJMD; OMIM 601553)^{S2, S3}. Some cases with CDH3 mutations also show severe digit anomalies (Ectodermal dysplasia, Ectrodactyly, and Macular dystrophy; EEM syndrome; OMIM 225280)^{S3, S4}. In non-syndromic forms of HH, hypotrichosis is the only finding detected in affected individuals. Of these, Marie Unna hypotrichosis (OMIM 146550) is an autosomal dominant disorder characterized by coarse, wiry and twisted hair shaft, and was recently reported to be caused by mutations in the 5′-regularly region of the hairless gene (HR) on chromosome 8p21^{S5}. In addition, monilethrix is characterized by a specific hair shaft anomaly known as a moniliform hair. This disease can show either an autosomal dominant (OMIM 158000) or recessive (OMIM 252200) inheritance trait, and several causative genes have been identified to date^{S6-S11}.

A rare form of hereditary hypotrichosis without any characteristic hair shaft anomalies is known as hereditary hypotrichosis simplex (HHS; OMIM 146520/605389)^{S12, S13}. Affected individuals with HHS typically show normal hair at birth, but hair loss and thinning of the hair shaft on the scalp start during early childhood and progress with age, frequently affecting the body hairs as well. Histologically, HHS is characterized by progressive HF miniaturization, which is a typical feature of androgenetic alopecia^{S12, S14}. HHS is known to be inherited as either an autosomal dominant (ADHHS)12-16 or autosomal recessive (ARHHS)17 trait. Mutations in corneodesmosin (CDSN) gene were identified in several families with ADHHS. In addition, an Italian family was previously analyzed with ADHHS and found linkage of the family to a 9.8 Mb interval on chromosome 18p11.32-p11.23^{S16}, in which the APCDD1/SWAMP gene resides (See infra at Examples). Most recently, mutations in the P2RY5 gene were reported to underlie ARHHS17 or autosomal recessive woolly hair^{S18}.

APCDD1

Human APCDD1 (adenomatosis polyposis coli down-regulated 1; also referred to as SWAMP in Example 2) is a gene assigned at chromosomal band 18p11.2, and is also referred to as B7323, DRAPC1, or FP7019. Various hair disorders, such as hypotrichosis, have been linked to genes located on chromosome 18 (for example, see Baumer et al., (2000) Eur J of Hum Genet 8: 443-8). APCDD1 is a direct target of the WNT/β-catenin signaling pathway and is regulated by the β-catenin/Tcf complex (Takahashi et al., (2002) Cancer Research, 62: 5651-56). It has been identified to be over-expressed in certain cancers, such as colon cancer and in CTNNB-1 mutated Wilms tumors (Takahashi et al., (2002) Cancer Research, 62: 5651-56; Zirn et al., (2006) Genes, Chrom, and Cancer 45: 565-74, each of which are incorporated by reference in their entireties). In one embodiment, APCDD1 is an inhibitor of the Wnt signaling pathway.

The mouse gene, Drapc1, is the ortholog of human APCDD1 and has been shown to be a target of Wnt/β-catenin signaling pathway in cancer cell lines (Jukkola et al., (2004) Gene Expression Patterns 4: 755-62). Sequence analysis of the mouse Drapc1 predicted a transcript of 1545 nucleotides that encodes a putative transmembrane (TM) protein of 514 amino acids having a molecular weight of about 58.6 kDa (Jukkola et al., (2004) Gene Expression Patterns 4: 755-62). Alignment of the putative amino acid sequences of mouse and human DRAPC1 revealed a 95% sequence similarity and DRAPC1 was found to be conserved in many vertebrate species. Jukkola et al. ((2004) Gene Expression Patterns 4: 755-62) also identified a Drapc1 related gene, Drapc2 (APCDD1-L (like)), in the human genome, having 67% similarity to Drapc1. Drapc1 was expressed in the hair follicles of the skin and was found expressed in the dermal papillae and matrix cells, which are inner root sheath (IRS) precursor cells (Jukkola et al., (2004) Gene Expression Patterns 4: 755-62).

As used herein, an “APCDD1 molecule” refers to an APCDD1 protein that includes a polypeptide that exhibits transmembrane topology. For example, an APCDD1 molecule can be the human APCDD1 protein (e.g., having the amino acid sequence shown in SEQ ID NO: 1). The APCDD1 molecule can be encoded by a nucleic acid (including, for example, genomic DNA, complementary DNA (cDNA), synthetic DNA, as well as any form of corresponding RNA). For example, an APCDD1 molecule can be encoded by a recombinant nucleic acid encoding human APCDD1 protein. The APCDD1 molecules of the invention can be obtained from various sources and can be produced according to various techniques known in the art. For example, a nucleic acid that encodes an APCDD1 molecule can be obtained by screening DNA libraries, or by amplification from a natural source. An APCDD1 molecule can include a fragment or portion of human APCDD1 protein that retains transmembrane topology. The APCDD1 molecules of the invention can be produced via recombinant DNA technology and such recombinant nucleic acids can be prepared by conventional techniques, including chemical synthesis, genetic engineering, enzymatic techniques, or a combination thereof. A non-limiting example of an APCDD1 molecule is the polypeptide encoded by the nucleic acid having the nucleotide sequence shown in SEQ ID NO: 2.

In another embodiment, an APCDD1 molecule can encompass orthologs of human APCDD1 protein. For example, an APCDD1 molecule can encompass the ortholog in mouse (such as DRAPC1), rat, non-human primates, canines, goat, rabbit, porcine, bovine, chickens, feline, and horses. An APCDD1 molecule can comprise a protein encoded by a nucleic acid sequence homologous to the human nucleic acid, wherein the nucleic acid is found in a different species and wherein that homolog encodes a protein similar to an APCDD1 protein.

An APCDD1 molecule can also encompass a variant of the human APCDD1 protein. Such a variant can comprise a naturally-occurring variant due to allelic variations between individuals (e.g., polymorphisms), mutated alleles related to hair growth, density, or pigmentation, or alternative splicing forms. In one embodiment, an APCDD1 molecule is encoded by a nucleic acid variant of the nucleic acid having the sequence shown in SEQ ID NO: 2, wherein the variant has a nucleotide sequence identity to SEQ ID NO:2 of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In another embodiment, a variant of the human APCDD1 protein comprises a protein or polypeptide encoded by an APCDD1 nucleic acid sequence, such as the sequence shown in SEQ ID NO: 5.

In one embodiment, an APCDD1 molecule comprises a protein or polypeptide encoded by an APCDD1 nucleic acid sequence, such as the sequence shown in SEQ ID NO: 1. In another embodiment, the polypeptide can be modified, such as by glycosylations and/or acetylations and/or chemical reaction or coupling, and can contain one or several non-natural or synthetic amino acids. An example of an APCDD1 molecule is the polypeptide having the amino acid sequence shown in SEQ ID NO: 1. In certain embodiments, the APCDD1 molecule of the invention includes variants of the human APCDD1 protein (having the amino acid sequence shown in SEQ ID NO: 1). Such variants can include those having at least from about 46% to about 50% identity to SEQ ID NO: 1, or having at least from about 50.1% to about 55% identity to SEQ ID NO: 1, or having at least from about 55.1% to about 60% identity to SEQ ID NO: 1, or having from at least about 60.1% to about 65% identity to SEQ ID NO: 1, or having from about 65.1% to about 70% identity to SEQ ID NO: 1, or having at least from about 70.1% to about 75% identity to SEQ ID NO: 1, or having at least from about 75.1% to about 80% identity to SEQ ID NO: 1, or having at least from about 80.1% to about 85% identity to SEQ ID NO: 1, or having at least from about 85.1% to about 90% identity to SEQ ID NO: 1, or having at least from about 90.1% to about 95% identity to SEQ ID NO: 1, or having at least from about 95.1% to about 97% identity to SEQ ID NO: 1, or having at least from about 97.1% to about 99% identity to SEQ ID NO: 1. In one embodiment, an APCDD1 molecule comprises a protein or polypeptide encoded by an APCDD1 nucleic acid sequence, such as the sequence shown in SEQ ID NO: 5.

The human APCDD1 polypeptide has been reported to include a putative 514 amino acid protein, while the APCDD1 cDNA comprises 2607 nucleotides that contain an open reading frame of 1542 nucleotides as set forth in SEQ ID NO: 2 (see U.S. Patent Application Publication No. 2006/0019252, which is incorporated by reference in its entirety). The open reading frame, which encodes the putative 514-amino acid protein, contains no known motif. Furthermore, APCDD1 expression is enhanced by the β-catenin/Tcf 4 complex through the binding of the complex to the two Tcf/LEF binding motifs in the transcriptional regulatory region of APCDD1 (Takahashi et al., (2002) Cancer Research, 62: 5651-56).

The polypeptide sequence of human APCDD1 is depicted in SEQ ID NO: 1. The nucleotide sequence of human APCDD1 is shown in SEQ ID NO: 2. Sequence information related to APCDD1 is accessible in public databases by GenBank Accession numbers NM_{—}153000 (for mRNA) and NP_{—}694545 (for protein).

SEQ ID NO: 1 is the human wild type amino acid sequence corresponding to APCDD1(residues 1-514):

MSWPRRLLLRYLFPALLLHGLGEGSALLHPDSRSHPRSLEKSAWRAFK
ESQCHHMLKHLHNGARITVQMPPTIEGHWVSTGCEVRSGPEFITRSYRF
YHNNTFKAYQFYYGSNRCTNPTYTLIIRGKIRLRQASWIIRGGTEADYQ
LHNVQVICHTEAVAEKLGQQVNRTCPGFLADGGPWVQDVAYDLWREEN
GCECTKAVNFAMHELQLIRVEKQYLHHNLDHLVEELFLGDIHTDATQRM
FYRPSSYQPPLQNAKNHDHACIACRIIYRSDEHHPPILPPKADLTIGL
HGEWVSQRCEVRPEVLFLTRHFIFHDNNNTWEGHYYHYSDPVCKHPTFS
IYARGRYSRGVLSSRVMGGTEFVFKVNHMKVTPMDAATASLLNVFNGNE
CGAEGSWQVGIQQDVTHTNGCVALGIKLPHTEYEIFKMEQDARGRYLLF
NGQRPSDGSSPDRPEKRATSYQMPLVQCASSSPRAEDLAEDSGSSLYGR
APGRHTWSLLLAALACLVPLLHWNIRR

The underlined amino acid sequence above in SEQ ID NO: 1 refers to a predicted transmembrane domain (TMD) region of the APCDD1 polypeptide molecule. TMD I of the human APCDD1 comprises amino acid residues from about position 493 to about position 512 of SEQ ID NO: 1.

SEQ ID NO: 2 is the human wild type nucleotide sequence corresponding to APCDD1 (nucleotides 1-2579), wherein the underscored ATG denotes the beginning of the open reading frame:

gaaatatgaa gagacgctgc agctgcggtg gcggtggcgg ccactgcagc tcagagcggc
gcacgcggcg gccggggcgg gacgcggggc cgggcgcgga gaagtcgggg cgggcggcag
agaggccggg acgcggaccg ggccggggcg cccacagccg cccgacggcg cccagagagc
gcgcgccccg cagccccgcg cctagcccgc cgggcatggg gcgcgcggca gccgcctgaa
gccccggcct ggcccggccg cacccggccg gaggcggagg gcagagcgcg cgcccagttg
cccgggcacc aaatcggagc gcggcgtgcg ggaggcccca gagcaggact ggaaatgtcc
tggccgcgcc gcctcctgct cagatacctg ttcccggccc tcctgcttca cgggctggga
gagggttctg ccctccttca tccagacagc aggtctcatc ctaggtcctt agagaaaagt
gcctggaggg cttttaagga gtcacagtgc catcacatgc tcaaacatct ccacaatggt
gcaaggatca cagtgcagat gccacctaca atcgagggcc actgggtctc cacaggctgt
gaagtaaggt caggcccaga gttcatcaca aggtcctaca gattctacca caataacacc
ttcaaggcct accaatttta ttatggcagc aaccggtgca caaatcccac ttatactctc
atcatccggg gcaagatccg cctccgccag gcctcctgga tcatccgagg gggcacggaa
gccgactacc agctgcacaa cgtccaggtg atctgccaca cagaggcggt ggccgagaag
ctcggccagc aggtgaaccg cacatgcccg ggcttcctcg cagacggggg tccctgggtg
caggacgtgg cctatgacct ctggcgagag gagaacggct gtgagtgcac caaggccgtg
aactttgcca tgcatgaact tcagctcatc cgggtggaga agcagtacct tcaccacaac
ctcgaccacc tggtcgagga gctcttcctt ggtgacattc acactgatgc cacccagagg
atgttctacc ggccctccag ttaccagccc cctctgcaga atgccaagaa ccacgaccat
gcctgcatcg cctgtcggat catctatcgg tcagacgagc accaccctcc catcctgccc
ccaaaggcag acctgaccat cggcctgcac ggggagtggg tgagccagcg ctgtgaggtg
cgccccgaag tcctcttcct cacccgccac ttcatcttcc atgacaacaa caacacctgg
gagggccact actaccacta ctcagacccg gtgtgcaagc accccacctt ctccatctac
gcccggggcc gctacagccg cggcgtcctc tcgtccaggg tcatgggagg caccgagttc
gtgttcaaag tgaatcacat gaaggtcacc cccatggatg cggccacagc ctcactgctc
aacgtcttca acgggaatga gtgcggggcc gagggctcct ggcaggtggg catccagcag
gatgtgaccc acaccaatgg ctgcgtggcc ctgggcatca aactacctca cacggagtac
gagatcttca aaatggaaca ggatgcccgg gggcgctatc tgctgttcaa cggtcagagg
cccagcgacg ggtccagccc agacaggcca gagaagagag ccacgtccta ccagatgccc
ttggtccagt gtgcctcctc ttcgccgagg gcagaggacc tcgcagaaga cagtggaagc
agcctgtatg gccgggcccc tgggaggcac acctggtccc tgctgctggc tgcacttgcc
tgccttgtcc ctctgctgca ttggaacatc cgcagataga agttttagaa agttctattt
ttccaaacca ggattcctta ctattgacag atttgcttta ccaaaagaaa agacatttat
tcttttgatg cacttgaatg ccagagaact gtccttcttt ttctcctctc cctccctccc
agcccctgag tcatgaacag caaggagtgt ttgaagtttc tgctttgaac tccgtccagc
ctgatccctg gcctgagcaa cttcacaaca gtaattgcac tttaagacag cctagagttc
tggacgagcg tgtttggtag cagggatgaa agctagggcc tcttattttt ttctcttaat
tattattata tttctgagtt aaacttagaa gaaacaacta tcaagctaca acttttcctg
ccattttcct gtggttgcag cctgtcttcc tttgaaattg ttttactctc tgagttttat
atgctggaat ccaatgcaga gttggtttgg gactgtgatc aagacacctt ttattaataa
agaagagaca caggtgtaga tatgtatata caaaaagatg tacggtctgg ccaaaccacc
ttcccagcct ttatgcaaaa aaaggggaga atcaaagctt tcatttcaga aatgttgcgt
ggaaaagtat ctgtaattaa agtttcgaag taatttaacc taaaaaaaaa aaaaaaaaa

The mouse polypeptide sequence of APCDD1 is depicted in SEQ ID NO: 3. The mouse nucleotide sequence of APCDD1 is shown in SEQ ID NO: 4. (accessible in public databases by GenBank accession number NM_{—}133237).

SEQ ID NO: 3 is the mouse wild type amino acid sequence corresponding to APCDD1 (residues 1-514):

MSRVRRLLLGYLFPALLLHGLGEGSALLHPDSRSHPRSLEKSAWRAFK
ESQCHHMLKHLHNGARITVQMPPTIEGHWVSTGCEVRSGPEFMTRSYR
FYNNNTFKAYQFYYGSNRCTNPTYTLIIRGKIRLRQASWIIRGGTEADY
QLHGVQVICHTEAVAEQLSRLVNRTCPGFLAPGGPWVQDVAYDLWQEES
NHECTKAVNFAMHELQLIRVEKQYPHHSLDHLVEELFLGDIHTDATQRV
FYRPSSYQPPLQNAKNHNHACIACRIIFRSDEHHPPILPPKADLTIGLH
GEWVSQRCEVRPEVLFLTRHFIFHDNNNTWEGHYYHYSDPVCKHPTFT
IYARGRYSRGVLSSKVMGGTEFVFKVNHMKVTPMDAATASLLNVFSGNE
CGAEGSWQVGIQQDVTHTNGCVALGIKLPHTEYEIFKMEQDTRGRYLLF
NGQRPSDGSSPDRPEKRATSYQMPLVQCASSSPRAEELLEDSQGHLYG
RAAGRTAGSLLLPAFVSLWTLPHWRILR

SEQ ID NO: 4 is the mouse wild type nucleotide sequence corresponding to APCDD1 (nucleotides 1-2799), wherein the underscored ATG denotes the beginning of the open reading frame:

agcggccact gtacctctga gctgtgcacg ccgcggccgg ggcgggcctc gggactgggg
ctgggagcca aggggccggg gcgggacgcg gagaggctgg gctgcggttc ggagtcccgc
gcggacaggg gccggacggc ggcgagggag cgcgcgcccc gcagtcccgc gctgcgccgg
ccggggatgg ggcgcgctgc tgcctgaggc ccggcctggc gggcgcccgc cgggggctgc
ggctgaggag ccgagggcgc cctgtaccgg agtggcccgc gcgcgcgctc ggagggggac
agagacggac tacagcgagg cccggaggag ccccgagatg tcccgtgtgc gccgccttct
gcttggatac ctgttcccag ccctcctgtt gcatgggctg ggagagggct ctgccctcct
tcatccagac agcagatcgc accctcggtc cttagagaaa agcgcctgga gggctttcaa
ggagtcacag tgtcatcaca tgctgaagca tctccacaac ggtgcgcgga tcacagtgca
gatgcccccg accatcgagg gccactgggt gtccacaggc tgtgaagtaa ggtcgggtcc
ggagttcatg acaaggtctt acaggttcta caacaataat accttcaagg cctaccagtt
ttactatggc agcaaccgct gtacaaaccc cacctacacc ctcatcatcc gaggcaagat
ccggcttcgc caggcgtcct ggatcatccg tgggggcacc gaagctgact accagcttca
cggcgtccaa gtcatctgcc acacagaggc agtcgctgaa cagctcagcc gactggtgaa
ccgaacttgc ccaggcttcc tggctcctgg tggtccctgg gtacaggacg tagcctatga
cctgtggcag gaggagagta accacgagtg caccaaggct gtgaactttg ccatgcacga
gctgcagctc atccgtgtgg agaagcagta tccccaccac agcctggacc acctggtgga
ggagctcttc ctgggcgaca tccacacgga cgctacccag agggtgttct accggccgtc
cagttaccag ccgcccctgc agaatgccaa gaaccacaac catgcgtgca tagcctgccg
catcattttc cggtcagatg aacaccaccc tcccatactg ccccccaagg ctgacctgac
cattggcctc cacggggaat gggtgagcca gcgctgcgag gtacgccccg aggtcctctt
cctcacccgc cacttcatct tccacgacaa caacaacacc tgggaagggc attactacca
ctactcagac cctgtctgca agcaccccac attcaccatc tacgctcgag gccgctacag
ccgcggtgtg ctctcatcta aggtcatggg tggcacggag tttgtgttca aagtgaatca
catgaaggtt actcccatgg acgcagccac agcctccctc ctcaatgtct tcagtgggaa
tgagtgtggg gctgagggct cctggcaggt gggtatccaa caggatgtga cacataccaa
tggctgcgtg gctctgggca tcaaactacc tcacacagaa tatgagatct tcaaaatgga
gcaagacacc cgaggccgct acctgctgtt caatggccag aggcccagcg atggctccag
cccagacaga ccagagaaga gagccacatc ctaccagatg cccttggtcc agtgtgcctc
ttcctcacca agagctgaag agttgttgga agacagtcaa ggtcatctgt atggcagggc
agcagggagg acagctgggt ccctgttgct tcctgccttt gtcagccttt ggactctccc
acattggcgc atcctcagat agaagacatt tcctgaacca agactcttta ccgtgtatga
ctttccttca cacagagaaa ggacattgat tcttttgatg cacttgaatg ccttgagacc
tgccgttgcc tctcctctcc ccacctcttc cagcctctgc gccatgagca gtgtgtttga
agtttcagct ttgaatgtct tcccgcccga tccgtggcct gagaacttca tagaggtgtg
attgcacttt atgtctgcag agagctgggg cgagtgtgtt taggggtggc agctgccttc
ttctctctgt cccctgactc ctgactgtgt ttctgagctg agcttaaaag atacaaatga
caagctgcag ctctctctgc cattgcttct ctttatctct ccaaatccct tccacactcg
aggttttcga cactggaacc cagtgcagcg ttggcttggg accgccatga agaccccatg
tttgcgacag gagagcctgg gttggtgttg agtacataag agacgaggca aggttcagct
aagtctttgt cccagcctta atgctaacca acactttcag aaatgttgag tagagaggtg
tccctaaagc ttcaatggaa cttaaactct gttgacaagc gagtgccggt tttcacttgt
tgagaagaga tgtgtgccat atacttggtt tggtggctac agagtacagc ctgctgctta
accctcagag gagactgatc cagttgggaa attcagagca gctcctgctc caggcagcca
gaagcagcag tggggggtgg gggtggggat tccttgtgtt aagtgccaca caactgacaa
ggagatctgt ggagtttttc tccaagtgaa ccaaatccct gtgtcctggc tcacactgtg
gttagggtgg gcacatccac tctgccatct ttaacacac

Mutations that affect hair growth or density regulation and pigmentation have been localized to the following amino acid residues of APCDD1 described below.

For example, the invention provides for isolated mutants of the human APCDD1. In one embodiment, the APCDD1 molecule can comprise at least 1 amino acid mutation in SEQ ID NO: 1. In one embodiment, the mutation comprises an amino acid substitution in the signal sequence of the APCDD1 Protein. In another embodiment, the mutation comprises a Leu to Arg substitution at amino acid position 9 (for example, SEQ ID NO: 5).

In one embodiment the amino acid mutation in the human APCDD1 can comprise a Leu>Arg mutation at amino acid position 9 of SEQ ID NO: 1. This mutation can comprise the amino acid sequence of SEQ ID NO: 5.

SEQ ID NO: 5 is the human APCDD1 amino acid sequence (residue at amino acid position 1 to residue at amino acid position 514) having a Leu>Arg substitution mutation at amino acid position 9, which is depicted in BOLD and underlined:

MSWPRRLLRRYLFPALLLHGLGEGSALLHPDSRSHPRSLEKSAWRAFKE
SQCHHMLKHLHNGARITVQMPPTIEGHWVSTGCEVRSGPEFITRSYRFY
HNNTFKAYQFYYGSNRCTNPTYTLIIRGKIRLRQASWIIRGGTEADYQL
HNVQVICHTEAVAEKLGQQVNRTCPGFLADGGPWVQDVAYDLWREENGC
ECTKAVNFAMHELQLIRVEKQYLHHNLDHLVEELFLGDIHTDATQRMFY
RPSSYQPPLQNAKNHDHACIACRIIYRSDEHHPPILPPKADLTIGLHGE
WVSQRCEVRPEVLFLTRHFIFHDNNNTWEGHYYHYSDPVCKHPTFSIYA
RGRYSRGVLSSRVMGGTEFVFKVNHMKVTPMDAATASLLNVFNGNECG
AEGSWQVGIQQDVTHTNGCVALGIKLPHTEYEIFKMEQDARGRYLLFNG
QRPSDGSSPDRPEKRATSYQMPLVQCASSSPRAEDLAEDSGSSLYGRAP
GRHTWSLLLAALACLVPLLHWNIRR

The invention also provides for isolated mutants of the human APCDD1, wherein the isolated mutant human APCDD1 is encoded by a nucleic acid sequence comprising at least about 50%, at least about 60%, at least about 65%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identify with SEQ ID NO: 2.

For example, SEQ ID NO: 6 (below) is the human nucleotide sequence corresponding to APCDD1 (nucleotides 1-2579), wherein the underscored ATG denotes the beginning of the open reading frame (ORF), and a thymine (T) to guanine (G) missense mutation is denoted at position 26 from the beginning of the ORF (italicized in red):

gaaatatgaa gagacgctgc agctgcggtg gcggtggcgg ccactgcagc tcagagcggc
gcacgcggcg gccggggcgg gacgcggggc cgggcgcgga gaagtcgggg cgggcggcag
agaggccggg acgcggaccg ggccggggcg cccacagccg cccgacggcg cccagagagc
gcgcgccccg cagccccgcg cctagcccgc cgggcatggg gcgcgcggca gccgcctgaa
gccccggcct ggcccggccg cacccggccg gaggcggagg gcagagcgcg cgcccagttg
cccgggcacc aaatcggagc gcggcgtgcg ggaggcccca gagcaggact ggaaatgtcc
tggccgcgcc gcctcctgc [symbol] cagatacctg ttcccggccc tcctgcttca cgggctggga
gagggttctg ccctccttca tccagacagc aggtctcatc ctaggtcctt agagaaaagt
gcctggaggg cttttaagga gtcacagtgc catcacatgc tcaaacatct ccacaatggt
gcaaggatca cagtgcagat gccacctaca atcgagggcc actgggtctc cacaggctgt
gaagtaaggt caggcccaga gttcatcaca aggtcctaca gattctacca caataacacc
ttcaaggcct accaatttta ttatggcagc aaccggtgca caaatcccac ttatactctc
atcatccggg gcaagatccg cctccgccag gcctcctgga tcatccgagg gggcacggaa
gccgactacc agctgcacaa cgtccaggtg atctgccaca cagaggcggt ggccgagaag
ctcggccagc aggtgaaccg cacatgcccg ggcttcctcg cagacggggg tccctgggtg
caggacgtgg cctatgacct ctggcgagag gagaacggct gtgagtgcac caaggccgtg
aactttgcca tgcatgaact tcagctcatc cgggtggaga agcagtacct tcaccacaac
ctcgaccacc tggtcgagga gctcttcctt ggtgacattc acactgatgc cacccagagg
atgttctacc ggccctccag ttaccagccc cctctgcaga atgccaagaa ccacgaccat
gcctgcatcg cctgtcggat catctatcgg tcagacgagc accaccctcc catcctgccc
ccaaaggcag acctgaccat cggcctgcac ggggagtggg tgagccagcg ctgtgaggtg
cgccccgaag tcctcttcct cacccgccac ttcatcttcc atgacaacaa caacacctgg
gagggccact actaccacta ctcagacccg gtgtgcaagc accccacctt ctccatctac
gcccggggcc gctacagccg cggcgtcctc tcgtccaggg tcatgggagg caccgagttc
gtgttcaaag tgaatcacat gaaggtcacc cccatggatg cggccacagc ctcactgctc
aacgtcttca acgggaatga gtgcggggcc gagggctcct ggcaggtggg catccagcag
gatgtgaccc acaccaatgg ctgcgtggcc ctgggcatca aactacctca cacggagtac
gagatcttca aaatggaaca ggatgcccgg gggcgctatc tgctgttcaa cggtcagagg
cccagcgacg ggtccagccc agacaggcca gagaagagag ccacgtccta ccagatgccc
ttggtccagt gtgcctcctc ttcgccgagg gcagaggacc tcgcagaaga cagtggaagc
agcctgtatg gccgggcccc tgggaggcac acctggtccc tgctgctggc tgcacttgcc
tgccttgtcc ctctgctgca ttggaacatc cgcagataga agttttagaa agttctattt
ttccaaacca ggattcctta ctattgacag atttgcttta ccaaaagaaa agacatttat
tcttttgatg cacttgaatg ccagagaact gtccttcttt ttctcctctc cctccctccc
agcccctgag tcatgaacag caaggagtgt ttgaagtttc tgctttgaac tccgtccagc
ctgatccctg gcctgagcaa cttcacaaca gtaattgcac tttaagacag cctagagttc
tggacgagcg tgtttggtag cagggatgaa agctagggcc tcttattttt ttctcttaat
tattattata tttctgagtt aaacttagaa gaaacaacta tcaagctaca acttttcctg
ccattttcct gtggttgcag cctgtcttcc tttgaaattg ttttactctc tgagttttat
atgctggaat ccaatgcaga gttggtttgg gactgtgatc aagacacctt ttattaataa
agaagagaca caggtgtaga tatgtatata caaaaagatg tacggtctgg ccaaaccacc
ttcccagcct ttatgcaaaa aaaggggaga atcaaagctt tcatttcaga aatgttgcgt
ggaaaagtat ctgtaattaa agtttcgaag taatttaacc taaaaaaaaa aaaaaaaaa

Substitution, insertion, and deletion mutants of the APCDD1 nucleic acid sequence or amino acid sequence can be generated as discussed below.

DNA and Amino Acid Manipulation Methods and Purification Thereof

The present invention utilizes conventional molecular biology, microbiology, and recombinant DNA techniques available to one of ordinary skill in the art. Such techniques are well known to the skilled worker and are explained fully in the literature. See, e.g., Maniatis, Fritsch & Sambrook, “Molecular Cloning: A Laboratory Manual” (1982): “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986): B. Perbal, “A Practical Guide to Molecular Cloning” (1984), and Sambrook, et al., “Molecular Cloning: a Laboratory Manual” (1989).

One skilled in the art can obtain an APCDD1 protein or a variant thereof, in several ways, which include, but are not limited to, isolating the protein via biochemical means or expressing a nucleotide sequence encoding the protein of interest by genetic engineering methods.

The invention provides for a nucleic acid encoding an APCDD1 molecule or variants thereof. In one embodiment, the nucleic acid is expressed in an expression cassette, for example, to achieve overexpression in a cell. The nucleic acids of the invention can be an RNA, cDNA, cDNA-like, or a DNA of interest in an expressible format, such as an expression cassette, which can be expressed from the natural promoter or an entirely heterologous promoter. The nucleic acid of interest can encode a protein, and may or may not include introns.

Protein variants can include amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture.

Techniques for making substitution mutations at predetermined sites in DNA having a known sequence are well known, for example M13 primer mutagenesis and PCR mutagenesis. Amino acid substitutions can be single residues, but can occur at a number of different locations at once. In one non-limiting embodiment, insertions can be on the order of about from 1 to about 10 amino acid residues, while deletions can range from about 1 to about 30 residues. Deletions or insertions can be made in adjacent pairs (for example, a deletion of about 2 residues or insertion of about 2 residues). Substitutions, deletions, insertions, or any combination thereof can be combined to arrive at a final construct. The mutations cannot place the sequence out of reading frame and should not create complementary regions that can produce secondary mRNA structure. Substitutional variants are those in which at least one residue has been removed and a different residue inserted in its place.

In one embodiment, an isolated mutant human APCDD1 polypeptide can contain a Leu>Arg mutation at amino acid position 9 of SEQ ID NO: 1. The APCDD1 Leu>Arg mutant can comprise the amino acid sequence of SEQ ID NO: 5.

The invention also provides for isolated human APCDD1 polypeptides that contain an insertional or deletional mutations at the nucleic acid level. In one embodiment, an isolated mutant human APCDD1 polypeptide can be encoded by a nucleic acid sequence comprising at least about 50%, at least about 60%, at least about 70%, at least about 75%, at least about 80%, at least about 90%, at least about 95%, or at least about 99% identify to SEQ ID NO: 2. In another embodiment, the isolated human APCDD1 polypeptide is encoded by a nucleotide sequence that comprises the nucleic acid sequence of SEQ ID NO: 6.

Substantial changes in function or immunological identity are made by selecting selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions that can produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Minor variations in the amino acid sequences of APCDD1 molecules is provided by the present invention. The variations in the amino acid sequence can be when the sequence maintains at least about 30%, at least about 40%, at least about 50%, at least about 60%, at least about 70%, at least about 75%, at least about 80%, at least about 90%, at least about 95%, or at least about 99% identity to SEQ ID NO:1. For example, conservative amino acid replacements can be utilized. Conservative replacements are those that take place within a family of amino acids that are related in their side chains, wherein the interchangeability of residues have similar side chains.

Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) a group of amino acids having aliphatic-hydroxyl side chains, such as serine and threonine; (ii) a group of amino acids having amide-containing side chains, such as asparagine and glutamine; (iii) a group of amino acids having aliphatic side chains such as glycine, alanine, valine, leucine, and isoleucine; (iv) a group of amino acids having aromatic side chains, such as phenylalanine, tyrosine, and tryptophan; and (v) a group of amino acids having sulfur-containing side chains, such as cysteine and methionine. Useful conservative amino acids substitution groups are: valine-leucine-isoleucine, phenylalanine-tyrosine, lysine-arginine, alanine valine, glutamic-aspartic, and asparagine-glutamine.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gin; Ser, Thr; Lys, Arg; and Phe, Tyr. Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Bacterial and Yeast Expression Systems

In bacterial systems, a number of expression vectors can be selected. For example, when a large quantity of APCDD1 protein is needed for the induction of antibodies, vectors which direct high level expression of proteins that are readily purified can be used. Non-limiting examples of such vectors include multifunctional E. coli cloning and expression vectors such as BLUESCRIPT (Stratagene). pIN vectors or pGEX vectors (Promega, Madison, Wis.) also can be used to express foreign polypeptide molecules as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

Plant and Insect Expression Systems

If plant expression vectors are used, the expression of sequences encoding an APCDD1 molecule can be driven by any of a number of promoters. For example, viral promoters such as the ³⁵S and 19S promoters of CaMV can be used alone or in combination with the omega leader sequence from TMV. Alternatively, plant promoters such as the small subunit of RUBISCO or heat shock promoters, can be used. These constructs can be introduced into plant cells by direct DNA transformation or by pathogen-mediated transfection.

An insect system also can be used to express APCDD1 molecules. For example, in one such system Autographa californica nuclear polyhedrosis virus (AcNPV) is used as a vector to express foreign genes in Spodoptera frugiperda cells or in Trichoplusia larvae. Sequences encoding an APCDD1 molecule can be cloned into a non-essential region of the virus, such as the polyhedrin gene, and placed under control of the polyhedrin promoter. Successful insertion of APCDD1 nucleic acid sequences will render the polyhedrin gene inactive and produce recombinant virus lacking coat protein. The recombinant viruses can then be used to infect S. frugiperda cells or Trichoplusia larvae in which APCDD1 or a variant thereof can be expressed.

Mammalian Expression Systems

An expression vector can include a nucleotide sequence that encodes an APCDD1 molecule linked to at least one regulatory sequence in a manner allowing expression of the nucleotide sequence in a host cell. A number of viral-based expression systems can be used to express an APCDD1 molecule or a variant thereof in mammalian host cells. For example, if an adenovirus is used as an expression vector, sequences encoding an APCDD1 molecule can be ligated into an adenovirus transcription/translation complex comprising the late promoter and tripartite leader sequence. Insertion into a non-essential E1 or E3 region of the viral genome can be used to obtain a viable virus which can express an APCDD1 molecule in infected host cells. Transcription enhancers, such as the Rous sarcoma virus (RSV) enhancer, can also be used to increase expression in mammalian host cells.

Regulatory sequences are well known in the art, and can be selected to direct the expression of a protein or polypeptide of interest (such as an APCDD1 molecule) in an appropriate host cell as described in Goeddel, Gene Expression Technology: Methods in Enzymology 185, Academic Press, San Diego, Calif. (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L, et al., Curr Opin Genet Dev, 2002, 12(2):137-41) enhancers, and other expression control elements.

Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (fir example, an APCDD1 molecule) is stably integrated into the genome of eukaryotic cells (for example mammalian cells, such as cells from the end bulb of the hair follicle), resulting in the stable expression of transfected genes. An exogenous nucleic acid sequence can be introduced into a cell (such as a mammalian cell, either a primary or secondary cell) by homologous recombination as disclosed in U.S. Pat. No. 5,641,670, the contents of which are herein incorporated by reference.

A gene that encodes a selectable marker (e.g., resistance to antibiotics or drugs, such as ampicillin, neomycin, G418, and hygromycin) can be introduced into host cells along with the gene of interest to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule (for example, APCDD1).

Cell Transfection

A eukaryotic expression vector can be used to transfect cells in order to produce proteins (for example, an APCDD1 molecule) encoded by nucleotide sequences of the vector. Mammalian cells (such as isolated cells from the hair bulb; for example dermal sheath cells and dermal papilla cells) can contain an expression vector (for example, one that contains a gene encoding APCDD1 molecule) via introducing the expression vector into an appropriate host cell via methods known in the art.

A host cell strain can be chosen for its ability to modulate the expression of the inserted sequences or to process the expressed APCDD1 polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293T, and WI38), are available from the American Type Culture Collection (ATCC; 10801 University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextran-mediated transfection, or electroporation. Electroporation is carried out at approximate voltage and capacitance to result in entry of the DNA construct(s) into cells of interest (such as cells of the end bulb of a hair follicle, for example dermal papilla cells or dermal sheath cells). Other methods used to transfect cells can also include modified calcium phosphate precipitation, polybrene precipitation, liposome fusion, and receptor-mediated gene delivery.

Cells that will be genetically engineered can be primary and secondary cells obtained from various tissues, and include cell types which can be maintained and propagated in culture. Non-limiting examples of primary and secondary cells include epithelial cells (for example, dermal papilla cells, hair follicle cells, inner root sheath cells, outer root sheath cells, sebaceous gland cells, epidermal matrix cells), neural cells, endothelial cells, glial cells, fibroblasts, muscle cells (such as myoblasts) keratinocytes, formed elements of the blood (e.g., lymphocytes, bone marrow cells), and precursors of these somatic cell types.

Vertebrate tissue can be obtained by methods known to one skilled in the art, such a punch biopsy or other surgical methods of obtaining a tissue source of the primary cell type of interest. In one embodiment, a punch biopsy or removal can be used to obtain a source of keratinocytes, fibroblasts, endothelial cells, or mesenchymal cells (for example, hair follicle cells or dermal papilla cells). In another embodiment, removal of a hair follicle can be used to obtain a source of fibroblasts, keratinocytes, endothelial cells, or mesenchymal cells (for example, hair follicle cells or dermal papilla cells). A mixture of primary cells can be obtained from the tissue, using methods readily practiced in the art, such as explanting or enzymatic digestion (for examples using enzymes such as pronase, trypsin, collagenase, elastase dispase, and chymotrypsin). Biopsy methods have also been described in United States Patent Application Publication 2004/0057937 and PCT application publication WO 2001/32840, and are hereby incorporated by reference.

Primary cells can be acquired from the individual to whom the genetically engineered primary or secondary cells are administered. However, primary cells can also be obtained from a donor, other than the recipient, of the same species. The cells can also be obtained from another species (for example, rabbit, cat, mouse, rat, sheep, goat, dog, horse, cow, bird, or pig). Primary cells can also include cells from an isolated vertebrate tissue source grown attached to a tissue culture substrate (for example, flask or dish) or grown in a suspension; cells present in an explant derived from tissue; both of the aforementioned cell types plated for the first time; and cell culture suspensions derived from these plated cells. Secondary cells can be plated primary cells that are removed from the culture substrate and replated, or passaged, in addition to cells from the subsequent passages. Secondary cells can be passaged one or more times. These primary or secondary cells can contain expression vectors having a gene that encodes a protein of interest (for example, an APCDD1 molecule).

Cell Culturing

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized. Non-limiting examples of medium include, for example, Minimal Essential Medium (MEM, Sigma, St. Louis, Mo.); Dulbecco's Modified Eagles Medium (DMEM, Sigma); Ham's F10 Medium (Sigma); HyClone cell culture medium (HyClone, Logan, Utah); RPMI-1640 Medium (Sigma); and chemically-defined (CD) media, which are formulated for various cell types, e.g., CD-CHO Medium (Invitrogen, Carlsbad, Calif.).

The cell culture media can be supplemented as necessary with supplementary components or ingredients, including optional components, in appropriate concentrations or amounts, as necessary or desired. Cell culture medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that can be required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, and epidermal growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example pluronic polyol; and (8) galactose. In one embodiment, soluble factors can be added to the culturing medium.

The mammalian cell culture that can be used with the present invention is prepared in a medium suitable for the type of cell being cultured. In one embodiment, the cell culture medium can be any one of those previously discussed (for example, MEM) that is supplemented with serum from a mammalian source (for example, fetal bovine serum (FBS)). In another embodiment, the medium can be a conditioned medium to sustain the growth of epithelial cells or cells obtained from the hair bulb of a hair follicle (such as dermal papilla cells or dermal sheath cells). For example, epithelial cells can be cultured according to Barnes and Mather in Animal Cell Culture Methods (Academic Press, 1998), which is hereby incorporated by reference in its entirety. In a further embodiment, epithelial cells or hair follicle cells can be transfected with DNA vectors containing genes that encode a polypeptide or protein of interest (for example, an APCDD1 molecule). In other embodiments of the invention, cells are grown in a suspension culture (for example, a three-dimensional culture such as a hanging drop culture) in the presence of an effective amount of enzyme, wherein the enzyme substrate is an extracellular matrix molecule in the suspension culture. For example, the enzyme can be a hyaluronidase. Epithelial cells or hair follicle cells can be cultivated according to methods practiced in the art, for example, as those described in PCT application publication WO 2004/044188 and in U.S. Patent Application Publication No. 2005/0272150, or as described by Harris in Handbook in Practical Animal Cell Biology: Epithelial Cell Culture (Cambridge Univ. Press, Great Britain; 1996; see Chapter 8), which are hereby incorporated by reference.

A suspension culture is a type of culture wherein cells, or aggregates of cells (such as aggregates of DP cells), multiply while suspended in liquid medium. A suspension culture comprising mammalian cells can be used for the maintenance of cell types that do not adhere or to enable cells to manifest specific cellular characteristics that are not seen in the adherent form. Some types of suspension cultures can include three-dimensional cultures or a hanging drop culture. A hanging-drop culture is a culture in which the material to be cultivated is inoculated into a drop of fluid attached to a flat surface (such as a coverglass, glass slide, Petri dish, flask, and the like), and can be inverted over a hollow surface. Cells in a hanging drop can aggregate toward the hanging center of a drop as a result of gravity. However, according to the methods of the invention, cells cultured in the presence of a protein that degrades the extracellular matrix (such as collagenase, chondroitinase, hyaluronidase, and the like) will become more compact and aggregated within the hanging drop culture, for degradation of the ECM will allow cells to become closer in proximity to one another since less of the ECM will be present. See also International PCT Publication No. WO2007/100870, which is incorporated by reference.

Cells obtained from the hair bulb of a hair follicle (such as dermal papilla cells or dermal sheath cells) can be cultured as a single, homogenous population (for example, comprising DP cells) in a hanging drop culture so as to generate an aggregate of DP cells. Cells can also be cultured as a heterogeneous population (for example, comprising DP and DS cells) in a hanging drop culture so as to generate a chimeric aggregate of DP and DS cells. Epithelial cells can be cultured as a monolayer to confluency as practiced in the art. Such culturing methods can be carried out essentially according to methods described in Chapter 8 of the Handbook in Practical Animal Cell Biology: Epithelial Cell Culture (Cambridge Univ. Press, Great Britain; 1996); Underhill C B, J Invest Dermatol, 1993, 101(6):820-6); in Armstrong and Armstrong, (1990) J Cell Biol 110:1439-55; or in Animal Cell Culture Methods (Academic Press, 1998), which are all hereby incorporated by reference in their entireties.

Three-dimensional cultures can be formed from agar (such as Gey's Agar), hydrogels (such as matrigel, agarose, and the like; Lee et al., (2004) Biomaterials 25: 2461-2466) or polymers that are cross-linked. These polymers can comprise natural polymers and their derivatives, synthetic polymers and their derivatives, or a combination thereof. Natural polymers can be anionic polymers, cationic polymers, amphipathic polymers, or neutral polymers. Non-limiting examples of anionic polymers can include hyaluronic acid, alginic acid (alginate), carageenan, chondroitin sulfate, dextran sulfate, and pectin. Some examples of cationic polymers, include but are not limited to, chitosan or polylysine. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73). Examples of amphipathic polymers can include, but are not limited to collagen, gelatin, fibrin, and carboxymethyl chitin. Non-limiting examples of neutral polymers can include dextran, agarose, or pullulan. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73).

Cells suitable for culturing according to methods of the invention can harbor introduced expression vectors, such as plasmids. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

Obtaining and Purifying Polypeptides

An APCDD1 polypeptide molecule or a variant thereof, can be obtained by purification from human cells expressing an APCDD1 molecule by in vitro or in vivo expression of a nucleic acid sequence encoding an APCDD1 molecule; or by direct chemical synthesis.

Detecting Polypeptide Expression

Host cells which contain a nucleic acid encoding an APCDD1 molecule, and which subsequently express APCDD1, can be identified by various procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a nucleic acid encoding an APCDD1 molecule can be detected by DNA-DNA or DNA-RNA hybridization or amplification using probes or fragments of nucleic acids encoding an APCDD1 molecule. In one embodiment, an APCDD1 fragment can encompass any portion of at least about 8 consecutive nucleotides of SEQ ID NO: 2. In another embodiment, the fragment can comprise at least about 10 consecutive nucleotides, at least about 15 consecutive nucleotides, at least about 20 consecutive nucleotides, or at least about 30 consecutive nucleotides of SEQ ID NO: 2. Fragments can include all possible nucleotide lengths between about 8 and about 100 nucleotides, for example, lengths between about 15 and about 100 nucleotides, or between about 20 and about 100 nucleotides. Nucleic acid amplification-based assays involve the use of oligonucleotides selected from sequences encoding an APCDD1 polypeptide to detect transformants which contain a nucleic acid encoding an APCDD1 molecule.

Protocols for detecting and measuring the expression of an APCDD1 polypeptide using either polyclonal or monoclonal antibodies specific for the polypeptide are well established. Non-limiting examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on an APCDD1 polypeptide can be used, or a competitive binding assay can be employed.

Labeling and conjugation techniques are known by those skilled in the art and can be used in various nucleic acid and amino acid assays. Methods for producing labeled hybridization or PCR probes for detecting sequences related to nucleic acid sequences encoding APCDD1 include, but are not limited to, oligolabeling, nick translation, end-labeling, or PCR amplification using a labeled nucleotide. Alternatively, nucleic acid sequences encoding an APCDD1 polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, and/or magnetic particles.

Expression and Purification of Polypeptides

Host cells transformed with a nucleic acid sequence encoding an APCDD1 molecule can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding an APCDD1 molecule can be designed to contain signal sequences which direct secretion of soluble APCDD1 polypeptide molecules or a variant thereof, through a prokaryotic or eukaryotic cell membrane or which direct the membrane insertion of membrane-bound APCDD1 polypeptide molecule or a variant thereof.

Other constructions can also be used to join a sequence encoding an APCDD1 polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Including cleavable linker sequences (i.e., those specific for Factor Xa or enterokinase (Invitrogen, San Diego, Calif.)) between the purification domain and an APCDD1 polypeptide also can be used to facilitate purification. One such expression vector provides for expression of a fusion protein containing APCDD1 and 6 histidine residues preceding a thioredoxin or an enterokinase cleavage site. The histidine residues facilitate purification by immobilized metal ion affinity chromatography, while the enterokinase cleavage site provides a means for purifying the APCDD1 polypeptide.

An APCDD1 polypeptide molecule can be purified from any human or non-human cell which expresses the polypeptide molecule, including those which have been transfected with expression constructs that express an APCDD1 molecule. A purified APCDD1 molecule can be separated from other compounds which normally associate with APCDD1 in the cell, such as certain proteins, carbohydrates, or lipids, using methods practiced in the art. Non-limiting methods include size exclusion chromatography, ammonium sulfate fractionation, ion exchange chromatography, affinity chromatography, and preparative gel electrophoresis.

Chemical Synthesis

Nucleic acid sequences encoding an APCDD1 polypeptide can be synthesized, in whole or in part, using chemical methods known in the art. Alternatively, an APCDD1 molecule can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of APCDD1 molecules (such as those comprising APCDD1 nucleic acid or amino acid sequences) can be separately synthesized and combined using chemical methods to produce a full-length molecule. In one embodiment, an APCDD1 fragment can encompass any portion of at least about 8 consecutive nucleotides of SEQ ID NO: 2. In one embodiment, the fragment can comprise at least about 10 nucleotides, at least about 15 nucleotides, at least about 20 nucleotides, or at least about 30 nucleotides of SEQ ID NO: 2. Fragments include all possible nucleotide lengths between about 8 and about 100 nucleotides, for example, lengths between about 15 and about 100 nucleotides, or between about 20 and about 100 nucleotides.

An APCDD1 fragment can also be a fragment of an APCDD1 protein. For example, the APCDD1 fragment can encompass any portion of at least about 8 consecutive amino acids of SEQ ID NO: 1. The fragment can comprise at least about 10 consecutive amino acids, at least about 20 consecutive amino acids, at least about 30 consecutive amino acids, at least about 40 consecutive amino acids, a least about 50 consecutive amino acids, at least about 60 consecutive amino acids, at least about 70 consecutive amino acids, or at least about 75 consecutive amino acids of SEQ ID NO: 1. Fragments include all possible amino acid lengths between about 8 and 100 about amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids.

The newly synthesized peptide can be substantially purified via high performance liquid chromatography (HPLC). The composition of a synthetic APCDD1 molecule can be confirmed by amino acid analysis or sequencing. Additionally, any portion of the amino acid sequence of APCDD1 can be altered during direct synthesis and/or combined using chemical methods with sequences from other proteins to produce a variant polypeptide or a fusion protein.

Identifying APCDD1 Modulating Compounds

The invention provides methods for identifying compounds which can be used for controlling and/or regulating hair growth (for example, hair density) or hair pigmentation in a subject. In addition, the invention provides methods for identifying compounds which can be used for the treatment of a hair loss disorder. The invention also provides methods for identifying compounds which can be used for the treatment of hypertrichosis. The invention also provides methods for identifying compounds which can be used for the treatment of hypotrichosis (for example, hereditary hypotrichosis simplex (HHS)). Non-limiting examples of hair loss disorders include: androgenetic alopecia, Alopecia greata, telogen effluvium, Alopecia greata, alopecia totalis, and alopecia universalis. The methods can comprise the identification of test compounds or agents (e.g., peptides (such as antibodies or fragments thereof), small molecules, nucleic acids (such as siRNA or antisense RNA), or other agents) that can bind to an APCDD1 polypeptide molecule and/or have a stimulatory or inhibitory effect on the biological activity of APCDD1 or its expression, and subsequently determining whether these compounds can regulate hair growth in a subject or can have an effect on symptoms associated with the hair loss disorders in an in vivo assay (i.e., examining an increase or reduction in hair growth).

As used herein, an “APCDD1 modulating compound” refers to a compound that interacts with an APCDD1 polypeptide molecule and modulates its Wnt/β-catenin signaling activity and/or its expression. The compound can either increase APCDD1's activity or expression. Conversely, the compound can decrease APCDD1's activity or expression. The compound can be an APCDD1 agonist or an APCDD1 antagonist. Some non-limiting examples of APCDD1 modulating compounds include peptides (such as APCDD1 peptide fragments, or antibodies or fragments thereof), small molecules, and nucleic acids (such as APCDD1 siRNA or antisense RNA specific for APCDD1 nucleic acid). Agonists of an APCDD1 molecule can be molecules which, when bound to APCDD1, increase or prolong the activity of an APCDD1 molecule. Agonists of APCDD1 include, but are not limited to, proteins, nucleic acids, small molecules, or any other molecule which activates APCDD1. Antagonists of an APCDD1 molecule can be molecules which, when bound to APCDD1 or a variant thereof, decrease the amount or the duration of the activity of an APCDD1 molecule. Antagonists include proteins, nucleic acids, antibodies, small molecules, or any other molecule which decrease the activity of APCDD1.

The term “modulate,” as it appears herein, refers to a change in the activity or expression of an APCDD1 molecule. For example, modulation can cause an increase or a decrease in protein activity, binding characteristics, or any other biological, functional, or immunological properties of an APCDD1 molecule.

In one embodiment, an APCDD1 modulating compound can be a peptide fragment of an APCDD1 protein that binds to the protein. For example, the APCDD1 molecule can encompass any portion of at least about 8 consecutive amino acids of SEQ ID NO: 1. The fragment can comprise at least about 10 consecutive amino acids, at least about 20 consecutive amino acids, at least about 30 consecutive amino acids, at least about 40 consecutive amino acids, at least about 50 consecutive amino acids, at least about 60 consecutive amino acids, or at least about 75 consecutive amino acids of SEQ ID NO: 1. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England). The APCDD1 peptide fragments can be isolated from a natural source, genetically engineered, or chemically prepared. These methods are well known in the art.

An APCDD1 modulating compound can also be a protein, such as an antibody (monoclonal, polyclonal, humanized, chimeric, or fully human), or a binding fragment thereof, directed against APCDD1. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

Inhibition of RNA encoding APCDD1 can effectively modulate the expression of the APCDD1 gene from which the RNA is transcribed. Inhibitors are selected from the group comprising: siRNA; interfering RNA or RNAi; dsRNA; RNA Polymerase III transcribed DNAs; ribozymes; and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding an APCDD1 polypeptide can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59). Antisense nucleotide sequences include, but are not limited to: morpholinos, 2′-O-methyl polynucleotides, DNA, RNA and the like.

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area. See also, McMnaus and Sharp (2002) Nat Rev Genetics, 3:737-47, and Sen and Blau (2006) FASEB J., 20:1293-99, the entire disclosures of which are herein incorporated by reference.

The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector (for example, see U.S. Pat. No. 7,294,504 and U.S. Pat. No. 7,422,896, the entire disclosures of which are herein incorporated by reference). Exemplary methods for producing and testing dsRNA or siRNA molecules are described in U.S. Patent Application Publication No. 2002/0173478 to Gewirtz, U.S. Patent Application Publication No. 2007/0072204 to Hannon et al., and in U.S. Patent Application Publication No. 2004/0018176 to Reich et al., the entire disclosures of which are herein incorporated by reference.

In one embodiment, an siRNA directed to human APCDD1 can comprise any one of SEQ ID NOS: 112-3776. Table 1 lists siRNA sequences comprising SEQ ID NOS: 112-3776.

In another embodiment, an siRNA directed to mouse APCDD1 can comprise any one of SEQ ID NOS: 3777-9338. Table 2 lists siRNA sequences comprising SEQ ID NOS: 3777-9338.

TABLE 1
List of siRNAs directed to human APCDD1.

| | | | | | | | |
|---|---|---|---|---|---|---|---|
| | SEQ | | | | | | |
| | ID | | SEQ ID | | SEQ | | SEQ ID |
| Sense (5′-3′) | NO: | Anti-sense (5′-3′) | NO: | Sense (5′-3′) | ID NO: | Anti-sense (5′-3′) | NO: |

| | | | | | | | |
|---|---|---|---|---|---|---|---|
| gaaatatgaagagacgctg | 112 | cagcgtctcttcatatttc | 1046 | acggctgtgagtgcaccaa | 1980 | ttggtgcactcacagccgt | 2879 |
| aaatatgaagagacgctgc | 113 | gcagcgtctcttcatattt | 1047 | cggctgtgagtgcaccaag | 1981 | cttggtgcactcacagccg | 2880 |
| aatatgaagagacgctgca | 114 | tgcagcgtctcttcatatt | 1048 | ggctgtgagtgcaccaagg | 1982 | ccttggtgcactcacagcc | 2881 |
| atatgaagagacgctgcag | 115 | ctgcagcgtctcttcatat | 1049 | gctgtgagtgcaccaaggc | 1983 | gccttggtgcactcacagc | 2882 |
| tatgaagagacgctgcagc | 116 | gctgcagcgtctcttcata | 1050 | ctgtgagtgcaccaaggcc | 1984 | ggccttggtgcactcacag | 2883 |
| atgaagagacgctgcagct | 117 | agctgcagcgtctcttcat | 1051 | tgtgagtgcaccaaggccg | 1985 | cggccttggtgcactcaca | 2884 |
| tgaagagacgctgcagctg | 118 | cagctgcagcgtctcttca | 1052 | gtgagtgcaccaaggccgt | 1986 | acggccttggtgcactcac | 2885 |
| gaagagacgctgcagctgc | 119 | gcagctgcagcgtctcttc | 1053 | tgagtgcaccaaggccgtg | 1987 | cacggccttggtgcactca | 2886 |
| aagagacgctgcagctgcg | 120 | cgcagctgcagcgtctctt | 1054 | gagtgcaccaaggccgtga | 1988 | tcacggccttggtgcactc | 2887 |
| agagacgctgcagctgcgg | 121 | ccgcagctgcagcgtctct | 1055 | agtgcaccaaggccgtgaa | 1989 | ttcacggccttggtgcact | 2888 |
| gagacgctgcagctgcggt | 122 | accgcagctgcagcgtctc | 1056 | gtgcaccaaggccgtgaac | 1990 | gttcacggccttggtgcac | 2889 |
| agacgctgcagctgcggtg | 123 | caccgcagctgcagcgtct | 1057 | tgcaccaaggccgtgaact | 1991 | agttcacggccttggtgca | 2890 |
| gacgctgcagctgcggtgg | 124 | ccaccgcagctgcagcgtc | 1058 | gcaccaaggccgtgaactt | 1992 | aagttcacggccttggtgc | 2891 |
| acgctgcagctgcggtggc | 125 | gccaccgcagctgcagcgt | 1059 | caccaaggccgtgaacttt | 1993 | aaagttcacggccttggtg | 2892 |
| cgctgcagctgcggtggcg | 126 | cgccaccgcagctgcagcg | 1060 | accaaggccgtgaactttg | 1994 | caaagttcacggccttggt | 2893 |
| gctgcagctgcggtggcgg | 127 | ccgccaccgcagctgcagc | 1061 | ccaaggccgtgaactttgc | 1995 | gcaaagttcacggccttgg | 2894 |
| ctgcagctgcggtggcggt | 128 | accgccaccgcagctgcag | 1062 | caaggccgtgaactttgcc | 1996 | ggcaaagttcacggccttg | 2895 |
| tgcagctgcggtggcggtg | 129 | caccgccaccgcagctgca | 1063 | aaggccgtgaactttgcca | 1997 | tggcaaagttcacggcctt | 2896 |
| gcagctgcggtggcggtgg | 130 | ccaccgccaccgcagctgc | 1064 | aggccgtgaactttgccat | 1998 | atggcaaagttcacggcct | 2897 |
| cagctgcggtggcggtggc | 131 | gccaccgccaccgcagctg | 1065 | ggccgtgaactttgccatg | 1999 | catggcaaagttcacggcc | 2898 |
| agctgcggtggcggtggcg | 132 | cgccaccgccaccgcagct | 1066 | gccgtgaactttgccatgc | 2000 | gcatggcaaagttcacggc | 2899 |
| gctgcggtggcggtggcgg | 133 | ccgccaccgccaccgcagc | 1067 | ccgtgaactttgccatgca | 2001 | tgcatggcaaagttcacgg | 2900 |
| ctgcggtggcggtggcggc | 134 | gccgccaccgccaccgcag | 1068 | cgtgaactttgccatgcat | 2002 | atgcatggcaaagttcacg | 2901 |
| tgcggtggcggtggcggcc | 135 | ggccgccaccgccaccgca | 1069 | gtgaactttgccatgcatg | 2003 | catgcatggcaaagttcac | 2902 |
| gcggtggcggtggcggcca | 136 | tggccgccaccgccaccgc | 1070 | tgaactttgccatgcatga | 2004 | tcatgcatggcaaagttca | 2903 |
| cggtggcggtggcggccac | 137 | gtggccgccaccgccaccg | 1071 | gaactttgccatgcatgaa | 2005 | ttcatgcatggcaaagttc | 2904 |
| ggtggcggtggcggccact | 138 | agtggccgccaccgccacc | 1072 | aactttgccatgcatgaac | 2006 | gttcatgcatggcaaagtt | 2905 |
| gtggcggtggcggccactg | 139 | cagtggccgccaccgccac | 1073 | actttgccatgcatgaact | 2007 | agttcatgcatggcaaagt | 2906 |
| tggcggtggcggccactgc | 140 | gcagtggccgccaccgcca | 1074 | ctttgccatgcatgaactt | 2008 | aagttcatgcatggcaaag | 2907 |
| ggcggtggcggccactgca | 141 | tgcagtggccgccaccgcc | 1075 | tttgccatgcatgaacttc | 2009 | gaagttcatgcatggcaaa | 2908 |
| gcggtggcggccactgcag | 142 | ctgcagtggccgccaccgc | 1076 | ttgccatgcatgaacttca | 2010 | tgaagttcatgcatggcaa | 2909 |
| cggtggcggccactgcagc | 143 | gctgcagtggccgccaccg | 1077 | tgccatgcatgaacttcag | 2011 | ctgaagttcatgcatggca | 2910 |
| ggtggcggccactgcagct | 144 | agctgcagtggccgccacc | 1078 | gccatgcatgaacttcagc | 2012 | gctgaagttcatgcatggc | 2911 |
| gtggcggccactgcagctc | 145 | gagctgcagtggccgccac | 1079 | ccatgcatgaacttcagct | 2013 | agctgaagttcatgcatgg | 2912 |
| tggcggccactgcagctca | 146 | tgagctgcagtggccgcca | 1080 | catgcatgaacttcagctc | 2014 | gagctgaagttcatgcatg | 2913 |
| ggcggccactgcagctcag | 147 | ctgagctgcagtggccgcc | 1081 | atgcatgaacttcagctca | 2015 | tgagctgaagttcatgcat | 2914 |
| gcggccactgcagctcaga | 148 | tctgagctgcagtggccgc | 1082 | tgcatgaacttcagctcat | 2016 | atgagctgaagttcatgca | 2915 |
| cggccactgcagctcagag | 149 | ctctgagctgcagtggccg | 1083 | gcatgaacttcagctcatc | 2017 | gatgagctgaagttcatgc | 2916 |
| ggccactgcagctcagagc | 150 | gctctgagctgcagtggcc | 1084 | catgaacttcagctcatcc | 2018 | ggatgagctgaagttcatg | 2917 |
| gccactgcagctcagagcg | 151 | cgctctgagctgcagtggc | 1085 | atgaacttcagctcatccg | 2019 | cggatgagctgaagttcat | 2918 |
| ccactgcagctcagagcgg | 152 | ccgctctgagctgcagtgg | 1086 | tgaacttcagctcatccgg | 2020 | ccggatgagctgaagttca | 2919 |
| cactgcagctcagagcggc | 153 | gccgctctgagctgcagtg | 1087 | gaacttcagctcatccggg | 2021 | cccggatgagctgaagttc | 2920 |
| actgcagctcagagcggcg | 154 | cgccgctctgagctgcagt | 1088 | aacttcagctcatccgggt | 2022 | acccggatgagctgaagtt | 2921 |
| ctgcagctcagagcggcgc | 155 | gcgccgctctgagctgcag | 1089 | acttcagctcatccgggtg | 2023 | cacccggatgagctgaagt | 2922 |
| tgcagctcagagcggcgca | 156 | tgcgccgctctgagctgca | 1090 | cttcagctcatccgggtgg | 2024 | ccacccggatgagctgaag | 2923 |
| gcagctcagagcggcgcac | 157 | gtgcgccgctctgagctgc | 1091 | ttcagctcatccgggtgga | 2025 | tccacccggatgagctgaa | 2924 |
| cagctcagagcggcgcacg | 158 | cgtgcgccgctctgagctg | 1092 | tcagctcatccgggtggag | 2026 | ctccacccggatgagctga | 2925 |
| agctcagagcggcgcacgc | 159 | gcgtgcgccgctctgagct | 1093 | cagctcatccgggtggaga | 2027 | tctccacccggatgagctg | 2926 |
| gctcagagcggcgcacgcg | 160 | cgcgtgcgccgctctgagc | 1094 | agctcatccgggtggagaa | 2028 | ttctccacccggatgagct | 2927 |
| ctcagagcggcgcacgcgg | 161 | ccgcgtgcgccgctctgag | 1095 | gctcatccgggtggagaag | 2029 | cttctccacccggatgagc | 2928 |
| tcagagcggcgcacgcggc | 162 | gccgcgtgcgccgctctga | 1096 | ctcatccgggtggagaagc | 2030 | gcttctccacccggatgag | 2929 |
| cagagcggcgcacgcggcg | 163 | cgccgcgtgcgccgctctg | 1097 | tcatccgggtggagaagca | 2031 | tgcttctccacccggatga | 2930 |
| agagcggcgcacgcggcgg | 164 | ccgccgcgtgcgccgctct | 1098 | catccgggtggagaagcag | 2032 | ctgcttctccacccggatg | 2931 |
| gagcggcgcacgcggcggc | 165 | gccgccgcgtgcgccgctc | 1099 | atccgggtggagaagcagt | 2033 | actgcttctccacccggat | 2932 |
| agcggcgcacgcggcggcc | 166 | ggccgccgcgtgcgccgct | 1100 | tccgggtggagaagcagta | 2034 | tactgcttctccacccgga | 2933 |
| gcggcgcacgcggcggccg | 167 | cggccgccgcgtgcgccgc | 1101 | ccgggtggagaagcagtac | 2035 | gtactgcttctccacccgg | 2934 |
| cggcgcacgcggcggccgg | 168 | ccggccgccgcgtgcgccg | 1102 | cgggtggagaagcagtacc | 2036 | ggtactgcttctccacccg | 2935 |
| ggcgcacgcggcggccggg | 169 | cccggccgccgcgtgcgcc | 1103 | gggtggagaagcagtacct | 2037 | aggtactgcttctccaccc | 2936 |
| gcgcacgcggcggccgggg | 170 | ccccggccgccgcgtgcgc | 1104 | ggtggagaagcagtacctt | 2038 | aaggtactgcttctccacc | 2937 |
| cgcacgcggcggccggggc | 171 | gccccggccgccgcgtgcg | 1105 | gtggagaagcagtaccttc | 2039 | gaaggtactgcttctccac | 2938 |
| gcacgcggcggccggggcg | 172 | cgccccggccgccgcgtgc | 1106 | tggagaagcagtaccttca | 2040 | tgaaggtactgcttctcca | 2939 |
| cacgcggcggccggggcgg | 173 | ccgccccggccgccgcgtg | 1107 | ggagaagcagtaccttcac | 2041 | gtgaaggtactgcttctcc | 2940 |
| acgcggcggccggggcggg | 174 | cccgccccggccgccgcgt | 1108 | gagaagcagtaccttcacc | 2042 | ggtgaaggtactgcttctc | 2941 |
| cgcggcggccggggcggga | 175 | tcccgccccggccgccgcg | 1109 | agaagcagtaccttcacca | 2043 | tggtgaaggtactgcttct | 2942 |
| gcggcggccggggcgggac | 176 | gtcccgccccggccgccgc | 1110 | gaagcagtaccttcaccac | 2044 | gtggtgaaggtactgcttc | 2943 |
| cggcggccggggcgggacg | 177 | cgtcccgccccggccgccg | 1111 | aagcagtaccttcaccaca | 2045 | tgtggtgaaggtactgctt | 2944 |
| ggcggccggggcgggacgc | 178 | gcgtcccgccccggccgcc | 1112 | agcagtaccttcaccacaa | 2046 | ttgtggtgaaggtactgct | 2945 |
| gcggccggggcgggacgcg | 179 | cgcgtcccgccccggccgc | 1113 | gcagtaccttcaccacaac | 2047 | gttgtggtgaaggtactgc | 2946 |
| cggccggggcgggacgcgg | 180 | ccgcgtcccgccccggccg | 1114 | cagtaccttcaccacaacc | 2048 | ggttgtggtgaaggtactg | 2947 |
| ggccggggcgggacgcggg | 181 | cccgcgtcccgccccggcc | 1115 | agtaccttcaccacaacct | 2049 | aggttgtggtgaaggtact | 2948 |
| gccggggcgggacgcgggg | 182 | ccccgcgtcccgccccggc | 1116 | gtaccttcaccacaacctc | 2050 | gaggttgtggtgaaggtac | 2949 |
| ccggggcgggacgcggggc | 183 | gccccgcgtcccgccccgg | 1117 | taccttcaccacaacctcg | 2051 | cgaggttgtggtgaaggta | 2950 |
| cggggcgggacgcggggcc | 184 | ggccccgcgtcccgccccg | 1118 | accttcaccacaacctcga | 2052 | tcgaggttgtggtgaaggt | 2951 |
| ggggcgggacgcggggccg | 185 | cggccccgcgtcccgcccc | 1119 | ccttcaccacaacctcgac | 2053 | gtcgaggttgtggtgaagg | 2952 |
| gggcgggacgcggggccgg | 186 | ccggccccgcgtcccgccc | 1120 | cttcaccacaacctcgacc | 2054 | ggtcgaggttgtggtgaag | 2953 |
| ggcgggacgcggggccggg | 187 | cccggccccgcgtcccgcc | 1121 | ttcaccacaacctcgacca | 2055 | tggtcgaggttgtggtgaa | 2954 |
| gcgggacgcggggccgggc | 188 | gcccggccccgcgtcccgc | 1122 | tcaccacaacctcgaccac | 2056 | gtggtcgaggttgtggtga | 2955 |
| cgggacgcggggccgggcg | 189 | cgcccggccccgcgtcccg | 1123 | caccacaacctcgaccacc | 2057 | ggtggtcgaggttgtggtg | 2956 |
| gggacgcggggccgggcgc | 190 | gcgcccggccccgcgtccc | 1124 | accacaacctcgaccacct | 2058 | aggtggtcgaggttgtggt | 2957 |
| ggacgcggggccgggcgcg | 191 | cgcgcccggccccgcgtcc | 1125 | ccacaacctcgaccacctg | 2059 | caggtggtcgaggttgtgg | 2958 |
| gacgcggggccgggcgcgg | 192 | ccgcgcccggccccgcgtc | 1126 | cacaacctcgaccacctgg | 2060 | ccaggtggtcgaggttgtg | 2959 |
| acgcggggccgggcgcgga | 193 | tccgcgcccggccccgcgt | 1127 | acaacctcgaccacctggt | 2061 | accaggtggtcgaggttgt | 2960 |
| cgcggggccgggcgcggag | 194 | ctccgcgcccggccccgcg | 1128 | caacctcgaccacctggtc | 2062 | gaccaggtggtcgaggttg | 2961 |
| gcggggccgggcgcggaga | 195 | tctccgcgcccggccccgc | 1129 | aacctcgaccacctggtcg | 2063 | cgaccaggtggtcgaggtt | 2962 |
| cggggccgggcgcggagaa | 196 | ttctccgcgcccggccccg | 1130 | acctcgaccacctggtcga | 2064 | tcgaccaggtggtcgaggt | 2963 |
| ggggccgggcgcggagaag | 197 | cttctccgcgcccggcccc | 1131 | cctcgaccacctggtcgag | 2065 | ctcgaccaggtggtcgagg | 2964 |
| gggccgggcgcggagaagt | 198 | acttctccgcgcccggccc | 1132 | ctcgaccacctggtcgagg | 2066 | cctcgaccaggtggtcgag | 2965 |
| ggccgggcgcggagaagtc | 199 | gacttctccgcgcccggcc | 1133 | tcgaccacctggtcgagga | 2067 | tcctcgaccaggtggtcga | 2966 |
| gccgggcgcggagaagtcg | 200 | cgacttctccgcgcccggc | 1134 | cgaccacctggtcgaggag | 2068 | ctcctcgaccaggtggtcg | 2967 |
| ccgggcgcggagaagtcgg | 201 | ccgacttctccgcgcccgg | 1135 | gaccacctggtcgaggagc | 2069 | gctcctcgaccaggtggtc | 2968 |
| cgggcgcggagaagtcggg | 202 | cccgacttctccgcgcccg | 1136 | accacctggtcgaggagct | 2070 | agctcctcgaccaggtggt | 2969 |
| gggcgcggagaagtcgggg | 203 | ccccgacttctccgcgccc | 1137 | ccacctggtcgaggagctc | 2071 | gagctcctcgaccaggtgg | 2970 |
| ggcgcggagaagtcggggc | 204 | gccccgacttctccgcgcc | 1138 | cacctggtcgaggagctct | 2072 | agagctcctcgaccaggtg | 2971 |
| gcgcggagaagtcggggcg | 205 | cgccccgacttctccgcgc | 1139 | acctggtcgaggagctctt | 2073 | aagagctcctcgaccaggt | 2972 |
| cgcggagaagtcggggcgg | 206 | ccgccccgacttctccgcg | 1140 | cctggtcgaggagctcttc | 2074 | gaagagctcctcgaccagg | 2973 |
| gcggagaagtcggggcggg | 207 | cccgccccgacttctccgc | 1141 | ctggtcgaggagctcttcc | 2075 | ggaagagctcctcgaccag | 2974 |
| cggagaagtcggggcgggc | 208 | gcccgccccgacttctccg | 1142 | tggtcgaggagctcttcct | 2076 | aggaagagctcctcgacca | 2975 |
| ggagaagtcggggcgggcg | 209 | cgcccgccccgacttctcc | 1143 | ggtcgaggagctcttcctt | 2077 | aaggaagagctcctcgacc | 2976 |
| gagaagtcggggcgggcgg | 210 | ccgcccgccccgacttctc | 1144 | gtcgaggagctcttccttg | 2078 | caaggaagagctcctcgac | 2977 |
| agaagtcggggcgggcggc | 211 | gccgcccgccccgacttct | 1145 | tcgaggagctcttccttgg | 2079 | ccaaggaagagctcctcga | 2978 |
| gaagtcggggcgggcggca | 212 | tgccgcccgccccgacttc | 1146 | cgaggagctcttccttggt | 2080 | accaaggaagagctcctcg | 2979 |
| aagtcggggcgggcggcag | 213 | ctgccgcccgccccgactt | 1147 | gaggagctcttccttggtg | 2081 | caccaaggaagagctcctc | 2980 |
| agtcggggcgggcggcaga | 214 | tctgccgcccgccccgact | 1148 | aggagctcttccttggtga | 2082 | tcaccaaggaagagctcct | 2981 |
| gtcggggcgggcggcagag | 215 | ctctgccgcccgccccgac | 1149 | ggagctcttccttggtgac | 2083 | gtcaccaaggaagagctcc | 2982 |
| tcggggcgggcggcagaga | 216 | tctctgccgcccgccccga | 1150 | gagctcttccttggtgaca | 2084 | tgtcaccaaggaagagctc | 2983 |
| cggggcgggcggcagagag | 217 | ctctctgccgcccgccccg | 1151 | agctcttccttggtgacat | 2085 | atgtcaccaaggaagagct | 2984 |
| ggggcgggcggcagagagg | 218 | cctctctgccgcccgcccc | 1152 | gctcttccttggtgacatt | 2086 | aatgtcaccaaggaagagc | 2985 |
| gggcgggcggcagagaggc | 219 | gcctctctgccgcccgccc | 1153 | ctcttccttggtgacattc | 2087 | gaatgtcaccaaggaagag | 2986 |
| ggcgggcggcagagaggcc | 220 | ggcctctctgccgcccgcc | 1154 | tcttccttggtgacattca | 2088 | tgaatgtcaccaaggaaga | 2987 |
| gcgggcggcagagaggccg | 221 | cggcctctctgccgcccgc | 1155 | cttccttggtgacattcac | 2089 | gtgaatgtcaccaaggaag | 2988 |
| cgggcggcagagaggccgg | 222 | ccggcctctctgccgcccg | 1156 | ttccttggtgacattcaca | 2090 | tgtgaatgtcaccaaggaa | 2989 |
| gggcggcagagaggccggg | 223 | cccggcctctctgccgccc | 1157 | tccttggtgacattcacac | 2091 | gtgtgaatgtcaccaagga | 2990 |
| ggcggcagagaggccggga | 224 | tcccggcctctctgccgcc | 1158 | ccttggtgacattcacact | 2092 | agtgtgaatgtcaccaagg | 2991 |
| gcggcagagaggccgggac | 225 | gtcccggcctctctgccgc | 1159 | cttggtgacattcacactg | 2093 | cagtgtgaatgtcaccaag | 2992 |
| cggcagagaggccgggacg | 226 | cgtcccggcctctctgccg | 1160 | ttggtgacattcacactga | 2094 | tcagtgtgaatgtcaccaa | 2993 |
| ggcagagaggccgggacgc | 227 | gcgtcccggcctctctgcc | 1161 | tggtgacattcacactgat | 2095 | atcagtgtgaatgtcacca | 2994 |
| gcagagaggccgggacgcg | 228 | cgcgtcccggcctctctgc | 1162 | ggtgacattcacactgatg | 2096 | catcagtgtgaatgtcacc | 2995 |
| cagagaggccgggacgcgg | 229 | ccgcgtcccggcctctctg | 1163 | gtgacattcacactgatgc | 2097 | gcatcagtgtgaatgtcac | 2996 |
| agagaggccgggacgcgga | 230 | tccgcgtcccggcctctct | 1164 | tgacattcacactgatgcc | 2098 | ggcatcagtgtgaatgtca | 2997 |
| gagaggccgggacgcggac | 231 | gtccgcgtcccggcctctc | 1165 | gacattcacactgatgcca | 2099 | tggcatcagtgtgaatgtc | 2998 |
| agaggccgggacgcggacc | 232 | ggtccgcgtcccggcctct | 1166 | acattcacactgatgccac | 2100 | gtggcatcagtgtgaatgt | 2999 |
| gaggccgggacgcggaccg | 233 | cggtccgcgtcccggcctc | 1167 | cattcacactgatgccacc | 2101 | ggtggcatcagtgtgaatg | 3000 |
| aggccgggacgcggaccgg | 234 | ccggtccgcgtcccggcct | 1168 | attcacactgatgccaccc | 2102 | gggtggcatcagtgtgaat | 3001 |
| ggccgggacgcggaccggg | 235 | cccggtccgcgtcccggcc | 1169 | ttcacactgatgccaccca | 2103 | tgggtggcatcagtgtgaa | 3002 |
| gccgggacgcggaccgggc | 236 | gcccggtccgcgtcccggc | 1170 | tcacactgatgccacccag | 2104 | ctgggtggcatcagtgtga | 3003 |
| ccgggacgcggaccgggcc | 237 | ggcccggtccgcgtcccgg | 1171 | cacactgatgccacccaga | 2105 | tctgggtggcatcagtgtg | 3004 |
| cgggacgcggaccgggccg | 238 | cggcccggtccgcgtcccg | 1172 | acactgatgccacccagag | 2106 | ctctgggtggcatcagtgt | 3005 |
| gggacgcggaccgggccgg | 239 | ccggcccggtccgcgtccc | 1173 | cactgatgccacccagagg | 2107 | cctctgggtggcatcagtg | 3006 |
| ggacgcggaccgggccggg | 240 | cccggcccggtccgcgtcc | 1174 | actgatgccacccagagga | 2108 | tcctctgggtggcatcagt | 3007 |
| gacgcggaccgggccgggg | 241 | ccccggcccggtccgcgtc | 1175 | ctgatgccacccagaggat | 2109 | atcctctgggtggcatcag | 3008 |
| acgcggaccgggccggggc | 242 | gccccggcccggtccgcgt | 1176 | tgatgccacccagaggatg | 2110 | catcctctgggtggcatca | 3009 |
| cgcggaccgggccggggcg | 243 | cgccccggcccggtccgcg | 1177 | gatgccacccagaggatgt | 2111 | acatcctctgggtggcatc | 3010 |
| gcggaccgggccggggcgc | 244 | gcgccccggcccggtccgc | 1178 | atgccacccagaggatgtt | 2112 | aacatcctctgggtggcat | 3011 |
| cggaccgggccggggcgcc | 245 | ggcgccccggcccggtccg | 1179 | tgccacccagaggatgttc | 2113 | gaacatcctctgggtggca | 3012 |
| ggaccgggccggggcgccc | 246 | gggcgccccggcccggtcc | 1180 | gccacccagaggatgttct | 2114 | agaacatcctctgggtggc | 3013 |
| gaccgggccggggcgccca | 247 | tgggcgccccggcccggtc | 1181 | ccacccagaggatgttcta | 2115 | tagaacatcctctgggtgg | 3014 |
| accgggccggggcgcccac | 248 | gtgggcgccccggcccggt | 1182 | cacccagaggatgttctac | 2116 | gtagaacatcctctgggtg | 3015 |
| ccgggccggggcgcccaca | 249 | tgtgggcgccccggcccgg | 1183 | acccagaggatgttctacc | 2117 | ggtagaacatcctctgggt | 3016 |
| cgggccggggcgcccacag | 250 | ctgtgggcgccccggcccg | 1184 | cccagaggatgttctaccg | 2118 | cggtagaacatcctctggg | 3017 |
| gggccggggcgcccacagc | 251 | gctgtgggcgccccggccc | 1185 | ccagaggatgttctaccgg | 2119 | ccggtagaacatcctctgg | 3018 |
| ggccggggcgcccacagcc | 252 | ggctgtgggcgccccggcc | 1186 | cagaggatgttctaccggc | 2120 | gccggtagaacatcctctg | 3019 |
| gccggggcgcccacagccg | 253 | cggctgtgggcgccccggc | 1187 | agaggatgttctaccggcc | 2121 | ggccggtagaacatcctct | 3020 |
| ccggggcgcccacagccgc | 254 | gcggctgtgggcgccccgg | 1188 | gaggatgttctaccggccc | 2122 | gggccggtagaacatcctc | 3021 |
| cggggcgcccacagccgcc | 255 | ggcggctgtgggcgccccg | 1189 | aggatgttctaccggccct | 2123 | agggccggtagaacatcct | 3022 |
| ggggcgcccacagccgccc | 256 | gggcggctgtgggcgcccc | 1190 | ggatgttctaccggccctc | 2124 | gagggccggtagaacatcc | 3023 |
| gggcgcccacagccgcccg | 257 | cgggcggctgtgggcgccc | 1191 | gatgttctaccggccctcc | 2125 | ggagggccggtagaacatc | 3024 |
| ggcgcccacagccgcccga | 258 | tcgggcggctgtgggcgcc | 1192 | atgttctaccggccctcca | 2126 | tggagggccggtagaacat | 3025 |
| gcgcccacagccgcccgac | 259 | gtcgggcggctgtgggcgc | 1193 | tgttctaccggccctccag | 2127 | ctggagggccggtagaaca | 3026 |
| cgcccacagccgcccgacg | 260 | cgtcgggcggctgtgggcg | 1194 | gttctaccggccctccagt | 2128 | actggagggccggtagaac | 3027 |
| gcccacagccgcccgacgg | 261 | ccgtcgggcggctgtgggc | 1195 | ttctaccggccctccagtt | 2129 | aactggagggccggtagaa | 3028 |
| cccacagccgcccgacggc | 262 | gccgtcgggcggctgtggg | 1196 | tctaccggccctccagtta | 2130 | taactggagggccggtaga | 3029 |
| ccacagccgcccgacggcg | 263 | cgccgtcgggcggctgtgg | 1197 | ctaccggccctccagttac | 2131 | gtaactggagggccggtag | 3030 |
| cacagccgcccgacggcgc | 264 | gcgccgtcgggcggctgtg | 1198 | taccggccctccagttacc | 2132 | ggtaactggagggccggta | 3031 |
| acagccgcccgacggcgcc | 265 | ggcgccgtcgggcggctgt | 1199 | accggccctccagttacca | 2133 | tggtaactggagggccggt | 3032 |
| cagccgcccgacggcgccc | 266 | gggcgccgtcgggcggctg | 1200 | ccggccctccagttaccag | 2134 | ctggtaactggagggccgg | 3033 |
| agccgcccgacggcgccca | 267 | tgggcgccgtcgggcggct | 1201 | cggccctccagttaccagc | 2135 | gctggtaactggagggccg | 3034 |
| gccgcccgacggcgcccag | 268 | ctgggcgccgtcgggcggc | 1202 | ggccctccagttaccagcc | 2136 | ggctggtaactggagggcc | 3035 |
| ccgcccgacggcgcccaga | 269 | tctgggcgccgtcgggcgg | 1203 | gccctccagttaccagccc | 2137 | gggctggtaactggagggc | 3036 |
| cgcccgacggcgcccagag | 270 | ctctgggcgccgtcgggcg | 1204 | ccctccagttaccagcccc | 2138 | ggggctggtaactggaggg | 3037 |
| gcccgacggcgcccagaga | 271 | tctctgggcgccgtcgggc | 1205 | cctccagttaccagccccc | 2139 | gggggctggtaactggagg | 3038 |
| cccgacggcgcccagagag | 272 | ctctctgggcgccgtcggg | 1206 | ctccagttaccagccccct | 2140 | agggggctggtaactggag | 3039 |
| ccgacggcgcccagagagc | 273 | gctctctgggcgccgtcgg | 1207 | tccagttaccagccccctc | 2141 | gagggggctggtaactgga | 3040 |
| cgacggcgcccagagagcg | 274 | cgctctctgggcgccgtcg | 1208 | ccagttaccagccccctct | 2142 | agagggggctggtaactgg | 3041 |
| gacggcgcccagagagcgc | 275 | gcgctctctgggcgccgtc | 1209 | cagttaccagccccctctg | 2143 | cagagggggctggtaactg | 3042 |
| acggcgcccagagagcgcg | 276 | cgcgctctctgggcgccgt | 1210 | agttaccagccccctctgc | 2144 | gcagagggggctggtaact | 3043 |
| cggcgcccagagagcgcgc | 277 | gcgcgctctctgggcgccg | 1211 | gttaccagccccctctgca | 2145 | tgcagagggggctggtaac | 3044 |
| ggcgcccagagagcgcgcg | 278 | cgcgcgctctctgggcgcc | 1212 | ttaccagccccctctgcag | 2146 | ctgcagagggggctggtaa | 3045 |
| gcgcccagagagcgcgcgc | 279 | gcgcgcgctctctgggcgc | 1213 | taccagccccctctgcaga | 2147 | tctgcagagggggctggta | 3046 |
| cgcccagagagcgcgcgcc | 280 | ggcgcgcgctctctgggcg | 1214 | accagccccctctgcagaa | 2148 | ttctgcagagggggctggt | 3047 |
| gcccagagagcgcgcgccc | 281 | gggcgcgcgctctctgggc | 1215 | ccagccccctctgcagaat | 2149 | attctgcagagggggctgg | 3048 |
| cccagagagcgcgcgcccc | 282 | ggggcgcgcgctctctggg | 1216 | cagccccctctgcagaatg | 2150 | cattctgcagagggggctg | 3049 |
| ccagagagcgcgcgccccg | 283 | cggggcgcgcgctctctgg | 1217 | agccccctctgcagaatgc | 2151 | gcattctgcagagggggct | 3050 |
| cagagagcgcgcgccccgc | 284 | gcggggcgcgcgctctctg | 1218 | gccccctctgcagaatgcc | 2152 | ggcattctgcagagggggc | 3051 |
| agagagcgcgcgccccgca | 285 | tgcggggcgcgcgctctct | 1219 | ccccctctgcagaatgcca | 2153 | tggcattctgcagaggggg | 3052 |
| gagagcgcgcgccccgcag | 286 | ctgcggggcgcgcgctctc | 1220 | cccctctgcagaatgccaa | 2154 | ttggcattctgcagagggg | 3053 |
| agagcgcgcgccccgcagc | 287 | gctgcggggcgcgcgctct | 1221 | ccctctgcagaatgccaag | 2155 | cttggcattctgcagaggg | 3054 |
| gagcgcgcgccccgcagcc | 288 | ggctgcggggcgcgcgctc | 1222 | cctctgcagaatgccaaga | 2156 | tcttggcattctgcagagg | 3055 |
| agcgcgcgccccgcagccc | 289 | gggctgcggggcgcgcgct | 1223 | ctctgcagaatgccaagaa | 2157 | ttcttggcattctgcagag | 3056 |
| gcgcgcgccccgcagcccc | 290 | ggggctgcggggcgcgcgc | 1224 | tctgcagaatgccaagaac | 2158 | gttcttggcattctgcaga | 3057 |
| cgcgcgccccgcagccccg | 291 | cggggctgcggggcgcgcg | 1225 | ctgcagaatgccaagaacc | 2159 | ggttcttggcattctgcag | 3058 |
| gcgcgccccgcagccccgc | 292 | gcggggctgcggggcgcgc | 1226 | tgcagaatgccaagaacca | 2160 | tggttcttggcattctgca | 3059 |
| cgcgccccgcagccccgcg | 293 | cgcggggctgcggggcgcg | 1227 | gcagaatgccaagaaccac | 2161 | gtggttcttggcattctgc | 3060 |
| gcgccccgcagccccgcgc | 294 | gcgcggggctgcggggcgc | 1228 | cagaatgccaagaaccacg | 2162 | cgtggttcttggcattctg | 3061 |
| cgccccgcagccccgcgcc | 295 | ggcgcggggctgcggggcg | 1229 | agaatgccaagaaccacga | 2163 | tcgtggttcttggcattct | 3062 |
| gccccgcagccccgcgcct | 296 | aggcgcggggctgcggggc | 1230 | gaatgccaagaaccacgac | 2164 | gtcgtggttcttggcattc | 3063 |
| ccccgcagccccgcgccta | 297 | taggcgcggggctgcgggg | 1231 | aatgccaagaaccacgacc | 2165 | ggtcgtggttcttggcatt | 3064 |
| cccgcagccccgcgcctag | 298 | ctaggcgcggggctgcggg | 1232 | atgccaagaaccacgacca | 2166 | tggtcgtggttcttggcat | 3065 |
| ccgcagccccgcgcctagc | 299 | gctaggcgcggggctgcgg | 1233 | tgccaagaaccacgaccat | 2167 | atggtcgtggttcttggca | 3066 |
| cgcagccccgcgcctagcc | 300 | ggctaggcgcggggctgcg | 1234 | gccaagaaccacgaccatg | 2168 | catggtcgtggttcttggc | 3067 |
| gcagccccgcgcctagccc | 301 | gggctaggcgcggggctgc | 1235 | ccaagaaccacgaccatgc | 2169 | gcatggtcgtggttcttgg | 3068 |
| cagccccgcgcctagcccg | 302 | cgggctaggcgcggggctg | 1236 | caagaaccacgaccatgcc | 2170 | ggcatggtcgtggttcttg | 3069 |
| agccccgcgcctagcccgc | 303 | gcgggctaggcgcggggct | 1237 | aagaaccacgaccatgcct | 2171 | aggcatggtcgtggttctt | 3070 |
| gccccgcgcctagcccgcc | 304 | ggcgggctaggcgcggggc | 1238 | agaaccacgaccatgcctg | 2172 | caggcatggtcgtggttct | 3071 |
| ccccgcgcctagcccgccg | 305 | cggcgggctaggcgcgggg | 1239 | gaaccacgaccatgcctgc | 2173 | gcaggcatggtcgtggttc | 3072 |
| cccgcgcctagcccgccgg | 306 | ccggcgggctaggcgcggg | 1240 | aaccacgaccatgcctgca | 2174 | tgcaggcatggtcgtggtt | 3073 |
| ccgcgcctagcccgccggg | 307 | cccggcgggctaggcgcgg | 1241 | accacgaccatgcctgcat | 2175 | atgcaggcatggtcgtggt | 3074 |
| cgcgcctagcccgccgggc | 308 | gcccggcgggctaggcgcg | 1242 | ccacgaccatgcctgcatc | 2176 | gatgcaggcatggtcgtgg | 3075 |
| gcgcctagcccgccgggca | 309 | tgcccggcgggctaggcgc | 1243 | cacgaccatgcctgcatcg | 2177 | cgatgcaggcatggtcgtg | 3076 |
| cgcctagcccgccgggcat | 310 | atgcccggcgggctaggcg | 1244 | acgaccatgcctgcatcgc | 2178 | gcgatgcaggcatggtcgt | 3077 |
| gcctagcccgccgggcatg | 311 | catgcccggcgggctaggc | 1245 | cgaccatgcctgcatcgcc | 2179 | ggcgatgcaggcatggtcg | 3078 |
| cctagcccgccgggcatgg | 312 | ccatgcccggcgggctagg | 1246 | gaccatgcctgcatcgcct | 2180 | aggcgatgcaggcatggtc | 3079 |
| ctagcccgccgggcatggg | 313 | cccatgcccggcgggctag | 1247 | accatgcctgcatcgcctg | 2181 | caggcgatgcaggcatggt | 3080 |
| tagcccgccgggcatgggg | 314 | ccccatgcccggcgggcta | 1248 | ccatgcctgcatcgcctgt | 2182 | acaggcgatgcaggcatgg | 3081 |
| agcccgccgggcatggggc | 315 | gccccatgcccggcgggct | 1249 | catgcctgcatcgcctgtc | 2183 | gacaggcgatgcaggcatg | 3082 |
| gcccgccgggcatggggcg | 316 | cgccccatgcccggcgggc | 1250 | atgcctgcatcgcctgtcg | 2184 | cgacaggcgatgcaggcat | 3083 |
| cccgccgggcatggggcgc | 317 | gcgccccatgcccggcggg | 1251 | tgcctgcatcgcctgtcgg | 2185 | ccgacaggcgatgcaggca | 3084 |
| ccgccgggcatggggcgcg | 318 | cgcgccccatgcccggcgg | 1252 | gcctgcatcgcctgtcgga | 2186 | tccgacaggcgatgcaggc | 3085 |
| cgccgggcatggggcgcgc | 319 | gcgcgccccatgcccggcg | 1253 | cctgcatcgcctgtcggat | 2187 | atccgacaggcgatgcagg | 3086 |
| gccgggcatggggcgcgcg | 320 | cgcgcgccccatgcccggc | 1254 | ctgcatcgcctgtcggatc | 2188 | gatccgacaggcgatgcag | 3087 |
| ccgggcatggggcgcgcgg | 321 | ccgcgcgccccatgcccgg | 1255 | tgcatcgcctgtcggatca | 2189 | tgatccgacaggcgatgca | 3088 |
| cgggcatggggcgcgcggc | 322 | gccgcgcgccccatgcccg | 1256 | gcatcgcctgtcggatcat | 2190 | atgatccgacaggcgatgc | 3089 |
| gggcatggggcgcgcggca | 323 | tgccgcgcgccccatgccc | 1257 | catcgcctgtcggatcatc | 2191 | gatgatccgacaggcgatg | 3090 |
| ggcatggggcgcgcggcag | 324 | ctgccgcgcgccccatgcc | 1258 | atcgcctgtcggatcatct | 2192 | agatgatccgacaggcgat | 3091 |
| gcatggggcgcgcggcagc | 325 | gctgccgcgcgccccatgc | 1259 | tcgcctgtcggatcatcta | 2193 | tagatgatccgacaggcga | 3092 |
| catggggcgcgcggcagcc | 326 | ggctgccgcgcgccccatg | 1260 | cgcctgtcggatcatctat | 2194 | atagatgatccgacaggcg | 3093 |
| atggggcgcgcggcagccg | 327 | cggctgccgcgcgccccat | 1261 | gcctgtcggatcatctatc | 2195 | gatagatgatccgacaggc | 3094 |
| tggggcgcgcggcagccgc | 328 | gcggctgccgcgcgcccca | 1262 | cctgtcggatcatctatcg | 2196 | cgatagatgatccgacagg | 3095 |
| ggggcgcgcggcagccgcc | 329 | ggcggctgccgcgcgcccc | 1263 | ctgtcggatcatctatcgg | 2197 | ccgatagatgatccgacag | 3096 |
| gggcgcgcggcagccgcct | 330 | aggcggctgccgcgcgccc | 1264 | tgtcggatcatctatcggt | 2198 | accgatagatgatccgaca | 3097 |
| ggcgcgcggcagccgcctg | 331 | caggcggctgccgcgcgcc | 1265 | gtcggatcatctatcggtc | 2199 | gaccgatagatgatccgac | 3098 |
| gcgcgcggcagccgcctga | 332 | tcaggcggctgccgcgcgc | 1266 | tcggatcatctatcggtca | 2200 | tgaccgatagatgatccga | 3099 |
| cgcgcggcagccgcctgaa | 333 | ttcaggcggctgccgcgcg | 1267 | cggatcatctatcggtcag | 2201 | ctgaccgatagatgatccg | 3100 |
| gcgcggcagccgcctgaag | 334 | cttcaggcggctgccgcgc | 1268 | ggatcatctatcggtcaga | 2202 | tctgaccgatagatgatcc | 3101 |
| cgcggcagccgcctgaagc | 335 | gcttcaggcggctgccgcg | 1269 | gatcatctatcggtcagac | 2203 | gtctgaccgatagatgatc | 3102 |
| gcggcagccgcctgaagcc | 336 | ggcttcaggcggctgccgc | 1270 | atcatctatcggtcagacg | 2204 | cgtctgaccgatagatgat | 3103 |
| cggcagccgcctgaagccc | 337 | gggcttcaggcggctgccg | 1271 | tcatctatcggtcagacga | 2205 | tcgtctgaccgatagatga | 3104 |
| ggcagccgcctgaagcccc | 338 | ggggcttcaggcggctgcc | 1272 | catctatcggtcagacgag | 2206 | ctcgtctgaccgatagatg | 3105 |
| gcagccgcctgaagccccg | 339 | cggggcttcaggcggctgc | 1273 | atctatcggtcagacgagc | 2207 | gctcgtctgaccgatagat | 3106 |
| cagccgcctgaagccccgg | 340 | ccggggcttcaggcggctg | 1274 | tctatcggtcagacgagca | 2208 | tgctcgtctgaccgataga | 3107 |
| agccgcctgaagccccggc | 341 | gccggggcttcaggcggct | 1275 | ctatcggtcagacgagcac | 2209 | gtgctcgtctgaccgatag | 3108 |
| gccgcctgaagccccggcc | 342 | ggccggggcttcaggcggc | 1276 | tatcggtcagacgagcacc | 2210 | ggtgctcgtctgaccgata | 3109 |
| ccgcctgaagccccggcct | 343 | aggccggggcttcaggcgg | 1277 | atcggtcagacgagcacca | 2211 | tggtgctcgtctgaccgat | 3110 |
| cgcctgaagccccggcctg | 344 | caggccggggcttcaggcg | 1278 | tcggtcagacgagcaccac | 2212 | gtggtgctcgtctgaccga | 3111 |
| gcctgaagccccggcctgg | 345 | ccaggccggggcttcaggc | 1279 | cggtcagacgagcaccacc | 2213 | ggtggtgctcgtctgaccg | 3112 |
| cctgaagccccggcctggc | 346 | gccaggccggggcttcagg | 1280 | ggtcagacgagcaccaccc | 2214 | gggtggtgctcgtctgacc | 3113 |
| ctgaagccccggcctggcc | 347 | ggccaggccggggcttcag | 1281 | gtcagacgagcaccaccct | 2215 | agggtggtgctcgtctgac | 3114 |
| tgaagccccggcctggccc | 348 | gggccaggccggggcttca | 1282 | tcagacgagcaccaccctc | 2216 | gagggtggtgctcgtctga | 3115 |
| gaagccccggcctggcccg | 349 | cgggccaggccggggcttc | 1283 | cagacgagcaccaccctcc | 2217 | ggagggtggtgctcgtctg | 3116 |
| aagccccggcctggcccgg | 350 | ccgggccaggccggggctt | 1284 | agacgagcaccaccctccc | 2218 | gggagggtggtgctcgtct | 3117 |
| agccccggcctggcccggc | 351 | gccgggccaggccggggct | 1285 | gacgagcaccaccctccca | 2219 | tgggagggtggtgctcgtc | 3118 |
| gccccggcctggcccggcc | 352 | ggccgggccaggccggggc | 1286 | acgagcaccaccctcccat | 2220 | atgggagggtggtgctcgt | 3119 |
| ccccggcctggcccggccg | 353 | cggccgggccaggccgggg | 1287 | cgagcaccaccctcccatc | 2221 | gatgggagggtggtgctcg | 3120 |
| cccggcctggcccggccgc | 354 | gcggccgggccaggccggg | 1288 | gagcaccaccctcccatcc | 2222 | ggatgggagggtggtgctc | 3121 |
| ccggcctggcccggccgca | 355 | tgcggccgggccaggccgg | 1289 | agcaccaccctcccatcct | 2223 | aggatgggagggtggtgct | 3122 |
| cggcctggcccggccgcac | 356 | gtgcggccgggccaggccg | 1290 | gcaccaccctcccatcctg | 2224 | caggatgggagggtggtgc | 3123 |
| ggcctggcccggccgcacc | 357 | ggtgcggccgggccaggcc | 1291 | caccaccctcccatcctgc | 2225 | gcaggatgggagggtggtg | 3124 |
| gcctggcccggccgcaccc | 358 | gggtgcggccgggccaggc | 1292 | accaccctcccatcctgcc | 2226 | ggcaggatgggagggtggt | 3125 |
| cctggcccggccgcacccg | 359 | cgggtgcggccgggccagg | 1293 | ccaccctcccatcctgccc | 2227 | gggcaggatgggagggtgg | 3126 |
| ctggcccggccgcacccgg | 360 | ccgggtgcggccgggccag | 1294 | caccctcccatcctgcccc | 2228 | ggggcaggatgggagggtg | 3127 |
| tggcccggccgcacccggc | 361 | gccgggtgcggccgggcca | 1295 | accctcccatcctgccccc | 2229 | gggggcaggatgggagggt | 3128 |
| ggcccggccgcacccggcc | 362 | ggccgggtgcggccgggcc | 1296 | ccctcccatcctgccccca | 2230 | tgggggcaggatgggaggg | 3129 |
| gcccggccgcacccggccg | 363 | cggccgggtgcggccgggc | 1297 | cctcccatcctgcccccaa | 2231 | ttgggggcaggatgggagg | 3130 |
| cccggccgcacccggccgg | 364 | ccggccgggtgcggccggg | 1298 | ctcccatcctgcccccaaa | 2232 | tttgggggcaggatgggag | 3131 |
| ccggccgcacccggccgga | 365 | tccggccgggtgcggccgg | 1299 | tcccatcctgcccccaaag | 2233 | ctttgggggcaggatggga | 3132 |
| cggccgcacccggccggag | 366 | ctccggccgggtgcggccg | 1300 | cccatcctgcccccaaagg | 2234 | cctttgggggcaggatggg | 3133 |
| ggccgcacccggccggagg | 367 | cctccggccgggtgcggcc | 1301 | ccatcctgcccccaaaggc | 2235 | gcctttgggggcaggatgg | 3134 |
| gccgcacccggccggaggc | 368 | gcctccggccgggtgcggc | 1302 | catcctgcccccaaaggca | 2236 | tgcctttgggggcaggatg | 3135 |
| ccgcacccggccggaggcg | 369 | cgcctccggccgggtgcgg | 1303 | atcctgcccccaaaggcag | 2237 | ctgcctttgggggcaggat | 3136 |
| cgcacccggccggaggcgg | 370 | ccgcctccggccgggtgcg | 1304 | tcctgcccccaaaggcaga | 2238 | tctgcctttgggggcagga | 3137 |
| gcacccggccggaggcgga | 371 | tccgcctccggccgggtgc | 1305 | cctgcccccaaaggcagac | 2239 | gtctgcctttgggggcagg | 3138 |
| cacccggccggaggcggag | 372 | ctccgcctccggccgggtg | 1306 | ctgcccccaaaggcagacc | 2240 | ggtctgcctttgggggcag | 3139 |
| acccggccggaggcggagg | 373 | cctccgcctccggccgggt | 1307 | tgcccccaaaggcagacct | 2241 | aggtctgcctttgggggca | 3140 |
| cccggccggaggcggaggg | 374 | ccctccgcctccggccggg | 1308 | gcccccaaaggcagacctg | 2242 | caggtctgcctttgggggc | 3141 |
| ccggccggaggcggagggc | 375 | gccctccgcctccggccgg | 1309 | cccccaaaggcagacctga | 2243 | tcaggtctgcctttggggg | 3142 |
| cggccggaggcggagggca | 376 | tgccctccgcctccggccg | 1310 | ccccaaaggcagacctgac | 2244 | gtcaggtctgcctttgggg | 3143 |
| ggccggaggcggagggcag | 377 | ctgccctccgcctccggcc | 1311 | cccaaaggcagacctgacc | 2245 | ggtcaggtctgcctttggg | 3144 |
| gccggaggcggagggcaga | 378 | tctgccctccgcctccggc | 1312 | ccaaaggcagacctgacca | 2246 | tggtcaggtctgcctttgg | 3145 |
| ccggaggcggagggcagag | 379 | ctctgccctccgcctccgg | 1313 | caaaggcagacctgaccat | 2247 | atggtcaggtctgcctttg | 3146 |
| cggaggcggagggcagagc | 380 | gctctgccctccgcctccg | 1314 | aaaggcagacctgaccatc | 2248 | gatggtcaggtctgccttt | 3147 |
| ggaggcggagggcagagcg | 381 | cgctctgccctccgcctcc | 1315 | aaggcagacctgaccatcg | 2249 | cgatggtcaggtctgcctt | 3148 |
| gaggcggagggcagagcgc | 382 | gcgctctgccctccgcctc | 1316 | aggcagacctgaccatcgg | 2250 | ccgatggtcaggtctgcct | 3149 |
| aggcggagggcagagcgcg | 383 | cgcgctctgccctccgcct | 1317 | ggcagacctgaccatcggc | 2251 | gccgatggtcaggtctgcc | 3150 |
| ggcggagggcagagcgcgc | 384 | gcgcgctctgccctccgcc | 1318 | gcagacctgaccatcggcc | 2252 | ggccgatggtcaggtctgc | 3151 |
| gcggagggcagagcgcgcg | 385 | cgcgcgctctgccctccgc | 1319 | cagacctgaccatcggcct | 2253 | aggccgatggtcaggtctg | 3152 |
| cggagggcagagcgcgcgc | 386 | gcgcgcgctctgccctccg | 1320 | agacctgaccatcggcctg | 2254 | caggccgatggtcaggtct | 3153 |
| ggagggcagagcgcgcgcc | 387 | ggcgcgcgctctgccctcc | 1321 | gacctgaccatcggcctgc | 2255 | gcaggccgatggtcaggtc | 3154 |
| gagggcagagcgcgcgccc | 388 | gggcgcgcgctctgccctc | 1322 | acctgaccatcggcctgca | 2256 | tgcaggccgatggtcaggt | 3155 |
| agggcagagcgcgcgccca | 389 | tgggcgcgcgctctgccct | 1323 | cctgaccatcggcctgcac | 2257 | gtgcaggccgatggtcagg | 3156 |
| gggcagagcgcgcgcccag | 390 | ctgggcgcgcgctctgccc | 1324 | ctgaccatcggcctgcacg | 2258 | cgtgcaggccgatggtcag | 3157 |
| ggcagagcgcgcgcccagt | 391 | actgggcgcgcgctctgcc | 1325 | tgaccatcggcctgcacgg | 2259 | ccgtgcaggccgatggtca | 3158 |
| gcagagcgcgcgcccagtt | 392 | aactgggcgcgcgctctgc | 1326 | gaccatcggcctgcacggg | 2260 | cccgtgcaggccgatggtc | 3159 |
| cagagcgcgcgcccagttg | 393 | caactgggcgcgcgctctg | 1327 | accatcggcctgcacgggg | 2261 | ccccgtgcaggccgatggt | 3160 |
| agagcgcgcgcccagttgc | 394 | gcaactgggcgcgcgctct | 1328 | ccatcggcctgcacgggga | 2262 | tccccgtgcaggccgatgg | 3161 |
| gagcgcgcgcccagttgcc | 395 | ggcaactgggcgcgcgctc | 1329 | catcggcctgcacggggag | 2263 | ctccccgtgcaggccgatg | 3162 |
| agcgcgcgcccagttgccc | 396 | gggcaactgggcgcgcgct | 1330 | atcggcctgcacggggagt | 2264 | actccccgtgcaggccgat | 3163 |
| gcgcgcgcccagttgcccg | 397 | cgggcaactgggcgcgcgc | 1331 | tcggcctgcacggggagtg | 2265 | cactccccgtgcaggccga | 3164 |
| cgcgcgcccagttgcccgg | 398 | ccgggcaactgggcgcgcg | 1332 | cggcctgcacggggagtgg | 2266 | ccactccccgtgcaggccg | 3165 |
| gcgcgcccagttgcccggg | 399 | cccgggcaactgggcgcgc | 1333 | ggcctgcacggggagtggg | 2267 | cccactccccgtgcaggcc | 3166 |
| cgcgcccagttgcccgggc | 400 | gcccgggcaactgggcgcg | 1334 | gcctgcacggggagtgggt | 2268 | acccactccccgtgcaggc | 3167 |
| gcgcccagttgcccgggca | 401 | tgcccgggcaactgggcgc | 1335 | cctgcacggggagtgggtg | 2269 | cacccactccccgtgcagg | 3168 |
| cgcccagttgcccgggcac | 402 | gtgcccgggcaactgggcg | 1336 | ctgcacggggagtgggtga | 2270 | tcacccactccccgtgcag | 3169 |
| gcccagttgcccgggcacc | 403 | ggtgcccgggcaactgggc | 1337 | tgcacggggagtgggtgag | 2271 | ctcacccactccccgtgca | 3170 |
| cccagttgcccgggcacca | 404 | tggtgcccgggcaactggg | 1338 | gcacggggagtgggtgagc | 2272 | gctcacccactccccgtgc | 3171 |
| ccagttgcccgggcaccaa | 405 | ttggtgcccgggcaactgg | 1339 | cacggggagtgggtgagcc | 2273 | ggctcacccactccccgtg | 3172 |
| cagttgcccgggcaccaaa | 406 | tttggtgcccgggcaactg | 1340 | acggggagtgggtgagcca | 2274 | tggctcacccactccccgt | 3173 |
| agttgcccgggcaccaaat | 407 | atttggtgcccgggcaact | 1341 | cggggagtgggtgagccag | 2275 | ctggctcacccactccccg | 3174 |
| gttgcccgggcaccaaatc | 408 | gatttggtgcccgggcaac | 1342 | ggggagtgggtgagccagc | 2276 | gctggctcacccactcccc | 3175 |
| ttgcccgggcaccaaatcg | 409 | cgatttggtgcccgggcaa | 1343 | gggagtgggtgagccagcg | 2277 | cgctggctcacccactccc | 3176 |
| tgcccgggcaccaaatcgg | 410 | ccgatttggtgcccgggca | 1344 | ggagtgggtgagccagcgc | 2278 | gcgctggctcacccactcc | 3177 |
| gcccgggcaccaaatcgga | 411 | tccgatttggtgcccgggc | 1345 | gagtgggtgagccagcgct | 2279 | agcgctggctcacccactc | 3178 |
| cccgggcaccaaatcggag | 412 | ctccgatttggtgcccggg | 1346 | agtgggtgagccagcgctg | 2280 | cagcgctggctcacccact | 3179 |
| ccgggcaccaaatcggagc | 413 | gctccgatttggtgcccgg | 1347 | gtgggtgagccagcgctgt | 2281 | acagcgctggctcacccac | 3180 |
| cgggcaccaaatcggagcg | 414 | cgctccgatttggtgcccg | 1348 | tgggtgagccagcgctgtg | 2282 | cacagcgctggctcaccca | 3181 |
| gggcaccaaatcggagcgc | 415 | gcgctccgatttggtgccc | 1349 | gggtgagccagcgctgtga | 2283 | tcacagcgctggctcaccc | 3182 |
| ggcaccaaatcggagcgcg | 416 | cgcgctccgatttggtgcc | 1350 | ggtgagccagcgctgtgag | 2284 | ctcacagcgctggctcacc | 3183 |
| gcaccaaatcggagcgcgg | 417 | ccgcgctccgatttggtgc | 1351 | gtgagccagcgctgtgagg | 2285 | cctcacagcgctggctcac | 3184 |
| caccaaatcggagcgcggc | 418 | gccgcgctccgatttggtg | 1352 | tgagccagcgctgtgaggt | 2286 | acctcacagcgctggctca | 3185 |
| accaaatcggagcgcggcg | 419 | cgccgcgctccgatttggt | 1353 | gagccagcgctgtgaggtg | 2287 | cacctcacagcgctggctc | 3186 |
| ccaaatcggagcgcggcgt | 420 | acgccgcgctccgatttgg | 1354 | agccagcgctgtgaggtgc | 2288 | gcacctcacagcgctggct | 3187 |
| caaatcggagcgcggcgtg | 421 | cacgccgcgctccgatttg | 1355 | gccagcgctgtgaggtgcg | 2289 | cgcacctcacagcgctggc | 3188 |
| aaatcggagcgcggcgtgc | 422 | gcacgccgcgctccgattt | 1356 | ccagcgctgtgaggtgcgc | 2290 | gcgcacctcacagcgctgg | 3189 |
| aatcggagcgcggcgtgcg | 423 | cgcacgccgcgctccgatt | 1357 | cagcgctgtgaggtgcgcc | 2291 | ggcgcacctcacagcgctg | 3190 |
| atcggagcgcggcgtgcgg | 424 | ccgcacgccgcgctccgat | 1358 | agcgctgtgaggtgcgccc | 2292 | gggcgcacctcacagcgct | 3191 |
| tcggagcgcggcgtgcggg | 425 | cccgcacgccgcgctccga | 1359 | gcgctgtgaggtgcgcccc | 2293 | ggggcgcacctcacagcgc | 3192 |
| cggagcgcggcgtgcggga | 426 | tcccgcacgccgcgctccg | 1360 | cgctgtgaggtgcgccccg | 2294 | cggggcgcacctcacagcg | 3193 |
| ggagcgcggcgtgcgggag | 427 | ctcccgcacgccgcgctcc | 1361 | gctgtgaggtgcgccccga | 2295 | tcggggcgcacctcacagc | 3194 |
| gagcgcggcgtgcgggagg | 428 | cctcccgcacgccgcgctc | 1362 | ctgtgaggtgcgccccgaa | 2296 | ttcggggcgcacctcacag | 3195 |
| agcgcggcgtgcgggaggc | 429 | gcctcccgcacgccgcgct | 1363 | tgtgaggtgcgccccgaag | 2297 | cttcggggcgcacctcaca | 3196 |
| gcgcggcgtgcgggaggcc | 430 | ggcctcccgcacgccgcgc | 1364 | gtgaggtgcgccccgaagt | 2298 | acttcggggcgcacctcac | 3197 |
| cgcggcgtgcgggaggccc | 431 | gggcctcccgcacgccgcg | 1365 | tgaggtgcgccccgaagtc | 2299 | gacttcggggcgcacctca | 3198 |
| gcggcgtgcgggaggcccc | 432 | ggggcctcccgcacgccgc | 1366 | gaggtgcgccccgaagtcc | 2300 | ggacttcggggcgcacctc | 3199 |
| cggcgtgcgggaggcccca | 433 | tggggcctcccgcacgccg | 1367 | aggtgcgccccgaagtcct | 2301 | aggacttcggggcgcacct | 3200 |
| ggcgtgcgggaggccccag | 434 | ctggggcctcccgcacgcc | 1368 | ggtgcgccccgaagtcctc | 2302 | gaggacttcggggcgcacc | 3201 |
| gcgtgcgggaggccccaga | 435 | tctggggcctcccgcacgc | 1369 | gtgcgccccgaagtcctct | 2303 | agaggacttcggggcgcac | 3202 |
| cgtgcgggaggccccagag | 436 | ctctggggcctcccgcacg | 1370 | tgcgccccgaagtcctctt | 2304 | aagaggacttcggggcgca | 3203 |
| gtgcgggaggccccagagc | 437 | gctctggggcctcccgcac | 1371 | gcgccccgaagtcctcttc | 2305 | gaagaggacttcggggcgc | 3204 |
| tgcgggaggccccagagca | 438 | tgctctggggcctcccgca | 1372 | cgccccgaagtcctcttcc | 2306 | ggaagaggacttcggggcg | 3205 |
| gcgggaggccccagagcag | 439 | ctgctctggggcctcccgc | 1373 | gccccgaagtcctcttcct | 2307 | aggaagaggacttcggggc | 3206 |
| cgggaggccccagagcagg | 440 | cctgctctggggcctcccg | 1374 | ccccgaagtcctcttcctc | 2308 | gaggaagaggacttcgggg | 3207 |
| gggaggccccagagcagga | 441 | tcctgctctggggcctccc | 1375 | cccgaagtcctcttcctca | 2309 | tgaggaagaggacttcggg | 3208 |
| ggaggccccagagcaggac | 442 | gtcctgctctggggcctcc | 1376 | ccgaagtcctcttcctcac | 2310 | gtgaggaagaggacttcgg | 3209 |
| gaggccccagagcaggact | 443 | agtcctgctctggggcctc | 1377 | cgaagtcctcttcctcacc | 2311 | ggtgaggaagaggacttcg | 3210 |
| aggccccagagcaggactg | 444 | cagtcctgctctggggcct | 1378 | gaagtcctcttcctcaccc | 2312 | gggtgaggaagaggacttc | 3211 |
| ggccccagagcaggactgg | 445 | ccagtcctgctctggggcc | 1379 | aagtcctcttcctcacccg | 2313 | cgggtgaggaagaggactt | 3212 |
| gccccagagcaggactgga | 446 | tccagtcctgctctggggc | 1380 | agtcctcttcctcacccgc | 2314 | gcgggtgaggaagaggact | 3213 |
| ccccagagcaggactggaa | 447 | ttccagtcctgctctgggg | 1381 | gtcctcttcctcacccgcc | 2315 | ggcgggtgaggaagaggac | 3214 |
| cccagagcaggactggaaa | 448 | tttccagtcctgctctggg | 1382 | tcctcttcctcacccgcca | 2316 | tggcgggtgaggaagagga | 3215 |
| ccagagcaggactggaaat | 449 | atttccagtcctgctctgg | 1383 | cctcttcctcacccgccac | 2317 | gtggcgggtgaggaagagg | 3216 |
| cagagcaggactggaaatg | 450 | catttccagtcctgctctg | 1384 | ctcttcctcacccgccact | 2318 | agtggcgggtgaggaagag | 3217 |
| agagcaggactggaaatgt | 451 | acatttccagtcctgctct | 1385 | tcttcctcacccgccactt | 2319 | aagtggcgggtgaggaaga | 3218 |
| gagcaggactggaaatgtc | 452 | gacatttccagtcctgctc | 1386 | cttcctcacccgccacttc | 2320 | gaagtggcgggtgaggaag | 3219 |
| agcaggactggaaatgtcc | 453 | ggacatttccagtcctgct | 1387 | ttcctcacccgccacttca | 2321 | tgaagtggcgggtgaggaa | 3220 |
| gcaggactggaaatgtcct | 454 | aggacatttccagtcctgc | 1388 | tcctcacccgccacttcat | 2322 | atgaagtggcgggtgagga | 3221 |
| caggactggaaatgtcctg | 455 | caggacatttccagtcctg | 1389 | cctcacccgccacttcatc | 2323 | gatgaagtggcgggtgagg | 3222 |
| aggactggaaatgtcctgg | 456 | ccaggacatttccagtcct | 1390 | ctcacccgccacttcatct | 2324 | agatgaagtggcgggtgag | 3223 |
| ggactggaaatgtcctggc | 457 | gccaggacatttccagtcc | 1391 | tcacccgccacttcatctt | 2325 | aagatgaagtggcgggtga | 3224 |
| gactggaaatgtcctggcc | 458 | ggccaggacatttccagtc | 1392 | cacccgccacttcatcttc | 2326 | gaagatgaagtggcgggtg | 3225 |
| actggaaatgtcctggccg | 459 | cggccaggacatttccagt | 1393 | acccgccacttcatcttcc | 2327 | ggaagatgaagtggcgggt | 3226 |
| ctggaaatgtcctggccgc | 460 | gcggccaggacatttccag | 1394 | cccgccacttcatcttcca | 2328 | tggaagatgaagtggcggg | 3227 |
| tggaaatgtcctggccgcg | 461 | cgcggccaggacatttcca | 1395 | ccgccacttcatcttccat | 2329 | atggaagatgaagtggcgg | 3228 |
| ggaaatgtcctggccgcgc | 462 | gcgcggccaggacatttcc | 1396 | cgccacttcatcttccatg | 2330 | catggaagatgaagtggcg | 3229 |
| gaaatgtcctggccgcgcc | 463 | ggcgcggccaggacatttc | 1397 | gccacttcatcttccatga | 2331 | tcatggaagatgaagtggc | 3230 |
| aaatgtcctggccgcgccg | 464 | cggcgcggccaggacattt | 1398 | ccacttcatcttccatgac | 2332 | gtcatggaagatgaagtgg | 3231 |
| aatgtcctggccgcgccgc | 465 | gcggcgcggccaggacatt | 1399 | cacttcatcttccatgaca | 2333 | tgtcatggaagatgaagtg | 3232 |
| atgtcctggccgcgccgcc | 466 | ggcggcgcggccaggacat | 1400 | acttcatcttccatgacaa | 2334 | ttgtcatggaagatgaagt | 3233 |
| tgtcctggccgcgccgcct | 467 | aggcggcgcggccaggaca | 1401 | cttcatcttccatgacaac | 2335 | gttgtcatggaagatgaag | 3234 |
| gtcctggccgcgccgcctc | 468 | gaggcggcgcggccaggac | 1402 | ttcatcttccatgacaaca | 2336 | tgttgtcatggaagatgaa | 3235 |
| tcctggccgcgccgcctcc | 469 | ggaggcggcgcggccagga | 1403 | tcatcttccatgacaacaa | 2337 | ttgttgtcatggaagatga | 3236 |
| cctggccgcgccgcctcct | 470 | aggaggcggcgcggccagg | 1404 | catcttccatgacaacaac | 2338 | gttgttgtcatggaagatg | 3237 |
| ctggccgcgccgcctcctg | 471 | caggaggcggcgcggccag | 1405 | atcttccatgacaacaaca | 2339 | tgttgttgtcatggaagat | 3238 |
| tggccgcgccgcctcctgc | 472 | gcaggaggcggcgcggcca | 1406 | tcttccatgacaacaacaa | 2340 | ttgttgttgtcatggaaga | 3239 |
| ggccgcgccgcctcctgct | 473 | agcaggaggcggcgcggcc | 1407 | cttccatgacaacaacaac | 2341 | gttgttgttgtcatggaag | 3240 |
| gccgcgccgcctcctgctc | 474 | gagcaggaggcggcgcggc | 1408 | ttccatgacaacaacaaca | 2342 | tgttgttgttgtcatggaa | 3241 |
| ccgcgccgcctcctgctca | 475 | tgagcaggaggcggcgcgg | 1409 | tccatgacaacaacaacac | 2343 | gtgttgttgttgtcatgga | 3242 |
| cgcgccgcctcctgctcag | 476 | ctgagcaggaggcggcgcg | 1410 | ccatgacaacaacaacacc | 2344 | ggtgttgttgttgtcatgg | 3243 |
| gcgccgcctcctgctcaga | 477 | tctgagcaggaggcggcgc | 1411 | catgacaacaacaacacct | 2345 | aggtgttgttgttgtcatg | 3244 |
| cgccgcctcctgctcagat | 478 | atctgagcaggaggcggcg | 1412 | atgacaacaacaacacctg | 2346 | caggtgttgttgttgtcat | 3245 |
| gccgcctcctgctcagata | 479 | tatctgagcaggaggcggc | 1413 | tgacaacaacaacacctgg | 2347 | ccaggtgttgttgttgtca | 3246 |
| ccgcctcctgctcagatac | 480 | gtatctgagcaggaggcgg | 1414 | gacaacaacaacacctggg | 2348 | cccaggtgttgttgttgtc | 3247 |
| cgcctcctgctcagatacc | 481 | ggtatctgagcaggaggcg | 1415 | acaacaacaacacctggga | 2349 | tcccaggtgttgttgttgt | 3248 |
| gcctcctgctcagatacct | 482 | aggtatctgagcaggaggc | 1416 | caacaacaacacctgggag | 2350 | ctcccaggtgttgttgttg | 3249 |
| cctcctgctcagatacctg | 483 | caggtatctgagcaggagg | 1417 | aacaacaacacctgggagg | 2351 | cctcccaggtgttgttgtt | 3250 |
| ctcctgctcagatacctgt | 484 | acaggtatctgagcaggag | 1418 | acaacaacacctgggaggg | 2352 | ccctcccaggtgttgttgt | 3251 |
| tcctgctcagatacctgtt | 485 | aacaggtatctgagcagga | 1419 | caacaacacctgggagggc | 2353 | gccctcccaggtgttgttg | 3252 |
| cctgctcagatacctgttc | 486 | gaacaggtatctgagcagg | 1420 | aacaacacctgggagggcc | 2354 | ggccctcccaggtgttgtt | 3253 |
| ctgctcagatacctgttcc | 487 | ggaacaggtatctgagcag | 1421 | acaacacctgggagggcca | 2355 | tggccctcccaggtgttgt | 3254 |
| tgctcagatacctgttccc | 488 | gggaacaggtatctgagca | 1422 | caacacctgggagggccac | 2356 | gtggccctcccaggtgttg | 3255 |
| gctcagatacctgttcccg | 489 | cgggaacaggtatctgagc | 1423 | aacacctgggagggccact | 2357 | agtggccctcccaggtgtt | 3256 |
| ctcagatacctgttcccgg | 490 | ccgggaacaggtatctgag | 1424 | acacctgggagggccacta | 2358 | tagtggccctcccaggtgt | 3257 |
| tcagatacctgttcccggc | 491 | gccgggaacaggtatctga | 1425 | cacctgggagggccactac | 2359 | gtagtggccctcccaggtg | 3258 |
| cagatacctgttcccggcc | 492 | ggccgggaacaggtatctg | 1426 | acctgggagggccactact | 2360 | agtagtggccctcccaggt | 3259 |
| agatacctgttcccggccc | 493 | gggccgggaacaggtatct | 1427 | cctgggagggccactacta | 2361 | tagtagtggccctcccagg | 3260 |
| gatacctgttcccggccct | 494 | agggccgggaacaggtatc | 1428 | ctgggagggccactactac | 2362 | gtagtagtggccctcccag | 3261 |
| atacctgttcccggccctc | 495 | gagggccgggaacaggtat | 1429 | tgggagggccactactacc | 2363 | ggtagtagtggccctccca | 3262 |
| tacctgttcccggccctcc | 496 | ggagggccgggaacaggta | 1430 | gggagggccactactacca | 2364 | tggtagtagtggccctccc | 3263 |
| acctgttcccggccctcct | 497 | aggagggccgggaacaggt | 1431 | ggagggccactactaccac | 2365 | gtggtagtagtggccctcc | 3264 |
| cctgttcccggccctcctg | 498 | caggagggccgggaacagg | 1432 | gagggccactactaccact | 2366 | agtggtagtagtggccctc | 3265 |
| ctgttcccggccctcctgc | 499 | gcaggagggccgggaacag | 1433 | agggccactactaccacta | 2367 | tagtggtagtagtggccct | 3266 |
| tgttcccggccctcctgct | 500 | agcaggagggccgggaaca | 1434 | gggccactactaccactac | 2368 | gtagtggtagtagtggccc | 3267 |
| gttcccggccctcctgctt | 501 | aagcaggagggccgggaac | 1435 | ggccactactaccactact | 2369 | agtagtggtagtagtggcc | 3268 |
| ttcccggccctcctgcttc | 502 | gaagcaggagggccgggaa | 1436 | gccactactaccactactc | 2370 | gagtagtggtagtagtggc | 3269 |
| tcccggccctcctgcttca | 503 | tgaagcaggagggccggga | 1437 | ccactactaccactactca | 2371 | tgagtagtggtagtagtgg | 3270 |
| cccggccctcctgcttcac | 504 | gtgaagcaggagggccggg | 1438 | cactactaccactactcag | 2372 | ctgagtagtggtagtagtg | 3271 |
| ccggccctcctgcttcacg | 505 | cgtgaagcaggagggccgg | 1439 | actactaccactactcaga | 2373 | tctgagtagtggtagtagt | 3272 |
| cggccctcctgcttcacgg | 506 | ccgtgaagcaggagggccg | 1440 | ctactaccactactcagac | 2374 | gtctgagtagtggtagtag | 3273 |
| ggccctcctgcttcacggg | 507 | cccgtgaagcaggagggcc | 1441 | tactaccactactcagacc | 2375 | ggtctgagtagtggtagta | 3274 |
| gccctcctgcttcacgggc | 508 | gcccgtgaagcaggagggc | 1442 | actaccactactcagaccc | 2376 | gggtctgagtagtggtagt | 3275 |
| ccctcctgcttcacgggct | 509 | agcccgtgaagcaggaggg | 1443 | ctaccactactcagacccg | 2377 | cgggtctgagtagtggtag | 3276 |
| cctcctgcttcacgggctg | 510 | cagcccgtgaagcaggagg | 1444 | taccactactcagacccgg | 2378 | ccgggtctgagtagtggta | 3277 |
| ctcctgcttcacgggctgg | 511 | ccagcccgtgaagcaggag | 1445 | accactactcagacccggt | 2379 | accgggtctgagtagtggt | 3278 |
| tcctgcttcacgggctggg | 512 | cccagcccgtgaagcagga | 1446 | ccactactcagacccggtg | 2380 | caccgggtctgagtagtgg | 3279 |
| cctgcttcacgggctggga | 513 | tcccagcccgtgaagcagg | 1447 | cactactcagacccggtgt | 2381 | acaccgggtctgagtagtg | 3280 |
| ctgcttcacgggctgggag | 514 | ctcccagcccgtgaagcag | 1448 | actactcagacccggtgtg | 2382 | cacaccgggtctgagtagt | 3281 |
| tgcttcacgggctgggaga | 515 | tctcccagcccgtgaagca | 1449 | ctactcagacccggtgtgc | 2383 | gcacaccgggtctgagtag | 3282 |
| gcttcacgggctgggagag | 516 | ctctcccagcccgtgaagc | 1450 | tactcagacccggtgtgca | 2384 | tgcacaccgggtctgagta | 3283 |
| cttcacgggctgggagagg | 517 | cctctcccagcccgtgaag | 1451 | actcagacccggtgtgcaa | 2385 | ttgcacaccgggtctgagt | 3284 |
| ttcacgggctgggagaggg | 518 | ccctctcccagcccgtgaa | 1452 | ctcagacccggtgtgcaag | 2386 | cttgcacaccgggtctgag | 3285 |
| tcacgggctgggagagggt | 519 | accctctcccagcccgtga | 1453 | tcagacccggtgtgcaagc | 2387 | gcttgcacaccgggtctga | 3286 |
| cacgggctgggagagggtt | 520 | aaccctctcccagcccgtg | 1454 | cagacccggtgtgcaagca | 2388 | tgcttgcacaccgggtctg | 3287 |
| acgggctgggagagggttc | 521 | gaaccctctcccagcccgt | 1455 | agacccggtgtgcaagcac | 2389 | gtgcttgcacaccgggtct | 3288 |
| cgggctgggagagggttct | 522 | agaaccctctcccagcccg | 1456 | gacccggtgtgcaagcacc | 2390 | ggtgcttgcacaccgggtc | 3289 |
| gggctgggagagggttctg | 523 | cagaaccctctcccagccc | 1457 | acccggtgtgcaagcaccc | 2391 | gggtgcttgcacaccgggt | 3290 |
| ggctgggagagggttctgc | 524 | gcagaaccctctcccagcc | 1458 | cccggtgtgcaagcacccc | 2392 | ggggtgcttgcacaccggg | 3291 |
| gctgggagagggttctgcc | 525 | ggcagaaccctctcccagc | 1459 | ccggtgtgcaagcacccca | 2393 | tggggtgcttgcacaccgg | 3292 |
| ctgggagagggttctgccc | 526 | gggcagaaccctctcccag | 1460 | cggtgtgcaagcaccccac | 2394 | gtggggtgcttgcacaccg | 3293 |
| tgggagagggttctgccct | 527 | agggcagaaccctctccca | 1461 | ggtgtgcaagcaccccacc | 2395 | ggtggggtgcttgcacacc | 3294 |
| gggagagggttctgccctc | 528 | gagggcagaaccctctccc | 1462 | gtgtgcaagcaccccacct | 2396 | aggtggggtgcttgcacac | 3295 |
| ggagagggttctgccctcc | 529 | ggagggcagaaccctctcc | 1463 | tgtgcaagcaccccacctt | 2397 | aaggtggggtgcttgcaca | 3296 |
| gagagggttctgccctcct | 530 | aggagggcagaaccctctc | 1464 | gtgcaagcaccccaccttc | 2398 | gaaggtggggtgcttgcac | 3297 |
| agagggttctgccctcctt | 531 | aaggagggcagaaccctct | 1465 | tgcaagcaccccaccttct | 2399 | agaaggtggggtgcttgca | 3298 |
| gagggttctgccctccttc | 532 | gaaggagggcagaaccctc | 1466 | gcaagcaccccaccttctc | 2400 | gagaaggtggggtgcttgc | 3299 |
| agggttctgccctccttca | 533 | tgaaggagggcagaaccct | 1467 | caagcaccccaccttctcc | 2401 | ggagaaggtggggtgcttg | 3300 |
| gggttctgccctccttcat | 534 | atgaaggagggcagaaccc | 1468 | aagcaccccaccttctcca | 2402 | tggagaaggtggggtgctt | 3301 |
| ggttctgccctccttcatc | 535 | gatgaaggagggcagaacc | 1469 | agcaccccaccttctccat | 2403 | atggagaaggtggggtgct | 3302 |
| gttctgccctccttcatcc | 536 | ggatgaaggagggcagaac | 1470 | gcaccccaccttctccatc | 2404 | gatggagaaggtggggtgc | 3303 |
| ttctgccctccttcatcca | 537 | tggatgaaggagggcagaa | 1471 | caccccaccttctccatct | 2405 | agatggagaaggtggggtg | 3304 |
| tctgccctccttcatccag | 538 | ctggatgaaggagggcaga | 1472 | accccaccttctccatcta | 2406 | tagatggagaaggtggggt | 3305 |
| ctgccctccttcatccaga | 539 | tctggatgaaggagggcag | 1473 | ccccaccttctccatctac | 2407 | gtagatggagaaggtgggg | 3306 |
| tgccctccttcatccagac | 540 | gtctggatgaaggagggca | 1474 | cccaccttctccatctacg | 2408 | cgtagatggagaaggtggg | 3307 |
| gccctccttcatccagaca | 541 | tgtctggatgaaggagggc | 1475 | ccaccttctccatctacgc | 2409 | gcgtagatggagaaggtgg | 3308 |
| ccctccttcatccagacag | 542 | ctgtctggatgaaggaggg | 1476 | caccttctccatctacgcc | 2410 | ggcgtagatggagaaggtg | 3309 |
| cctccttcatccagacagc | 543 | gctgtctggatgaaggagg | 1477 | accttctccatctacgccc | 2411 | gggcgtagatggagaaggt | 3310 |
| ctccttcatccagacagca | 544 | tgctgtctggatgaaggag | 1478 | ccttctccatctacgcccg | 2412 | cgggcgtagatggagaagg | 3311 |
| tccttcatccagacagcag | 545 | ctgctgtctggatgaagga | 1479 | cttctccatctacgcccgg | 2413 | ccgggcgtagatggagaag | 3312 |
| ccttcatccagacagcagg | 546 | cctgctgtctggatgaagg | 1480 | ttctccatctacgcccggg | 2414 | cccgggcgtagatggagaa | 3313 |
| cttcatccagacagcaggt | 547 | acctgctgtctggatgaag | 1481 | tctccatctacgcccgggg | 2415 | ccccgggcgtagatggaga | 3314 |
| ttcatccagacagcaggtc | 548 | gacctgctgtctggatgaa | 1482 | ctccatctacgcccggggc | 2416 | gccccgggcgtagatggag | 3315 |
| tcatccagacagcaggtct | 549 | agacctgctgtctggatga | 1483 | tccatctacgcccggggcc | 2417 | ggccccgggcgtagatgga | 3316 |
| catccagacagcaggtctc | 550 | gagacctgctgtctggatg | 1484 | ccatctacgcccggggccg | 2418 | cggccccgggcgtagatgg | 3317 |
| atccagacagcaggtctca | 551 | tgagacctgctgtctggat | 1485 | catctacgcccggggccgc | 2419 | gcggccccgggcgtagatg | 3318 |
| tccagacagcaggtctcat | 552 | atgagacctgctgtctgga | 1486 | atctacgcccggggccgct | 2420 | agcggccccgggcgtagat | 3319 |
| ccagacagcaggtctcatc | 553 | gatgagacctgctgtctgg | 1487 | tctacgcccggggccgcta | 2421 | tagcggccccgggcgtaga | 3320 |
| cagacagcaggtctcatcc | 554 | ggatgagacctgctgtctg | 1488 | ctacgcccggggccgctac | 2422 | gtagcggccccgggcgtag | 3321 |
| agacagcaggtctcatcct | 555 | aggatgagacctgctgtct | 1489 | tacgcccggggccgctaca | 2423 | tgtagcggccccgggcgta | 3322 |
| gacagcaggtctcatccta | 556 | taggatgagacctgctgtc | 1490 | acgcccggggccgctacag | 2424 | ctgtagcggccccgggcgt | 3323 |
| acagcaggtctcatcctag | 557 | ctaggatgagacctgctgt | 1491 | cgcccggggccgctacagc | 2425 | gctgtagcggccccgggcg | 3324 |
| cagcaggtctcatcctagg | 558 | cctaggatgagacctgctg | 1492 | gcccggggccgctacagcc | 2426 | ggctgtagcggccccgggc | 3325 |
| agcaggtctcatcctaggt | 559 | acctaggatgagacctgct | 1493 | cccggggccgctacagccg | 2427 | cggctgtagcggccccggg | 3326 |
| gcaggtctcatcctaggtc | 560 | gacctaggatgagacctgc | 1494 | ccggggccgctacagccgc | 2428 | gcggctgtagcggccccgg | 3327 |
| caggtctcatcctaggtcc | 561 | ggacctaggatgagacctg | 1495 | cggggccgctacagccgcg | 2429 | cgcggctgtagcggccccg | 3328 |
| aggtctcatcctaggtcct | 562 | aggacctaggatgagacct | 1496 | ggggccgctacagccgcgg | 2430 | ccgcggctgtagcggcccc | 3329 |
| ggtctcatcctaggtcctt | 563 | aaggacctaggatgagacc | 1497 | gggccgctacagccgcggc | 2431 | gccgcggctgtagcggccc | 3330 |
| gtctcatcctaggtcctta | 564 | taaggacctaggatgagac | 1498 | ggccgctacagccgcggcg | 2432 | cgccgcggctgtagcggcc | 3331 |
| tctcatcctaggtccttag | 565 | ctaaggacctaggatgaga | 1499 | gccgctacagccgcggcgt | 2433 | acgccgcggctgtagcggc | 3332 |
| ctcatcctaggtccttaga | 566 | tctaaggacctaggatgag | 1500 | ccgctacagccgcggcgtc | 2434 | gacgccgcggctgtagcgg | 3333 |
| tcatcctaggtccttagag | 567 | ctctaaggacctaggatga | 1501 | cgctacagccgcggcgtcc | 2435 | ggacgccgcggctgtagcg | 3334 |
| catcctaggtccttagaga | 568 | tctctaaggacctaggatg | 1502 | gctacagccgcggcgtcct | 2436 | aggacgccgcggctgtagc | 3335 |
| atcctaggtccttagagaa | 569 | ttctctaaggacctaggat | 1503 | ctacagccgcggcgtcctc | 2437 | gaggacgccgcggctgtag | 3336 |
| tcctaggtccttagagaaa | 570 | tttctctaaggacctagga | 1504 | tacagccgcggcgtcctct | 2438 | agaggacgccgcggctgta | 3337 |
| cctaggtccttagagaaaa | 571 | ttttctctaaggacctagg | 1505 | acagccgcggcgtcctctc | 2439 | gagaggacgccgcggctgt | 3338 |
| ctaggtccttagagaaaag | 572 | cttttctctaaggacctag | 1506 | cagccgcggcgtcctctcg | 2440 | cgagaggacgccgcggctg | 3339 |
| taggtccttagagaaaagt | 573 | acttttctctaaggaccta | 1507 | agccgcggcgtcctctcgt | 2441 | acgagaggacgccgcggct | 3340 |
| aggtccttagagaaaagtg | 574 | cacttttctctaaggacct | 1508 | gccgcggcgtcctctcgtc | 2442 | gacgagaggacgccgcggc | 3341 |
| ggtccttagagaaaagtgc | 575 | gcacttttctctaaggacc | 1509 | ccgcggcgtcctctcgtcc | 2443 | ggacgagaggacgccgcgg | 3342 |
| gtccttagagaaaagtgcc | 576 | ggcacttttctctaaggac | 1510 | cgcggcgtcctctcgtcca | 2444 | tggacgagaggacgccgcg | 3343 |
| tccttagagaaaagtgcct | 577 | aggcacttttctctaagga | 1511 | gcggcgtcctctcgtccag | 2445 | ctggacgagaggacgccgc | 3344 |
| ccttagagaaaagtgcctg | 578 | caggcacttttctctaagg | 1512 | cggcgtcctctcgtccagg | 2446 | cctggacgagaggacgccg | 3345 |
| cttagagaaaagtgcctgg | 579 | ccaggcacttttctctaag | 1513 | ggcgtcctctcgtccaggg | 2447 | ccctggacgagaggacgcc | 3346 |
| ttagagaaaagtgcctgga | 580 | tccaggcacttttctctaa | 1514 | gcgtcctctcgtccagggt | 2448 | accctggacgagaggacgc | 3347 |
| tagagaaaagtgcctggag | 581 | ctccaggcacttttctcta | 1515 | cgtcctctcgtccagggtc | 2449 | gaccctggacgagaggacg | 3348 |
| agagaaaagtgcctggagg | 582 | cctccaggcacttttctct | 1516 | gtcctctcgtccagggtca | 2450 | tgaccctggacgagaggac | 3349 |
| gagaaaagtgcctggaggg | 583 | ccctccaggcacttttctc | 1517 | tcctctcgtccagggtcat | 2451 | atgaccctggacgagagga | 3350 |
| agaaaagtgcctggagggc | 584 | gccctccaggcacttttct | 1518 | cctctcgtccagggtcatg | 2452 | catgaccctggacgagagg | 3351 |
| gaaaagtgcctggagggct | 585 | agccctccaggcacttttc | 1519 | ctctcgtccagggtcatgg | 2453 | ccatgaccctggacgagag | 3352 |
| aaaagtgcctggagggctt | 586 | aagccctccaggcactttt | 1520 | tctcgtccagggtcatggg | 2454 | cccatgaccctggacgaga | 3353 |
| aaagtgcctggagggcttt | 587 | aaagccctccaggcacttt | 1521 | ctcgtccagggtcatggga | 2455 | tcccatgaccctggacgag | 3354 |
| aagtgcctggagggctttt | 588 | aaaagccctccaggcactt | 1522 | tcgtccagggtcatgggag | 2456 | ctcccatgaccctggacga | 3355 |
| agtgcctggagggctttta | 589 | taaaagccctccaggcact | 1523 | cgtccagggtcatgggagg | 2457 | cctcccatgaccctggacg | 3356 |
| gtgcctggagggcttttaa | 590 | ttaaaagccctccaggcac | 1524 | gtccagggtcatgggaggc | 2458 | gcctcccatgaccctggac | 3357 |
| tgcctggagggcttttaag | 591 | cttaaaagccctccaggca | 1525 | tccagggtcatgggaggca | 2459 | tgcctcccatgaccctgga | 3358 |
| gcctggagggcttttaagg | 592 | ccttaaaagccctccaggc | 1526 | ccagggtcatgggaggcac | 2460 | gtgcctcccatgaccctgg | 3359 |
| cctggagggcttttaagga | 593 | tccttaaaagccctccagg | 1527 | cagggtcatgggaggcacc | 2461 | ggtgcctcccatgaccctg | 3360 |
| ctggagggcttttaaggag | 594 | ctccttaaaagccctccag | 1528 | agggtcatgggaggcaccg | 2462 | cggtgcctcccatgaccct | 3361 |
| tggagggcttttaaggagt | 595 | actccttaaaagccctcca | 1529 | gggtcatgggaggcaccga | 2463 | tcggtgcctcccatgaccc | 3362 |
| ggagggcttttaaggagtc | 596 | gactccttaaaagccctcc | 1530 | ggtcatgggaggcaccgag | 2464 | ctcggtgcctcccatgacc | 3363 |
| gagggcttttaaggagtca | 597 | tgactccttaaaagccctc | 1531 | gtcatgggaggcaccgagt | 2465 | actcggtgcctcccatgac | 3364 |
| agggcttttaaggagtcac | 598 | gtgactccttaaaagccct | 1532 | tcatgggaggcaccgagtt | 2466 | aactcggtgcctcccatga | 3365 |
| gggcttttaaggagtcaca | 599 | tgtgactccttaaaagccc | 1533 | catgggaggcaccgagttc | 2467 | gaactcggtgcctcccatg | 3366 |
| ggcttttaaggagtcacag | 600 | ctgtgactccttaaaagcc | 1534 | atgggaggcaccgagttcg | 2468 | cgaactcggtgcctcccat | 3367 |
| gcttttaaggagtcacagt | 601 | actgtgactccttaaaagc | 1535 | tgggaggcaccgagttcgt | 2469 | acgaactcggtgcctccca | 3368 |
| cttttaaggagtcacagtg | 602 | cactgtgactccttaaaag | 1536 | gggaggcaccgagttcgtg | 2470 | cacgaactcggtgcctccc | 3369 |
| ttttaaggagtcacagtgc | 603 | gcactgtgactccttaaaa | 1537 | ggaggcaccgagttcgtgt | 2471 | acacgaactcggtgcctcc | 3370 |
| tttaaggagtcacagtgcc | 604 | ggcactgtgactccttaaa | 1538 | gaggcaccgagttcgtgtt | 2472 | aacacgaactcggtgcctc | 3371 |
| ttaaggagtcacagtgcca | 605 | tggcactgtgactccttaa | 1539 | aggcaccgagttcgtgttc | 2473 | gaacacgaactcggtgcct | 3372 |
| taaggagtcacagtgccat | 606 | atggcactgtgactcctta | 1540 | ggcaccgagttcgtgttca | 2474 | tgaacacgaactcggtgcc | 3373 |
| aaggagtcacagtgccatc | 607 | gatggcactgtgactcctt | 1541 | gcaccgagttcgtgttcaa | 2475 | ttgaacacgaactcggtgc | 3374 |
| aggagtcacagtgccatca | 608 | tgatggcactgtgactcct | 1542 | caccgagttcgtgttcaaa | 2476 | tttgaacacgaactcggtg | 3375 |
| ggagtcacagtgccatcac | 609 | gtgatggcactgtgactcc | 1543 | accgagttcgtgttcaaag | 2477 | ctttgaacacgaactcggt | 3376 |
| gagtcacagtgccatcaca | 610 | tgtgatggcactgtgactc | 1544 | ccgagttcgtgttcaaagt | 2478 | actttgaacacgaactcgg | 3377 |
| agtcacagtgccatcacat | 611 | atgtgatggcactgtgact | 1545 | cgagttcgtgttcaaagtg | 2479 | cactttgaacacgaactcg | 3378 |
| gtcacagtgccatcacatg | 612 | catgtgatggcactgtgac | 1546 | gagttcgtgttcaaagtga | 2480 | tcactttgaacacgaactc | 3379 |
| tcacagtgccatcacatgc | 613 | gcatgtgatggcactgtga | 1547 | agttcgtgttcaaagtgaa | 2481 | ttcactttgaacacgaact | 3380 |
| cacagtgccatcacatgct | 614 | agcatgtgatggcactgtg | 1548 | gttcgtgttcaaagtgaat | 2482 | attcactttgaacacgaac | 3381 |
| acagtgccatcacatgctc | 615 | gagcatgtgatggcactgt | 1549 | ttcgtgttcaaagtgaatc | 2483 | gattcactttgaacacgaa | 3382 |
| cagtgccatcacatgctca | 616 | tgagcatgtgatggcactg | 1550 | tcgtgttcaaagtgaatca | 2484 | tgattcactttgaacacga | 3383 |
| agtgccatcacatgctcaa | 617 | ttgagcatgtgatggcact | 1551 | cgtgttcaaagtgaatcac | 2485 | gtgattcactttgaacacg | 3384 |
| gtgccatcacatgctcaaa | 618 | tttgagcatgtgatggcac | 1552 | gtgttcaaagtgaatcaca | 2486 | tgtgattcactttgaacac | 3385 |
| tgccatcacatgctcaaac | 619 | gtttgagcatgtgatggca | 1553 | tgttcaaagtgaatcacat | 2487 | atgtgattcactttgaaca | 3386 |
| gccatcacatgctcaaaca | 620 | tgtttgagcatgtgatggc | 1554 | gttcaaagtgaatcacatg | 2488 | catgtgattcactttgaac | 3387 |
| ccatcacatgctcaaacat | 621 | atgtttgagcatgtgatgg | 1555 | ttcaaagtgaatcacatga | 2489 | tcatgtgattcactttgaa | 3388 |
| catcacatgctcaaacatc | 622 | gatgtttgagcatgtgatg | 1556 | tcaaagtgaatcacatgaa | 2490 | ttcatgtgattcactttga | 3389 |
| atcacatgctcaaacatct | 623 | agatgtttgagcatgtgat | 1557 | caaagtgaatcacatgaag | 2491 | cttcatgtgattcactttg | 3390 |
| tcacatgctcaaacatctc | 624 | gagatgtttgagcatgtga | 1558 | aaagtgaatcacatgaagg | 2492 | ccttcatgtgattcacttt | 3391 |
| cacatgctcaaacatctcc | 625 | ggagatgtttgagcatgtg | 1559 | aagtgaatcacatgaaggt | 2493 | accttcatgtgattcactt | 3392 |
| acatgctcaaacatctcca | 626 | tggagatgtttgagcatgt | 1560 | agtgaatcacatgaaggtc | 2494 | gaccttcatgtgattcact | 3393 |
| catgctcaaacatctccac | 627 | gtggagatgtttgagcatg | 1561 | gtgaatcacatgaaggtca | 2495 | tgaccttcatgtgattcac | 3394 |
| atgctcaaacatctccaca | 628 | tgtggagatgtttgagcat | 1562 | tgaatcacatgaaggtcac | 2496 | gtgaccttcatgtgattca | 3395 |
| tgctcaaacatctccacaa | 629 | ttgtggagatgtttgagca | 1563 | gaatcacatgaaggtcacc | 2497 | ggtgaccttcatgtgattc | 3396 |
| gctcaaacatctccacaat | 630 | attgtggagatgtttgagc | 1564 | aatcacatgaaggtcaccc | 2498 | gggtgaccttcatgtgatt | 3397 |
| ctcaaacatctccacaatg | 631 | cattgtggagatgtttgag | 1565 | atcacatgaaggtcacccc | 2499 | ggggtgaccttcatgtgat | 3398 |
| tcaaacatctccacaatgg | 632 | ccattgtggagatgtttga | 1566 | tcacatgaaggtcaccccc | 2500 | gggggtgaccttcatgtga | 3399 |
| caaacatctccacaatggt | 633 | accattgtggagatgtttg | 1567 | cacatgaaggtcaccccca | 2501 | tgggggtgaccttcatgtg | 3400 |
| aaacatctccacaatggtg | 634 | caccattgtggagatgttt | 1568 | acatgaaggtcacccccat | 2502 | atgggggtgaccttcatgt | 3401 |
| aacatctccacaatggtgc | 635 | gcaccattgtggagatgtt | 1569 | catgaaggtcacccccatg | 2503 | catgggggtgaccttcatg | 3402 |
| acatctccacaatggtgca | 636 | tgcaccattgtggagatgt | 1570 | atgaaggtcacccccatgg | 2504 | ccatgggggtgaccttcat | 3403 |
| catctccacaatggtgcaa | 637 | ttgcaccattgtggagatg | 1571 | tgaaggtcacccccatgga | 2505 | tccatgggggtgaccttca | 3404 |
| atctccacaatggtgcaag | 638 | cttgcaccattgtggagat | 1572 | gaaggtcacccccatggat | 2506 | atccatgggggtgaccttc | 3405 |
| tctccacaatggtgcaagg | 639 | ccttgcaccattgtggaga | 1573 | aaggtcacccccatggatg | 2507 | catccatgggggtgacctt | 3406 |
| ctccacaatggtgcaagga | 640 | tccttgcaccattgtggag | 1574 | aggtcacccccatggatgc | 2508 | gcatccatgggggtgacct | 3407 |
| tccacaatggtgcaaggat | 641 | atccttgcaccattgtgga | 1575 | ggtcacccccatggatgcg | 2509 | cgcatccatgggggtgacc | 3408 |
| ccacaatggtgcaaggatc | 642 | gatccttgcaccattgtgg | 1576 | gtcacccccatggatgcgg | 2510 | ccgcatccatgggggtgac | 3409 |
| cacaatggtgcaaggatca | 643 | tgatccttgcaccattgtg | 1577 | tcacccccatggatgcggc | 2511 | gccgcatccatgggggtga | 3410 |
| acaatggtgcaaggatcac | 644 | gtgatccttgcaccattgt | 1578 | cacccccatggatgcggcc | 2512 | ggccgcatccatgggggtg | 3411 |
| caatggtgcaaggatcaca | 645 | tgtgatccttgcaccattg | 1579 | acccccatggatgcggcca | 2513 | tggccgcatccatgggggt | 3412 |
| aatggtgcaaggatcacag | 646 | ctgtgatccttgcaccatt | 1580 | cccccatggatgcggccac | 2514 | gtggccgcatccatggggg | 3413 |
| atggtgcaaggatcacagt | 647 | actgtgatccttgcaccat | 1581 | ccccatggatgcggccaca | 2515 | tgtggccgcatccatgggg | 3414 |
| tggtgcaaggatcacagtg | 648 | cactgtgatccttgcacca | 1582 | cccatggatgcggccacag | 2516 | ctgtggccgcatccatggg | 3415 |
| ggtgcaaggatcacagtgc | 649 | gcactgtgatccttgcacc | 1583 | ccatggatgcggccacagc | 2517 | gctgtggccgcatccatgg | 3416 |
| gtgcaaggatcacagtgca | 650 | tgcactgtgatccttgcac | 1584 | catggatgcggccacagcc | 2518 | ggctgtggccgcatccatg | 3417 |
| tgcaaggatcacagtgcag | 651 | ctgcactgtgatccttgca | 1585 | atggatgcggccacagcct | 2519 | aggctgtggccgcatccat | 3418 |
| gcaaggatcacagtgcaga | 652 | tctgcactgtgatccttgc | 1586 | tggatgcggccacagcctc | 2520 | gaggctgtggccgcatcca | 3419 |
| caaggatcacagtgcagat | 653 | atctgcactgtgatccttg | 1587 | ggatgcggccacagcctca | 2521 | tgaggctgtggccgcatcc | 3420 |
| aaggatcacagtgcagatg | 654 | catctgcactgtgatcctt | 1588 | gatgcggccacagcctcac | 2522 | gtgaggctgtggccgcatc | 3421 |
| aggatcacagtgcagatgc | 655 | gcatctgcactgtgatcct | 1589 | atgcggccacagcctcact | 2523 | agtgaggctgtggccgcat | 3422 |
| ggatcacagtgcagatgcc | 656 | ggcatctgcactgtgatcc | 1590 | tgcggccacagcctcactg | 2524 | cagtgaggctgtggccgca | 3423 |
| gatcacagtgcagatgcca | 657 | tggcatctgcactgtgatc | 1591 | gcggccacagcctcactgc | 2525 | gcagtgaggctgtggccgc | 3424 |
| atcacagtgcagatgccac | 658 | gtggcatctgcactgtgat | 1592 | cggccacagcctcactgct | 2526 | agcagtgaggctgtggcc | 3425 |
| tcacagtgcagatgccacc | 659 | ggtggcatctgcactgtga | 1593 | taataataattaagagaaa | 2527 | | |
| cacagtgcagatgccacct | 660 | aggtggcatctgcactgtg | 1594 | ttctcttaattattattat | 2528 | ataataataattaagagaa | 3426 |
| acagtgcagatgccaccta | 661 | taggtggcatctgcactgt | 1595 | tctcttaattattattata | 2529 | tataataataattaagaga | 3427 |
| cagtgcagatgccacctac | 662 | gtaggtggcatctgcactg | 1596 | ctcttaattattattatat | 2530 | atataataataattaagag | 3428 |
| agtgcagatgccacctaca | 663 | tgtaggtggcatctgcact | 1597 | tcttaattattattatatt | 2531 | aatataataataattaaga | 3429 |
| gtgcagatgccacctacaa | 664 | ttgtaggtggcatctgcac | 1598 | cttaattattattatattt | 2532 | aaatataataataattaag | 3430 |
| tgcagatgccacctacaat | 665 | attgtaggtggcatctgca | 1599 | ttaattattattatatttc | 2533 | gaaatataataataattaa | 3431 |
| gcagatgccacctacaatc | 666 | gattgtaggtggcatctgc | 1600 | taattattattatatttct | 2534 | agaaatataataataatta | 3432 |
| cagatgccacctacaatcg | 667 | cgattgtaggtggcatctg | 1601 | aattattattatatttctg | 2535 | cagaaatataataataatt | 3433 |
| agatgccacctacaatcga | 668 | tcgattgtaggtggcatct | 1602 | attattattatatttctga | 2536 | tcagaaatataataataat | 3434 |
| gatgccacctacaatcgag | 669 | ctcgattgtaggtggcatc | 1603 | ttattattatatttctgag | 2537 | ctcagaaatataataataa | 3435 |
| atgccacctacaatcgagg | 670 | cctcgattgtaggtggcat | 1604 | tattattatatttctgagt | 2538 | actcagaaatataataata | 3436 |
| tgccacctacaatcgaggg | 671 | ccctcgattgtaggtggca | 1605 | attattatatttctgagtt | 2539 | aactcagaaatataataat | 3437 |
| gccacctacaatcgagggc | 672 | gccctcgattgtaggtggc | 1606 | ttattatatttctgagtta | 2540 | taactcagaaatataataa | 3438 |
| ccacctacaatcgagggcc | 673 | ggccctcgattgtaggtgg | 1607 | tattatatttctgagttaa | 2541 | ttaactcagaaatataata | 3439 |
| cacctacaatcgagggcca | 674 | tggccctcgattgtaggtg | 1608 | attatatttctgagttaaa | 2542 | tttaactcagaaatataat | 3440 |
| acctacaatcgagggccac | 675 | gtggccctcgattgtaggt | 1609 | ttatatttctgagttaaac | 2543 | gtttaactcagaaatataa | 3441 |
| cctacaatcgagggccact | 676 | agtggccctcgattgtagg | 1610 | tatatttctgagttaaact | 2544 | agtttaactcagaaatata | 3442 |
| ctacaatcgagggccactg | 677 | cagtggccctcgattgtag | 1611 | atatttctgagttaaactt | 2545 | aagtttaactcagaaatat | 3443 |
| tacaatcgagggccactgg | 678 | ccagtggccctcgattgta | 1612 | tatttctgagttaaactta | 2546 | taagtttaactcagaaata | 3444 |
| acaatcgagggccactggg | 679 | cccagtggccctcgattgt | 1613 | atttctgagttaaacttag | 2547 | ctaagtttaactcagaaat | 3445 |
| caatcgagggccactgggt | 680 | acccagtggccctcgattg | 1614 | tttctgagttaaacttaga | 2548 | tctaagtttaactcagaaa | 3446 |
| aatcgagggccactgggtc | 681 | gacccagtggccctcgatt | 1615 | ttctgagttaaacttagaa | 2549 | ttctaagtttaactcagaa | 3447 |
| atcgagggccactgggtct | 682 | agacccagtggccctcgat | 1616 | tctgagttaaacttagaag | 2550 | cttctaagtttaactcaga | 3448 |
| tcgagggccactgggtctc | 683 | gagacccagtggccctcga | 1617 | ctgagttaaacttagaaga | 2551 | tcttctaagtttaactcag | 3449 |
| cgagggccactgggtctcc | 684 | ggagacccagtggccctcg | 1618 | tgagttaaacttagaagaa | 2552 | ttcttctaagtttaactca | 3450 |
| gagggccactgggtctcca | 685 | tggagacccagtggccctc | 1619 | gagttaaacttagaagaaa | 2553 | tttcttctaagtttaactc | 3451 |
| agggccactgggtctccac | 686 | gtggagacccagtggccct | 1620 | agttaaacttagaagaaac | 2554 | gtttcttctaagtttaact | 3452 |
| gggccactgggtctccaca | 687 | tgtggagacccagtggccc | 1621 | gttaaacttagaagaaaca | 2555 | tgtttcttctaagtttaac | 3453 |
| ggccactgggtctccacag | 688 | ctgtggagacccagtggcc | 1622 | ttaaacttagaagaaacaa | 2556 | ttgtttcttctaagtttaa | 3454 |
| gccactgggtctccacagg | 689 | cctgtggagacccagtggc | 1623 | taaacttagaagaaacaac | 2557 | gttgtttcttctaagttta | 3455 |
| ccactgggtctccacaggc | 690 | gcctgtggagacccagtgg | 1624 | aaacttagaagaaacaact | 2558 | agttgtttcttctaagttt | 3456 |
| cactgggtctccacaggct | 691 | agcctgtggagacccagtg | 1625 | aacttagaagaaacaacta | 2559 | tagttgtttcttctaagtt | 3457 |
| actgggtctccacaggctg | 692 | cagcctgtggagacccagt | 1626 | acttagaagaaacaactat | 2560 | atagttgtttcttctaagt | 3458 |
| ctgggtctccacaggctgt | 693 | acagcctgtggagacccag | 1627 | cttagaagaaacaactatc | 2561 | gatagttgtttcttctaag | 3459 |
| tgggtctccacaggctgtg | 694 | cacagcctgtggagaccca | 1628 | ttagaagaaacaactatca | 2562 | tgatagttgtttcttctaa | 3460 |
| gggtctccacaggctgtga | 695 | tcacagcctgtggagaccc | 1629 | tagaagaaacaactatcaa | 2563 | ttgatagttgtttcttcta | 3461 |
| ggtctccacaggctgtgaa | 696 | ttcacagcctgtggagacc | 1630 | agaagaaacaactatcaag | 2564 | cttgatagttgtttcttct | 3462 |
| gtctccacaggctgtgaag | 697 | cttcacagcctgtggagac | 1631 | gaagaaacaactatcaagc | 2565 | gcttgatagttgtttcttc | 3463 |
| tctccacaggctgtgaagt | 698 | acttcacagcctgtggaga | 1632 | aagaaacaactatcaagct | 2566 | agcttgatagttgtttctt | 3464 |
| ctccacaggctgtgaagta | 699 | tacttcacagcctgtggag | 1633 | agaaacaactatcaagcta | 2567 | tagcttgatagttgtttct | 3465 |
| tccacaggctgtgaagtaa | 700 | ttacttcacagcctgtgga | 1634 | gaaacaactatcaagctac | 2568 | gtagcttgatagttgtttc | 3466 |
| ccacaggctgtgaagtaag | 701 | cttacttcacagcctgtgg | 1635 | aaacaactatcaagctaca | 2569 | tgtagcttgatagttgttt | 3467 |
| cacaggctgtgaagtaagg | 702 | ccttacttcacagcctgtg | 1636 | aacaactatcaagctacaa | 2570 | ttgtagcttgatagttgtt | 3468 |
| acaggctgtgaagtaaggt | 703 | accttacttcacagcctgt | 1637 | acaactatcaagctacaac | 2571 | gttgtagcttgatagttgt | 3469 |
| caggctgtgaagtaaggtc | 704 | gaccttacttcacagcctg | 1638 | caactatcaagctacaact | 2572 | agttgtagcttgatagttg | 3470 |
| aggctgtgaagtaaggtca | 705 | tgaccttacttcacagcct | 1639 | aactatcaagctacaactt | 2573 | aagttgtagcttgatagtt | 3471 |
| ggctgtgaagtaaggtcag | 706 | ctgaccttacttcacagcc | 1640 | actatcaagctacaacttt | 2574 | aaagttgtagcttgatagt | 3472 |
| gctgtgaagtaaggtcagg | 707 | cctgaccttacttcacagc | 1641 | ctatcaagctacaactttt | 2575 | aaaagttgtagcttgatag | 3473 |
| ctgtgaagtaaggtcaggc | 708 | gcctgaccttacttcacag | 1642 | tatcaagctacaacttttc | 2576 | gaaaagttgtagcttgata | 3474 |
| tgtgaagtaaggtcaggcc | 709 | ggcctgaccttacttcaca | 1643 | atcaagctacaacttttcc | 2577 | ggaaaagttgtagcttgat | 3475 |
| gtgaagtaaggtcaggccc | 710 | gggcctgaccttacttcac | 1644 | tcaagctacaacttttcct | 2578 | aggaaaagttgtagcttga | 3476 |
| tgaagtaaggtcaggccca | 711 | tgggcctgaccttacttca | 1645 | caagctacaacttttcctg | 2579 | caggaaaagttgtagcttg | 3477 |
| gaagtaaggtcaggcccag | 712 | ctgggcctgaccttacttc | 1646 | aagctacaacttttcctgc | 2580 | gcaggaaaagttgtagctt | 3478 |
| aagtaaggtcaggcccaga | 713 | tctgggcctgaccttactt | 1647 | agctacaacttttcctgcc | 2581 | ggcaggaaaagttgtagct | 3479 |
| agtaaggtcaggcccagag | 714 | ctctgggcctgaccttact | 1648 | gctacaacttttcctgcca | 2582 | tggcaggaaaagttgtagc | 3480 |
| gtaaggtcaggcccagagt | 715 | actctgggcctgaccttac | 1649 | ctacaacttttcctgccat | 2583 | atggcaggaaaagttgtag | 3481 |
| taaggtcaggcccagagtt | 716 | aactctgggcctgacctta | 1650 | tacaacttttcctgccatt | 2584 | aatggcaggaaaagttgta | 3482 |
| aaggtcaggcccagagttc | 717 | gaactctgggcctgacctt | 1651 | acaacttttcctgccattt | 2585 | aaatggcaggaaaagttgt | 3483 |
| aggtcaggcccagagttca | 718 | tgaactctgggcctgacct | 1652 | caacttttcctgccatttt | 2586 | aaaatggcaggaaaagttg | 3484 |
| ggtcaggcccagagttcat | 719 | atgaactctgggcctgacc | 1653 | aacttttcctgccattttc | 2587 | gaaaatggcaggaaaagtt | 3485 |
| gtcaggcccagagttcatc | 720 | gatgaactctgggcctgac | 1654 | acttttcctgccattttcc | 2588 | ggaaaatggcaggaaaagt | 3486 |
| tcaggcccagagttcatca | 721 | tgatgaactctgggcctga | 1655 | cttttcctgccattttcct | 2589 | aggaaaatggcaggaaaag | 3487 |
| caggcccagagttcatcac | 722 | gtgatgaactctgggcctg | 1656 | ttttcctgccattttcctg | 2590 | caggaaaatggcaggaaaa | 3488 |
| aggcccagagttcatcaca | 723 | tgtgatgaactctgggcct | 1657 | tttcctgccattttcctgt | 2591 | acaggaaaatggcaggaaa | 3489 |
| ggcccagagttcatcacaa | 724 | ttgtgatgaactctgggcc | 1658 | ttcctgccattttcctgtg | 2592 | cacaggaaaatggcaggaa | 3490 |
| gcccagagttcatcacaag | 725 | cttgtgatgaactctgggc | 1659 | tcctgccattttcctgtgg | 2593 | ccacaggaaaatggcagga | 3491 |
| cccagagttcatcacaagg | 726 | ccttgtgatgaactctggg | 1660 | cctgccattttcctgtggt | 2594 | accacaggaaaatggcagg | 3492 |
| ccagagttcatcacaaggt | 727 | accttgtgatgaactctgg | 1661 | ctgccattttcctgtggtt | 2595 | aaccacaggaaaatggcag | 3493 |
| cagagttcatcacaaggtc | 728 | gaccttgtgatgaactctg | 1662 | tgccattttcctgtggttg | 2596 | caaccacaggaaaatggca | 3494 |
| agagttcatcacaaggtcc | 729 | ggaccttgtgatgaactct | 1663 | gccattttcctgtggttgc | 2597 | gcaaccacaggaaaatggc | 3495 |
| gagttcatcacaaggtcct | 730 | aggaccttgtgatgaactc | 1664 | ccattttcctgtggttgca | 2598 | tgcaaccacaggaaaatgg | 3496 |
| agttcatcacaaggtccta | 731 | taggaccttgtgatgaact | 1665 | cattttcctgtggttgcag | 2599 | ctgcaaccacaggaaaatg | 3497 |
| gttcatcacaaggtcctac | 732 | gtaggaccttgtgatgaac | 1666 | attttcctgtggttgcagc | 2600 | gctgcaaccacaggaaaat | 3498 |
| ttcatcacaaggtcctaca | 733 | tgtaggaccttgtgatgaa | 1667 | ttttcctgtggttgcagcc | 2601 | ggctgcaaccacaggaaaa | 3499 |
| tcatcacaaggtcctacag | 734 | ctgtaggaccttgtgatga | 1668 | tttcctgtggttgcagcct | 2602 | aggctgcaaccacaggaaa | 3500 |
| catcacaaggtcctacaga | 735 | tctgtaggaccttgtgatg | 1669 | ttcctgtggttgcagcctg | 2603 | caggctgcaaccacaggaa | 3501 |
| atcacaaggtcctacagat | 736 | atctgtaggaccttgtgat | 1670 | tcctgtggttgcagcctgt | 2604 | acaggctgcaaccacagga | 3502 |
| tcacaaggtcctacagatt | 737 | aatctgtaggaccttgtga | 1671 | cctgtggttgcagcctgtc | 2605 | gacaggctgcaaccacagg | 3503 |
| cacaaggtcctacagattc | 738 | gaatctgtaggaccttgtg | 1672 | ctgtggttgcagcctgtct | 2606 | agacaggctgcaaccacag | 3504 |
| acaaggtcctacagattct | 739 | agaatctgtaggaccttgt | 1673 | tgtggttgcagcctgtctt | 2607 | aagacaggctgcaaccaca | 3505 |
| caaggtcctacagattcta | 740 | tagaatctgtaggaccttg | 1674 | gtggttgcagcctgtcttc | 2608 | gaagacaggctgcaaccac | 3506 |
| aaggtcctacagattctac | 741 | gtagaatctgtaggacctt | 1675 | tggttgcagcctgtcttcc | 2609 | ggaagacaggctgcaacca | 3507 |
| aggtcctacagattctacc | 742 | ggtagaatctgtaggacct | 1676 | ggttgcagcctgtcttcct | 2610 | aggaagacaggctgcaacc | 3508 |
| ggtcctacagattctacca | 743 | tggtagaatctgtaggacc | 1677 | gttgcagcctgtcttcctt | 2611 | aaggaagacaggctgcaac | 3509 |
| gtcctacagattctaccac | 744 | gtggtagaatctgtaggac | 1678 | ttgcagcctgtcttccttt | 2612 | aaaggaagacaggctgcaa | 3510 |
| tcctacagattctaccaca | 745 | tgtggtagaatctgtagga | 1679 | tgcagcctgtcttcctttg | 2613 | caaaggaagacaggctgca | 3511 |
| cctacagattctaccacaa | 746 | ttgtggtagaatctgtagg | 1680 | gcagcctgtcttcctttga | 2614 | tcaaaggaagacaggctgc | 3512 |
| ctacagattctaccacaat | 747 | attgtggtagaatctgtag | 1681 | cagcctgtcttcctttgaa | 2615 | ttcaaaggaagacaggctg | 3513 |
| tacagattctaccacaata | 748 | tattgtggtagaatctgta | 1682 | agcctgtcttcctttgaaa | 2616 | tttcaaaggaagacaggct | 3514 |
| acagattctaccacaataa | 749 | ttattgtggtagaatctgt | 1683 | gcctgtcttcctttgaaat | 2617 | atttcaaaggaagacaggc | 3515 |
| cagattctaccacaataac | 750 | gttattgtggtagaatctg | 1684 | cctgtcttcctttgaaatt | 2618 | aatttcaaaggaagacagg | 3516 |
| agattctaccacaataaca | 751 | tgttattgtggtagaatct | 1685 | ctgtcttcctttgaaattg | 2619 | caatttcaaaggaagacag | 3517 |
| gattctaccacaataacac | 752 | gtgttattgtggtagaatc | 1686 | tgtcttcctttgaaattgt | 2620 | acaatttcaaaggaagaca | 3518 |
| attctaccacaataacacc | 753 | ggtgttattgtggtagaat | 1687 | gtcttcctttgaaattgtt | 2621 | aacaatttcaaaggaagac | 3519 |
| ttctaccacaataacacct | 754 | aggtgttattgtggtagaa | 1688 | tcttcctttgaaattgttt | 2622 | aaacaatttcaaaggaaga | 3520 |
| tctaccacaataacacctt | 755 | aaggtgttattgtggtaga | 1689 | cttcctttgaaattgtttt | 2623 | aaaacaatttcaaaggaag | 3521 |
| ctaccacaataacaccttc | 756 | gaaggtgttattgtggtag | 1690 | ttcctttgaaattgtttta | 2624 | taaaacaatttcaaaggaa | 3522 |
| taccacaataacaccttca | 757 | tgaaggtgttattgtggta | 1691 | tcctttgaaattgttttac | 2625 | gtaaaacaatttcaaagga | 3523 |
| accacaataacaccttcaa | 758 | ttgaaggtgttattgtggt | 1692 | cctttgaaattgttttact | 2626 | agtaaaacaatttcaaagg | 3524 |
| ccacaataacaccttcaag | 759 | cttgaaggtgttattgtgg | 1693 | ctttgaaattgttttactc | 2627 | gagtaaaacaatttcaaag | 3525 |
| cacaataacaccttcaagg | 760 | ccttgaaggtgttattgtg | 1694 | tttgaaattgttttactct | 2628 | agagtaaaacaatttcaaa | 3526 |
| acaataacaccttcaaggc | 761 | gccttgaaggtgttattgt | 1695 | ttgaaattgttttactctc | 2629 | gagagtaaaacaatttcaa | 3527 |
| caataacaccttcaaggcc | 762 | ggccttgaaggtgttattg | 1696 | tgaaattgttttactctct | 2630 | agagagtaaaacaatttca | 3528 |
| aataacaccttcaaggcct | 763 | aggccttgaaggtgttatt | 1697 | gaaattgttttactctctg | 2631 | cagagagtaaaacaatttc | 3529 |
| ataacaccttcaaggccta | 764 | taggccttgaaggtgttat | 1698 | aaattgttttactctctga | 2632 | tcagagagtaaaacaattt | 3530 |
| taacaccttcaaggcctac | 765 | gtaggccttgaaggtgtta | 1699 | aattgttttactctctgag | 2633 | ctcagagagtaaaacaatt | 3531 |
| aacaccttcaaggcctacc | 766 | ggtaggccttgaaggtgtt | 1700 | attgttttactctctgagt | 2634 | actcagagagtaaaacaat | 3532 |
| acaccttcaaggcctacca | 767 | tggtaggccttgaaggtgt | 1701 | ttgttttactctctgagtt | 2635 | aactcagagagtaaaacaa | 3533 |
| caccttcaaggcctaccaa | 768 | ttggtaggccttgaaggtg | 1702 | tgttttactctctgagttt | 2636 | aaactcagagagtaaaaca | 3534 |
| accttcaaggcctaccaat | 769 | attggtaggccttgaaggt | 1703 | gttttactctctgagtttt | 2637 | aaaactcagagagtaaaac | 3535 |
| ccttcaaggcctaccaatt | 770 | aattggtaggccttgaagg | 1704 | ttttactctctgagtttta | 2638 | taaaactcagagagtaaaa | 3536 |
| cttcaaggcctaccaattt | 771 | aaattggtaggccttgaag | 1705 | tttactctctgagttttat | 2639 | ataaaactcagagagtaaa | 3537 |
| ttcaaggcctaccaatttt | 772 | aaaattggtaggccttgaa | 1706 | ttactctctgagttttata | 2640 | tataaaactcagagagtaa | 3538 |
| tcaaggcctaccaatttta | 773 | taaaattggtaggccttga | 1707 | tactctctgagttttatat | 2641 | atataaaactcagagagta | 3539 |
| caaggcctaccaattttat | 774 | ataaaattggtaggccttg | 1708 | actctctgagttttatatg | 2642 | catataaaactcagagagt | 3540 |
| aaggcctaccaattttatt | 775 | aataaaattggtaggcctt | 1709 | ctctctgagttttatatgc | 2643 | gcatataaaactcagagag | 3541 |
| aggcctaccaattttatta | 776 | taataaaattggtaggcct | 1710 | tctctgagttttatatgct | 2644 | agcatataaaactcagaga | 3542 |
| ggcctaccaattttattat | 777 | ataataaaattggtaggcc | 1711 | ctctgagttttatatgctg | 2645 | cagcatataaaactcagag | 3543 |
| gcctaccaattttattatg | 778 | cataataaaattggtaggc | 1712 | tctgagttttatatgctgg | 2646 | ccagcatataaaactcaga | 3544 |
| cctaccaattttattatgg | 779 | ccataataaaattggtagg | 1713 | ctgagttttatatgctgga | 2647 | tccagcatataaaactcag | 3545 |
| ctaccaattttattatggc | 780 | gccataataaaattggtag | 1714 | tgagttttatatgctggaa | 2648 | ttccagcatataaaactca | 3546 |
| taccaattttattatggca | 781 | tgccataataaaattggta | 1715 | gagttttatatgctggaat | 2649 | attccagcatataaaactc | 3547 |
| accaattttattatggcag | 782 | ctgccataataaaattggt | 1716 | agttttatatgctggaatc | 2650 | gattccagcatataaaact | 3548 |
| ccaattttattatggcagc | 783 | gctgccataataaaattgg | 1717 | gttttatatgctggaatcc | 2651 | ggattccagcatataaaac | 3549 |
| caattttattatggcagca | 784 | tgctgccataataaaattg | 1718 | ttttatatgctggaatcca | 2652 | tggattccagcatataaaa | 3550 |
| aattttattatggcagcaa | 785 | ttgctgccataataaaatt | 1719 | tttatatgctggaatccaa | 2653 | ttggattccagcatataaa | 3551 |
| attttattatggcagcaac | 786 | gttgctgccataataaaat | 1720 | ttatatgctggaatccaat | 2654 | attggattccagcatataa | 3552 |
| ttttattatggcagcaacc | 787 | ggttgctgccataataaaa | 1721 | tatatgctggaatccaatg | 2655 | cattggattccagcatata | 3553 |
| tttattatggcagcaaccg | 788 | cggttgctgccataataaa | 1722 | atatgctggaatccaatgc | 2656 | gcattggattccagcatat | 3554 |
| ttattatggcagcaaccgg | 789 | ccggttgctgccataataa | 1723 | tatgctggaatccaatgca | 2657 | tgcattggattccagcata | 3555 |
| tattatggcagcaaccggt | 790 | accggttgctgccataata | 1724 | atgctggaatccaatgcag | 2658 | ctgcattggattccagcat | 3556 |
| attatggcagcaaccggtg | 791 | caccggttgctgccataat | 1725 | tgctggaatccaatgcaga | 2659 | tctgcattggattccagca | 3557 |
| ttatggcagcaaccggtgc | 792 | gcaccggttgctgccataa | 1726 | gctggaatccaatgcagag | 2660 | ctctgcattggattccagc | 3558 |
| tatggcagcaaccggtgca | 793 | tgcaccggttgctgccata | 1727 | ctggaatccaatgcagagt | 2661 | actctgcattggattccag | 3559 |
| atggcagcaaccggtgcac | 794 | gtgcaccggttgctgccat | 1728 | tggaatccaatgcagagtt | 2662 | aactctgcattggattcca | 3560 |
| tggcagcaaccggtgcaca | 795 | tgtgcaccggttgctgcca | 1729 | ggaatccaatgcagagttg | 2663 | caactctgcattggattcc | 3561 |
| ggcagcaaccggtgcacaa | 796 | ttgtgcaccggttgctgcc | 1730 | gaatccaatgcagagttgg | 2664 | ccaactctgcattggattc | 3562 |
| gcagcaaccggtgcacaaa | 797 | tttgtgcaccggttgctgc | 1731 | aatccaatgcagagttggt | 2665 | accaactctgcattggatt | 3563 |
| cagcaaccggtgcacaaat | 798 | atttgtgcaccggttgctg | 1732 | atccaatgcagagttggtt | 2666 | aaccaactctgcattggat | 3564 |
| agcaaccggtgcacaaatc | 799 | gatttgtgcaccggttgct | 1733 | tccaatgcagagttggttt | 2667 | aaaccaactctgcattgga | 3565 |
| gcaaccggtgcacaaatcc | 800 | ggatttgtgcaccggttgc | 1734 | ccaatgcagagttggtttg | 2668 | caaaccaactctgcattgg | 3566 |
| caaccggtgcacaaatccc | 801 | gggatttgtgcaccggttg | 1735 | caatgcagagttggtttgg | 2669 | ccaaaccaactctgcattg | 3567 |
| aaccggtgcacaaatccca | 802 | tgggatttgtgcaccggtt | 1736 | aatgcagagttggtttggg | 2670 | cccaaaccaactctgcatt | 3568 |
| accggtgcacaaatcccac | 803 | gtgggatttgtgcaccggt | 1737 | atgcagagttggtttggga | 2671 | tcccaaaccaactctgcat | 3569 |
| ccggtgcacaaatcccact | 804 | agtgggatttgtgcaccgg | 1738 | tgcagagttggtttgggac | 2672 | gtcccaaaccaactctgca | 3570 |
| cggtgcacaaatcccactt | 805 | aagtgggatttgtgcaccg | 1739 | gcagagttggtttgggact | 2673 | agtcccaaaccaactctgc | 3571 |
| ggtgcacaaatcccactta | 806 | taagtgggatttgtgcacc | 1740 | cagagttggtttgggactg | 2674 | cagtcccaaaccaactctg | 3572 |
| gtgcacaaatcccacttat | 807 | ataagtgggatttgtgcac | 1741 | agagttggtttgggactgt | 2675 | acagtcccaaaccaactct | 3573 |
| tgcacaaatcccacttata | 808 | tataagtgggatttgtgca | 1742 | gagttggtttgggactgtg | 2676 | cacagtcccaaaccaactc | 3574 |
| gcacaaatcccacttatac | 809 | gtataagtgggatttgtgc | 1743 | agttggtttgggactgtga | 2677 | tcacagtcccaaaccaact | 3575 |
| cacaaatcccacttatact | 810 | agtataagtgggatttgtg | 1744 | gttggtttgggactgtgat | 2678 | atcacagtcccaaaccaac | 3576 |
| acaaatcccacttatactc | 811 | gagtataagtgggatttgt | 1745 | ttggtttgggactgtgatc | 2679 | gatcacagtcccaaaccaa | 3577 |
| caaatcccacttatactct | 812 | agagtataagtgggatttg | 1746 | tggtttgggactgtgatca | 2680 | tgatcacagtcccaaacca | 3578 |
| aaatcccacttatactctc | 813 | gagagtataagtgggattt | 1747 | ggtttgggactgtgatcaa | 2681 | ttgatcacagtcccaaacc | 3579 |
| aatcccacttatactctca | 814 | tgagagtataagtgggatt | 1748 | gtttgggactgtgatcaag | 2682 | cttgatcacagtcccaaac | 3580 |
| atcccacttatactctcat | 815 | atgagagtataagtgggat | 1749 | tttgggactgtgatcaaga | 2683 | tcttgatcacagtcccaaa | 3581 |
| tcccacttatactctcatc | 816 | gatgagagtataagtggga | 1750 | ttgggactgtgatcaagac | 2684 | gtcttgatcacagtcccaa | 3582 |
| cccacttatactctcatca | 817 | tgatgagagtataagtggg | 1751 | tgggactgtgatcaagaca | 2685 | tgtcttgatcacagtccca | 3583 |
| ccacttatactctcatcat | 818 | atgatgagagtataagtgg | 1752 | gggactgtgatcaagacac | 2686 | gtgtcttgatcacagtccc | 3584 |
| cacttatactctcatcatc | 819 | gatgatgagagtataagtg | 1753 | ggactgtgatcaagacacc | 2687 | ggtgtcttgatcacagtcc | 3585 |
| acttatactctcatcatcc | 820 | ggatgatgagagtataagt | 1754 | gactgtgatcaagacacct | 2688 | aggtgtcttgatcacagtc | 3586 |
| cttatactctcatcatccg | 821 | cggatgatgagagtataag | 1755 | actgtgatcaagacacctt | 2689 | aaggtgtcttgatcacagt | 3587 |
| ttatactctcatcatccgg | 822 | ccggatgatgagagtataa | 1756 | ctgtgatcaagacaccttt | 2690 | aaaggtgtcttgatcacag | 3588 |
| tatactctcatcatccggg | 823 | cccggatgatgagagtata | 1757 | tgtgatcaagacacctttt | 2691 | aaaaggtgtcttgatcaca | 3589 |
| atactctcatcatccgggg | 824 | ccccggatgatgagagtat | 1758 | gtgatcaagacacctttta | 2692 | taaaaggtgtcttgatcac | 3590 |
| tactctcatcatccggggc | 825 | gccccggatgatgagagta | 1759 | tgatcaagacaccttttat | 2693 | ataaaaggtgtcttgatca | 3591 |
| actctcatcatccggggca | 826 | tgccccggatgatgagagt | 1760 | gatcaagacaccttttatt | 2694 | aataaaaggtgtcttgatc | 3592 |
| ctctcatcatccggggcaa | 827 | ttgccccggatgatgagag | 1761 | atcaagacaccttttatta | 2695 | taataaaaggtgtcttgat | 3593 |
| tctcatcatccggggcaag | 828 | cttgccccggatgatgaga | 1762 | tcaagacaccttttattaa | 2696 | ttaataaaaggtgtcttga | 3594 |
| ctcatcatccggggcaaga | 829 | tcttgccccggatgatgag | 1763 | caagacaccttttattaat | 2697 | attaataaaaggtgtcttg | 3595 |
| tcatcatccggggcaagat | 830 | atcttgccccggatgatga | 1764 | aagacaccttttattaata | 2698 | tattaataaaaggtgtctt | 3596 |
| catcatccggggcaagatc | 831 | gatcttgccccggatgatg | 1765 | agacaccttttattaataa | 2699 | ttattaataaaaggtgtct | 3597 |
| atcatccggggcaagatcc | 832 | ggatcttgccccggatgat | 1766 | gacaccttttattaataaa | 2700 | tttattaataaaaggtgtc | 3598 |
| tcatccggggcaagatccg | 833 | cggatcttgccccggatga | 1767 | acaccttttattaataaag | 2701 | ctttattaataaaaggtgt | 3599 |
| catccggggcaagatccgc | 834 | gcggatcttgccccggatg | 1768 | caccttttattaataaaga | 2702 | tctttattaataaaaggtg | 3600 |
| atccggggcaagatccgcc | 835 | ggcggatcttgccccggat | 1769 | accttttattaataaagaa | 2703 | ttctttattaataaaaggt | 3601 |
| tccggggcaagatccgcct | 836 | aggcggatcttgccccgga | 1770 | ccttttattaataaagaag | 2704 | cttctttattaataaaagg | 3602 |
| ccggggcaagatccgcctc | 837 | gaggcggatcttgccccgg | 1771 | cttttattaataaagaaga | 2705 | tcttctttattaataaaag | 3603 |
| cggggcaagatccgcctcc | 838 | ggaggcggatcttgccccg | 1772 | ttttattaataaagaagag | 2706 | ctcttctttattaataaaa | 3604 |
| ggggcaagatccgcctccg | 839 | cggaggcggatcttgcccc | 1773 | tttattaataaagaagaga | 2707 | tctcttctttattaataaa | 3605 |
| gggcaagatccgcctccgc | 840 | gcggaggcggatcttgccc | 1774 | ttattaataaagaagagac | 2708 | gtctcttctttattaataa | 3606 |
| ggcaagatccgcctccgcc | 841 | ggcggaggcggatcttgcc | 1775 | tattaataaagaagagaca | 2709 | tgtctcttctttattaata | 3607 |
| gcaagatccgcctccgcca | 842 | tggcggaggcggatcttgc | 1776 | attaataaagaagagacac | 2710 | gtgtctcttctttattaat | 3608 |
| caagatccgcctccgccag | 843 | ctggcggaggcggatcttg | 1777 | ttaataaagaagagacaca | 2711 | tgtgtctcttctttattaa | 3609 |
| aagatccgcctccgccagg | 844 | cctggcggaggcggatctt | 1778 | taataaagaagagacacag | 2712 | ctgtgtctcttctttatta | 3610 |
| agatccgcctccgccaggc | 845 | gcctggcggaggcggatct | 1779 | aataaagaagagacacagg | 2713 | cctgtgtctcttctttatt | 3611 |
| gatccgcctccgccaggcc | 846 | ggcctggcggaggcggatc | 1780 | ataaagaagagacacaggt | 2714 | acctgtgtctcttctttat | 3612 |
| atccgcctccgccaggcct | 847 | aggcctggcggaggcggat | 1781 | taaagaagagacacaggtg | 2715 | cacctgtgtctcttcttta | 3613 |
| tccgcctccgccaggcctc | 848 | gaggcctggcggaggcgga | 1782 | aaagaagagacacaggtgt | 2716 | acacctgtgtctcttcttt | 3614 |
| ccgcctccgccaggcctcc | 849 | ggaggcctggcggaggcgg | 1783 | aagaagagacacaggtgta | 2717 | tacacctgtgtctcttctt | 3615 |
| cgcctccgccaggcctcct | 850 | aggaggcctggcggaggcg | 1784 | agaagagacacaggtgtag | 2718 | ctacacctgtgtctcttct | 3616 |
| gcctccgccaggcctcctg | 851 | caggaggcctggcggaggc | 1785 | gaagagacacaggtgtaga | 2719 | tctacacctgtgtctcttc | 3617 |
| cctccgccaggcctcctgg | 852 | ccaggaggcctggcggagg | 1786 | aagagacacaggtgtagat | 2720 | atctacacctgtgtctctt | 3618 |
| ctccgccaggcctcctgga | 853 | tccaggaggcctggcggag | 1787 | agagacacaggtgtagata | 2721 | tatctacacctgtgtctct | 3619 |
| tccgccaggcctcctggat | 854 | atccaggaggcctggcgga | 1788 | gagacacaggtgtagatat | 2722 | atatctacacctgtgtctc | 3620 |
| ccgccaggcctcctggatc | 855 | gatccaggaggcctggcgg | 1789 | agacacaggtgtagatatg | 2723 | catatctacacctgtgtct | 3621 |
| cgccaggcctcctggatca | 856 | tgatccaggaggcctggcg | 1790 | gacacaggtgtagatatgt | 2724 | acatatctacacctgtgtc | 3622 |
| gccaggcctcctggatcat | 857 | atgatccaggaggcctggc | 1791 | acacaggtgtagatatgta | 2725 | tacatatctacacctgtgt | 3623 |
| ccaggcctcctggatcatc | 858 | gatgatccaggaggcctgg | 1792 | cacaggtgtagatatgtat | 2726 | atacatatctacacctgtg | 3624 |
| caggcctcctggatcatcc | 859 | ggatgatccaggaggcctg | 1793 | acaggtgtagatatgtata | 2727 | tatacatatctacacctgt | 3625 |
| aggcctcctggatcatccg | 860 | cggatgatccaggaggcct | 1794 | caggtgtagatatgtatat | 2728 | atatacatatctacacctg | 3626 |
| ggcctcctggatcatccga | 861 | tcggatgatccaggaggcc | 1795 | aggtgtagatatgtatata | 2729 | tatatacatatctacacct | 3627 |
| gcctcctggatcatccgag | 862 | ctcggatgatccaggaggc | 1796 | ggtgtagatatgtatatac | 2730 | gtatatacatatctacacc | 3628 |
| cctcctggatcatccgagg | 863 | cctcggatgatccaggagg | 1797 | gtgtagatatgtatataca | 2731 | tgtatatacatatctacac | 3629 |
| ctcctggatcatccgaggg | 864 | ccctcggatgatccaggag | 1798 | tgtagatatgtatatacaa | 2732 | ttgtatatacatatctaca | 3630 |
| tcctggatcatccgagggg | 865 | cccctcggatgatccagga | 1799 | gtagatatgtatatacaaa | 2733 | tttgtatatacatatctac | 3631 |
| cctggatcatccgaggggg | 866 | ccccctcggatgatccagg | 1800 | tagatatgtatatacaaaa | 2734 | ttttgtatatacatatcta | 3632 |
| ctggatcatccgagggggc | 867 | gccccctcggatgatccag | 1801 | agatatgtatatacaaaaa | 2735 | tttttgtatatacatatct | 3633 |
| tggatcatccgagggggca | 868 | tgccccctcggatgatcca | 1802 | gatatgtatatacaaaaag | 2736 | ctttttgtatatacatatc | 3634 |
| ggatcatccgagggggcac | 869 | gtgccccctcggatgatcc | 1803 | atatgtatatacaaaaaga | 2737 | tctttttgtatatacatat | 3635 |
| gatcatccgagggggcacg | 870 | cgtgccccctcggatgatc | 1804 | tatgtatatacaaaaagat | 2738 | atctttttgtatatacata | 3636 |
| atcatccgagggggcacgg | 871 | ccgtgccccctcggatgat | 1805 | atgtatatacaaaaagatg | 2739 | catctttttgtatatacat | 3637 |
| tcatccgagggggcacgga | 872 | tccgtgccccctcggatga | 1806 | tgtatatacaaaaagatgt | 2740 | acatctttttgtatataca | 3638 |
| catccgagggggcacggaa | 873 | ttccgtgccccctcggatg | 1807 | gtatatacaaaaagatgta | 2741 | tacatctttttgtatatac | 3639 |
| atccgagggggcacggaag | 874 | cttccgtgccccctcggat | 1808 | tatatacaaaaagatgtac | 2742 | gtacatctttttgtatata | 3640 |
| tccgagggggcacggaagc | 875 | gcttccgtgccccctcgga | 1809 | atatacaaaaagatgtacg | 2743 | cgtacatctttttgtatat | 3641 |
| ccgagggggcacggaagcc | 876 | ggcttccgtgccccctcgg | 1810 | tatacaaaaagatgtacgg | 2744 | ccgtacatctttttgtata | 3642 |
| cgagggggcacggaagccg | 877 | cggcttccgtgccccctcg | 1811 | atacaaaaagatgtacggt | 2745 | accgtacatctttttgtat | 3643 |
| gagggggcacggaagccga | 878 | tcggcttccgtgccccctc | 1812 | tacaaaaagatgtacggtc | 2746 | gaccgtacatctttttgta | 3644 |
| agggggcacggaagccgac | 879 | gtcggcttccgtgccccct | 1813 | acaaaaagatgtacggtct | 2747 | agaccgtacatctttttgt | 3645 |
| gggggcacggaagccgact | 880 | agtcggcttccgtgccccc | 1814 | caaaaagatgtacggtctg | 2748 | cagaccgtacatctttttg | 3646 |
| ggggcacggaagccgacta | 881 | tagtcggcttccgtgcccc | 1815 | aaaaagatgtacggtctgg | 2749 | ccagaccgtacatcttttt | 3647 |
| gggcacggaagccgactac | 882 | gtagtcggcttccgtgccc | 1816 | aaaagatgtacggtctggc | 2750 | gccagaccgtacatctttt | 3648 |
| ggcacggaagccgactacc | 883 | ggtagtcggcttccgtgcc | 1817 | aaagatgtacggtctggcc | 2751 | ggccagaccgtacatcttt | 3649 |
| gcacggaagccgactacca | 884 | tggtagtcggcttccgtgc | 1818 | aagatgtacggtctggcca | 2752 | tggccagaccgtacatctt | 3650 |
| cacggaagccgactaccag | 885 | ctggtagtcggcttccgtg | 1819 | agatgtacggtctggccaa | 2753 | ttggccagaccgtacatct | 3651 |
| acggaagccgactaccagc | 886 | gctggtagtcggcttccgt | 1820 | gatgtacggtctggccaaa | 2754 | tttggccagaccgtacatc | 3652 |
| cggaagccgactaccagct | 887 | agctggtagtcggcttccg | 1821 | atgtacggtctggccaaac | 2755 | gtttggccagaccgtacat | 3653 |
| ggaagccgactaccagctg | 888 | cagctggtagtcggcttcc | 1822 | tgtacggtctggccaaacc | 2756 | ggtttggccagaccgtaca | 3654 |
| gaagccgactaccagctgc | 889 | gcagctggtagtcggcttc | 1823 | gtacggtctggccaaacca | 2757 | tggtttggccagaccgtac | 3655 |
| aagccgactaccagctgca | 890 | tgcagctggtagtcggctt | 1824 | tacggtctggccaaaccac | 2758 | gtggtttggccagaccgta | 3656 |
| agccgactaccagctgcac | 891 | gtgcagctggtagtcggct | 1825 | acggtctggccaaaccacc | 2759 | ggtggtttggccagaccgt | 3657 |
| gccgactaccagctgcaca | 892 | tgtgcagctggtagtcggc | 1826 | cggtctggccaaaccacct | 2760 | aggtggtttggccagaccg | 3658 |
| ccgactaccagctgcacaa | 893 | ttgtgcagctggtagtcgg | 1827 | ggtctggccaaaccacctt | 2761 | aaggtggtttggccagacc | 3659 |
| cgactaccagctgcacaac | 894 | gttgtgcagctggtagtcg | 1828 | gtctggccaaaccaccttc | 2762 | gaaggtggtttggccagac | 3660 |
| gactaccagctgcacaacg | 895 | cgttgtgcagctggtagtc | 1829 | tctggccaaaccaccttcc | 2763 | ggaaggtggtttggccaga | 3661 |
| actaccagctgcacaacgt | 896 | acgttgtgcagctggtagt | 1830 | ctggccaaaccaccttccc | 2764 | gggaaggtggtttggccag | 3662 |
| ctaccagctgcacaacgtc | 897 | gacgttgtgcagctggtag | 1831 | tggccaaaccaccttccca | 2765 | tgggaaggtggtttggcca | 3663 |
| taccagctgcacaacgtcc | 898 | ggacgttgtgcagctggta | 1832 | ggccaaaccaccttcccag | 2766 | ctgggaaggtggtttggcc | 3664 |
| accagctgcacaacgtcca | 899 | tggacgttgtgcagctggt | 1833 | gccaaaccaccttcccagc | 2767 | gctgggaaggtggtttggc | 3665 |
| ccagctgcacaacgtccag | 900 | ctggacgttgtgcagctgg | 1834 | ccaaaccaccttcccagcc | 2768 | ggctgggaaggtggtttgg | 3666 |
| cagctgcacaacgtccagg | 901 | cctggacgttgtgcagctg | 1835 | caaaccaccttcccagcct | 2769 | aggctgggaaggtggtttg | 3667 |
| agctgcacaacgtccaggt | 902 | acctggacgttgtgcagct | 1836 | aaaccaccttcccagcctt | 2770 | aaggctgggaaggtggttt | 3668 |
| gctgcacaacgtccaggtg | 903 | cacctggacgttgtgcagc | 1837 | aaccaccttcccagccttt | 2771 | aaaggctgggaaggtggtt | 3669 |
| ctgcacaacgtccaggtga | 904 | tcacctggacgttgtgcag | 1838 | accaccttcccagccttta | 2772 | taaaggctgggaaggtggt | 3670 |
| tgcacaacgtccaggtgat | 905 | atcacctggacgttgtgca | 1839 | ccaccttcccagcctttat | 2773 | ataaaggctgggaaggtgg | 3671 |
| gcacaacgtccaggtgatc | 906 | gatcacctggacgttgtgc | 1840 | caccttcccagcctttatg | 2774 | cataaaggctgggaaggtg | 3672 |
| cacaacgtccaggtgatct | 907 | agatcacctggacgttgtg | 1841 | accttcccagcctttatgc | 2775 | gcataaaggctgggaaggt | 3673 |
| acaacgtccaggtgatctg | 908 | cagatcacctggacgttgt | 1842 | ccttcccagcctttatgca | 2776 | tgcataaaggctgggaagg | 3674 |
| caacgtccaggtgatctgc | 909 | gcagatcacctggacgttg | 1843 | cttcccagcctttatgcaa | 2777 | ttgcataaaggctgggaag | 3675 |
| aacgtccaggtgatctgcc | 910 | ggcagatcacctggacgtt | 1844 | ttcccagcctttatgcaaa | 2778 | tttgcataaaggctgggaa | 3676 |
| acgtccaggtgatctgcca | 911 | tggcagatcacctggacgt | 1845 | tcccagcctttatgcaaaa | 2779 | ttttgcataaaggctggga | 3677 |
| cgtccaggtgatctgccac | 912 | gtggcagatcacctggacg | 1846 | cccagcctttatgcaaaaa | 2780 | tttttgcataaaggctggg | 3678 |
| gtccaggtgatctgccaca | 913 | tgtggcagatcacctggac | 1847 | ccagcctttatgcaaaaaa | 2781 | ttttttgcataaaggctgg | 3679 |
| tccaggtgatctgccacac | 914 | gtgtggcagatcacctgga | 1848 | cagcctttatgcaaaaaaa | 2782 | tttttttgcataaaggctg | 3680 |
| ccaggtgatctgccacaca | 915 | tgtgtggcagatcacctgg | 1849 | agcctttatgcaaaaaaag | 2783 | ctttttttgcataaaggct | 3681 |
| caggtgatctgccacacag | 916 | ctgtgtggcagatcacctg | 1850 | gcctttatgcaaaaaaagg | 2784 | cctttttttgcataaaggc | 3682 |
| aggtgatctgccacacaga | 917 | tctgtgtggcagatcacct | 1851 | cctttatgcaaaaaaaggg | 2785 | ccctttttttgcataaagg | 3683 |
| ggtgatctgccacacagag | 918 | ctctgtgtggcagatcacc | 1852 | ctttatgcaaaaaaagggg | 2786 | cccctttttttgcataaag | 3684 |
| gtgatctgccacacagagg | 919 | cctctgtgtggcagatcac | 1853 | tttatgcaaaaaaagggga | 2787 | tcccctttttttgcataaa | 3685 |
| tgatctgccacacagaggc | 920 | gcctctgtgtggcagatca | 1854 | ttatgcaaaaaaaggggag | 2788 | ctcccctttttttgcataa | 3686 |
| gatctgccacacagaggcg | 921 | cgcctctgtgtggcagatc | 1855 | tatgcaaaaaaaggggaga | 2789 | tctcccctttttttgcata | 3687 |
| atctgccacacagaggcgg | 922 | ccgcctctgtgtggcagat | 1856 | atgcaaaaaaaggggagaa | 2790 | ttctcccctttttttgcat | 3688 |
| tctgccacacagaggcggt | 923 | accgcctctgtgtggcaga | 1857 | tgcaaaaaaaggggagaat | 2791 | attctcccctttttttgca | 3689 |
| ctgccacacagaggcggtg | 924 | caccgcctctgtgtggcag | 1858 | gcaaaaaaaggggagaatc | 2792 | gattctcccctttttttgc | 3690 |
| tgccacacagaggcggtgg | 925 | ccaccgcctctgtgtggca | 1859 | caaaaaaaggggagaatca | 2793 | tgattctcccctttttttg | 3691 |
| gccacacagaggcggtggc | 926 | gccaccgcctctgtgtggc | 1860 | aaaaaaaggggagaatcaa | 2794 | ttgattctccccttttttt | 3692 |
| ccacacagaggcggtggcc | 927 | ggccaccgcctctgtgtgg | 1861 | aaaaaaggggagaatcaaa | 2795 | tttgattctcccctttttt | 3693 |
| cacacagaggcggtggccg | 928 | cggccaccgcctctgtgtg | 1862 | aaaaaggggagaatcaaag | 2796 | ctttgattctccccttttt | 3694 |
| acacagaggcggtggccga | 929 | tcggccaccgcctctgtgt | 1863 | aaaaggggagaatcaaagc | 2797 | gctttgattctcccctttt | 3695 |
| cacagaggcggtggccgag | 930 | ctcggccaccgcctctgtg | 1864 | aaaggggagaatcaaagct | 2798 | agctttgattctccccttt | 3696 |
| acagaggcggtggccgaga | 931 | tctcggccaccgcctctgt | 1865 | aaggggagaatcaaagctt | 2799 | aagctttgattctcccctt | 3697 |
| cagaggcggtggccgagaa | 932 | ttctcggccaccgcctctg | 1866 | aggggagaatcaaagcttt | 2800 | aaagctttgattctcccct | 3698 |
| agaggcggtggccgagaag | 933 | cttctcggccaccgcctct | 1867 | ggggagaatcaaagctttc | 2801 | gaaagctttgattctcccc | 3699 |
| gaggcggtggccgagaagc | 934 | gcttctcggccaccgcctc | 1868 | gggagaatcaaagctttca | 2802 | tgaaagctttgattctccc | 3700 |
| aggcggtggccgagaagct | 935 | agcttctcggccaccgcct | 1869 | ggagaatcaaagctttcat | 2803 | atgaaagctttgattctcc | 3701 |
| ggcggtggccgagaagctc | 936 | gagcttctcggccaccgcc | 1870 | gagaatcaaagctttcatt | 2804 | aatgaaagctttgattctc | 3702 |
| gcggtggccgagaagctcg | 937 | cgagcttctcggccaccgc | 1871 | agaatcaaagctttcattt | 2805 | aaatgaaagctttgattct | 3703 |
| cggtggccgagaagctcgg | 938 | ccgagcttctcggccaccg | 1872 | gaatcaaagctttcatttc | 2806 | gaaatgaaagctttgattc | 3704 |
| ggtggccgagaagctcggc | 939 | gccgagcttctcggccacc | 1873 | aatcaaagctttcatttca | 2807 | tgaaatgaaagctttgatt | 3705 |
| gtggccgagaagctcggcc | 940 | ggccgagcttctcggccac | 1874 | atcaaagctttcatttcag | 2808 | ctgaaatgaaagctttgat | 3706 |
| tggccgagaagctcggcca | 941 | tggccgagcttctcggcca | 1875 | tcaaagctttcatttcaga | 2809 | tctgaaatgaaagctttga | 3707 |
| ggccgagaagctcggccag | 942 | ctggccgagcttctcggcc | 1876 | caaagctttcatttcagaa | 2810 | ttctgaaatgaaagctttg | 3708 |
| gccgagaagctcggccagc | 943 | gctggccgagcttctcggc | 1877 | aaagctttcatttcagaaa | 2811 | tttctgaaatgaaagcttt | 3709 |
| ccgagaagctcggccagca | 944 | tgctggccgagcttctcgg | 1878 | aagctttcatttcagaaat | 2812 | atttctgaaatgaaagctt | 3710 |
| cgagaagctcggccagcag | 945 | ctgctggccgagcttctcg | 1879 | agctttcatttcagaaatg | 2813 | catttctgaaatgaaagct | 3711 |
| gagaagctcggccagcagg | 946 | cctgctggccgagcttctc | 1880 | gctttcatttcagaaatgt | 2814 | acatttctgaaatgaaagc | 3712 |
| agaagctcggccagcaggt | 947 | acctgctggccgagcttct | 1881 | ctttcatttcagaaatgtt | 2815 | aacatttctgaaatgaaag | 3713 |
| gaagctcggccagcaggtg | 948 | cacctgctggccgagcttc | 1882 | tttcatttcagaaatgttg | 2816 | caacatttctgaaatgaaa | 3714 |
| aagctcggccagcaggtga | 949 | tcacctgctggccgagctt | 1883 | ttcatttcagaaatgttgc | 2817 | gcaacatttctgaaatgaa | 3715 |
| agctcggccagcaggtgaa | 950 | ttcacctgctggccgagct | 1884 | tcatttcagaaatgttgcg | 2818 | cgcaacatttctgaaatga | 3716 |
| gctcggccagcaggtgaac | 951 | gttcacctgctggccgagc | 1885 | catttcagaaatgttgcgt | 2819 | acgcaacatttctgaaatg | 3717 |
| ctcggccagcaggtgaacc | 952 | ggttcacctgctggccgag | 1886 | atttcagaaatgttgcgtg | 2820 | cacgcaacatttctgaaat | 3718 |
| tcggccagcaggtgaaccg | 953 | cggttcacctgctggccga | 1887 | tttcagaaatgttgcgtgg | 2821 | ccacgcaacatttctgaaa | 3719 |
| cggccagcaggtgaaccgc | 954 | gcggttcacctgctggccg | 1888 | ttcagaaatgttgcgtgga | 2822 | tccacgcaacatttctgaa | 3720 |
| ggccagcaggtgaaccgca | 955 | tgcggttcacctgctggcc | 1889 | tcagaaatgttgcgtggaa | 2823 | ttccacgcaacatttctga | 3721 |
| gccagcaggtgaaccgcac | 956 | gtgcggttcacctgctggc | 1890 | cagaaatgttgcgtggaaa | 2824 | tttccacgcaacatttctg | 3722 |
| ccagcaggtgaaccgcaca | 957 | tgtgcggttcacctgctgg | 1891 | agaaatgttgcgtggaaaa | 2825 | ttttccacgcaacatttct | 3723 |
| cagcaggtgaaccgcacat | 958 | atgtgcggttcacctgctg | 1892 | gaaatgttgcgtggaaaag | 2826 | cttttccacgcaacatttc | 3724 |
| agcaggtgaaccgcacatg | 959 | catgtgcggttcacctgct | 1893 | aaatgttgcgtggaaaagt | 2827 | acttttccacgcaacattt | 3725 |
| gcaggtgaaccgcacatgc | 960 | gcatgtgcggttcacctgc | 1894 | aatgttgcgtggaaaagta | 2828 | tacttttccacgcaacatt | 3726 |
| caggtgaaccgcacatgcc | 961 | ggcatgtgcggttcacctg | 1895 | atgttgcgtggaaaagtat | 2829 | atacttttccacgcaacat | 3727 |
| aggtgaaccgcacatgccc | 962 | gggcatgtgcggttcacct | 1896 | tgttgcgtggaaaagtatc | 2830 | gatacttttccacgcaaca | 3728 |
| ggtgaaccgcacatgcccg | 963 | cgggcatgtgcggttcacc | 1897 | gttgcgtggaaaagtatct | 2831 | agatacttttccacgcaac | 3729 |
| gtgaaccgcacatgcccgg | 964 | ccgggcatgtgcggttcac | 1898 | ttgcgtggaaaagtatctg | 2832 | cagatacttttccacgcaa | 3730 |
| tgaaccgcacatgcccggg | 965 | cccgggcatgtgcggttca | 1899 | tgcgtggaaaagtatctgt | 2833 | acagatacttttccacgca | 3731 |
| gaaccgcacatgcccgggc | 966 | gcccgggcatgtgcggttc | 1900 | gcgtggaaaagtatctgta | 2834 | tacagatacttttccacgc | 3732 |
| aaccgcacatgcccgggct | 967 | agcccgggcatgtgcggtt | 1901 | cgtggaaaagtatctgtaa | 2835 | ttacagatacttttccacg | 3733 |
| accgcacatgcccgggctt | 968 | aagcccgggcatgtgcggt | 1902 | gtggaaaagtatctgtaat | 2836 | attacagatacttttccac | 3734 |
| ccgcacatgcccgggcttc | 969 | gaagcccgggcatgtgcgg | 1903 | tggaaaagtatctgtaatt | 2837 | aattacagatacttttcca | 3735 |
| cgcacatgcccgggcttcc | 970 | ggaagcccgggcatgtgcg | 1904 | ggaaaagtatctgtaatta | 2838 | taattacagatacttttcc | 3736 |
| gcacatgcccgggcttcct | 971 | aggaagcccgggcatgtgc | 1905 | gaaaagtatctgtaattaa | 2839 | ttaattacagatacttttc | 3737 |
| cacatgcccgggcttcctc | 972 | gaggaagcccgggcatgtg | 1906 | aaaagtatctgtaattaaa | 2840 | tttaattacagatactttt | 3738 |
| acatgcccgggcttcctcg | 973 | cgaggaagcccgggcatgt | 1907 | aaagtatctgtaattaaag | 2841 | ctttaattacagatacttt | 3739 |
| catgcccgggcttcctcgc | 974 | gcgaggaagcccgggcatg | 1908 | aagtatctgtaattaaagt | 2842 | actttaattacagatactt | 3740 |
| atgcccgggcttcctcgca | 975 | tgcgaggaagcccgggcat | 1909 | agtatctgtaattaaagtt | 2843 | aactttaattacagatact | 3741 |
| tgcccgggcttcctcgcag | 976 | ctgcgaggaagcccgggca | 1910 | gtatctgtaattaaagttt | 2844 | aaactttaattacagatac | 3742 |
| gcccgggcttcctcgcaga | 977 | tctgcgaggaagcccgggc | 1911 | tatctgtaattaaagtttc | 2845 | gaaactttaattacagata | 3743 |
| cccgggcttcctcgcagac | 978 | gtctgcgaggaagcccggg | 1912 | atctgtaattaaagtttcg | 2846 | cgaaactttaattacagat | 3744 |
| ccgggcttcctcgcagacg | 979 | cgtctgcgaggaagcccgg | 1913 | tctgtaattaaagtttcga | 2847 | tcgaaactttaattacaga | 3745 |
| cgggcttcctcgcagacgg | 980 | ccgtctgcgaggaagcccg | 1914 | ctgtaattaaagtttcgaa | 2848 | ttcgaaactttaattacag | 3746 |
| gggcttcctcgcagacggg | 981 | cccgtctgcgaggaagccc | 1915 | tgtaattaaagtttcgaag | 2849 | cttcgaaactttaattaca | 3747 |
| ggcttcctcgcagacgggg | 982 | ccccgtctgcgaggaagcc | 1916 | gtaattaaagtttcgaagt | 2850 | acttcgaaactttaattac | 3748 |
| gcttcctcgcagacggggg | 983 | cccccgtctgcgaggaagc | 1917 | taattaaagtttcgaagta | 2851 | tacttcgaaactttaatta | 3749 |
| cttcctcgcagacgggggt | 984 | acccccgtctgcgaggaag | 1918 | aattaaagtttcgaagtaa | 2852 | ttacttcgaaactttaatt | 3750 |
| ttcctcgcagacgggggtc | 985 | gacccccgtctgcgaggaa | 1919 | attaaagtttcgaagtaat | 2853 | attacttcgaaactttaat | 3751 |
| tcctcgcagacgggggtcc | 986 | ggacccccgtctgcgagga | 1920 | ttaaagtttcgaagtaatt | 2854 | aattacttcgaaactttaa | 3752 |
| cctcgcagacgggggtccc | 987 | gggacccccgtctgcgagg | 1921 | taaagtttcgaagtaattt | 2855 | aaattacttcgaaacttta | 3753 |
| ctcgcagacgggggtccct | 988 | agggacccccgtctgcgag | 1922 | aaagtttcgaagtaattta | 2856 | taaattacttcgaaacttt | 3754 |
| tcgcagacgggggtccctg | 989 | cagggacccccgtctgcga | 1923 | aagtttcgaagtaatttaa | 2857 | ttaaattacttcgaaactt | 3755 |
| cgcagacgggggtccctgg | 990 | ccagggacccccgtctgcg | 1924 | agtttcgaagtaatttaac | 2858 | gttaaattacttcgaaact | 3756 |
| gcagacgggggtccctggg | 991 | cccagggacccccgtctgc | 1925 | gtttcgaagtaatttaacc | 2859 | ggttaaattacttcgaaac | 3757 |
| cagacgggggtccctgggt | 992 | acccagggacccccgtctg | 1926 | tttcgaagtaatttaacct | 2860 | aggttaaattacttcgaaa | 3758 |
| agacgggggtccctgggtg | 993 | cacccagggacccccgtct | 1927 | ttcgaagtaatttaaccta | 2861 | taggttaaattacttcgaa | 3759 |
| gacgggggtccctgggtgc | 994 | gcacccagggacccccgtc | 1928 | tcgaagtaatttaacctaa | 2862 | ttaggttaaattacttcga | 3760 |
| acgggggtccctgggtgca | 995 | tgcacccagggacccccgt | 1929 | cgaagtaatttaacctaaa | 2863 | tttaggttaaattacttcg | 3761 |
| cgggggtccctgggtgcag | 996 | ctgcacccagggacccccg | 1930 | gaagtaatttaacctaaaa | 2864 | ttttaggttaaattacttc | 3762 |
| gggggtccctgggtgcagg | 997 | cctgcacccagggaccccc | 1931 | aagtaatttaacctaaaaa | 2865 | tttttaggttaaattactt | 3763 |
| ggggtccctgggtgcagga | 998 | tcctgcacccagggacccc | 1932 | agtaatttaacctaaaaaa | 2866 | ttttttaggttaaattact | 3764 |
| gggtccctgggtgcaggac | 999 | gtcctgcacccagggaccc | 1933 | gtaatttaacctaaaaaaa | 2867 | tttttttaggttaaattac | 3765 |
| ggtccctgggtgcaggacg | 1000 | cgtcctgcacccagggacc | 1934 | taatttaacctaaaaaaaa | 2868 | ttttttttaggttaaatta | 3766 |
| gtccctgggtgcaggacgt | 1001 | acgtcctgcacccagggac | 1935 | aatttaacctaaaaaaaaa | 2869 | tttttttttaggttaaatt | 3767 |
| tccctgggtgcaggacgtg | 1002 | cacgtcctgcacccaggga | 1936 | atttaacctaaaaaaaaaa | 2870 | ttttttttttaggttaaat | 3768 |
| ccctgggtgcaggacgtgg | 1003 | ccacgtcctgcacccaggg | 1937 | tttaacctaaaaaaaaaaa | 2871 | tttttttttttaggttaaa | 3769 |
| cctgggtgcaggacgtggc | 1004 | gccacgtcctgcacccagg | 1938 | ttaacctaaaaaaaaaaaa | 2872 | ttttttttttttaggttaa | 3770 |
| ctgggtgcaggacgtggcc | 1005 | ggccacgtcctgcacccag | 1939 | taacctaaaaaaaaaaaaa | 2873 | tttttttttttttaggtta | 3771 |
| tgggtgcaggacgtggcct | 1006 | aggccacgtcctgcaccca | 1940 | aacctaaaaaaaaaaaaaa | 2874 | ttttttttttttttaggtt | 3772 |
| gggtgcaggacgtggccta | 1007 | taggccacgtcctgcaccc | 1941 | acctaaaaaaaaaaaaaaa | 2875 | tttttttttttttttaggt | 3773 |
| ggtgcaggacgtggcctat | 1008 | ataggccacgtcctgcacc | 1942 | cctaaaaaaaaaaaaaaaa | 2876 | ttttttttttttttttagg | 3774 |
| gtgcaggacgtggcctatg | 1009 | cataggccacgtcctgcac | 1943 | ctaaaaaaaaaaaaaaaaa | 2877 | tttttttttttttttttag | 3775 |
| tgcaggacgtggcctatga | 1010 | tcataggccacgtcctgca | 1944 | taaaaaaaaaaaaaaaaaa | 2878 | tttttttttttttttttta | 3776 |
| gcaggacgtggcctatgac | 1011 | gtcataggccacgtcctgc | 1945 | gaacggctgtgagtgcacc | 1044 | ggtgcactcacagccgttc | 1978 |
| caggacgtggcctatgacc | 1012 | ggtcataggccacgtcctg | 1946 | aacggctgtgagtgcacca | 1045 | tggtgcactcacagccgtt | 1979 |
| aggacgtggcctatgacct | 1013 | aggtcataggccacgtcct | 1947 | | | | |
| ggacgtggcctatgacctc | 1014 | gaggtcataggccacgtcc | 1948 | | | | |
| gacgtggcctatgacctct | 1015 | agaggtcataggccacgtc | 1949 | | | | |
| acgtggcctatgacctctg | 1016 | cagaggtcataggccacgt | 1950 | | | | |
| cgtggcctatgacctctgg | 1017 | ccagaggtcataggccacg | 1951 | | | | |
| gtggcctatgacctctggc | 1018 | gccagaggtcataggccac | 1952 | | | | |
| tggcctatgacctctggcg | 1019 | cgccagaggtcataggcca | 1953 | | | | |
| ggcctatgacctctggcga | 1020 | tcgccagaggtcataggcc | 1954 | | | | |
| gcctatgacctctggcgag | 1021 | ctcgccagaggtcataggc | 1955 | | | | |
| cctatgacctctggcgaga | 1022 | tctcgccagaggtcatagg | 1956 | | | | |
| ctatgacctctggcgagag | 1023 | ctctcgccagaggtcatag | 1957 | | | | |
| tatgacctctggcgagagg | 1024 | cctctcgccagaggtcata | 1958 | | | | |
| atgacctctggcgagagga | 1025 | tcctctcgccagaggtcat | 1959 | | | | |
| tgacctctggcgagaggag | 1026 | ctcctctcgccagaggtca | 1960 | | | | |
| gacctctggcgagaggaga | 1027 | tctcctctcgccagaggtc | 1961 | | | | |
| acctctggcgagaggagaa | 1028 | ttctcctctcgccagaggt | 1962 | | | | |
| cctctggcgagaggagaac | 1029 | gttctcctctcgccagagg | 1963 | | | | |
| ctctggcgagaggagaacg | 1030 | cgttctcctctcgccagag | 1964 | | | | |
| tctggcgagaggagaacgg | 1031 | ccgttctcctctcgccaga | 1965 | | | | |
| ctggcgagaggagaacggc | 1032 | gccgttctcctctcgccag | 1966 | | | | |
| tggcgagaggagaacggct | 1033 | agccgttctcctctcgcca | 1967 | | | | |
| ggcgagaggagaacggctg | 1034 | cagccgttctcctctcgcc | 1968 | | | | |
| gcgagaggagaacggctgt | 1035 | acagccgttctcctctcgc | 1969 | | | | |
| cgagaggagaacggctgtg | 1036 | cacagccgttctcctctcg | 1970 | | | | |
| gagaggagaacggctgtga | 1037 | tcacagccgttctcctctc | 1971 | | | | |
| agaggagaacggctgtgag | 1038 | ctcacagccgttctcctct | 1972 | | | | |
| gaggagaacggctgtgagt | 1039 | actcacagccgttctcctc | 1973 | | | | |
| aggagaacggctgtgagtg | 1040 | cactcacagccgttctcct | 1974 | | | | |
| ggagaacggctgtgagtgc | 1041 | gcactcacagccgttctcc | 1975 | | | | |
| gagaacggctgtgagtgca | 1042 | tgcactcacagccgttctc | 1976 | | | | |
| agaacggctgtgagtgcac | 1043 | gtgcactcacagccgttct | 1977 | | | | |

TABLE 2
List of siRNAs directed to mouse APCDD1.

| | | | | | | | SEQ |
|---|---|---|---|---|---|---|---|
| | SEQ ID | | SEQ ID | | SEQ ID | | ID |
| Sense (5′-3′) | NO: | Anti-sense (5′-3′) | NO: | Sense (5′-3′) | NO: | Anti-sense (5′-3′) | NO: |
| agcggccactgtacctctg | 3777 | cagaggtacagtggccgct | 5168 | tctcatctaaggtcatggg | 6559 | cccatgaccttagatgaga | 7949 |
| gcggccactgtacctctga | 3778 | tcagaggtacagtggccgc | 5169 | ctcatctaaggtcatgggt | 6560 | acccatgaccttagatgag | 7950 |
| cggccactgtacctctgag | 3779 | ctcagaggtacagtggccg | 5170 | tcatctaaggtcatgggtg | 6561 | cacccatgaccttagatga | 7951 |
| ggccactgtacctctgagc | 3780 | gctcagaggtacagtggcc | 5171 | catctaaggtcatgggtgg | 6562 | ccacccatgaccttagatg | 7952 |
| gccactgtacctctgagct | 3781 | agctcagaggtacagtggc | 5172 | atctaaggtcatgggtggc | 6563 | gccacccatgaccttagat | 7953 |
| ccactgtacctctgagctg | 3782 | cagctcagaggtacagtgg | 5173 | tctaaggtcatgggtggca | 6564 | tgccacccatgaccttaga | 7954 |
| cactgtacctctgagctgt | 3783 | acagctcagaggtacagtg | 5174 | ctaaggtcatgggtggcac | 6565 | gtgccacccatgaccttag | 7955 |
| actgtacctctgagctgtg | 3784 | cacagctcagaggtacagt | 5175 | taaggtcatgggtggcacg | 6566 | cgtgccacccatgacctta | 7956 |
| ctgtacctctgagctgtgc | 3785 | gcacagctcagaggtacag | 5176 | aaggtcatgggtggcacgg | 6567 | ccgtgccacccatgacctt | 7957 |
| tgtacctctgagctgtgca | 3786 | tgcacagctcagaggtaca | 5177 | aggtcatgggtggcacgga | 6568 | tccgtgccacccatgacct | 7958 |
| gtacctctgagctgtgcac | 3787 | gtgcacagctcagaggtac | 5178 | ggtcatgggtggcacggag | 6569 | ctccgtgccacccatgacc | 7959 |
| tacctctgagctgtgcacg | 3788 | cgtgcacagctcagaggta | 5179 | gtcatgggtggcacggagt | 6570 | actccgtgccacccatgac | 7960 |
| acctctgagctgtgcacgc | 3789 | gcgtgcacagctcagaggt | 5180 | tcatgggtggcacggagtt | 6571 | aactccgtgccacccatga | 7961 |
| cctctgagctgtgcacgcc | 3790 | ggcgtgcacagctcagagg | 5181 | catgggtggcacggagttt | 6572 | aaactccgtgccacccatg | 7962 |
| ctctgagctgtgcacgccg | 3791 | cggcgtgcacagctcagag | 5182 | atgggtggcacggagtttg | 6573 | caaactccgtgccacccat | 7963 |
| tctgagctgtgcacgccgc | 3792 | gcggcgtgcacagctcaga | 5183 | tgggtggcacggagtttgt | 6574 | acaaactccgtgccaccca | 7964 |
| ctgagctgtgcacgccgcg | 3793 | cgcggcgtgcacagctcag | 5184 | gggtggcacggagtttgtg | 6575 | cacaaactccgtgccaccc | 7965 |
| tgagctgtgcacgccgcgg | 3794 | ccgcggcgtgcacagctca | 5185 | ggtggcacggagtttgtgt | 6576 | acacaaactccgtgccacc | 7966 |
| gagctgtgcacgccgcggc | 3795 | gccgcggcgtgcacagctc | 5186 | gtggcacggagtttgtgtt | 6577 | aacacaaactccgtgccac | 7967 |
| agctgtgcacgccgcggcc | 3796 | ggccgcggcgtgcacagct | 5187 | tggcacggagtttgtgttc | 6578 | gaacacaaactccgtgcca | 7968 |
| gctgtgcacgccgcggccg | 3797 | cggccgcggcgtgcacagc | 5188 | ggcacggagtttgtgttca | 6579 | tgaacacaaactccgtgcc | 7969 |
| ctgtgcacgccgcggccgg | 3798 | ccggccgcggcgtgcacag | 5189 | gcacggagtttgtgttcaa | 6580 | ttgaacacaaactccgtgc | 7970 |
| tgtgcacgccgcggccggg | 3799 | cccggccgcggcgtgcaca | 5190 | cacggagtttgtgttcaaa | 6581 | tttgaacacaaactccgtg | 7971 |
| gtgcacgccgcggccgggg | 3800 | ccccggccgcggcgtgcac | 5191 | acggagtttgtgttcaaag | 6582 | ctttgaacacaaactccgt | 7972 |
| tgcacgccgcggccggggc | 3801 | gccccggccgcggcgtgca | 5192 | cggagtttgtgttcaaagt | 6583 | actttgaacacaaactccg | 7973 |
| gcacgccgcggccggggcg | 3802 | cgccccggccgcggcgtgc | 5193 | ggagtttgtgttcaaagtg | 6584 | cactttgaacacaaactcc | 7974 |
| cacgccgcggccggggcgg | 3803 | ccgccccggccgcggcgtg | 5194 | gagtttgtgttcaaagtga | 6585 | tcactttgaacacaaactc | 7975 |
| acgccgcggccggggcggg | 3804 | cccgccccggccgcggcgt | 5195 | agtttgtgttcaaagtgaa | 6586 | ttcactttgaacacaaact | 7976 |
| cgccgcggccggggcgggc | 3805 | gcccgccccggccgcggcg | 5196 | gtttgtgttcaaagtgaat | 6587 | attcactttgaacacaaac | 7977 |
| gccgcggccggggcgggcc | 3806 | ggcccgccccggccgcggc | 5197 | tttgtgttcaaagtgaatc | 6588 | gattcactttgaacacaaa | 7978 |
| ccgcggccggggcgggcct | 3807 | aggcccgccccggccgcgg | 5198 | ttgtgttcaaagtgaatca | 6589 | tgattcactttgaacacaa | 7979 |
| cgcggccggggcgggcctc | 3808 | gaggcccgccccggccgcg | 5199 | tgtgttcaaagtgaatcac | 6590 | gtgattcactttgaacaca | 7980 |
| gcggccggggcgggcctcg | 3809 | cgaggcccgccccggccgc | 5200 | gtgttcaaagtgaatcaca | 6591 | tgtgattcactttgaacac | 7981 |
| cggccggggcgggcctcgg | 3810 | ccgaggcccgccccggccg | 5201 | tgttcaaagtgaatcacat | 6592 | atgtgattcactttgaaca | 7982 |
| ggccggggcgggcctcggg | 3811 | cccgaggcccgccccggcc | 5202 | gttcaaagtgaatcacatg | 6593 | catgtgattcactttgaac | 7983 |
| gccggggcgggcctcggga | 3812 | tcccgaggcccgccccggc | 5203 | ttcaaagtgaatcacatga | 6594 | tcatgtgattcactttgaa | 7984 |
| ccggggcgggcctcgggac | 3813 | gtcccgaggcccgccccgg | 5204 | tcaaagtgaatcacatgaa | 6595 | ttcatgtgattcactttga | 7985 |
| cggggcgggcctcgggact | 3814 | agtcccgaggcccgccccg | 5205 | caaagtgaatcacatgaag | 6596 | cttcatgtgattcactttg | 7986 |
| ggggcgggcctcgggactg | 3815 | cagtcccgaggcccgcccc | 5206 | aaagtgaatcacatgaagg | 6597 | ccttcatgtgattcacttt | 7987 |
| gggcgggcctcgggactgg | 3816 | ccagtcccgaggcccgccc | 5207 | aagtgaatcacatgaaggt | 6598 | accttcatgtgattcactt | 7988 |
| ggcgggcctcgggactggg | 3817 | cccagtcccgaggcccgcc | 5208 | agtgaatcacatgaaggtt | 6599 | aaccttcatgtgattcact | 7989 |
| gcgggcctcgggactgggg | 3818 | ccccagtcccgaggcccgc | 5209 | gtgaatcacatgaaggtta | 6600 | taaccttcatgtgattcac | 7990 |
| cgggcctcgggactggggc | 3819 | gccccagtcccgaggcccg | 5210 | tgaatcacatgaaggttac | 6601 | gtaaccttcatgtgattca | 7991 |
| gggcctcgggactggggct | 3820 | agccccagtcccgaggccc | 5211 | gaatcacatgaaggttact | 6602 | agtaaccttcatgtgattc | 7992 |
| ggcctcgggactggggctg | 3821 | cagccccagtcccgaggcc | 5212 | aatcacatgaaggttactc | 6603 | gagtaaccttcatgtgatt | 7993 |
| gcctcgggactggggctgg | 3822 | ccagccccagtcccgaggc | 5213 | atcacatgaaggttactcc | 6604 | ggagtaaccttcatgtgat | 7994 |
| cctcgggactggggctggg | 3823 | cccagccccagtcccgagg | 5214 | tcacatgaaggttactccc | 6605 | gggagtaaccttcatgtga | 7995 |
| ctcgggactggggctggga | 3824 | tcccagccccagtcccgag | 5215 | cacatgaaggttactccca | 6606 | tgggagtaaccttcatgtg | 7996 |
| tcgggactggggctgggag | 3825 | ctcccagccccagtcccga | 5216 | acatgaaggttactcccat | 6607 | atgggagtaaccttcatgt | 7997 |
| cgggactggggctgggagc | 3826 | gctcccagccccagtcccg | 5217 | catgaaggttactcccatg | 6608 | catgggagtaaccttcatg | 7998 |
| gggactggggctgggagcc | 3827 | ggctcccagccccagtccc | 5218 | atgaaggttactcccatgg | 6609 | ccatgggagtaaccttcat | 7999 |
| ggactggggctgggagcca | 3828 | tggctcccagccccagtcc | 5219 | tgaaggttactcccatgga | 6610 | tccatgggagtaaccttca | 8000 |
| gactggggctgggagccaa | 3829 | ttggctcccagccccagtc | 5220 | gaaggttactcccatggac | 6611 | gtccatgggagtaaccttc | 8001 |
| actggggctgggagccaag | 3830 | cttggctcccagccccagt | 5221 | aaggttactcccatggacg | 6612 | cgtccatgggagtaacctt | 8002 |
| ctggggctgggagccaagg | 3831 | ccttggctcccagccccag | 5222 | aggttactcccatggacgc | 6613 | gcgtccatgggagtaacct | 8003 |
| tggggctgggagccaaggg | 3832 | cccttggctcccagcccca | 5223 | ggttactcccatggacgca | 6614 | tgcgtccatgggagtaacc | 8004 |
| ggggctgggagccaagggg | 3833 | ccccttggctcccagcccc | 5224 | gttactcccatggacgcag | 6615 | ctgcgtccatgggagtaac | 8005 |
| gggctgggagccaaggggc | 3834 | gccccttggctcccagccc | 5225 | ttactcccatggacgcagc | 6616 | gctgcgtccatgggagtaa | 8006 |
| ggctgggagccaaggggcc | 3835 | ggccccttggctcccagcc | 5226 | tactcccatggacgcagcc | 6617 | ggctgcgtccatgggagta | 8007 |
| gctgggagccaaggggccg | 3836 | cggccccttggctcccagc | 5227 | actcccatggacgcagcca | 6618 | tggctgcgtccatgggagt | 8008 |
| ctgggagccaaggggccgg | 3837 | ccggccccttggctcccag | 5228 | ctcccatggacgcagccac | 6619 | gtggctgcgtccatgggag | 8009 |
| tgggagccaaggggccggg | 3838 | cccggccccttggctccca | 5229 | tcccatggacgcagccaca | 6620 | tgtggctgcgtccatggga | 8010 |
| gggagccaaggggccgggg | 3839 | ccccggccccttggctccc | 5230 | cccatggacgcagccacag | 6621 | ctgtggctgcgtccatggg | 8011 |
| ggagccaaggggccggggc | 3840 | gccccggccccttggctcc | 5231 | ccatggacgcagccacagc | 6622 | gctgtggctgcgtccatgg | 8012 |
| gagccaaggggccggggcg | 3841 | cgccccggccccttggctc | 5232 | catggacgcagccacagcc | 6623 | ggctgtggctgcgtccatg | 8013 |
| agccaaggggccggggcgg | 3842 | ccgccccggccccttggct | 5233 | atggacgcagccacagcct | 6624 | aggctgtggctgcgtccat | 8014 |
| gccaaggggccggggcggg | 3843 | cccgccccggccccttggc | 5234 | tggacgcagccacagcctc | 6625 | gaggctgtggctgcgtcca | 8015 |
| ccaaggggccggggcggga | 3844 | tcccgccccggccccttgg | 5235 | ggacgcagccacagcctcc | 6626 | ggaggctgtggctgcgtcc | 8016 |
| caaggggccggggcgggac | 3845 | gtcccgccccggccccttg | 5236 | gacgcagccacagcctccc | 6627 | gggaggctgtggctgcgtc | 8017 |
| aaggggccggggcgggacg | 3846 | cgtcccgccccggcccctt | 5237 | acgcagccacagcctccct | 6628 | agggaggctgtggctgcgt | 8018 |
| aggggccggggcgggacgc | 3847 | gcgtcccgccccggcccct | 5238 | cgcagccacagcctccctc | 6629 | gagggaggctgtggctgcg | 8019 |
| ggggccggggcgggacgcg | 3848 | cgcgtcccgccccggcccc | 5239 | gcagccacagcctccctcc | 6630 | ggagggaggctgtggctgc | 8020 |
| gggccggggcgggacgcgg | 3849 | ccgcgtcccgccccggccc | 5240 | cagccacagcctccctcct | 6631 | aggagggaggctgtggctg | 8021 |
| ggccggggcgggacgcgga | 3850 | tccgcgtcccgccccggcc | 5241 | agccacagcctccctcctc | 6632 | gaggagggaggctgtggct | 8022 |
| gccggggcgggacgcggag | 3851 | ctccgcgtcccgccccggc | 5242 | gccacagcctccctcctca | 6633 | tgaggagggaggctgtggc | 8023 |
| ccggggcgggacgcggaga | 3852 | tctccgcgtcccgccccgg | 5243 | ccacagcctccctcctcaa | 6634 | ttgaggagggaggctgtgg | 8024 |
| cggggcgggacgcggagag | 3853 | ctctccgcgtcccgccccg | 5244 | cacagcctccctcctcaat | 6635 | attgaggagggaggctgtg | 8025 |
| ggggcgggacgcggagagg | 3854 | cctctccgcgtcccgcccc | 5245 | acagcctccctcctcaatg | 6636 | cattgaggagggaggctgt | 8026 |
| gggcgggacgcggagaggc | 3855 | gcctctccgcgtcccgccc | 5246 | cagcctccctcctcaatgt | 6637 | acattgaggagggaggctg | 8027 |
| ggcgggacgcggagaggct | 3856 | agcctctccgcgtcccgcc | 5247 | agcctccctcctcaatgtc | 6638 | gacattgaggagggaggct | 8028 |
| gcgggacgcggagaggctg | 3857 | cagcctctccgcgtcccgc | 5248 | gcctccctcctcaatgtct | 6639 | agacattgaggagggaggc | 8029 |
| cgggacgcggagaggctgg | 3858 | ccagcctctccgcgtcccg | 5249 | cctccctcctcaatgtctt | 6640 | aagacattgaggagggagg | 8030 |
| gggacgcggagaggctggg | 3859 | cccagcctctccgcgtccc | 5250 | ctccctcctcaatgtcttc | 6641 | gaagacattgaggagggag | 8031 |
| ggacgcggagaggctgggc | 3860 | gcccagcctctccgcgtcc | 5251 | tccctcctcaatgtcttca | 6642 | tgaagacattgaggaggga | 8032 |
| gacgcggagaggctgggct | 3861 | agcccagcctctccgcgtc | 5252 | ccctcctcaatgtcttcag | 6643 | ctgaagacattgaggaggg | 8033 |
| acgcggagaggctgggctg | 3862 | cagcccagcctctccgcgt | 5253 | cctcctcaatgtcttcagt | 6644 | actgaagacattgaggagg | 8034 |
| cgcggagaggctgggctgc | 3863 | gcagcccagcctctccgcg | 5254 | ctcctcaatgtcttcagtg | 6645 | cactgaagacattgaggag | 8035 |
| gcggagaggctgggctgcg | 3864 | cgcagcccagcctctccgc | 5255 | tcctcaatgtcttcagtgg | 6646 | ccactgaagacattgagga | 8036 |
| cggagaggctgggctgcgg | 3865 | ccgcagcccagcctctccg | 5256 | cctcaatgtcttcagtggg | 6647 | cccactgaagacattgagg | 8037 |
| ggagaggctgggctgcggt | 3866 | accgcagcccagcctctcc | 5257 | ctcaatgtcttcagtggga | 6648 | tcccactgaagacattgag | 8038 |
| gagaggctgggctgcggtt | 3867 | aaccgcagcccagcctctc | 5258 | tcaatgtcttcagtgggaa | 6649 | ttcccactgaagacattga | 8039 |
| agaggctgggctgcggttc | 3868 | gaaccgcagcccagcctct | 5259 | caatgtcttcagtgggaat | 6650 | attcccactgaagacattg | 8040 |
| gaggctgggctgcggttcg | 3869 | cgaaccgcagcccagcctc | 5260 | aatgtcttcagtgggaatg | 6651 | cattcccactgaagacatt | 8041 |
| aggctgggctgcggttcgg | 3870 | ccgaaccgcagcccagcct | 5261 | atgtcttcagtgggaatga | 6652 | tcattcccactgaagacat | 8042 |
| ggctgggctgcggttcgga | 3871 | tccgaaccgcagcccagcc | 5262 | tgtcttcagtgggaatgag | 6653 | ctcattcccactgaagaca | 8043 |
| gctgggctgcggttcggag | 3872 | ctccgaaccgcagcccagc | 5263 | gtcttcagtgggaatgagt | 6654 | actcattcccactgaagac | 8044 |
| ctgggctgcggttcggagt | 3873 | actccgaaccgcagcccag | 5264 | tcttcagtgggaatgagtg | 6655 | cactcattcccactgaaga | 8045 |
| tgggctgcggttcggagtc | 3874 | gactccgaaccgcagccca | 5265 | cttcagtgggaatgagtgt | 6656 | acactcattcccactgaag | 8046 |
| gggctgcggttcggagtcc | 3875 | ggactccgaaccgcagccc | 5266 | ttcagtgggaatgagtgtg | 6657 | cacactcattcccactgaa | 8047 |
| ggctgcggttcggagtccc | 3876 | gggactccgaaccgcagcc | 5267 | tcagtgggaatgagtgtgg | 6658 | ccacactcattcccactga | 8048 |
| gctgcggttcggagtcccg | 3877 | cgggactccgaaccgcagc | 5268 | cagtgggaatgagtgtggg | 6659 | cccacactcattcccactg | 8049 |
| ctgcggttcggagtcccgc | 3878 | gcgggactccgaaccgcag | 5269 | agtgggaatgagtgtgggg | 6660 | ccccacactcattcccact | 8050 |
| tgcggttcggagtcccgcg | 3879 | cgcgggactccgaaccgca | 5270 | gtgggaatgagtgtggggc | 6661 | gccccacactcattcccac | 8051 |
| gcggttcggagtcccgcgc | 3880 | gcgcgggactccgaaccgc | 5271 | tgggaatgagtgtggggct | 6662 | agccccacactcattccca | 8052 |
| cggttcggagtcccgcgcg | 3881 | cgcgcgggactccgaaccg | 5272 | gggaatgagtgtggggctg | 6663 | cagccccacactcattccc | 8053 |
| ggttcggagtcccgcgcgg | 3882 | ccgcgcgggactccgaacc | 5273 | ggaatgagtgtggggctga | 6664 | tcagccccacactcattcc | 8054 |
| gttcggagtcccgcgcgga | 3883 | tccgcgcgggactccgaac | 5274 | gaatgagtgtggggctgag | 6665 | ctcagccccacactcattc | 8055 |
| ttcggagtcccgcgcggac | 3884 | gtccgcgcgggactccgaa | 5275 | aatgagtgtggggctgagg | 6666 | cctcagccccacactcatt | 8056 |
| tcggagtcccgcgcggaca | 3885 | tgtccgcgcgggactccga | 5276 | atgagtgtggggctgaggg | 6667 | ccctcagccccacactcat | 8057 |
| cggagtcccgcgcggacag | 3886 | ctgtccgcgcgggactccg | 5277 | tgagtgtggggctgagggc | 6668 | gccctcagccccacactca | 8058 |
| ggagtcccgcgcggacagg | 3887 | cctgtccgcgcgggactcc | 5278 | gagtgtggggctgagggct | 6669 | agccctcagccccacactc | 8059 |
| gagtcccgcgcggacaggg | 3888 | ccctgtccgcgcgggactc | 5279 | agtgtggggctgagggctc | 6670 | gagccctcagccccacact | 8060 |
| agtcccgcgcggacagggg | 3889 | cccctgtccgcgcgggact | 5280 | gtgtggggctgagggctcc | 6671 | ggagccctcagccccacac | 8061 |
| gtcccgcgcggacaggggc | 3890 | gcccctgtccgcgcgggac | 5281 | tgtggggctgagggctcct | 6672 | aggagccctcagccccaca | 8062 |
| tcccgcgcggacaggggcc | 3891 | ggcccctgtccgcgcggga | 5282 | gtggggctgagggctcctg | 6673 | caggagccctcagccccac | 8063 |
| cccgcgcggacaggggccg | 3892 | cggcccctgtccgcgcggg | 5283 | tggggctgagggctcctgg | 6674 | ccaggagccctcagcccca | 8064 |
| ccgcgcggacaggggccgg | 3893 | ccggcccctgtccgcgcgg | 5284 | ggggctgagggctcctggc | 6675 | gccaggagccctcagcccc | 8065 |
| cgcgcggacaggggccgga | 3894 | tccggcccctgtccgcgcg | 5285 | gggctgagggctcctggca | 6676 | tgccaggagccctcagccc | 8066 |
| gcgcggacaggggccggac | 3895 | gtccggcccctgtccgcgc | 5286 | ggctgagggctcctggcag | 6677 | ctgccaggagccctcagcc | 8067 |
| cgcggacaggggccggacg | 3896 | cgtccggcccctgtccgcg | 5287 | gctgagggctcctggcagg | 6678 | cctgccaggagccctcagc | 8068 |
| gcggacaggggccggacgg | 3897 | ccgtccggcccctgtccgc | 5288 | ctgagggctcctggcaggt | 6679 | acctgccaggagccctcag | 8069 |
| cggacaggggccggacggc | 3898 | gccgtccggcccctgtccg | 5289 | tgagggctcctggcaggtg | 6680 | cacctgccaggagccctca | 8070 |
| ggacaggggccggacggcg | 3899 | cgccgtccggcccctgtcc | 5290 | gagggctcctggcaggtgg | 6681 | ccacctgccaggagccctc | 8071 |
| gacaggggccggacggcgg | 3900 | ccgccgtccggcccctgtc | 5291 | agggctcctggcaggtggg | 6682 | cccacctgccaggagccct | 8072 |
| acaggggccggacggcggc | 3901 | gccgccgtccggcccctgt | 5292 | gggctcctggcaggtgggt | 6683 | acccacctgccaggagccc | 8073 |
| caggggccggacggcggcg | 3902 | cgccgccgtccggcccctg | 5293 | ggctcctggcaggtgggta | 6684 | tacccacctgccaggagcc | 8074 |
| aggggccggacggcggcga | 3903 | tcgccgccgtccggcccct | 5294 | gctcctggcaggtgggtat | 6685 | atacccacctgccaggagc | 8075 |
| ggggccggacggcggcgag | 3904 | ctcgccgccgtccggcccc | 5295 | ctcctggcaggtgggtatc | 6686 | gatacccacctgccaggag | 8076 |
| gggccggacggcggcgagg | 3905 | cctcgccgccgtccggccc | 5296 | tcctggcaggtgggtatcc | 6687 | ggatacccacctgccagga | 8077 |
| ggccggacggcggcgaggg | 3906 | ccctcgccgccgtccggcc | 5297 | cctggcaggtgggtatcca | 6688 | tggatacccacctgccagg | 8078 |
| gccggacggcggcgaggga | 3907 | tccctcgccgccgtccggc | 5298 | ctggcaggtgggtatccaa | 6689 | ttggatacccacctgccag | 8079 |
| ccggacggcggcgagggag | 3908 | ctccctcgccgccgtccgg | 5299 | tggcaggtgggtatccaac | 6690 | gttggatacccacctgcca | 8080 |
| cggacggcggcgagggagc | 3909 | gctccctcgccgccgtccg | 5300 | ggcaggtgggtatccaaca | 6691 | tgttggatacccacctgcc | 8081 |
| ggacggcggcgagggagcg | 3910 | cgctccctcgccgccgtcc | 5301 | gcaggtgggtatccaacag | 6692 | ctgttggatacccacctgc | 8082 |
| gacggcggcgagggagcgc | 3911 | gcgctccctcgccgccgtc | 5302 | caggtgggtatccaacagg | 6693 | cctgttggatacccacctg | 8083 |
| acggcggcgagggagcgcg | 3912 | cgcgctccctcgccgccgt | 5303 | aggtgggtatccaacagga | 6694 | tcctgttggatacccacct | 8084 |
| cggcggcgagggagcgcgc | 3913 | gcgcgctccctcgccgccg | 5304 | ggtgggtatccaacaggat | 6695 | atcctgttggatacccacc | 8085 |
| ggcggcgagggagcgcgcg | 3914 | cgcgcgctccctcgccgcc | 5305 | gtgggtatccaacaggatg | 6696 | catcctgttggatacccac | 8086 |
| gcggcgagggagcgcgcgc | 3915 | gcgcgcgctccctcgccgc | 5306 | tgggtatccaacaggatgt | 6697 | acatcctgttggataccca | 8087 |
| cggcgagggagcgcgcgcc | 3916 | ggcgcgcgctccctcgccg | 5307 | gggtatccaacaggatgtg | 6698 | cacatcctgttggataccc | 8088 |
| ggcgagggagcgcgcgccc | 3917 | gggcgcgcgctccctcgcc | 5308 | ggtatccaacaggatgtga | 6699 | tcacatcctgttggatacc | 8089 |
| gcgagggagcgcgcgcccc | 3918 | ggggcgcgcgctccctcgc | 5309 | gtatccaacaggatgtgac | 6700 | gtcacatcctgttggatac | 8090 |
| cgagggagcgcgcgccccg | 3919 | cggggcgcgcgctccctcg | 5310 | tatccaacaggatgtgaca | 6701 | tgtcacatcctgttggata | 8091 |
| gagggagcgcgcgccccgc | 3920 | gcggggcgcgcgctccctc | 5311 | atccaacaggatgtgacac | 6702 | gtgtcacatcctgttggat | 8092 |
| agggagcgcgcgccccgca | 3921 | tgcggggcgcgcgctccct | 5312 | tccaacaggatgtgacaca | 6703 | tgtgtcacatcctgttgga | 8093 |
| gggagcgcgcgccccgcag | 3922 | ctgcggggcgcgcgctccc | 5313 | ccaacaggatgtgacacat | 6704 | atgtgtcacatcctgttgg | 8094 |
| ggagcgcgcgccccgcagt | 3923 | actgcggggcgcgcgctcc | 5314 | caacaggatgtgacacata | 6705 | tatgtgtcacatcctgttg | 8095 |
| gagcgcgcgccccgcagtc | 3924 | gactgcggggcgcgcgctc | 5315 | aacaggatgtgacacatac | 6706 | gtatgtgtcacatcctgtt | 8096 |
| agcgcgcgccccgcagtcc | 3925 | ggactgcggggcgcgcgct | 5316 | acaggatgtgacacatacc | 6707 | ggtatgtgtcacatcctgt | 8097 |
| gcgcgcgccccgcagtccc | 3926 | gggactgcggggcgcgcgc | 5317 | caggatgtgacacatacca | 6708 | tggtatgtgtcacatcctg | 8098 |
| cgcgcgccccgcagtcccg | 3927 | cgggactgcggggcgcgcg | 5318 | aggatgtgacacataccaa | 6709 | ttggtatgtgtcacatcct | 8099 |
| gcgcgccccgcagtcccgc | 3928 | gcgggactgcggggcgcgc | 5319 | ggatgtgacacataccaat | 6710 | attggtatgtgtcacatcc | 8100 |
| cgcgccccgcagtcccgcg | 3929 | cgcgggactgcggggcgcg | 5320 | gatgtgacacataccaatg | 6711 | cattggtatgtgtcacatc | 8101 |
| gcgccccgcagtcccgcgc | 3930 | gcgcgggactgcggggcgc | 5321 | atgtgacacataccaatgg | 6712 | ccattggtatgtgtcacat | 8102 |
| cgccccgcagtcccgcgct | 3931 | agcgcgggactgcggggcg | 5322 | tgtgacacataccaatggc | 6713 | gccattggtatgtgtcaca | 8103 |
| gccccgcagtcccgcgctg | 3932 | cagcgcgggactgcggggc | 5323 | gtgacacataccaatggct | 6714 | agccattggtatgtgtcac | 8104 |
| ccccgcagtcccgcgctgc | 3933 | gcagcgcgggactgcgggg | 5324 | tgacacataccaatggctg | 6715 | cagccattggtatgtgtca | 8105 |
| cccgcagtcccgcgctgcg | 3934 | cgcagcgcgggactgcggg | 5325 | gacacataccaatggctgc | 6716 | gcagccattggtatgtgtc | 8106 |
| ccgcagtcccgcgctgcgc | 3935 | gcgcagcgcgggactgcgg | 5326 | acacataccaatggctgcg | 6717 | cgcagccattggtatgtgt | 8107 |
| cgcagtcccgcgctgcgcc | 3936 | ggcgcagcgcgggactgcg | 5327 | cacataccaatggctgcgt | 6718 | acgcagccattggtatgtg | 8108 |
| gcagtcccgcgctgcgccg | 3937 | cggcgcagcgcgggactgc | 5328 | acataccaatggctgcgtg | 6719 | cacgcagccattggtatgt | 8109 |
| cagtcccgcgctgcgccgg | 3938 | ccggcgcagcgcgggactg | 5329 | cataccaatggctgcgtgg | 6720 | ccacgcagccattggtatg | 8110 |
| agtcccgcgctgcgccggc | 3939 | gccggcgcagcgcgggact | 5330 | ataccaatggctgcgtggc | 6721 | gccacgcagccattggtat | 8111 |
| gtcccgcgctgcgccggcc | 3940 | ggccggcgcagcgcgggac | 5331 | taccaatggctgcgtggct | 6722 | agccacgcagccattggta | 8112 |
| tcccgcgctgcgccggccg | 3941 | cggccggcgcagcgcggga | 5332 | accaatggctgcgtggctc | 6723 | gagccacgcagccattggt | 8113 |
| cccgcgctgcgccggccgg | 3942 | ccggccggcgcagcgcggg | 5333 | ccaatggctgcgtggctct | 6724 | agagccacgcagccattgg | 8114 |
| ccgcgctgcgccggccggg | 3943 | cccggccggcgcagcgcgg | 5334 | caatggctgcgtggctctg | 6725 | cagagccacgcagccattg | 8115 |
| cgcgctgcgccggccgggg | 3944 | ccccggccggcgcagcgcg | 5335 | aatggctgcgtggctctgg | 6726 | ccagagccacgcagccatt | 8116 |
| gcgctgcgccggccgggga | 3945 | tccccggccggcgcagcgc | 5336 | atggctgcgtggctctggg | 6727 | cccagagccacgcagccat | 8117 |
| cgctgcgccggccggggat | 3946 | atccccggccggcgcagcg | 5337 | tggctgcgtggctctgggc | 6728 | gcccagagccacgcagcca | 8118 |
| gctgcgccggccggggatg | 3947 | catccccggccggcgcagc | 5338 | ggctgcgtggctctgggca | 6729 | tgcccagagccacgcagcc | 8119 |
| ctgcgccggccggggatgg | 3948 | ccatccccggccggcgcag | 5339 | gctgcgtggctctgggcat | 6730 | atgcccagagccacgcagc | 8120 |
| tgcgccggccggggatggg | 3949 | cccatccccggccggcgca | 5340 | ctgcgtggctctgggcatc | 6731 | gatgcccagagccacgcag | 8121 |
| gcgccggccggggatgggg | 3950 | ccccatccccggccggcgc | 5341 | tgcgtggctctgggcatca | 6732 | tgatgcccagagccacgca | 8122 |
| cgccggccggggatggggc | 3951 | gccccatccccggccggcg | 5342 | gcgtggctctgggcatcaa | 6733 | ttgatgcccagagccacgc | 8123 |
| gccggccggggatggggcg | 3952 | cgccccatccccggccggc | 5343 | cgtggctctgggcatcaaa | 6734 | tttgatgcccagagccacg | 8124 |
| ccggccggggatggggcgc | 3953 | gcgccccatccccggccgg | 5344 | gtggctctgggcatcaaac | 6735 | gtttgatgcccagagccac | 8125 |
| cggccggggatggggcgcg | 3954 | cgcgccccatccccggccg | 5345 | tggctctgggcatcaaact | 6736 | agtttgatgcccagagcca | 8126 |
| ggccggggatggggcgcgc | 3955 | gcgcgccccatccccggcc | 5346 | ggctctgggcatcaaacta | 6737 | tagtttgatgcccagagcc | 8127 |
| gccggggatggggcgcgct | 3956 | agcgcgccccatccccggc | 5347 | gctctgggcatcaaactac | 6738 | gtagtttgatgcccagagc | 8128 |
| ccggggatggggcgcgctg | 3957 | cagcgcgccccatccccgg | 5348 | ctctgggcatcaaactacc | 6739 | ggtagtttgatgcccagag | 8129 |
| cggggatggggcgcgctgc | 3958 | gcagcgcgccccatccccg | 5349 | tctgggcatcaaactacct | 6740 | aggtagtttgatgcccaga | 8130 |
| ggggatggggcgcgctgct | 3959 | agcagcgcgccccatcccc | 5350 | ctgggcatcaaactacctc | 6741 | gaggtagtttgatgcccag | 8131 |
| gggatggggcgcgctgctg | 3960 | cagcagcgcgccccatccc | 5351 | tgggcatcaaactacctca | 6742 | tgaggtagtttgatgccca | 8132 |
| ggatggggcgcgctgctgc | 3961 | gcagcagcgcgccccatcc | 5352 | gggcatcaaactacctcac | 6743 | gtgaggtagtttgatgccc | 8133 |
| gatggggcgcgctgctgcc | 3962 | ggcagcagcgcgccccatc | 5353 | ggcatcaaactacctcaca | 6744 | tgtgaggtagtttgatgcc | 8134 |
| atggggcgcgctgctgcct | 3963 | aggcagcagcgcgccccat | 5354 | gcatcaaactacctcacac | 6745 | gtgtgaggtagtttgatgc | 8135 |
| tggggcgcgctgctgcctg | 3964 | caggcagcagcgcgcccca | 5355 | catcaaactacctcacaca | 6746 | tgtgtgaggtagtttgatg | 8136 |
| ggggcgcgctgctgcctga | 3965 | tcaggcagcagcgcgcccc | 5356 | atcaaactacctcacacag | 6747 | ctgtgtgaggtagtttgat | 8137 |
| gggcgcgctgctgcctgag | 3966 | ctcaggcagcagcgcgccc | 5357 | tcaaactacctcacacaga | 6748 | tctgtgtgaggtagtttga | 8138 |
| ggcgcgctgctgcctgagg | 3967 | cctcaggcagcagcgcgcc | 5358 | caaactacctcacacagaa | 6749 | ttctgtgtgaggtagtttg | 8139 |
| gcgcgctgctgcctgaggc | 3968 | gcctcaggcagcagcgcgc | 5359 | aaactacctcacacagaat | 6750 | attctgtgtgaggtagttt | 8140 |
| cgcgctgctgcctgaggcc | 3969 | ggcctcaggcagcagcgcg | 5360 | aactacctcacacagaata | 6751 | tattctgtgtgaggtagtt | 8141 |
| gcgctgctgcctgaggccc | 3970 | gggcctcaggcagcagcgc | 5361 | actacctcacacagaatat | 6752 | atattctgtgtgaggtagt | 8142 |
| cgctgctgcctgaggcccg | 3971 | cgggcctcaggcagcagcg | 5362 | ctacctcacacagaatatg | 6753 | catattctgtgtgaggtag | 8143 |
| gctgctgcctgaggcccgg | 3972 | ccgggcctcaggcagcagc | 5363 | tacctcacacagaatatga | 6754 | tcatattctgtgtgaggta | 8144 |
| ctgctgcctgaggcccggc | 3973 | gccgggcctcaggcagcag | 5364 | acctcacacagaatatgag | 6755 | ctcatattctgtgtgaggt | 8145 |
| tgctgcctgaggcccggcc | 3974 | ggccgggcctcaggcagca | 5365 | cctcacacagaatatgaga | 6756 | tctcatattctgtgtgagg | 8146 |
| gctgcctgaggcccggcct | 3975 | aggccgggcctcaggcagc | 5366 | ctcacacagaatatgagat | 6757 | atctcatattctgtgtgag | 8147 |
| ctgcctgaggcccggcctg | 3976 | caggccgggcctcaggcag | 5367 | tcacacagaatatgagatc | 6758 | gatctcatattctgtgtga | 8148 |
| tgcctgaggcccggcctgg | 3977 | ccaggccgggcctcaggca | 5368 | cacacagaatatgagatct | 6759 | agatctcatattctgtgtg | 8149 |
| gcctgaggcccggcctggc | 3978 | gccaggccgggcctcaggc | 5369 | acacagaatatgagatctt | 6760 | aagatctcatattctgtgt | 8150 |
| cctgaggcccggcctggcg | 3979 | cgccaggccgggcctcagg | 5370 | cacagaatatgagatcttc | 6761 | gaagatctcatattctgtg | 8151 |
| ctgaggcccggcctggcgg | 3980 | ccgccaggccgggcctcag | 5371 | acagaatatgagatcttca | 6762 | tgaagatctcatattctgt | 8152 |
| tgaggcccggcctggcggg | 3981 | cccgccaggccgggcctca | 5372 | cagaatatgagatcttcaa | 6763 | ttgaagatctcatattctg | 8153 |
| gaggcccggcctggcgggc | 3982 | gcccgccaggccgggcctc | 5373 | agaatatgagatcttcaaa | 6764 | tttgaagatctcatattct | 8154 |
| aggcccggcctggcgggcg | 3983 | cgcccgccaggccgggcct | 5374 | gaatatgagatcttcaaaa | 6765 | ttttgaagatctcatattc | 8155 |
| ggcccggcctggcgggcgc | 3984 | gcgcccgccaggccgggcc | 5375 | aatatgagatcttcaaaat | 6766 | attttgaagatctcatatt | 8156 |
| gcccggcctggcgggcgcc | 3985 | ggcgcccgccaggccgggc | 5376 | atatgagatcttcaaaatg | 6767 | cattttgaagatctcatat | 8157 |
| cccggcctggcgggcgccc | 3986 | gggcgcccgccaggccggg | 5377 | tatgagatcttcaaaatgg | 6768 | ccattttgaagatctcata | 8158 |
| ccggcctggcgggcgcccg | 3987 | cgggcgcccgccaggccgg | 5378 | atgagatcttcaaaatgga | 6769 | tccattttgaagatctcat | 8159 |
| cggcctggcgggcgcccgc | 3988 | gcgggcgcccgccaggccg | 5379 | tgagatcttcaaaatggag | 6770 | ctccattttgaagatctca | 8160 |
| ggcctggcgggcgcccgcc | 3989 | ggcgggcgcccgccaggcc | 5380 | gagatcttcaaaatggagc | 6771 | gctccattttgaagatctc | 8161 |
| gcctggcgggcgcccgccg | 3990 | cggcgggcgcccgccaggc | 5381 | agatcttcaaaatggagca | 6772 | tgctccattttgaagatct | 8162 |
| cctggcgggcgcccgccgg | 3991 | ccggcgggcgcccgccagg | 5382 | gatcttcaaaatggagcaa | 6773 | ttgctccattttgaagatc | 8163 |
| ctggcgggcgcccgccggg | 3992 | cccggcgggcgcccgccag | 5383 | atcttcaaaatggagcaag | 6774 | cttgctccattttgaagat | 8164 |
| tggcgggcgcccgccgggg | 3993 | ccccggcgggcgcccgcca | 5384 | tcttcaaaatggagcaaga | 6775 | tcttgctccattttgaaga | 8165 |
| ggcgggcgcccgccggggg | 3994 | cccccggcgggcgcccgcc | 5385 | cttcaaaatggagcaagac | 6776 | gtcttgctccattttgaag | 8166 |
| gcgggcgcccgccgggggc | 3995 | gcccccggcgggcgcccgc | 5386 | ttcaaaatggagcaagaca | 6777 | tgtcttgctccattttgaa | 8167 |
| cgggcgcccgccgggggct | 3996 | agcccccggcgggcgcccg | 5387 | tcaaaatggagcaagacac | 6778 | gtgtcttgctccattttga | 8168 |
| gggcgcccgccgggggctg | 3997 | cagcccccggcgggcgccc | 5388 | caaaatggagcaagacacc | 6779 | ggtgtcttgctccattttg | 8169 |
| ggcgcccgccgggggctgc | 3998 | gcagcccccggcgggcgcc | 5389 | aaaatggagcaagacaccc | 6780 | gggtgtcttgctccatttt | 8170 |
| gcgcccgccgggggctgcg | 3999 | cgcagcccccggcgggcgc | 5390 | aaatggagcaagacacccg | 6781 | cgggtgtcttgctccattt | 8171 |
| cgcccgccgggggctgcgg | 4000 | ccgcagcccccggcgggcg | 5391 | aatggagcaagacacccga | 6782 | tcgggtgtcttgctccatt | 8172 |
| gcccgccgggggctgcggc | 4001 | gccgcagcccccggcgggc | 5392 | atggagcaagacacccgag | 6783 | ctcgggtgtcttgctccat | 8173 |
| cccgccgggggctgcggct | 4002 | agccgcagcccccggcggg | 5393 | tggagcaagacacccgagg | 6784 | cctcgggtgtcttgctcca | 8174 |
| ccgccgggggctgcggctg | 4003 | cagccgcagcccccggcgg | 5394 | ggagcaagacacccgaggc | 6785 | gcctcgggtgtcttgctcc | 8175 |
| cgccgggggctgcggctga | 4004 | tcagccgcagcccccggcg | 5395 | gagcaagacacccgaggcc | 6786 | ggcctcgggtgtcttgctc | 8176 |
| gccgggggctgcggctgag | 4005 | ctcagccgcagcccccggc | 5396 | agcaagacacccgaggccg | 6787 | cggcctcgggtgtcttgct | 8177 |
| ccgggggctgcggctgagg | 4006 | cctcagccgcagcccccgg | 5397 | gcaagacacccgaggccgc | 6788 | gcggcctcgggtgtcttgc | 8178 |
| cgggggctgcggctgagga | 4007 | tcctcagccgcagcccccg | 5398 | caagacacccgaggccgct | 6789 | agcggcctcgggtgtcttg | 8179 |
| gggggctgcggctgaggag | 4008 | ctcctcagccgcagccccc | 5399 | aagacacccgaggccgcta | 6790 | tagcggcctcgggtgtctt | 8180 |
| ggggctgcggctgaggagc | 4009 | gctcctcagccgcagcccc | 5400 | agacacccgaggccgctac | 6791 | gtagcggcctcgggtgtct | 8181 |
| gggctgcggctgaggagcc | 4010 | ggctcctcagccgcagccc | 5401 | gacacccgaggccgctacc | 6792 | ggtagcggcctcgggtgtc | 8182 |
| ggctgcggctgaggagccg | 4011 | cggctcctcagccgcagcc | 5402 | acacccgaggccgctacct | 6793 | aggtagcggcctcgggtgt | 8183 |
| gctgcggctgaggagccga | 4012 | tcggctcctcagccgcagc | 5403 | cacccgaggccgctacctg | 6794 | caggtagcggcctcgggtg | 8184 |
| ctgcggctgaggagccgag | 4013 | ctcggctcctcagccgcag | 5404 | acccgaggccgctacctgc | 6795 | gcaggtagcggcctcgggt | 8185 |
| tgcggctgaggagccgagg | 4014 | cctcggctcctcagccgca | 5405 | cccgaggccgctacctgct | 6796 | agcaggtagcggcctcggg | 8186 |
| gcggctgaggagccgaggg | 4015 | ccctcggctcctcagccgc | 5406 | ccgaggccgctacctgctg | 6797 | cagcaggtagcggcctcgg | 8187 |
| cggctgaggagccgagggc | 4016 | gccctcggctcctcagccg | 5407 | cgaggccgctacctgctgt | 6798 | acagcaggtagcggcctcg | 8188 |
| ggctgaggagccgagggcg | 4017 | cgccctcggctcctcagcc | 5408 | gaggccgctacctgctgtt | 6799 | aacagcaggtagcggcctc | 8189 |
| gctgaggagccgagggcgc | 4018 | gcgccctcggctcctcagc | 5409 | aggccgctacctgctgttc | 6800 | gaacagcaggtagcggcct | 8190 |
| ctgaggagccgagggcgcc | 4019 | ggcgccctcggctcctcag | 5410 | ggccgctacctgctgttca | 6801 | tgaacagcaggtagcggcc | 8191 |
| tgaggagccgagggcgccc | 4020 | gggcgccctcggctcctca | 5411 | gccgctacctgctgttcaa | 6802 | ttgaacagcaggtagcggc | 8192 |
| gaggagccgagggcgccct | 4021 | agggcgccctcggctcctc | 5412 | ccgctacctgctgttcaat | 6803 | attgaacagcaggtagcgg | 8193 |
| aggagccgagggcgccctg | 4022 | cagggcgccctcggctcct | 5413 | cgctacctgctgttcaatg | 6804 | cattgaacagcaggtagcg | 8194 |
| ggagccgagggcgccctgt | 4023 | acagggcgccctcggctcc | 5414 | gctacctgctgttcaatgg | 6805 | ccattgaacagcaggtagc | 8195 |
| gagccgagggcgccctgta | 4024 | tacagggcgccctcggctc | 5415 | ctacctgctgttcaatggc | 6806 | gccattgaacagcaggtag | 8196 |
| agccgagggcgccctgtac | 4025 | gtacagggcgccctcggct | 5416 | tacctgctgttcaatggcc | 6807 | ggccattgaacagcaggta | 8197 |
| gccgagggcgccctgtacc | 4026 | ggtacagggcgccctcggc | 5417 | acctgctgttcaatggcca | 6808 | tggccattgaacagcaggt | 8198 |
| ccgagggcgccctgtaccg | 4027 | cggtacagggcgccctcgg | 5418 | cctgctgttcaatggccag | 6809 | ctggccattgaacagcagg | 8199 |
| cgagggcgccctgtaccgg | 4028 | ccggtacagggcgccctcg | 5419 | ctgctgttcaatggccaga | 6810 | tctggccattgaacagcag | 8200 |
| gagggcgccctgtaccgga | 4029 | tccggtacagggcgccctc | 5420 | tgctgttcaatggccagag | 6811 | ctctggccattgaacagca | 8201 |
| agggcgccctgtaccggag | 4030 | ctccggtacagggcgccct | 5421 | gctgttcaatggccagagg | 6812 | cctctggccattgaacagc | 8202 |
| gggcgccctgtaccggagt | 4031 | actccggtacagggcgccc | 5422 | ctgttcaatggccagaggc | 6813 | gcctctggccattgaacag | 8203 |
| ggcgccctgtaccggagtg | 4032 | cactccggtacagggcgcc | 5423 | tgttcaatggccagaggcc | 6814 | ggcctctggccattgaaca | 8204 |
| gcgccctgtaccggagtgg | 4033 | ccactccggtacagggcgc | 5424 | gttcaatggccagaggccc | 6815 | gggcctctggccattgaac | 8205 |
| cgccctgtaccggagtggc | 4034 | gccactccggtacagggcg | 5425 | ttcaatggccagaggccca | 6816 | tgggcctctggccattgaa | 8206 |
| gccctgtaccggagtggcc | 4035 | ggccactccggtacagggc | 5426 | tcaatggccagaggcccag | 6817 | ctgggcctctggccattga | 8207 |
| ccctgtaccggagtggccc | 4036 | gggccactccggtacaggg | 5427 | caatggccagaggcccagc | 6818 | gctgggcctctggccattg | 8208 |
| cctgtaccggagtggcccg | 4037 | cgggccactccggtacagg | 5428 | aatggccagaggcccagcg | 6819 | cgctgggcctctggccatt | 8209 |
| ctgtaccggagtggcccgc | 4038 | gcgggccactccggtacag | 5429 | atggccagaggcccagcga | 6820 | tcgctgggcctctggccat | 8210 |
| tgtaccggagtggcccgcg | 4039 | cgcgggccactccggtaca | 5430 | tggccagaggcccagcgat | 6821 | atcgctgggcctctggcca | 8211 |
| gtaccggagtggcccgcgc | 4040 | gcgcgggccactccggtac | 5431 | ggccagaggcccagcgatg | 6822 | catcgctgggcctctggcc | 8212 |
| taccggagtggcccgcgcg | 4041 | cgcgcgggccactccggta | 5432 | gccagaggcccagcgatgg | 6823 | ccatcgctgggcctctggc | 8213 |
| accggagtggcccgcgcgc | 4042 | gcgcgcgggccactccggt | 5433 | ccagaggcccagcgatggc | 6824 | gccatcgctgggcctctgg | 8214 |
| ccggagtggcccgcgcgcg | 4043 | cgcgcgcgggccactccgg | 5434 | cagaggcccagcgatggct | 6825 | agccatcgctgggcctctg | 8215 |
| cggagtggcccgcgcgcgc | 4044 | gcgcgcgcgggccactccg | 5435 | agaggcccagcgatggctc | 6826 | gagccatcgctgggcctct | 8216 |
| ggagtggcccgcgcgcgcg | 4045 | cgcgcgcgcgggccactcc | 5436 | gaggcccagcgatggctcc | 6827 | ggagccatcgctgggcctc | 8217 |
| gagtggcccgcgcgcgcgc | 4046 | gcgcgcgcgcgggccactc | 5437 | aggcccagcgatggctcca | 6828 | tggagccatcgctgggcct | 8218 |
| agtggcccgcgcgcgcgct | 4047 | agcgcgcgcgcgggccact | 5438 | ggcccagcgatggctccag | 6829 | ctggagccatcgctgggcc | 8219 |
| gtggcccgcgcgcgcgctc | 4048 | gagcgcgcgcgcgggccac | 5439 | gcccagcgatggctccagc | 6830 | gctggagccatcgctgggc | 8220 |
| tggcccgcgcgcgcgctcg | 4049 | cgagcgcgcgcgcgggcca | 5440 | cccagcgatggctccagcc | 6831 | ggctggagccatcgctggg | 8221 |
| ggcccgcgcgcgcgctcgg | 4050 | ccgagcgcgcgcgcgggcc | 5441 | ccagcgatggctccagccc | 6832 | gggctggagccatcgctgg | 8222 |
| gcccgcgcgcgcgctcgga | 4051 | tccgagcgcgcgcgcgggc | 5442 | cagcgatggctccagccca | 6833 | tgggctggagccatcgctg | 8223 |
| cccgcgcgcgcgctcggag | 4052 | ctccgagcgcgcgcgcggg | 5443 | agcgatggctccagcccag | 6834 | ctgggctggagccatcgct | 8224 |
| ccgcgcgcgcgctcggagg | 4053 | cctccgagcgcgcgcgcgg | 5444 | gcgatggctccagcccaga | 6835 | tctgggctggagccatcgc | 8225 |
| cgcgcgcgcgctcggaggg | 4054 | ccctccgagcgcgcgcgcg | 5445 | cgatggctccagcccagac | 6836 | gtctgggctggagccatcg | 8226 |
| gcgcgcgcgctcggagggg | 4055 | cccctccgagcgcgcgcgc | 5446 | gatggctccagcccagaca | 6837 | tgtctgggctggagccatc | 8227 |
| cgcgcgcgctcggaggggg | 4056 | ccccctccgagcgcgcgcg | 5447 | atggctccagcccagacag | 6838 | ctgtctgggctggagccat | 8228 |
| gcgcgcgctcggaggggga | 4057 | tccccctccgagcgcgcgc | 5448 | tggctccagcccagacaga | 6839 | tctgtctgggctggagcca | 8229 |
| cgcgcgctcggagggggac | 4058 | gtccccctccgagcgcgcg | 5449 | ggctccagcccagacagac | 6840 | gtctgtctgggctggagcc | 8230 |
| gcgcgctcggagggggaca | 4059 | tgtccccctccgagcgcgc | 5450 | gctccagcccagacagacc | 6841 | ggtctgtctgggctggagc | 8231 |
| cgcgctcggagggggacag | 4060 | ctgtccccctccgagcgcg | 5451 | ctccagcccagacagacca | 6842 | tggtctgtctgggctggag | 8232 |
| gcgctcggagggggacaga | 4061 | tctgtccccctccgagcgc | 5452 | tccagcccagacagaccag | 6843 | ctggtctgtctgggctgga | 8233 |
| cgctcggagggggacagag | 4062 | ctctgtccccctccgagcg | 5453 | ccagcccagacagaccaga | 6844 | tctggtctgtctgggctgg | 8234 |
| gctcggagggggacagaga | 4063 | tctctgtccccctccgagc | 5454 | cagcccagacagaccagag | 6845 | ctctggtctgtctgggctg | 8235 |
| ctcggagggggacagagac | 4064 | gtctctgtccccctccgag | 5455 | agcccagacagaccagaga | 6846 | tctctggtctgtctgggct | 8236 |
| tcggagggggacagagacg | 4065 | cgtctctgtccccctccga | 5456 | gcccagacagaccagagaa | 6847 | ttctctggtctgtctgggc | 8237 |
| cggagggggacagagacgg | 4066 | ccgtctctgtccccctccg | 5457 | cccagacagaccagagaag | 6848 | cttctctggtctgtctggg | 8238 |
| ggagggggacagagacgga | 4067 | tccgtctctgtccccctcc | 5458 | ccagacagaccagagaaga | 6849 | tcttctctggtctgtctgg | 8239 |
| gagggggacagagacggac | 4068 | gtccgtctctgtccccctc | 5459 | cagacagaccagagaagag | 6850 | ctcttctctggtctgtctg | 8240 |
| agggggacagagacggact | 4069 | agtccgtctctgtccccct | 5460 | agacagaccagagaagaga | 6851 | tctcttctctggtctgtct | 8241 |
| gggggacagagacggacta | 4070 | tagtccgtctctgtccccc | 5461 | gacagaccagagaagagag | 6852 | ctctcttctctggtctgtc | 8242 |
| ggggacagagacggactac | 4071 | gtagtccgtctctgtcccc | 5462 | acagaccagagaagagagc | 6853 | gctctcttctctggtctgt | 8243 |
| gggacagagacggactaca | 4072 | tgtagtccgtctctgtccc | 5463 | cagaccagagaagagagcc | 6854 | ggctctcttctctggtctg | 8244 |
| ggacagagacggactacag | 4073 | ctgtagtccgtctctgtcc | 5464 | agaccagagaagagagcca | 6855 | tggctctcttctctggtct | 8245 |
| gacagagacggactacagc | 4074 | gctgtagtccgtctctgtc | 5465 | gaccagagaagagagccac | 6856 | gtggctctcttctctggtc | 8246 |
| acagagacggactacagcg | 4075 | cgctgtagtccgtctctgt | 5466 | accagagaagagagccaca | 6857 | tgtggctctcttctctggt | 8247 |
| cagagacggactacagcga | 4076 | tcgctgtagtccgtctctg | 5467 | ccagagaagagagccacat | 6858 | atgtggctctcttctctgg | 8248 |
| agagacggactacagcgag | 4077 | ctcgctgtagtccgtctct | 5468 | cagagaagagagccacatc | 6859 | gatgtggctctcttctctg | 8249 |
| gagacggactacagcgagg | 4078 | cctcgctgtagtccgtctc | 5469 | agagaagagagccacatcc | 6860 | ggatgtggctctcttctct | 8250 |
| agacggactacagcgaggc | 4079 | gcctcgctgtagtccgtct | 5470 | gagaagagagccacatcct | 6861 | aggatgtggctctcttctc | 8251 |
| gacggactacagcgaggcc | 4080 | ggcctcgctgtagtccgtc | 5471 | agaagagagccacatccta | 6862 | taggatgtggctctcttct | 8252 |
| acggactacagcgaggccc | 4081 | gggcctcgctgtagtccgt | 5472 | gaagagagccacatcctac | 6863 | gtaggatgtggctctcttc | 8253 |
| cggactacagcgaggcccg | 4082 | cgggcctcgctgtagtccg | 5473 | aagagagccacatcctacc | 6864 | ggtaggatgtggctctctt | 8254 |
| ggactacagcgaggcccgg | 4083 | ccgggcctcgctgtagtcc | 5474 | agagagccacatcctacca | 6865 | tggtaggatgtggctctct | 8255 |
| gactacagcgaggcccgga | 4084 | tccgggcctcgctgtagtc | 5475 | gagagccacatcctaccag | 6866 | ctggtaggatgtggctctc | 8256 |
| actacagcgaggcccggag | 4085 | ctccgggcctcgctgtagt | 5476 | agagccacatcctaccaga | 6867 | tctggtaggatgtggctct | 8257 |
| ctacagcgaggcccggagg | 4086 | cctccgggcctcgctgtag | 5477 | gagccacatcctaccagat | 6868 | atctggtaggatgtggctc | 8258 |
| tacagcgaggcccggagga | 4087 | tcctccgggcctcgctgta | 5478 | agccacatcctaccagatg | 6869 | catctggtaggatgtggct | 8259 |
| acagcgaggcccggaggag | 4088 | ctcctccgggcctcgctgt | 5479 | gccacatcctaccagatgc | 6870 | gcatctggtaggatgtggc | 8260 |
| cagcgaggcccggaggagc | 4089 | gctcctccgggcctcgctg | 5480 | ccacatcctaccagatgcc | 6871 | ggcatctggtaggatgtgg | 8261 |
| agcgaggcccggaggagcc | 4090 | ggctcctccgggcctcgct | 5481 | cacatcctaccagatgccc | 6872 | gggcatctggtaggatgtg | 8262 |
| gcgaggcccggaggagccc | 4091 | gggctcctccgggcctcgc | 5482 | acatcctaccagatgccct | 6873 | agggcatctggtaggatgt | 8263 |
| cgaggcccggaggagcccc | 4092 | ggggctcctccgggcctcg | 5483 | catcctaccagatgccctt | 6874 | aagggcatctggtaggatg | 8264 |
| gaggcccggaggagccccg | 4093 | cggggctcctccgggcctc | 5484 | atcctaccagatgcccttg | 6875 | caagggcatctggtaggat | 8265 |
| aggcccggaggagccccga | 4094 | tcggggctcctccgggcct | 5485 | tcctaccagatgcccttgg | 6876 | ccaagggcatctggtagga | 8266 |
| ggcccggaggagccccgag | 4095 | ctcggggctcctccgggcc | 5486 | cctaccagatgcccttggt | 6877 | accaagggcatctggtagg | 8267 |
| gcccggaggagccccgaga | 4096 | tctcggggctcctccgggc | 5487 | ctaccagatgcccttggtc | 6878 | gaccaagggcatctggtag | 8268 |
| cccggaggagccccgagat | 4097 | atctcggggctcctccggg | 5488 | taccagatgcccttggtcc | 6879 | ggaccaagggcatctggta | 8269 |
| ccggaggagccccgagatg | 4098 | catctcggggctcctccgg | 5489 | accagatgcccttggtcca | 6880 | tggaccaagggcatctggt | 8270 |
| cggaggagccccgagatgt | 4099 | acatctcggggctcctccg | 5490 | ccagatgcccttggtccag | 6881 | ctggaccaagggcatctgg | 8271 |
| ggaggagccccgagatgtc | 4100 | gacatctcggggctcctcc | 5491 | cagatgcccttggtccagt | 6882 | actggaccaagggcatctg | 8272 |
| gaggagccccgagatgtcc | 4101 | ggacatctcggggctcctc | 5492 | agatgcccttggtccagtg | 6883 | cactggaccaagggcatct | 8273 |
| aggagccccgagatgtccc | 4102 | gggacatctcggggctcct | 5493 | gatgcccttggtccagtgt | 6884 | acactggaccaagggcatc | 8274 |
| ggagccccgagatgtcccg | 4103 | cgggacatctcggggctcc | 5494 | atgcccttggtccagtgtg | 6885 | cacactggaccaagggcat | 8275 |
| gagccccgagatgtcccgt | 4104 | acgggacatctcggggctc | 5495 | tgcccttggtccagtgtgc | 6886 | gcacactggaccaagggca | 8276 |
| agccccgagatgtcccgtg | 4105 | cacgggacatctcggggct | 5496 | gcccttggtccagtgtgcc | 6887 | ggcacactggaccaagggc | 8277 |
| gccccgagatgtcccgtgt | 4106 | acacgggacatctcggggc | 5497 | cccttggtccagtgtgcct | 6888 | aggcacactggaccaaggg | 8278 |
| ccccgagatgtcccgtgtg | 4107 | cacacgggacatctcgggg | 5498 | ccttggtccagtgtgcctc | 6889 | gaggcacactggaccaagg | 8279 |
| cccgagatgtcccgtgtgc | 4108 | gcacacgggacatctcggg | 5499 | cttggtccagtgtgcctct | 6890 | agaggcacactggaccaag | 8280 |
| ccgagatgtcccgtgtgcg | 4109 | cgcacacgggacatctcgg | 5500 | ttggtccagtgtgcctctt | 6891 | aagaggcacactggaccaa | 8281 |
| cgagatgtcccgtgtgcgc | 4110 | gcgcacacgggacatctcg | 5501 | tggtccagtgtgcctcttc | 6892 | gaagaggcacactggacca | 8282 |
| gagatgtcccgtgtgcgcc | 4111 | ggcgcacacgggacatctc | 5502 | ggtccagtgtgcctcttcc | 6893 | ggaagaggcacactggacc | 8283 |
| agatgtcccgtgtgcgccg | 4112 | cggcgcacacgggacatct | 5503 | gtccagtgtgcctcttcct | 6894 | aggaagaggcacactggac | 8284 |
| gatgtcccgtgtgcgccgc | 4113 | gcggcgcacacgggacatc | 5504 | tccagtgtgcctcttcctc | 6895 | gaggaagaggcacactgga | 8285 |
| atgtcccgtgtgcgccgcc | 4114 | ggcggcgcacacgggacat | 5505 | ccagtgtgcctcttcctca | 6896 | tgaggaagaggcacactgg | 8286 |
| tgtcccgtgtgcgccgcct | 4115 | aggcggcgcacacgggaca | 5506 | cagtgtgcctcttcctcac | 6897 | gtgaggaagaggcacactg | 8287 |
| gtcccgtgtgcgccgcctt | 4116 | aaggcggcgcacacgggac | 5507 | agtgtgcctcttcctcacc | 6898 | ggtgaggaagaggcacact | 8288 |
| tcccgtgtgcgccgccttc | 4117 | gaaggcggcgcacacggga | 5508 | gtgtgcctcttcctcacca | 6899 | tggtgaggaagaggcacac | 8289 |
| cccgtgtgcgccgccttct | 4118 | agaaggcggcgcacacggg | 5509 | tgtgcctcttcctcaccaa | 6900 | ttggtgaggaagaggcaca | 8290 |
| ccgtgtgcgccgccttctg | 4119 | cagaaggcggcgcacacgg | 5510 | gtgcctcttcctcaccaag | 6901 | cttggtgaggaagaggcac | 8291 |
| cgtgtgcgccgccttctgc | 4120 | gcagaaggcggcgcacacg | 5511 | tgcctcttcctcaccaaga | 6902 | tcttggtgaggaagaggca | 8292 |
| gtgtgcgccgccttctgct | 4121 | agcagaaggcggcgcacac | 5512 | gcctcttcctcaccaagag | 6903 | ctcttggtgaggaagaggc | 8293 |
| tgtgcgccgccttctgctt | 4122 | aagcagaaggcggcgcaca | 5513 | cctcttcctcaccaagagc | 6904 | gctcttggtgaggaagagg | 8294 |
| gtgcgccgccttctgcttg | 4123 | caagcagaaggcggcgcac | 5514 | ctcttcctcaccaagagct | 6905 | agctcttggtgaggaagag | 8295 |
| tgcgccgccttctgcttgg | 4124 | ccaagcagaaggcggcgca | 5515 | tcttcctcaccaagagctg | 6906 | cagctcttggtgaggaaga | 8296 |
| gcgccgccttctgcttgga | 4125 | tccaagcagaaggcggcgc | 5516 | cttcctcaccaagagctga | 6907 | tcagctcttggtgaggaag | 8297 |
| cgccgccttctgcttggat | 4126 | atccaagcagaaggcggcg | 5517 | ttcctcaccaagagctgaa | 6908 | ttcagctcttggtgaggaa | 8298 |
| gccgccttctgcttggata | 4127 | tatccaagcagaaggcggc | 5518 | tcctcaccaagagctgaag | 6909 | cttcagctcttggtgagga | 8299 |
| ccgccttctgcttggatac | 4128 | gtatccaagcagaaggcgg | 5519 | cctcaccaagagctgaaga | 6910 | tcttcagctcttggtgagg | 8300 |
| cgccttctgcttggatacc | 4129 | ggtatccaagcagaaggcg | 5520 | ctcaccaagagctgaagag | 6911 | ctcttcagctcttggtgag | 8301 |
| gccttctgcttggatacct | 4130 | aggtatccaagcagaaggc | 5521 | tcaccaagagctgaagagt | 6912 | actcttcagctcttggtga | 8302 |
| ccttctgcttggatacctg | 4131 | caggtatccaagcagaagg | 5522 | caccaagagctgaagagtt | 6913 | aactcttcagctcttggtg | 8303 |
| cttctgcttggatacctgt | 4132 | acaggtatccaagcagaag | 5523 | accaagagctgaagagttg | 6914 | caactcttcagctcttggt | 8304 |
| ttctgcttggatacctgtt | 4133 | aacaggtatccaagcagaa | 5524 | ccaagagctgaagagttgt | 6915 | acaactcttcagctcttgg | 8305 |
| tctgcttggatacctgttc | 4134 | gaacaggtatccaagcaga | 5525 | caagagctgaagagttgtt | 6916 | aacaactcttcagctcttg | 8306 |
| ctgcttggatacctgttcc | 4135 | ggaacaggtatccaagcag | 5526 | aagagctgaagagttgttg | 6917 | caacaactcttcagctctt | 8307 |
| tgcttggatacctgttccc | 4136 | gggaacaggtatccaagca | 5527 | agagctgaagagttgttgg | 6918 | ccaacaactcttcagctct | 8308 |
| gcttggatacctgttccca | 4137 | tgggaacaggtatccaagc | 5528 | gagctgaagagttgttgga | 6919 | tccaacaactcttcagctc | 8309 |
| cttggatacctgttcccag | 4138 | ctgggaacaggtatccaag | 5529 | agctgaagagttgttggaa | 6920 | ttccaacaactcttcagct | 8310 |
| ttggatacctgttcccagc | 4139 | gctgggaacaggtatccaa | 5530 | gctgaagagttgttggaag | 6921 | cttccaacaactcttcagc | 8311 |
| tggatacctgttcccagcc | 4140 | ggctgggaacaggtatcca | 5531 | ctgaagagttgttggaaga | 6922 | tcttccaacaactcttcag | 8312 |
| ggatacctgttcccagccc | 4141 | gggctgggaacaggtatcc | 5532 | tgaagagttgttggaagac | 6923 | gtcttccaacaactcttca | 8313 |
| gatacctgttcccagccct | 4142 | agggctgggaacaggtatc | 5533 | gaagagttgttggaagaca | 6924 | tgtcttccaacaactcttc | 8314 |
| atacctgttcccagccctc | 4143 | gagggctgggaacaggtat | 5534 | aagagttgttggaagacag | 6925 | ctgtcttccaacaactctt | 8315 |
| tacctgttcccagccctcc | 4144 | ggagggctgggaacaggta | 5535 | agagttgttggaagacagt | 6926 | actgtcttccaacaactct | 8316 |
| acctgttcccagccctcct | 4145 | aggagggctgggaacaggt | 5536 | gagttgttggaagacagtc | 6927 | gactgtcttccaacaactc | 8317 |
| cctgttcccagccctcctg | 4146 | caggagggctgggaacagg | 5537 | agttgttggaagacagtca | 6928 | tgactgtcttccaacaact | 8318 |
| ctgttcccagccctcctgt | 4147 | acaggagggctgggaacag | 5538 | gttgttggaagacagtcaa | 6929 | ttgactgtcttccaacaac | 8319 |
| tgttcccagccctcctgtt | 4148 | aacaggagggctgggaaca | 5539 | ttgttggaagacagtcaag | 6930 | cttgactgtcttccaacaa | 8320 |
| gttcccagccctcctgttg | 4149 | caacaggagggctgggaac | 5540 | tgttggaagacagtcaagg | 6931 | ccttgactgtcttccaaca | 8321 |
| ttcccagccctcctgttgc | 4150 | gcaacaggagggctgggaa | 5541 | gttggaagacagtcaaggt | 6932 | accttgactgtcttccaac | 8322 |
| tcccagccctcctgttgca | 4151 | tgcaacaggagggctggga | 5542 | ttggaagacagtcaaggtc | 6933 | gaccttgactgtcttccaa | 8323 |
| cccagccctcctgttgcat | 4152 | atgcaacaggagggctggg | 5543 | tggaagacagtcaaggtca | 6934 | tgaccttgactgtcttcca | 8324 |
| ccagccctcctgttgcatg | 4153 | catgcaacaggagggctgg | 5544 | ggaagacagtcaaggtcat | 6935 | atgaccttgactgtcttcc | 8325 |
| cagccctcctgttgcatgg | 4154 | ccatgcaacaggagggctg | 5545 | gaagacagtcaaggtcatc | 6936 | gatgaccttgactgtcttc | 8326 |
| agccctcctgttgcatggg | 4155 | cccatgcaacaggagggct | 5546 | aagacagtcaaggtcatct | 6937 | agatgaccttgactgtctt | 8327 |
| gccctcctgttgcatgggc | 4156 | gcccatgcaacaggagggc | 5547 | agacagtcaaggtcatctg | 6938 | cagatgaccttgactgtct | 8328 |
| ccctcctgttgcatgggct | 4157 | agcccatgcaacaggaggg | 5548 | gacagtcaaggtcatctgt | 6939 | acagatgaccttgactgtc | 8329 |
| cctcctgttgcatgggctg | 4158 | cagcccatgcaacaggagg | 5549 | acagtcaaggtcatctgta | 6940 | tacagatgaccttgactgt | 8330 |
| ctcctgttgcatgggctgg | 4159 | ccagcccatgcaacaggag | 5550 | cagtcaaggtcatctgtat | 6941 | atacagatgaccttgactg | 8331 |
| tcctgttgcatgggctggg | 4160 | cccagcccatgcaacagga | 5551 | agtcaaggtcatctgtatg | 6942 | catacagatgaccttgact | 8332 |
| cctgttgcatgggctggga | 4161 | tcccagcccatgcaacagg | 5552 | gtcaaggtcatctgtatgg | 6943 | ccatacagatgaccttgac | 8333 |
| ctgttgcatgggctgggag | 4162 | ctcccagcccatgcaacag | 5553 | tcaaggtcatctgtatggc | 6944 | gccatacagatgaccttga | 8334 |
| tgttgcatgggctgggaga | 4163 | tctcccagcccatgcaaca | 5554 | caaggtcatctgtatggca | 6945 | tgccatacagatgaccttg | 8335 |
| gttgcatgggctgggagag | 4164 | ctctcccagcccatgcaac | 5555 | aaggtcatctgtatggcag | 6946 | ctgccatacagatgacctt | 8336 |
| ttgcatgggctgggagagg | 4165 | cctctcccagcccatgcaa | 5556 | aggtcatctgtatggcagg | 6947 | cctgccatacagatgacct | 8337 |
| tgcatgggctgggagaggg | 4166 | ccctctcccagcccatgca | 5557 | ggtcatctgtatggcaggg | 6948 | ccctgccatacagatgacc | 8338 |
| gcatgggctgggagagggc | 4167 | gccctctcccagcccatgc | 5558 | gtcatctgtatggcagggc | 6949 | gccctgccatacagatgac | 8339 |
| catgggctgggagagggct | 4168 | agccctctcccagcccatg | 5559 | tcatctgtatggcagggca | 6950 | tgccctgccatacagatga | 8340 |
| atgggctgggagagggctc | 4169 | gagccctctcccagcccat | 5560 | catctgtatggcagggcag | 6951 | ctgccctgccatacagatg | 8341 |
| tgggctgggagagggctct | 4170 | agagccctctcccagccca | 5561 | atctgtatggcagggcagc | 6952 | gctgccctgccatacagat | 8342 |
| gggctgggagagggctctg | 4171 | cagagccctctcccagccc | 5562 | tctgtatggcagggcagca | 6953 | tgctgccctgccatacaga | 8343 |
| ggctgggagagggctctgc | 4172 | gcagagccctctcccagcc | 5563 | ctgtatggcagggcagcag | 6954 | ctgctgccctgccatacag | 8344 |
| gctgggagagggctctgcc | 4173 | ggcagagccctctcccagc | 5564 | tgtatggcagggcagcagg | 6955 | cctgctgccctgccataca | 8345 |
| ctgggagagggctctgccc | 4174 | gggcagagccctctcccag | 5565 | gtatggcagggcagcaggg | 6956 | ccctgctgccctgccatac | 8346 |
| tgggagagggctctgccct | 4175 | agggcagagccctctccca | 5566 | tatggcagggcagcaggga | 6957 | tccctgctgccctgccata | 8347 |
| gggagagggctctgccctc | 4176 | gagggcagagccctctccc | 5567 | atggcagggcagcagggag | 6958 | ctccctgctgccctgccat | 8348 |
| ggagagggctctgccctcc | 4177 | ggagggcagagccctctcc | 5568 | tggcagggcagcagggagg | 6959 | cctccctgctgccctgcca | 8349 |
| gagagggctctgccctcct | 4178 | aggagggcagagccctctc | 5569 | ggcagggcagcagggagga | 6960 | tcctccctgctgccctgcc | 8350 |
| agagggctctgccctcctt | 4179 | aaggagggcagagccctct | 5570 | gcagggcagcagggaggac | 6961 | gtcctccctgctgccctgc | 8351 |
| gagggctctgccctccttc | 4180 | gaaggagggcagagccctc | 5571 | cagggcagcagggaggaca | 6962 | tgtcctccctgctgccctg | 8352 |
| agggctctgccctccttca | 4181 | tgaaggagggcagagccct | 5572 | agggcagcagggaggacag | 6963 | ctgtcctccctgctgccct | 8353 |
| gggctctgccctccttcat | 4182 | atgaaggagggcagagccc | 5573 | gggcagcagggaggacagc | 6964 | gctgtcctccctgctgccc | 8354 |
| ggctctgccctccttcatc | 4183 | gatgaaggagggcagagcc | 5574 | ggcagcagggaggacagct | 6965 | agctgtcctccctgctgcc | 8355 |
| gctctgccctccttcatcc | 4184 | ggatgaaggagggcagagc | 5575 | gcagcagggaggacagctg | 6966 | cagctgtcctccctgctgc | 8356 |
| ctctgccctccttcatcca | 4185 | tggatgaaggagggcagag | 5576 | cagcagggaggacagctgg | 6967 | ccagctgtcctccctgctg | 8357 |
| tctgccctccttcatccag | 4186 | ctggatgaaggagggcaga | 5577 | agcagggaggacagctggg | 6968 | cccagctgtcctccctgct | 8358 |
| ctgccctccttcatccaga | 4187 | tctggatgaaggagggcag | 5578 | gcagggaggacagctgggt | 6969 | acccagctgtcctccctgc | 8359 |
| tgccctccttcatccagac | 4188 | gtctggatgaaggagggca | 5579 | cagggaggacagctgggtc | 6970 | gacccagctgtcctccctg | 8360 |
| gccctccttcatccagaca | 4189 | tgtctggatgaaggagggc | 5580 | agggaggacagctgggtcc | 6971 | ggacccagctgtcctccct | 8361 |
| ccctccttcatccagacag | 4190 | ctgtctggatgaaggaggg | 5581 | gggaggacagctgggtccc | 6972 | gggacccagctgtcctccc | 8362 |
| cctccttcatccagacagc | 4191 | gctgtctggatgaaggagg | 5582 | ggaggacagctgggtccct | 6973 | agggacccagctgtcctcc | 8363 |
| ctccttcatccagacagca | 4192 | tgctgtctggatgaaggag | 5583 | gaggacagctgggtccctg | 6974 | cagggacccagctgtcctc | 8364 |
| tccttcatccagacagcag | 4193 | ctgctgtctggatgaagga | 5584 | aggacagctgggtccctgt | 6975 | acagggacccagctgtcct | 8365 |
| ccttcatccagacagcaga | 4194 | tctgctgtctggatgaagg | 5585 | ggacagctgggtccctgtt | 6976 | aacagggacccagctgtcc | 8366 |
| cttcatccagacagcagat | 4195 | atctgctgtctggatgaag | 5586 | gacagctgggtccctgttg | 6977 | caacagggacccagctgtc | 8367 |
| ttcatccagacagcagatc | 4196 | gatctgctgtctggatgaa | 5587 | acagctgggtccctgttgc | 6978 | gcaacagggacccagctgt | 8368 |
| tcatccagacagcagatcg | 4197 | cgatctgctgtctggatga | 5588 | cagctgggtccctgttgct | 6979 | agcaacagggacccagctg | 8369 |
| catccagacagcagatcgc | 4198 | gcgatctgctgtctggatg | 5589 | agctgggtccctgttgctt | 6980 | aagcaacagggacccagct | 8370 |
| atccagacagcagatcgca | 4199 | tgcgatctgctgtctggat | 5590 | gctgggtccctgttgcttc | 6981 | gaagcaacagggacccagc | 8371 |
| tccagacagcagatcgcac | 4200 | gtgcgatctgctgtctgga | 5591 | ctgggtccctgttgcttcc | 6982 | ggaagcaacagggacccag | 8372 |
| ccagacagcagatcgcacc | 4201 | ggtgcgatctgctgtctgg | 5592 | tgggtccctgttgcttcct | 6983 | aggaagcaacagggaccca | 8373 |
| cagacagcagatcgcaccc | 4202 | gggtgcgatctgctgtctg | 5593 | gggtccctgttgcttcctg | 6984 | caggaagcaacagggaccc | 8374 |
| agacagcagatcgcaccct | 4203 | agggtgcgatctgctgtct | 5594 | ggtccctgttgcttcctgc | 6985 | gcaggaagcaacagggacc | 8375 |
| gacagcagatcgcaccctc | 4204 | gagggtgcgatctgctgtc | 5595 | gtccctgttgcttcctgcc | 6986 | ggcaggaagcaacagggac | 8376 |
| acagcagatcgcaccctcg | 4205 | cgagggtgcgatctgctgt | 5596 | tccctgttgcttcctgcct | 6987 | aggcaggaagcaacaggga | 8377 |
| cagcagatcgcaccctcgg | 4206 | ccgagggtgcgatctgctg | 5597 | ccctgttgcttcctgcctt | 6988 | aaggcaggaagcaacaggg | 8378 |
| agcagatcgcaccctcggt | 4207 | accgagggtgcgatctgct | 5598 | cctgttgcttcctgccttt | 6989 | aaaggcaggaagcaacagg | 8379 |
| gcagatcgcaccctcggtc | 4208 | gaccgagggtgcgatctgc | 5599 | ctgttgcttcctgcctttg | 6990 | caaaggcaggaagcaacag | 8380 |
| cagatcgcaccctcggtcc | 4209 | ggaccgagggtgcgatctg | 5600 | tgttgcttcctgcctttgt | 6991 | acaaaggcaggaagcaaca | 8381 |
| agatcgcaccctcggtcct | 4210 | aggaccgagggtgcgatct | 5601 | gttgcttcctgcctttgtc | 6992 | gacaaaggcaggaagcaac | 8382 |
| gatcgcaccctcggtcctt | 4211 | aaggaccgagggtgcgatc | 5602 | ttgcttcctgcctttgtca | 6993 | tgacaaaggcaggaagcaa | 8383 |
| atcgcaccctcggtcctta | 4212 | taaggaccgagggtgcgat | 5603 | tgcttcctgcctttgtcag | 6994 | ctgacaaaggcaggaagca | 8384 |
| tcgcaccctcggtccttag | 4213 | ctaaggaccgagggtgcga | 5604 | gcttcctgcctttgtcagc | 6995 | gctgacaaaggcaggaagc | 8385 |
| cgcaccctcggtccttaga | 4214 | tctaaggaccgagggtgcg | 5605 | cttcctgcctttgtcagcc | 6996 | ggctgacaaaggcaggaag | 8386 |
| gcaccctcggtccttagag | 4215 | ctctaaggaccgagggtgc | 5606 | ttcctgcctttgtcagcct | 6997 | aggctgacaaaggcaggaa | 8387 |
| caccctcggtccttagaga | 4216 | tctctaaggaccgagggtg | 5607 | tcctgcctttgtcagcctt | 6998 | aaggctgacaaaggcagga | 8388 |
| accctcggtccttagagaa | 4217 | ttctctaaggaccgagggt | 5608 | cctgcctttgtcagccttt | 6999 | aaaggctgacaaaggcagg | 8389 |
| ccctcggtccttagagaaa | 4218 | tttctctaaggaccgaggg | 5609 | ctgcctttgtcagcctttg | 7000 | caaaggctgacaaaggcag | 8390 |
| cctcggtccttagagaaaa | 4219 | ttttctctaaggaccgagg | 5610 | tgcctttgtcagcctttgg | 7001 | ccaaaggctgacaaaggca | 8391 |
| ctcggtccttagagaaaag | 4220 | cttttctctaaggaccgag | 5611 | gcctttgtcagcctttgga | 7002 | tccaaaggctgacaaaggc | 8392 |
| tcggtccttagagaaaagc | 4221 | gcttttctctaaggaccga | 5612 | cctttgtcagcctttggac | 7003 | gtccaaaggctgacaaagg | 8393 |
| cggtccttagagaaaagcg | 4222 | cgcttttctctaaggaccg | 5613 | ctttgtcagcctttggact | 7004 | agtccaaaggctgacaaag | 8394 |
| ggtccttagagaaaagcgc | 4223 | gcgcttttctctaaggacc | 5614 | tttgtcagcctttggactc | 7005 | gagtccaaaggctgacaaa | 8395 |
| gtccttagagaaaagcgcc | 4224 | ggcgcttttctctaaggac | 5615 | ttgtcagcctttggactct | 7006 | agagtccaaaggctgacaa | 8396 |
| tccttagagaaaagcgcct | 4225 | aggcgcttttctctaagga | 5616 | tgtcagcctttggactctc | 7007 | gagagtccaaaggctgaca | 8397 |
| ccttagagaaaagcgcctg | 4226 | caggcgcttttctctaagg | 5617 | gtcagcctttggactctcc | 7008 | ggagagtccaaaggctgac | 8398 |
| cttagagaaaagcgcctgg | 4227 | ccaggcgcttttctctaag | 5618 | tcagcctttggactctccc | 7009 | gggagagtccaaaggctga | 8399 |
| ttagagaaaagcgcctgga | 4228 | tccaggcgcttttctctaa | 5619 | cagcctttggactctccca | 7010 | tgggagagtccaaaggctg | 8400 |
| tagagaaaagcgcctggag | 4229 | ctccaggcgcttttctcta | 5620 | agcctttggactctcccac | 7011 | gtgggagagtccaaaggct | 8401 |
| agagaaaagcgcctggagg | 4230 | cctccaggcgcttttctct | 5621 | gcctttggactctcccaca | 7012 | tgtgggagagtccaaaggc | 8402 |
| gagaaaagcgcctggaggg | 4231 | ccctccaggcgcttttctc | 5622 | cctttggactctcccacat | 7013 | atgtgggagagtccaaagg | 8403 |
| agaaaagcgcctggagggc | 4232 | gccctccaggcgcttttct | 5623 | ctttggactctcccacatt | 7014 | aatgtgggagagtccaaag | 8404 |
| gaaaagcgcctggagggct | 4233 | agccctccaggcgcttttc | 5624 | tttggactctcccacattg | 7015 | caatgtgggagagtccaaa | 8405 |
| aaaagcgcctggagggctt | 4234 | aagccctccaggcgctttt | 5625 | ttggactctcccacattgg | 7016 | ccaatgtgggagagtccaa | 8406 |
| aaagcgcctggagggcttt | 4235 | aaagccctccaggcgcttt | 5626 | tggactctcccacattggc | 7017 | gccaatgtgggagagtcca | 8407 |
| aagcgcctggagggctttc | 4236 | gaaagccctccaggcgctt | 5627 | ggactctcccacattggcg | 7018 | cgccaatgtgggagagtcc | 8408 |
| agcgcctggagggctttca | 4237 | tgaaagccctccaggcgct | 5628 | gactctcccacattggcgc | 7019 | gcgccaatgtgggagagtc | 8409 |
| gcgcctggagggctttcaa | 4238 | ttgaaagccctccaggcgc | 5629 | actctcccacattggcgca | 7020 | tgcgccaatgtgggagagt | 8410 |
| cgcctggagggctttcaag | 4239 | cttgaaagccctccaggcg | 5630 | ctctcccacattggcgcat | 7021 | atgcgccaatgtgggagag | 8411 |
| gcctggagggctttcaagg | 4240 | ccttgaaagccctccaggc | 5631 | tctcccacattggcgcatc | 7022 | gatgcgccaatgtgggaga | 8412 |
| cctggagggctttcaagga | 4241 | tccttgaaagccctccagg | 5632 | ctcccacattggcgcatcc | 7023 | ggatgcgccaatgtgggag | 8413 |
| ctggagggctttcaaggag | 4242 | ctccttgaaagccctccag | 5633 | tcccacattggcgcatcct | 7024 | aggatgcgccaatgtggga | 8414 |
| tggagggctttcaaggagt | 4243 | actccttgaaagccctcca | 5634 | cccacattggcgcatcctc | 7025 | gaggatgcgccaatgtggg | 8415 |
| ggagggctttcaaggagtc | 4244 | gactccttgaaagccctcc | 5635 | ccacattggcgcatcctca | 7026 | tgaggatgcgccaatgtgg | 8416 |
| gagggctttcaaggagtca | 4245 | tgactccttgaaagccctc | 5636 | cacattggcgcatcctcag | 7027 | ctgaggatgcgccaatgtg | 8417 |
| agggctttcaaggagtcac | 4246 | gtgactccttgaaagccct | 5637 | acattggcgcatcctcaga | 7028 | tctgaggatgcgccaatgt | 8418 |
| gggctttcaaggagtcaca | 4247 | tgtgactccttgaaagccc | 5638 | cattggcgcatcctcagat | 7029 | atctgaggatgcgccaatg | 8419 |
| ggctttcaaggagtcacag | 4248 | ctgtgactccttgaaagcc | 5639 | attggcgcatcctcagata | 7030 | tatctgaggatgcgccaat | 8420 |
| gctttcaaggagtcacagt | 4249 | actgtgactccttgaaagc | 5640 | ttggcgcatcctcagatag | 7031 | ctatctgaggatgcgccaa | 8421 |
| ctttcaaggagtcacagtg | 4250 | cactgtgactccttgaaag | 5641 | tggcgcatcctcagataga | 7032 | tctatctgaggatgcgcca | 8422 |
| tttcaaggagtcacagtgt | 4251 | acactgtgactccttgaaa | 5642 | ggcgcatcctcagatagaa | 7033 | ttctatctgaggatgcgcc | 8423 |
| ttcaaggagtcacagtgtc | 4252 | gacactgtgactccttgaa | 5643 | gcgcatcctcagatagaag | 7034 | cttctatctgaggatgcgc | 8424 |
| tcaaggagtcacagtgtca | 4253 | tgacactgtgactccttga | 5644 | cgcatcctcagatagaaga | 7035 | tcttctatctgaggatgcg | 8425 |
| caaggagtcacagtgtcat | 4254 | atgacactgtgactccttg | 5645 | gcatcctcagatagaagac | 7036 | gtcttctatctgaggatgc | 8426 |
| aaggagtcacagtgtcatc | 4255 | gatgacactgtgactcctt | 5646 | catcctcagatagaagaca | 7037 | tgtcttctatctgaggatg | 8427 |
| aggagtcacagtgtcatca | 4256 | tgatgacactgtgactcct | 5647 | atcctcagatagaagacat | 7038 | atgtcttctatctgaggat | 8428 |
| ggagtcacagtgtcatcac | 4257 | gtgatgacactgtgactcc | 5648 | tcctcagatagaagacatt | 7039 | aatgtcttctatctgagga | 8429 |
| gagtcacagtgtcatcaca | 4258 | tgtgatgacactgtgactc | 5649 | cctcagatagaagacattt | 7040 | aaatgtcttctatctgagg | 8430 |
| agtcacagtgtcatcacat | 4259 | atgtgatgacactgtgact | 5650 | ctcagatagaagacatttc | 7041 | gaaatgtcttctatctgag | 8431 |
| gtcacagtgtcatcacatg | 4260 | catgtgatgacactgtgac | 5651 | tcagatagaagacatttcc | 7042 | ggaaatgtcttctatctga | 8432 |
| tcacagtgtcatcacatgc | 4261 | gcatgtgatgacactgtga | 5652 | cagatagaagacatttcct | 7043 | aggaaatgtcttctatctg | 8433 |
| cacagtgtcatcacatgct | 4262 | agcatgtgatgacactgtg | 5653 | agatagaagacatttcctg | 7044 | caggaaatgtcttctatct | 8434 |
| acagtgtcatcacatgctg | 4263 | cagcatgtgatgacactgt | 5654 | gatagaagacatttcctga | 7045 | tcaggaaatgtcttctatc | 8435 |
| cagtgtcatcacatgctga | 4264 | tcagcatgtgatgacactg | 5655 | atagaagacatttcctgaa | 7046 | ttcaggaaatgtcttctat | 8436 |
| agtgtcatcacatgctgaa | 4265 | ttcagcatgtgatgacact | 5656 | tagaagacatttcctgaac | 7047 | gttcaggaaatgtcttcta | 8437 |
| gtgtcatcacatgctgaag | 4266 | cttcagcatgtgatgacac | 5657 | agaagacatttcctgaacc | 7048 | ggttcaggaaatgtcttct | 8438 |
| tgtcatcacatgctgaagc | 4267 | gcttcagcatgtgatgaca | 5658 | gaagacatttcctgaacca | 7049 | tggttcaggaaatgtcttc | 8439 |
| gtcatcacatgctgaagca | 4268 | tgcttcagcatgtgatgac | 5659 | aagacatttcctgaaccaa | 7050 | ttggttcaggaaatgtctt | 8440 |
| tcatcacatgctgaagcat | 4269 | atgcttcagcatgtgatga | 5660 | agacatttcctgaaccaag | 7051 | cttggttcaggaaatgtct | 8441 |
| catcacatgctgaagcatc | 4270 | gatgcttcagcatgtgatg | 5661 | gacatttcctgaaccaaga | 7052 | tcttggttcaggaaatgtc | 8442 |
| atcacatgctgaagcatct | 4271 | agatgcttcagcatgtgat | 5662 | acatttcctgaaccaagac | 7053 | gtcttggttcaggaaatgt | 8443 |
| tcacatgctgaagcatctc | 4272 | gagatgcttcagcatgtga | 5663 | catttcctgaaccaagact | 7054 | agtcttggttcaggaaatg | 8444 |
| cacatgctgaagcatctcc | 4273 | ggagatgcttcagcatgtg | 5664 | atttcctgaaccaagactc | 7055 | gagtcttggttcaggaaat | 8445 |
| acatgctgaagcatctcca | 4274 | tggagatgcttcagcatgt | 5665 | tttcctgaaccaagactct | 7056 | agagtcttggttcaggaaa | 8446 |
| catgctgaagcatctccac | 4275 | gtggagatgcttcagcatg | 5666 | ttcctgaaccaagactctt | 7057 | aagagtcttggttcaggaa | 8447 |
| atgctgaagcatctccaca | 4276 | tgtggagatgcttcagcat | 5667 | tcctgaaccaagactcttt | 7058 | aaagagtcttggttcagga | 8448 |
| tgctgaagcatctccacaa | 4277 | ttgtggagatgcttcagca | 5668 | cctgaaccaagactcttta | 7059 | taaagagtcttggttcagg | 8449 |
| gctgaagcatctccacaac | 4278 | gttgtggagatgcttcagc | 5669 | ctgaaccaagactctttac | 7060 | gtaaagagtcttggttcag | 8450 |
| ctgaagcatctccacaacg | 4279 | cgttgtggagatgcttcag | 5670 | tgaaccaagactctttacc | 7061 | ggtaaagagtcttggttca | 8451 |
| tgaagcatctccacaacgg | 4280 | ccgttgtggagatgcttca | 5671 | gaaccaagactctttaccg | 7062 | cggtaaagagtcttggttc | 8452 |
| gaagcatctccacaacggt | 4281 | accgttgtggagatgcttc | 5672 | aaccaagactctttaccgt | 7063 | acggtaaagagtcttggtt | 8453 |
| aagcatctccacaacggtg | 4282 | caccgttgtggagatgctt | 5673 | accaagactctttaccgtg | 7064 | cacggtaaagagtcttggt | 8454 |
| agcatctccacaacggtgc | 4283 | gcaccgttgtggagatgct | 5674 | ccaagactctttaccgtgt | 7065 | acacggtaaagagtcttgg | 8455 |
| gcatctccacaacggtgcg | 4284 | cgcaccgttgtggagatgc | 5675 | caagactctttaccgtgta | 7066 | tacacggtaaagagtcttg | 8456 |
| catctccacaacggtgcgc | 4285 | gcgcaccgttgtggagatg | 5676 | aagactctttaccgtgtat | 7067 | atacacggtaaagagtctt | 8457 |
| atctccacaacggtgcgcg | 4286 | cgcgcaccgttgtggagat | 5677 | agactctttaccgtgtatg | 7068 | catacacggtaaagagtct | 8458 |
| tctccacaacggtgcgcgg | 4287 | ccgcgcaccgttgtggaga | 5678 | gactctttaccgtgtatga | 7069 | tcatacacggtaaagagtc | 8459 |
| ctccacaacggtgcgcgga | 4288 | tccgcgcaccgttgtggag | 5679 | actctttaccgtgtatgac | 7070 | gtcatacacggtaaagagt | 8460 |
| tccacaacggtgcgcggat | 4289 | atccgcgcaccgttgtgga | 5680 | ctctttaccgtgtatgact | 7071 | agtcatacacggtaaagag | 8461 |
| ccacaacggtgcgcggatc | 4290 | gatccgcgcaccgttgtgg | 5681 | tctttaccgtgtatgactt | 7072 | aagtcatacacggtaaaga | 8462 |
| cacaacggtgcgcggatca | 4291 | tgatccgcgcaccgttgtg | 5682 | ctttaccgtgtatgacttt | 7073 | aaagtcatacacggtaaag | 8463 |
| acaacggtgcgcggatcac | 4292 | gtgatccgcgcaccgttgt | 5683 | tttaccgtgtatgactttc | 7074 | gaaagtcatacacggtaaa | 8464 |
| caacggtgcgcggatcaca | 4293 | tgtgatccgcgcaccgttg | 5684 | ttaccgtgtatgactttcc | 7075 | ggaaagtcatacacggtaa | 8465 |
| aacggtgcgcggatcacag | 4294 | ctgtgatccgcgcaccgtt | 5685 | taccgtgtatgactttcct | 7076 | aggaaagtcatacacggta | 8466 |
| acggtgcgcggatcacagt | 4295 | actgtgatccgcgcaccgt | 5686 | accgtgtatgactttcctt | 7077 | aaggaaagtcatacacggt | 8467 |
| cggtgcgcggatcacagtg | 4296 | cactgtgatccgcgcaccg | 5687 | ccgtgtatgactttccttc | 7078 | gaaggaaagtcatacacgg | 8468 |
| ggtgcgcggatcacagtgc | 4297 | gcactgtgatccgcgcacc | 5688 | cgtgtatgactttccttca | 7079 | tgaaggaaagtcatacacg | 8469 |
| gtgcgcggatcacagtgca | 4298 | tgcactgtgatccgcgcac | 5689 | gtgtatgactttccttcac | 7080 | gtgaaggaaagtcatacac | 8470 |
| tgcgcggatcacagtgcag | 4299 | ctgcactgtgatccgcgca | 5690 | tgtatgactttccttcaca | 7081 | tgtgaaggaaagtcataca | 8471 |
| gcgcggatcacagtgcaga | 4300 | tctgcactgtgatccgcgc | 5691 | gtatgactttccttcacac | 7082 | gtgtgaaggaaagtcatac | 8472 |
| cgcggatcacagtgcagat | 4301 | atctgcactgtgatccgcg | 5692 | tatgactttccttcacaca | 7083 | tgtgtgaaggaaagtcata | 8473 |
| gcggatcacagtgcagatg | 4302 | catctgcactgtgatccgc | 5693 | atgactttccttcacacag | 7084 | ctgtgtgaaggaaagtcat | 8474 |
| cggatcacagtgcagatgc | 4303 | gcatctgcactgtgatccg | 5694 | tgactttccttcacacaga | 7085 | tctgtgtgaaggaaagtca | 8475 |
| ggatcacagtgcagatgcc | 4304 | ggcatctgcactgtgatcc | 5695 | gactttccttcacacagag | 7086 | ctctgtgtgaaggaaagtc | 8476 |
| gatcacagtgcagatgccc | 4305 | gggcatctgcactgtgatc | 5696 | actttccttcacacagaga | 7087 | tctctgtgtgaaggaaagt | 8477 |
| atcacagtgcagatgcccc | 4306 | ggggcatctgcactgtgat | 5697 | ctttccttcacacagagaa | 7088 | ttctctgtgtgaaggaaag | 8478 |
| tcacagtgcagatgccccc | 4307 | gggggcatctgcactgtga | 5698 | tttccttcacacagagaaa | 7089 | tttctctgtgtgaaggaaa | 8479 |
| cacagtgcagatgcccccg | 4308 | cgggggcatctgcactgtg | 5699 | ttccttcacacagagaaag | 7090 | ctttctctgtgtgaaggaa | 8480 |
| acagtgcagatgcccccga | 4309 | tcgggggcatctgcactgt | 5700 | tccttcacacagagaaagg | 7091 | cctttctctgtgtgaagga | 8481 |
| cagtgcagatgcccccgac | 4310 | gtcgggggcatctgcactg | 5701 | ccttcacacagagaaagga | 7092 | tcctttctctgtgtgaagg | 8482 |
| agtgcagatgcccccgacc | 4311 | ggtcgggggcatctgcact | 5702 | cttcacacagagaaaggac | 7093 | gtcctttctctgtgtgaag | 8483 |
| gtgcagatgcccccgacca | 4312 | tggtcgggggcatctgcac | 5703 | ttcacacagagaaaggaca | 7094 | tgtcctttctctgtgtgaa | 8484 |
| tgcagatgcccccgaccat | 4313 | atggtcgggggcatctgca | 5704 | tcacacagagaaaggacat | 7095 | atgtcctttctctgtgtga | 8485 |
| gcagatgcccccgaccatc | 4314 | gatggtcgggggcatctgc | 5705 | cacacagagaaaggacatt | 7096 | aatgtcctttctctgtgtg | 8486 |
| cagatgcccccgaccatcg | 4315 | cgatggtcgggggcatctg | 5706 | acacagagaaaggacattg | 7097 | caatgtcctttctctgtgt | 8487 |
| agatgcccccgaccatcga | 4316 | tcgatggtcgggggcatct | 5707 | cacagagaaaggacattga | 7098 | tcaatgtcctttctctgtg | 8488 |
| gatgcccccgaccatcgag | 4317 | ctcgatggtcgggggcatc | 5708 | acagagaaaggacattgat | 7099 | atcaatgtcctttctctgt | 8489 |
| atgcccccgaccatcgagg | 4318 | cctcgatggtcgggggcat | 5709 | cagagaaaggacattgatt | 7100 | aatcaatgtcctttctctg | 8490 |
| tgcccccgaccatcgaggg | 4319 | ccctcgatggtcgggggca | 5710 | agagaaaggacattgattc | 7101 | gaatcaatgtcctttctct | 8491 |
| gcccccgaccatcgagggc | 4320 | gccctcgatggtcgggggc | 5711 | gagaaaggacattgattct | 7102 | agaatcaatgtcctttctc | 8492 |
| cccccgaccatcgagggcc | 4321 | ggccctcgatggtcggggg | 5712 | agaaaggacattgattctt | 7103 | aagaatcaatgtcctttct | 8493 |
| ccccgaccatcgagggcca | 4322 | tggccctcgatggtcgggg | 5713 | gaaaggacattgattcttt | 7104 | aaagaatcaatgtcctttc | 8494 |
| cccgaccatcgagggccac | 4323 | gtggccctcgatggtcggg | 5714 | aaaggacattgattctttt | 7105 | aaaagaatcaatgtccttt | 8495 |
| ccgaccatcgagggccact | 4324 | agtggccctcgatggtcgg | 5715 | aaggacattgattcttttg | 7106 | caaaagaatcaatgtcctt | 8496 |
| cgaccatcgagggccactg | 4325 | cagtggccctcgatggtcg | 5716 | aggacattgattcttttga | 7107 | tcaaaagaatcaatgtcct | 8497 |
| gaccatcgagggccactgg | 4326 | ccagtggccctcgatggtc | 5717 | ggacattgattcttttgat | 7108 | atcaaaagaatcaatgtcc | 8498 |
| accatcgagggccactggg | 4327 | cccagtggccctcgatggt | 5718 | gacattgattcttttgatg | 7109 | catcaaaagaatcaatgtc | 8499 |
| ccatcgagggccactgggt | 4328 | acccagtggccctcgatgg | 5719 | acattgattcttttgatgc | 7110 | gcatcaaaagaatcaatgt | 8500 |
| catcgagggccactgggtg | 4329 | cacccagtggccctcgatg | 5720 | cattgattcttttgatgca | 7111 | tgcatcaaaagaatcaatg | 8501 |
| atcgagggccactgggtgt | 4330 | acacccagtggccctcgat | 5721 | attgattcttttgatgcac | 7112 | gtgcatcaaaagaatcaat | 8502 |
| tcgagggccactgggtgtc | 4331 | gacacccagtggccctcga | 5722 | ttgattcttttgatgcact | 7113 | agtgcatcaaaagaatcaa | 8503 |
| cgagggccactgggtgtcc | 4332 | ggacacccagtggccctcg | 5723 | tgattcttttgatgcactt | 7114 | aagtgcatcaaaagaatca | 8504 |
| gagggccactgggtgtcca | 4333 | tggacacccagtggccctc | 5724 | gattcttttgatgcacttg | 7115 | caagtgcatcaaaagaatc | 8505 |
| agggccactgggtgtccac | 4334 | gtggacacccagtggccct | 5725 | attcttttgatgcacttga | 7116 | tcaagtgcatcaaaagaat | 8506 |
| gggccactgggtgtccaca | 4335 | tgtggacacccagtggccc | 5726 | ttcttttgatgcacttgaa | 7117 | ttcaagtgcatcaaaagaa | 8507 |
| ggccactgggtgtccacag | 4336 | ctgtggacacccagtggcc | 5727 | tcttttgatgcacttgaat | 7118 | attcaagtgcatcaaaaga | 8508 |
| gccactgggtgtccacagg | 4337 | cctgtggacacccagtggc | 5728 | cttttgatgcacttgaatg | 7119 | cattcaagtgcatcaaaag | 8509 |
| ccactgggtgtccacaggc | 4338 | gcctgtggacacccagtgg | 5729 | ttttgatgcacttgaatgc | 7120 | gcattcaagtgcatcaaaa | 8510 |
| cactgggtgtccacaggct | 4339 | agcctgtggacacccagtg | 5730 | tttgatgcacttgaatgcc | 7121 | ggcattcaagtgcatcaaa | 8511 |
| actgggtgtccacaggctg | 4340 | cagcctgtggacacccagt | 5731 | ttgatgcacttgaatgcct | 7122 | aggcattcaagtgcatcaa | 8512 |
| ctgggtgtccacaggctgt | 4341 | acagcctgtggacacccag | 5732 | tgatgcacttgaatgcctt | 7123 | aaggcattcaagtgcatca | 8513 |
| tgggtgtccacaggctgtg | 4342 | cacagcctgtggacaccca | 5733 | gatgcacttgaatgccttg | 7124 | caaggcattcaagtgcatc | 8514 |
| gggtgtccacaggctgtga | 4343 | tcacagcctgtggacaccc | 5734 | atgcacttgaatgccttga | 7125 | tcaaggcattcaagtgcat | 8515 |
| ggtgtccacaggctgtgaa | 4344 | ttcacagcctgtggacacc | 5735 | tgcacttgaatgccttgag | 7126 | ctcaaggcattcaagtgca | 8516 |
| gtgtccacaggctgtgaag | 4345 | cttcacagcctgtggacac | 5736 | gcacttgaatgccttgaga | 7127 | tctcaaggcattcaagtgc | 8517 |
| tgtccacaggctgtgaagt | 4346 | acttcacagcctgtggaca | 5737 | cacttgaatgccttgagac | 7128 | gtctcaaggcattcaagtg | 8518 |
| gtccacaggctgtgaagta | 4347 | tacttcacagcctgtggac | 5738 | acttgaatgccttgagacc | 7129 | ggtctcaaggcattcaagt | 8519 |
| tccacaggctgtgaagtaa | 4348 | ttacttcacagcctgtgga | 5739 | cttgaatgccttgagacct | 7130 | aggtctcaaggcattcaag | 8520 |
| ccacaggctgtgaagtaag | 4349 | cttacttcacagcctgtgg | 5740 | ttgaatgccttgagacctg | 7131 | caggtctcaaggcattcaa | 8521 |
| cacaggctgtgaagtaagg | 4350 | ccttacttcacagcctgtg | 5741 | tgaatgccttgagacctgc | 7132 | gcaggtctcaaggcattca | 8522 |
| acaggctgtgaagtaaggt | 4351 | accttacttcacagcctgt | 5742 | gaatgccttgagacctgcc | 7133 | ggcaggtctcaaggcattc | 8523 |
| caggctgtgaagtaaggtc | 4352 | gaccttacttcacagcctg | 5743 | aatgccttgagacctgccg | 7134 | cggcaggtctcaaggcatt | 8524 |
| aggctgtgaagtaaggtcg | 4353 | cgaccttacttcacagcct | 5744 | atgccttgagacctgccgt | 7135 | acggcaggtctcaaggcat | 8525 |
| ggctgtgaagtaaggtcgg | 4354 | ccgaccttacttcacagcc | 5745 | tgccttgagacctgccgtt | 7136 | aacggcaggtctcaaggca | 8526 |
| gctgtgaagtaaggtcggg | 4355 | cccgaccttacttcacagc | 5746 | gccttgagacctgccgttg | 7137 | caacggcaggtctcaaggc | 8527 |
| ctgtgaagtaaggtcgggt | 4356 | acccgaccttacttcacag | 5747 | ccttgagacctgccgttgc | 7138 | gcaacggcaggtctcaagg | 8528 |
| tgtgaagtaaggtcgggtc | 4357 | gacccgaccttacttcaca | 5748 | cttgagacctgccgttgcc | 7139 | ggcaacggcaggtctcaag | 8529 |
| gtgaagtaaggtcgggtcc | 4358 | ggacccgaccttacttcac | 5749 | ttgagacctgccgttgcct | 7140 | aggcaacggcaggtctcaa | 8530 |
| tgaagtaaggtcgggtccg | 4359 | cggacccgaccttacttca | 5750 | tgagacctgccgttgcctc | 7141 | gaggcaacggcaggtctca | 8531 |
| gaagtaaggtcgggtccgg | 4360 | ccggacccgaccttacttc | 5751 | gagacctgccgttgcctct | 7142 | agaggcaacggcaggtctc | 8532 |
| aagtaaggtcgggtccgga | 4361 | tccggacccgaccttactt | 5752 | agacctgccgttgcctctc | 7143 | gagaggcaacggcaggtct | 8533 |
| agtaaggtcgggtccggag | 4362 | ctccggacccgaccttact | 5753 | gacctgccgttgcctctcc | 7144 | ggagaggcaacggcaggtc | 8534 |
| gtaaggtcgggtccggagt | 4363 | actccggacccgaccttac | 5754 | acctgccgttgcctctcct | 7145 | aggagaggcaacggcaggt | 8535 |
| taaggtcgggtccggagtt | 4364 | aactccggacccgacctta | 5755 | cctgccgttgcctctcctc | 7146 | gaggagaggcaacggcagg | 8536 |
| aaggtcgggtccggagttc | 4365 | gaactccggacccgacctt | 5756 | ctgccgttgcctctcctct | 7147 | agaggagaggcaacggcag | 8537 |
| aggtcgggtccggagttca | 4366 | tgaactccggacccgacct | 5757 | tgccgttgcctctcctctc | 7148 | gagaggagaggcaacggca | 8538 |
| ggtcgggtccggagttcat | 4367 | atgaactccggacccgacc | 5758 | gccgttgcctctcctctcc | 7149 | ggagaggagaggcaacggc | 8539 |
| gtcgggtccggagttcatg | 4368 | catgaactccggacccgac | 5759 | ccgttgcctctcctctccc | 7150 | gggagaggagaggcaacgg | 8540 |
| tcgggtccggagttcatga | 4369 | tcatgaactccggacccga | 5760 | cgttgcctctcctctcccc | 7151 | ggggagaggagaggcaacg | 8541 |
| cgggtccggagttcatgac | 4370 | gtcatgaactccggacccg | 5761 | gttgcctctcctctcccca | 7152 | tggggagaggagaggcaac | 8542 |
| gggtccggagttcatgaca | 4371 | tgtcatgaactccggaccc | 5762 | ttgcctctcctctccccac | 7153 | gtggggagaggagaggcaa | 8543 |
| ggtccggagttcatgacaa | 4372 | ttgtcatgaactccggacc | 5763 | tgcctctcctctccccacc | 7154 | ggtggggagaggagaggca | 8544 |
| gtccggagttcatgacaag | 4373 | cttgtcatgaactccggac | 5764 | gcctctcctctccccacct | 7155 | aggtggggagaggagaggc | 8545 |
| tccggagttcatgacaagg | 4374 | ccttgtcatgaactccgga | 5765 | cctctcctctccccacctc | 7156 | gaggtggggagaggagagg | 8546 |
| ccggagttcatgacaaggt | 4375 | accttgtcatgaactccgg | 5766 | ctctcctctccccacctct | 7157 | agaggtggggagaggagag | 8547 |
| cggagttcatgacaaggtc | 4376 | gaccttgtcatgaactccg | 5767 | tctcctctccccacctctt | 7158 | aagaggtggggagaggaga | 8548 |
| ggagttcatgacaaggtct | 4377 | agaccttgtcatgaactcc | 5768 | ctcctctccccacctcttc | 7159 | gaagaggtggggagaggag | 8549 |
| gagttcatgacaaggtctt | 4378 | aagaccttgtcatgaactc | 5769 | tcctctccccacctcttcc | 7160 | ggaagaggtggggagagga | 8550 |
| agttcatgacaaggtctta | 4379 | taagaccttgtcatgaact | 5770 | cctctccccacctcttcca | 7161 | tggaagaggtggggagagg | 8551 |
| gttcatgacaaggtcttac | 4380 | gtaagaccttgtcatgaac | 5771 | ctctccccacctcttccag | 7162 | ctggaagaggtggggagag | 8552 |
| ttcatgacaaggtcttaca | 4381 | tgtaagaccttgtcatgaa | 5772 | tctccccacctcttccagc | 7163 | gctggaagaggtggggaga | 8553 |
| tcatgacaaggtcttacag | 4382 | ctgtaagaccttgtcatga | 5773 | ctccccacctcttccagcc | 7164 | ggctggaagaggtggggag | 8554 |
| catgacaaggtcttacagg | 4383 | cctgtaagaccttgtcatg | 5774 | tccccacctcttccagcct | 7165 | aggctggaagaggtgggga | 8555 |
| atgacaaggtcttacaggt | 4384 | acctgtaagaccttgtcat | 5775 | ccccacctcttccagcctc | 7166 | gaggctggaagaggtgggg | 8556 |
| tgacaaggtcttacaggtt | 4385 | aacctgtaagaccttgtca | 5776 | cccacctcttccagcctct | 7167 | agaggctggaagaggtggg | 8557 |
| gacaaggtcttacaggttc | 4386 | gaacctgtaagaccttgtc | 5777 | ccacctcttccagcctctg | 7168 | cagaggctggaagaggtgg | 8558 |
| acaaggtcttacaggttct | 4387 | agaacctgtaagaccttgt | 5778 | cacctcttccagcctctgc | 7169 | gcagaggctggaagaggtg | 8559 |
| caaggtcttacaggttcta | 4388 | tagaacctgtaagaccttg | 5779 | acctcttccagcctctgcg | 7170 | cgcagaggctggaagaggt | 8560 |
| aaggtcttacaggttctac | 4389 | gtagaacctgtaagacctt | 5780 | cctcttccagcctctgcgc | 7171 | gcgcagaggctggaagagg | 8561 |
| aggtcttacaggttctaca | 4390 | tgtagaacctgtaagacct | 5781 | ctcttccagcctctgcgcc | 7172 | ggcgcagaggctggaagag | 8562 |
| ggtcttacaggttctacaa | 4391 | ttgtagaacctgtaagacc | 5782 | tcttccagcctctgcgcca | 7173 | tggcgcagaggctggaaga | 8563 |
| gtcttacaggttctacaac | 4392 | gttgtagaacctgtaagac | 5783 | cttccagcctctgcgccat | 7174 | atggcgcagaggctggaag | 8564 |
| tcttacaggttctacaaca | 4393 | tgttgtagaacctgtaaga | 5784 | ttccagcctctgcgccatg | 7175 | catggcgcagaggctggaa | 8565 |
| cttacaggttctacaacaa | 4394 | ttgttgtagaacctgtaag | 5785 | tccagcctctgcgccatga | 7176 | tcatggcgcagaggctgga | 8566 |
| ttacaggttctacaacaat | 4395 | attgttgtagaacctgtaa | 5786 | ccagcctctgcgccatgag | 7177 | ctcatggcgcagaggctgg | 8567 |
| tacaggttctacaacaata | 4396 | tattgttgtagaacctgta | 5787 | cagcctctgcgccatgagc | 7178 | gctcatggcgcagaggctg | 8568 |
| acaggttctacaacaataa | 4397 | ttattgttgtagaacctgt | 5788 | agcctctgcgccatgagca | 7179 | tgctcatggcgcagaggct | 8569 |
| caggttctacaacaataat | 4398 | attattgttgtagaacctg | 5789 | gcctctgcgccatgagcag | 7180 | ctgctcatggcgcagaggc | 8570 |
| aggttctacaacaataata | 4399 | tattattgttgtagaacct | 5790 | cctctgcgccatgagcagt | 7181 | actgctcatggcgcagagg | 8571 |
| ggttctacaacaataatac | 4400 | gtattattgttgtagaacc | 5791 | ctctgcgccatgagcagtg | 7182 | cactgctcatggcgcagag | 8572 |
| gttctacaacaataatacc | 4401 | ggtattattgttgtagaac | 5792 | tctgcgccatgagcagtgt | 7183 | acactgctcatggcgcaga | 8573 |
| ttctacaacaataatacct | 4402 | aggtattattgttgtagaa | 5793 | ctgcgccatgagcagtgtg | 7184 | cacactgctcatggcgcag | 8574 |
| tctacaacaataatacctt | 4403 | aaggtattattgttgtaga | 5794 | tgcgccatgagcagtgtgt | 7185 | acacactgctcatggcgca | 8575 |
| ctacaacaataataccttc | 4404 | gaaggtattattgttgtag | 5795 | gcgccatgagcagtgtgtt | 7186 | aacacactgctcatggcgc | 8576 |
| tacaacaataataccttca | 4405 | tgaaggtattattgttgta | 5796 | cgccatgagcagtgtgttt | 7187 | aaacacactgctcatggcg | 8577 |
| acaacaataataccttcaa | 4406 | ttgaaggtattattgttgt | 5797 | gccatgagcagtgtgtttg | 7188 | caaacacactgctcatggc | 8578 |
| caacaataataccttcaag | 4407 | cttgaaggtattattgttg | 5798 | ccatgagcagtgtgtttga | 7189 | tcaaacacactgctcatgg | 8579 |
| aacaataataccttcaagg | 4408 | ccttgaaggtattattgtt | 5799 | catgagcagtgtgtttgaa | 7190 | ttcaaacacactgctcatg | 8580 |
| acaataataccttcaaggc | 4409 | gccttgaaggtattattgt | 5800 | atgagcagtgtgtttgaag | 7191 | cttcaaacacactgctcat | 8581 |
| caataataccttcaaggcc | 4410 | ggccttgaaggtattattg | 5801 | tgagcagtgtgtttgaagt | 7192 | acttcaaacacactgctca | 8582 |
| aataataccttcaaggcct | 4411 | aggccttgaaggtattatt | 5802 | gagcagtgtgtttgaagtt | 7193 | aacttcaaacacactgctc | 8583 |
| ataataccttcaaggccta | 4412 | taggccttgaaggtattat | 5803 | agcagtgtgtttgaagttt | 7194 | aaacttcaaacacactgct | 8584 |
| taataccttcaaggcctac | 4413 | gtaggccttgaaggtatta | 5804 | gcagtgtgtttgaagtttc | 7195 | gaaacttcaaacacactgc | 8585 |
| aataccttcaaggcctacc | 4414 | ggtaggccttgaaggtatt | 5805 | cagtgtgtttgaagtttca | 7196 | tgaaacttcaaacacactg | 8586 |
| ataccttcaaggcctacca | 4415 | tggtaggccttgaaggtat | 5806 | agtgtgtttgaagtttcag | 7197 | ctgaaacttcaaacacact | 8587 |
| taccttcaaggcctaccag | 4416 | ctggtaggccttgaaggta | 5807 | gtgtgtttgaagtttcagc | 7198 | gctgaaacttcaaacacac | 8588 |
| accttcaaggcctaccagt | 4417 | actggtaggccttgaaggt | 5808 | tgtgtttgaagtttcagct | 7199 | agctgaaacttcaaacaca | 8589 |
| ccttcaaggcctaccagtt | 4418 | aactggtaggccttgaagg | 5809 | gtgtttgaagtttcagctt | 7200 | aagctgaaacttcaaacac | 8590 |
| cttcaaggcctaccagttt | 4419 | aaactggtaggccttgaag | 5810 | tgtttgaagtttcagcttt | 7201 | aaagctgaaacttcaaaca | 8591 |
| ttcaaggcctaccagtttt | 4420 | aaaactggtaggccttgaa | 5811 | gtttgaagtttcagctttg | 7202 | caaagctgaaacttcaaac | 8592 |
| tcaaggcctaccagtttta | 4421 | taaaactggtaggccttga | 5812 | tttgaagtttcagctttga | 7203 | tcaaagctgaaacttcaaa | 8593 |
| caaggcctaccagttttac | 4422 | gtaaaactggtaggccttg | 5813 | ttgaagtttcagctttgaa | 7204 | ttcaaagctgaaacttcaa | 8594 |
| aaggcctaccagttttact | 4423 | agtaaaactggtaggcctt | 5814 | tgaagtttcagctttgaat | 7205 | attcaaagctgaaacttca | 8595 |
| aggcctaccagttttacta | 4424 | tagtaaaactggtaggcct | 5815 | gaagtttcagctttgaatg | 7206 | cattcaaagctgaaacttc | 8596 |
| ggcctaccagttttactat | 4425 | atagtaaaactggtaggcc | 5816 | aagtttcagctttgaatgt | 7207 | acattcaaagctgaaactt | 8597 |
| gcctaccagttttactatg | 4426 | catagtaaaactggtaggc | 5817 | agtttcagctttgaatgtc | 7208 | gacattcaaagctgaaact | 8598 |
| cctaccagttttactatgg | 4427 | ccatagtaaaactggtagg | 5818 | gtttcagctttgaatgtct | 7209 | agacattcaaagctgaaac | 8599 |
| ctaccagttttactatggc | 4428 | gccatagtaaaactggtag | 5819 | tttcagctttgaatgtctt | 7210 | aagacattcaaagctgaaa | 8600 |
| taccagttttactatggca | 4429 | tgccatagtaaaactggta | 5820 | ttcagctttgaatgtcttc | 7211 | gaagacattcaaagctgaa | 8601 |
| accagttttactatggcag | 4430 | ctgccatagtaaaactggt | 5821 | tcagctttgaatgtcttcc | 7212 | ggaagacattcaaagctga | 8602 |
| ccagttttactatggcagc | 4431 | gctgccatagtaaaactgg | 5822 | cagctttgaatgtcttccc | 7213 | gggaagacattcaaagctg | 8603 |
| cagttttactatggcagca | 4432 | tgctgccatagtaaaactg | 5823 | agctttgaatgtcttcccg | 7214 | cgggaagacattcaaagct | 8604 |
| agttttactatggcagcaa | 4433 | ttgctgccatagtaaaact | 5824 | gctttgaatgtcttcccgc | 7215 | gcgggaagacattcaaagc | 8605 |
| gttttactatggcagcaac | 4434 | gttgctgccatagtaaaac | 5825 | ctttgaatgtcttcccgcc | 7216 | ggcgggaagacattcaaag | 8606 |
| ttttactatggcagcaacc | 4435 | ggttgctgccatagtaaaa | 5826 | tttgaatgtcttcccgccc | 7217 | gggcgggaagacattcaaa | 8607 |
| tttactatggcagcaaccg | 4436 | cggttgctgccatagtaaa | 5827 | ttgaatgtcttcccgcccg | 7218 | cgggcgggaagacattcaa | 8608 |
| ttactatggcagcaaccgc | 4437 | gcggttgctgccatagtaa | 5828 | tgaatgtcttcccgcccga | 7219 | tcgggcgggaagacattca | 8609 |
| tactatggcagcaaccgct | 4438 | agcggttgctgccatagta | 5829 | gaatgtcttcccgcccgat | 7220 | atcgggcgggaagacattc | 8610 |
| actatggcagcaaccgctg | 4439 | cagcggttgctgccatagt | 5830 | aatgtcttcccgcccgatc | 7221 | gatcgggcgggaagacatt | 8611 |
| ctatggcagcaaccgctgt | 4440 | acagcggttgctgccatag | 5831 | atgtcttcccgcccgatcc | 7222 | ggatcgggcgggaagacat | 8612 |
| tatggcagcaaccgctgta | 4441 | tacagcggttgctgccata | 5832 | tgtcttcccgcccgatccg | 7223 | cggatcgggcgggaagaca | 8613 |
| atggcagcaaccgctgtac | 4442 | gtacagcggttgctgccat | 5833 | gtcttcccgcccgatccgt | 7224 | acggatcgggcgggaagac | 8614 |
| tggcagcaaccgctgtaca | 4443 | tgtacagcggttgctgcca | 5834 | tcttcccgcccgatccgtg | 7225 | cacggatcgggcgggaaga | 8615 |
| ggcagcaaccgctgtacaa | 4444 | ttgtacagcggttgctgcc | 5835 | cttcccgcccgatccgtgg | 7226 | ccacggatcgggcgggaag | 8616 |
| gcagcaaccgctgtacaaa | 4445 | tttgtacagcggttgctgc | 5836 | ttcccgcccgatccgtggc | 7227 | gccacggatcgggcgggaa | 8617 |
| cagcaaccgctgtacaaac | 4446 | gtttgtacagcggttgctg | 5837 | tcccgcccgatccgtggcc | 7228 | ggccacggatcgggcggga | 8618 |
| agcaaccgctgtacaaacc | 4447 | ggtttgtacagcggttgct | 5838 | cccgcccgatccgtggcct | 7229 | aggccacggatcgggcggg | 8619 |
| gcaaccgctgtacaaaccc | 4448 | gggtttgtacagcggttgc | 5839 | ccgcccgatccgtggcctg | 7230 | caggccacggatcgggcgg | 8620 |
| caaccgctgtacaaacccc | 4449 | ggggtttgtacagcggttg | 5840 | cgcccgatccgtggcctga | 7231 | tcaggccacggatcgggcg | 8621 |
| aaccgctgtacaaacccca | 4450 | tggggtttgtacagcggtt | 5841 | gcccgatccgtggcctgag | 7232 | ctcaggccacggatcgggc | 8622 |
| accgctgtacaaaccccac | 4451 | gtggggtttgtacagcggt | 5842 | cccgatccgtggcctgaga | 7233 | tctcaggccacggatcggg | 8623 |
| ccgctgtacaaaccccacc | 4452 | ggtggggtttgtacagcgg | 5843 | ccgatccgtggcctgagaa | 7234 | ttctcaggccacggatcgg | 8624 |
| cgctgtacaaaccccacct | 4453 | aggtggggtttgtacagcg | 5844 | cgatccgtggcctgagaac | 7235 | gttctcaggccacggatcg | 8625 |
| gctgtacaaaccccaccta | 4454 | taggtggggtttgtacagc | 5845 | gatccgtggcctgagaact | 7236 | agttctcaggccacggatc | 8626 |
| ctgtacaaaccccacctac | 4455 | gtaggtggggtttgtacag | 5846 | atccgtggcctgagaactt | 7237 | aagttctcaggccacggat | 8627 |
| tgtacaaaccccacctaca | 4456 | tgtaggtggggtttgtaca | 5847 | tccgtggcctgagaacttc | 7238 | gaagttctcaggccacgga | 8628 |
| gtacaaaccccacctacac | 4457 | gtgtaggtggggtttgtac | 5848 | ccgtggcctgagaacttca | 7239 | tgaagttctcaggccacgg | 8629 |
| tacaaaccccacctacacc | 4458 | ggtgtaggtggggtttgta | 5849 | cgtggcctgagaacttcat | 7240 | atgaagttctcaggccacg | 8630 |
| acaaaccccacctacaccc | 4459 | gggtgtaggtggggtttgt | 5850 | gtggcctgagaacttcata | 7241 | tatgaagttctcaggccac | 8631 |
| caaaccccacctacaccct | 4460 | agggtgtaggtggggtttg | 5851 | tggcctgagaacttcatag | 7242 | ctatgaagttctcaggcca | 8632 |
| aaaccccacctacaccctc | 4461 | gagggtgtaggtggggttt | 5852 | ggcctgagaacttcataga | 7243 | tctatgaagttctcaggcc | 8633 |
| aaccccacctacaccctca | 4462 | tgagggtgtaggtggggtt | 5853 | gcctgagaacttcatagag | 7244 | ctctatgaagttctcaggc | 8634 |
| accccacctacaccctcat | 4463 | atgagggtgtaggtggggt | 5854 | cctgagaacttcatagagg | 7245 | cctctatgaagttctcagg | 8635 |
| ccccacctacaccctcatc | 4464 | gatgagggtgtaggtgggg | 5855 | ctgagaacttcatagaggt | 7246 | acctctatgaagttctcag | 8636 |
| cccacctacaccctcatca | 4465 | tgatgagggtgtaggtggg | 5856 | tgagaacttcatagaggtg | 7247 | cacctctatgaagttctca | 8637 |
| ccacctacaccctcatcat | 4466 | atgatgagggtgtaggtgg | 5857 | gagaacttcatagaggtgt | 7248 | acacctctatgaagttctc | 8638 |
| cacctacaccctcatcatc | 4467 | gatgatgagggtgtaggtg | 5858 | agaacttcatagaggtgtg | 7249 | cacacctctatgaagttct | 8639 |
| acctacaccctcatcatcc | 4468 | ggatgatgagggtgtaggt | 5859 | gaacttcatagaggtgtga | 7250 | tcacacctctatgaagttc | 8640 |
| cctacaccctcatcatccg | 4469 | cggatgatgagggtgtagg | 5860 | aacttcatagaggtgtgat | 7251 | atcacacctctatgaagtt | 8641 |
| ctacaccctcatcatccga | 4470 | tcggatgatgagggtgtag | 5861 | acttcatagaggtgtgatt | 7252 | aatcacacctctatgaagt | 8642 |
| tacaccctcatcatccgag | 4471 | ctcggatgatgagggtgta | 5862 | cttcatagaggtgtgattg | 7253 | caatcacacctctatgaag | 8643 |
| acaccctcatcatccgagg | 4472 | cctcggatgatgagggtgt | 5863 | ttcatagaggtgtgattgc | 7254 | gcaatcacacctctatgaa | 8644 |
| caccctcatcatccgaggc | 4473 | gcctcggatgatgagggtg | 5864 | tcatagaggtgtgattgca | 7255 | tgcaatcacacctctatga | 8645 |
| accctcatcatccgaggca | 4474 | tgcctcggatgatgagggt | 5865 | catagaggtgtgattgcac | 7256 | gtgcaatcacacctctatg | 8646 |
| ccctcatcatccgaggcaa | 4475 | ttgcctcggatgatgaggg | 5866 | atagaggtgtgattgcact | 7257 | agtgcaatcacacctctat | 8647 |
| cctcatcatccgaggcaag | 4476 | cttgcctcggatgatgagg | 5867 | tagaggtgtgattgcactt | 7258 | aagtgcaatcacacctcta | 8648 |
| ctcatcatccgaggcaaga | 4477 | tcttgcctcggatgatgag | 5868 | agaggtgtgattgcacttt | 7259 | aaagtgcaatcacacctct | 8649 |
| tcatcatccgaggcaagat | 4478 | atcttgcctcggatgatga | 5869 | gaggtgtgattgcacttta | 7260 | taaagtgcaatcacacctc | 8650 |
| catcatccgaggcaagatc | 4479 | gatcttgcctcggatgatg | 5870 | aggtgtgattgcactttat | 7261 | ataaagtgcaatcacacct | 8651 |
| atcatccgaggcaagatcc | 4480 | ggatcttgcctcggatgat | 5871 | ggtgtgattgcactttatg | 7262 | cataaagtgcaatcacacc | 8652 |
| tcatccgaggcaagatccg | 4481 | cggatcttgcctcggatga | 5872 | gtgtgattgcactttatgt | 7263 | acataaagtgcaatcacac | 8653 |
| catccgaggcaagatccgg | 4482 | ccggatcttgcctcggatg | 5873 | tgtgattgcactttatgtc | 7264 | gacataaagtgcaatcaca | 8654 |
| atccgaggcaagatccggc | 4483 | gccggatcttgcctcggat | 5874 | gtgattgcactttatgtct | 7265 | agacataaagtgcaatcac | 8655 |
| tccgaggcaagatccggct | 4484 | agccggatcttgcctcgga | 5875 | tgattgcactttatgtctg | 7266 | cagacataaagtgcaatca | 8656 |
| ccgaggcaagatccggctt | 4485 | aagccggatcttgcctcgg | 5876 | gattgcactttatgtctgc | 7267 | gcagacataaagtgcaatc | 8657 |
| cgaggcaagatccggcttc | 4486 | gaagccggatcttgcctcg | 5877 | attgcactttatgtctgca | 7268 | tgcagacataaagtgcaat | 8658 |
| gaggcaagatccggcttcg | 4487 | cgaagccggatcttgcctc | 5878 | ttgcactttatgtctgcag | 7269 | ctgcagacataaagtgcaa | 8659 |
| aggcaagatccggcttcgc | 4488 | gcgaagccggatcttgcct | 5879 | tgcactttatgtctgcaga | 7270 | tctgcagacataaagtgca | 8660 |
| ggcaagatccggcttcgcc | 4489 | ggcgaagccggatcttgcc | 5880 | gcactttatgtctgcagag | 7271 | ctctgcagacataaagtgc | 8661 |
| gcaagatccggcttcgcca | 4490 | tggcgaagccggatcttgc | 5881 | cactttatgtctgcagaga | 7272 | tctctgcagacataaagtg | 8662 |
| caagatccggcttcgccag | 4491 | ctggcgaagccggatcttg | 5882 | actttatgtctgcagagag | 7273 | ctctctgcagacataaagt | 8663 |
| aagatccggcttcgccagg | 4492 | cctggcgaagccggatctt | 5883 | ctttatgtctgcagagagc | 7274 | gctctctgcagacataaag | 8664 |
| agatccggcttcgccaggc | 4493 | gcctggcgaagccggatct | 5884 | tttatgtctgcagagagct | 7275 | agctctctgcagacataaa | 8665 |
| gatccggcttcgccaggcg | 4494 | cgcctggcgaagccggatc | 5885 | ttatgtctgcagagagctg | 7276 | cagctctctgcagacataa | 8666 |
| atccggcttcgccaggcgt | 4495 | acgcctggcgaagccggat | 5886 | tatgtctgcagagagctgg | 7277 | ccagctctctgcagacata | 8667 |
| tccggcttcgccaggcgtc | 4496 | gacgcctggcgaagccgga | 5887 | atgtctgcagagagctggg | 7278 | cccagctctctgcagacat | 8668 |
| ccggcttcgccaggcgtcc | 4497 | ggacgcctggcgaagccgg | 5888 | tgtctgcagagagctgggg | 7279 | ccccagctctctgcagaca | 8669 |
| cggcttcgccaggcgtcct | 4498 | aggacgcctggcgaagccg | 5889 | gtctgcagagagctggggc | 7280 | gccccagctctctgcagac | 8670 |
| ggcttcgccaggcgtcctg | 4499 | caggacgcctggcgaagcc | 5890 | tctgcagagagctggggcg | 7281 | cgccccagctctctgcaga | 8671 |
| gcttcgccaggcgtcctgg | 4500 | ccaggacgcctggcgaagc | 5891 | ctgcagagagctggggcga | 7282 | tcgccccagctctctgcag | 8672 |
| cttcgccaggcgtcctgga | 4501 | tccaggacgcctggcgaag | 5892 | tgcagagagctggggcgag | 7283 | ctcgccccagctctctgca | 8673 |
| ttcgccaggcgtcctggat | 4502 | atccaggacgcctggcgaa | 5893 | gcagagagctggggcgagt | 7284 | actcgccccagctctctgc | 8674 |
| tcgccaggcgtcctggatc | 4503 | gatccaggacgcctggcga | 5894 | cagagagctggggcgagtg | 7285 | cactcgccccagctctctg | 8675 |
| cgccaggcgtcctggatca | 4504 | tgatccaggacgcctggcg | 5895 | agagagctggggcgagtgt | 7286 | acactcgccccagctctct | 8676 |
| gccaggcgtcctggatcat | 4505 | atgatccaggacgcctggc | 5896 | gagagctggggcgagtgtg | 7287 | cacactcgccccagctctc | 8677 |
| ccaggcgtcctggatcatc | 4506 | gatgatccaggacgcctgg | 5897 | agagctggggcgagtgtgt | 7288 | acacactcgccccagctct | 8678 |
| caggcgtcctggatcatcc | 4507 | ggatgatccaggacgcctg | 5898 | gagctggggcgagtgtgtt | 7289 | aacacactcgccccagctc | 8679 |
| aggcgtcctggatcatccg | 4508 | cggatgatccaggacgcct | 5899 | agctggggcgagtgtgttt | 7290 | aaacacactcgccccagct | 8680 |
| ggcgtcctggatcatccgt | 4509 | acggatgatccaggacgcc | 5900 | gctggggcgagtgtgttta | 7291 | taaacacactcgccccagc | 8681 |
| gcgtcctggatcatccgtg | 4510 | cacggatgatccaggacgc | 5901 | ctggggcgagtgtgtttag | 7292 | ctaaacacactcgccccag | 8682 |
| cgtcctggatcatccgtgg | 4511 | ccacggatgatccaggacg | 5902 | tggggcgagtgtgtttagg | 7293 | cctaaacacactcgcccca | 8683 |
| gtcctggatcatccgtggg | 4512 | cccacggatgatccaggac | 5903 | ggggcgagtgtgtttaggg | 7294 | ccctaaacacactcgcccc | 8684 |
| tcctggatcatccgtgggg | 4513 | ccccacggatgatccagga | 5904 | gggcgagtgtgtttagggg | 7295 | cccctaaacacactcgccc | 8685 |
| cctggatcatccgtggggg | 4514 | cccccacggatgatccagg | 5905 | ggcgagtgtgtttaggggt | 7296 | acccctaaacacactcgcc | 8686 |
| ctggatcatccgtgggggc | 4515 | gcccccacggatgatccag | 5906 | gcgagtgtgtttaggggtg | 7297 | cacccctaaacacactcgc | 8687 |
| tggatcatccgtgggggca | 4516 | tgcccccacggatgatcca | 5907 | cgagtgtgtttaggggtgg | 7298 | ccacccctaaacacactcg | 8688 |
| ggatcatccgtgggggcac | 4517 | gtgcccccacggatgatcc | 5908 | gagtgtgtttaggggtggc | 7299 | gccacccctaaacacactc | 8689 |
| gatcatccgtgggggcacc | 4518 | ggtgcccccacggatgatc | 5909 | agtgtgtttaggggtggca | 7300 | tgccacccctaaacacact | 8690 |
| atcatccgtgggggcaccg | 4519 | cggtgcccccacggatgat | 5910 | gtgtgtttaggggtggcag | 7301 | ctgccacccctaaacacac | 8691 |
| tcatccgtgggggcaccga | 4520 | tcggtgcccccacggatga | 5911 | tgtgtttaggggtggcagc | 7302 | gctgccacccctaaacaca | 8692 |
| catccgtgggggcaccgaa | 4521 | ttcggtgcccccacggatg | 5912 | gtgtttaggggtggcagct | 7303 | agctgccacccctaaacac | 8693 |
| atccgtgggggcaccgaag | 4522 | cttcggtgcccccacggat | 5913 | tgtttaggggtggcagctg | 7304 | cagctgccacccctaaaca | 8694 |
| tccgtgggggcaccgaagc | 4523 | gcttcggtgcccccacgga | 5914 | gtttaggggtggcagctgc | 7305 | gcagctgccacccctaaac | 8695 |
| ccgtgggggcaccgaagct | 4524 | agcttcggtgcccccacgg | 5915 | tttaggggtggcagctgcc | 7306 | ggcagctgccacccctaaa | 8696 |
| cgtgggggcaccgaagctg | 4525 | cagcttcggtgcccccacg | 5916 | ttaggggtggcagctgcct | 7307 | aggcagctgccacccctaa | 8697 |
| gtgggggcaccgaagctga | 4526 | tcagcttcggtgcccccac | 5917 | taggggtggcagctgcctt | 7308 | aaggcagctgccaccccta | 8698 |
| tgggggcaccgaagctgac | 4527 | gtcagcttcggtgccccca | 5918 | aggggtggcagctgccttc | 7309 | gaaggcagctgccacccct | 8699 |
| gggggcaccgaagctgact | 4528 | agtcagcttcggtgccccc | 5919 | ggggtggcagctgccttct | 7310 | agaaggcagctgccacccc | 8700 |
| ggggcaccgaagctgacta | 4529 | tagtcagcttcggtgcccc | 5920 | gggtggcagctgccttctt | 7311 | aagaaggcagctgccaccc | 8701 |
| gggcaccgaagctgactac | 4530 | gtagtcagcttcggtgccc | 5921 | ggtggcagctgccttcttc | 7312 | gaagaaggcagctgccacc | 8702 |
| ggcaccgaagctgactacc | 4531 | ggtagtcagcttcggtgcc | 5922 | gtggcagctgccttcttct | 7313 | agaagaaggcagctgccac | 8703 |
| gcaccgaagctgactacca | 4532 | tggtagtcagcttcggtgc | 5923 | tggcagctgccttcttctc | 7314 | gagaagaaggcagctgcca | 8704 |
| caccgaagctgactaccag | 4533 | ctggtagtcagcttcggtg | 5924 | ggcagctgccttcttctct | 7315 | agagaagaaggcagctgcc | 8705 |
| accgaagctgactaccagc | 4534 | gctggtagtcagcttcggt | 5925 | gcagctgccttcttctctc | 7316 | gagagaagaaggcagctgc | 8706 |
| ccgaagctgactaccagct | 4535 | agctggtagtcagcttcgg | 5926 | cagctgccttcttctctct | 7317 | agagagaagaaggcagctg | 8707 |
| cgaagctgactaccagctt | 4536 | aagctggtagtcagcttcg | 5927 | agctgccttcttctctctg | 7318 | cagagagaagaaggcagct | 8708 |
| gaagctgactaccagcttc | 4537 | gaagctggtagtcagcttc | 5928 | gctgccttcttctctctgt | 7319 | acagagagaagaaggcagc | 8709 |
| aagctgactaccagcttca | 4538 | tgaagctggtagtcagctt | 5929 | ctgccttcttctctctgtc | 7320 | gacagagagaagaaggcag | 8710 |
| agctgactaccagcttcac | 4539 | gtgaagctggtagtcagct | 5930 | tgccttcttctctctgtcc | 7321 | ggacagagagaagaaggca | 8711 |
| gctgactaccagcttcacg | 4540 | cgtgaagctggtagtcagc | 5931 | gccttcttctctctgtccc | 7322 | gggacagagagaagaaggc | 8712 |
| ctgactaccagcttcacgg | 4541 | ccgtgaagctggtagtcag | 5932 | ccttcttctctctgtcccc | 7323 | ggggacagagagaagaagg | 8713 |
| tgactaccagcttcacggc | 4542 | gccgtgaagctggtagtca | 5933 | cttcttctctctgtcccct | 7324 | aggggacagagagaagaag | 8714 |
| gactaccagcttcacggcg | 4543 | cgccgtgaagctggtagtc | 5934 | ttcttctctctgtcccctg | 7325 | caggggacagagagaagaa | 8715 |
| actaccagcttcacggcgt | 4544 | acgccgtgaagctggtagt | 5935 | tcttctctctgtcccctga | 7326 | tcaggggacagagagaaga | 8716 |
| ctaccagcttcacggcgtc | 4545 | gacgccgtgaagctggtag | 5936 | cttctctctgtcccctgac | 7327 | gtcaggggacagagagaag | 8717 |
| taccagcttcacggcgtcc | 4546 | ggacgccgtgaagctggta | 5937 | ttctctctgtcccctgact | 7328 | agtcaggggacagagagaa | 8718 |
| accagcttcacggcgtcca | 4547 | tggacgccgtgaagctggt | 5938 | tctctctgtcccctgactc | 7329 | gagtcaggggacagagaga | 8719 |
| ccagcttcacggcgtccaa | 4548 | ttggacgccgtgaagctgg | 5939 | ctctctgtcccctgactcc | 7330 | ggagtcaggggacagagag | 8720 |
| cagcttcacggcgtccaag | 4549 | cttggacgccgtgaagctg | 5940 | tctctgtcccctgactcct | 7331 | aggagtcaggggacagaga | 8721 |
| agcttcacggcgtccaagt | 4550 | acttggacgccgtgaagct | 5941 | ctctgtcccctgactcctg | 7332 | caggagtcaggggacagag | 8722 |
| gcttcacggcgtccaagtc | 4551 | gacttggacgccgtgaagc | 5942 | tctgtcccctgactcctga | 7333 | tcaggagtcaggggacaga | 8723 |
| cttcacggcgtccaagtca | 4552 | tgacttggacgccgtgaag | 5943 | ctgtcccctgactcctgac | 7334 | gtcaggagtcaggggacag | 8724 |
| ttcacggcgtccaagtcat | 4553 | atgacttggacgccgtgaa | 5944 | tgtcccctgactcctgact | 7335 | agtcaggagtcaggggaca | 8725 |
| tcacggcgtccaagtcatc | 4554 | gatgacttggacgccgtga | 5945 | gtcccctgactcctgactg | 7336 | cagtcaggagtcaggggac | 8726 |
| cacggcgtccaagtcatct | 4555 | agatgacttggacgccgtg | 5946 | tcccctgactcctgactgt | 7337 | acagtcaggagtcagggga | 8727 |
| acggcgtccaagtcatctg | 4556 | cagatgacttggacgccgt | 5947 | cccctgactcctgactgtg | 7338 | cacagtcaggagtcagggg | 8728 |
| cggcgtccaagtcatctgc | 4557 | gcagatgacttggacgccg | 5948 | ccctgactcctgactgtgt | 7339 | acacagtcaggagtcaggg | 8729 |
| ggcgtccaagtcatctgcc | 4558 | ggcagatgacttggacgcc | 5949 | cctgactcctgactgtgtt | 7340 | aacacagtcaggagtcagg | 8730 |
| gcgtccaagtcatctgcca | 4559 | tggcagatgacttggacgc | 5950 | ctgactcctgactgtgttt | 7341 | aaacacagtcaggagtcag | 8731 |
| cgtccaagtcatctgccac | 4560 | gtggcagatgacttggacg | 5951 | tgactcctgactgtgtttc | 7342 | gaaacacagtcaggagtca | 8732 |
| gtccaagtcatctgccaca | 4561 | tgtggcagatgacttggac | 5952 | gactcctgactgtgtttct | 7343 | agaaacacagtcaggagtc | 8733 |
| tccaagtcatctgccacac | 4562 | gtgtggcagatgacttgga | 5953 | actcctgactgtgtttctg | 7344 | cagaaacacagtcaggagt | 8734 |
| ccaagtcatctgccacaca | 4563 | tgtgtggcagatgacttgg | 5954 | ctcctgactgtgtttctga | 7345 | tcagaaacacagtcaggag | 8735 |
| caagtcatctgccacacag | 4564 | ctgtgtggcagatgacttg | 5955 | tcctgactgtgtttctgag | 7346 | ctcagaaacacagtcagga | 8736 |
| aagtcatctgccacacaga | 4565 | tctgtgtggcagatgactt | 5956 | cctgactgtgtttctgagc | 7347 | gctcagaaacacagtcagg | 8737 |
| agtcatctgccacacagag | 4566 | ctctgtgtggcagatgact | 5957 | ctgactgtgtttctgagct | 7348 | agctcagaaacacagtcag | 8738 |
| gtcatctgccacacagagg | 4567 | cctctgtgtggcagatgac | 5958 | tgactgtgtttctgagctg | 7349 | cagctcagaaacacagtca | 8739 |
| tcatctgccacacagaggc | 4568 | gcctctgtgtggcagatga | 5959 | gactgtgtttctgagctga | 7350 | tcagctcagaaacacagtc | 8740 |
| catctgccacacagaggca | 4569 | tgcctctgtgtggcagatg | 5960 | actgtgtttctgagctgag | 7351 | ctcagctcagaaacacagt | 8741 |
| atctgccacacagaggcag | 4570 | ctgcctctgtgtggcagat | 5961 | ctgtgtttctgagctgagc | 7352 | gctcagctcagaaacacag | 8742 |
| tctgccacacagaggcagt | 4571 | actgcctctgtgtggcaga | 5962 | tgtgtttctgagctgagct | 7353 | agctcagctcagaaacaca | 8743 |
| ctgccacacagaggcagtc | 4572 | gactgcctctgtgtggcag | 5963 | gtgtttctgagctgagctt | 7354 | aagctcagctcagaaacac | 8744 |
| tgccacacagaggcagtcg | 4573 | cgactgcctctgtgtggca | 5964 | tgtttctgagctgagctta | 7355 | taagctcagctcagaaaca | 8745 |
| gccacacagaggcagtcgc | 4574 | gcgactgcctctgtgtggc | 5965 | gtttctgagctgagcttaa | 7356 | ttaagctcagctcagaaac | 8746 |
| ccacacagaggcagtcgct | 4575 | agcgactgcctctgtgtgg | 5966 | tttctgagctgagcttaaa | 7357 | tttaagctcagctcagaaa | 8747 |
| cacacagaggcagtcgctg | 4576 | cagcgactgcctctgtgtg | 5967 | ttctgagctgagcttaaaa | 7358 | ttttaagctcagctcagaa | 8748 |
| acacagaggcagtcgctga | 4577 | tcagcgactgcctctgtgt | 5968 | tctgagctgagcttaaaag | 7359 | cttttaagctcagctcaga | 8749 |
| cacagaggcagtcgctgaa | 4578 | ttcagcgactgcctctgtg | 5969 | ctgagctgagcttaaaaga | 7360 | tcttttaagctcagctcag | 8750 |
| acagaggcagtcgctgaac | 4579 | gttcagcgactgcctctgt | 5970 | tgagctgagcttaaaagat | 7361 | atcttttaagctcagctca | 8751 |
| cagaggcagtcgctgaaca | 4580 | tgttcagcgactgcctctg | 5971 | gagctgagcttaaaagata | 7362 | tatcttttaagctcagctc | 8752 |
| agaggcagtcgctgaacag | 4581 | ctgttcagcgactgcctct | 5972 | agctgagcttaaaagatac | 7363 | gtatcttttaagctcagct | 8753 |
| gaggcagtcgctgaacagc | 4582 | gctgttcagcgactgcctc | 5973 | gctgagcttaaaagataca | 7364 | tgtatcttttaagctcagc | 8754 |
| aggcagtcgctgaacagct | 4583 | agctgttcagcgactgcct | 5974 | ctgagcttaaaagatacaa | 7365 | ttgtatcttttaagctcag | 8755 |
| ggcagtcgctgaacagctc | 4584 | gagctgttcagcgactgcc | 5975 | tgagcttaaaagatacaaa | 7366 | tttgtatcttttaagctca | 8756 |
| gcagtcgctgaacagctca | 4585 | tgagctgttcagcgactgc | 5976 | gagcttaaaagatacaaat | 7367 | atttgtatcttttaagctc | 8757 |
| cagtcgctgaacagctcag | 4586 | ctgagctgttcagcgactg | 5977 | agcttaaaagatacaaatg | 7368 | catttgtatcttttaagct | 8758 |
| agtcgctgaacagctcagc | 4587 | gctgagctgttcagcgact | 5978 | gcttaaaagatacaaatga | 7369 | tcatttgtatcttttaagc | 8759 |
| gtcgctgaacagctcagcc | 4588 | ggctgagctgttcagcgac | 5979 | cttaaaagatacaaatgac | 7370 | gtcatttgtatcttttaag | 8760 |
| tcgctgaacagctcagccg | 4589 | cggctgagctgttcagcga | 5980 | ttaaaagatacaaatgaca | 7371 | tgtcatttgtatcttttaa | 8761 |
| cgctgaacagctcagccga | 4590 | tcggctgagctgttcagcg | 5981 | taaaagatacaaatgacaa | 7372 | ttgtcatttgtatctttta | 8762 |
| gctgaacagctcagccgac | 4591 | gtcggctgagctgttcagc | 5982 | aaaagatacaaatgacaag | 7373 | cttgtcatttgtatctttt | 8763 |
| ctgaacagctcagccgact | 4592 | agtcggctgagctgttcag | 5983 | aaagatacaaatgacaagc | 7374 | gcttgtcatttgtatcttt | 8764 |
| tgaacagctcagccgactg | 4593 | cagtcggctgagctgttca | 5984 | aagatacaaatgacaagct | 7375 | agcttgtcatttgtatctt | 8765 |
| gaacagctcagccgactgg | 4594 | ccagtcggctgagctgttc | 5985 | agatacaaatgacaagctg | 7376 | cagcttgtcatttgtatct | 8766 |
| aacagctcagccgactggt | 4595 | accagtcggctgagctgtt | 5986 | gatacaaatgacaagctgc | 7377 | gcagcttgtcatttgtatc | 8767 |
| acagctcagccgactggtg | 4596 | caccagtcggctgagctgt | 5987 | atacaaatgacaagctgca | 7378 | tgcagcttgtcatttgtat | 8768 |
| cagctcagccgactggtga | 4597 | tcaccagtcggctgagctg | 5988 | tacaaatgacaagctgcag | 7379 | ctgcagcttgtcatttgta | 8769 |
| agctcagccgactggtgaa | 4598 | ttcaccagtcggctgagct | 5989 | acaaatgacaagctgcagc | 7380 | gctgcagcttgtcatttgt | 8770 |
| gctcagccgactggtgaac | 4599 | gttcaccagtcggctgagc | 5990 | caaatgacaagctgcagct | 7381 | agctgcagcttgtcatttg | 8771 |
| ctcagccgactggtgaacc | 4600 | ggttcaccagtcggctgag | 5991 | aaatgacaagctgcagctc | 7382 | gagctgcagcttgtcattt | 8772 |
| tcagccgactggtgaaccg | 4601 | cggttcaccagtcggctga | 5992 | aatgacaagctgcagctct | 7383 | agagctgcagcttgtcatt | 8773 |
| cagccgactggtgaaccga | 4602 | tcggttcaccagtcggctg | 5993 | atgacaagctgcagctctc | 7384 | gagagctgcagcttgtcat | 8774 |
| agccgactggtgaaccgaa | 4603 | ttcggttcaccagtcggct | 5994 | tgacaagctgcagctctct | 7385 | agagagctgcagcttgtca | 8775 |
| gccgactggtgaaccgaac | 4604 | gttcggttcaccagtcggc | 5995 | gacaagctgcagctctctc | 7386 | gagagagctgcagcttgtc | 8776 |
| ccgactggtgaaccgaact | 4605 | agttcggttcaccagtcgg | 5996 | acaagctgcagctctctct | 7387 | agagagagctgcagcttgt | 8777 |
| cgactggtgaaccgaactt | 4606 | aagttcggttcaccagtcg | 5997 | caagctgcagctctctctg | 7388 | cagagagagctgcagcttg | 8778 |
| gactggtgaaccgaacttg | 4607 | caagttcggttcaccagtc | 5998 | aagctgcagctctctctgc | 7389 | gcagagagagctgcagctt | 8779 |
| actggtgaaccgaacttgc | 4608 | gcaagttcggttcaccagt | 5999 | agctgcagctctctctgcc | 7390 | ggcagagagagctgcagct | 8780 |
| ctggtgaaccgaacttgcc | 4609 | ggcaagttcggttcaccag | 6000 | gctgcagctctctctgcca | 7391 | tggcagagagagctgcagc | 8781 |
| tggtgaaccgaacttgccc | 4610 | gggcaagttcggttcacca | 6001 | ctgcagctctctctgccat | 7392 | atggcagagagagctgcag | 8782 |
| ggtgaaccgaacttgccca | 4611 | tgggcaagttcggttcacc | 6002 | tgcagctctctctgccatt | 7393 | aatggcagagagagctgca | 8783 |
| gtgaaccgaacttgcccag | 4612 | ctgggcaagttcggttcac | 6003 | gcagctctctctgccattg | 7394 | caatggcagagagagctgc | 8784 |
| tgaaccgaacttgcccagg | 4613 | cctgggcaagttcggttca | 6004 | cagctctctctgccattgc | 7395 | gcaatggcagagagagctg | 8785 |
| gaaccgaacttgcccaggc | 4614 | gcctgggcaagttcggttc | 6005 | agctctctctgccattgct | 7396 | agcaatggcagagagagct | 8786 |
| aaccgaacttgcccaggct | 4615 | agcctgggcaagttcggtt | 6006 | gctctctctgccattgctt | 7397 | aagcaatggcagagagagc | 8787 |
| accgaacttgcccaggctt | 4616 | aagcctgggcaagttcggt | 6007 | ctctctctgccattgcttc | 7398 | gaagcaatggcagagagag | 8788 |
| ccgaacttgcccaggcttc | 4617 | gaagcctgggcaagttcgg | 6008 | tctctctgccattgcttct | 7399 | agaagcaatggcagagaga | 8789 |
| cgaacttgcccaggcttcc | 4618 | ggaagcctgggcaagttcg | 6009 | ctctctgccattgcttctc | 7400 | gagaagcaatggcagagag | 8790 |
| gaacttgcccaggcttcct | 4619 | aggaagcctgggcaagttc | 6010 | tctctgccattgcttctct | 7401 | agagaagcaatggcagaga | 8791 |
| aacttgcccaggcttcctg | 4620 | caggaagcctgggcaagtt | 6011 | ctctgccattgcttctctt | 7402 | aagagaagcaatggcagag | 8792 |
| acttgcccaggcttcctgg | 4621 | ccaggaagcctgggcaagt | 6012 | tctgccattgcttctcttt | 7403 | aaagagaagcaatggcaga | 8793 |
| cttgcccaggcttcctggc | 4622 | gccaggaagcctgggcaag | 6013 | ctgccattgcttctcttta | 7404 | taaagagaagcaatggcag | 8794 |
| ttgcccaggcttcctggct | 4623 | agccaggaagcctgggcaa | 6014 | tgccattgcttctctttat | 7405 | ataaagagaagcaatggca | 8795 |
| tgcccaggcttcctggctc | 4624 | gagccaggaagcctgggca | 6015 | gccattgcttctctttatc | 7406 | gataaagagaagcaatggc | 8796 |
| gcccaggcttcctggctcc | 4625 | ggagccaggaagcctgggc | 6016 | ccattgcttctctttatct | 7407 | agataaagagaagcaatgg | 8797 |
| cccaggcttcctggctcct | 4626 | aggagccaggaagcctggg | 6017 | cattgcttctctttatctc | 7408 | gagataaagagaagcaatg | 8798 |
| ccaggcttcctggctcctg | 4627 | caggagccaggaagcctgg | 6018 | attgcttctctttatctct | 7409 | agagataaagagaagcaat | 8799 |
| caggcttcctggctcctgg | 4628 | ccaggagccaggaagcctg | 6019 | ttgcttctctttatctctc | 7410 | gagagataaagagaagcaa | 8800 |
| aggcttcctggctcctggt | 4629 | accaggagccaggaagcct | 6020 | tgcttctctttatctctcc | 7411 | ggagagataaagagaagca | 8801 |
| ggcttcctggctcctggtg | 4630 | caccaggagccaggaagcc | 6021 | gcttctctttatctctcca | 7412 | tggagagataaagagaagc | 8802 |
| gcttcctggctcctggtgg | 4631 | ccaccaggagccaggaagc | 6022 | cttctctttatctctccaa | 7413 | ttggagagataaagagaag | 8803 |
| cttcctggctcctggtggt | 4632 | accaccaggagccaggaag | 6023 | ttctctttatctctccaaa | 7414 | tttggagagataaagagaa | 8804 |
| ttcctggctcctggtggtc | 4633 | gaccaccaggagccaggaa | 6024 | tctctttatctctccaaat | 7415 | atttggagagataaagaga | 8805 |
| tcctggctcctggtggtcc | 4634 | ggaccaccaggagccagga | 6025 | ctctttatctctccaaatc | 7416 | gatttggagagataaagag | 8806 |
| cctggctcctggtggtccc | 4635 | gggaccaccaggagccagg | 6026 | tctttatctctccaaatcc | 7417 | ggatttggagagataaaga | 8807 |
| ctggctcctggtggtccct | 4636 | agggaccaccaggagccag | 6027 | ctttatctctccaaatccc | 7418 | gggatttggagagataaag | 8808 |
| tggctcctggtggtccctg | 4637 | cagggaccaccaggagcca | 6028 | tttatctctccaaatccct | 7419 | agggatttggagagataaa | 8809 |
| ggctcctggtggtccctgg | 4638 | ccagggaccaccaggagcc | 6029 | ttatctctccaaatccctt | 7420 | aagggatttggagagataa | 8810 |
| gctcctggtggtccctggg | 4639 | cccagggaccaccaggagc | 6030 | tatctctccaaatcccttc | 7421 | gaagggatttggagagata | 8811 |
| ctcctggtggtccctgggt | 4640 | acccagggaccaccaggag | 6031 | atctctccaaatcccttcc | 7422 | ggaagggatttggagagat | 8812 |
| tcctggtggtccctgggta | 4641 | tacccagggaccaccagga | 6032 | tctctccaaatcccttcca | 7423 | tggaagggatttggagaga | 8813 |
| cctggtggtccctgggtac | 4642 | gtacccagggaccaccagg | 6033 | ctctccaaatcccttccac | 7424 | gtggaagggatttggagag | 8814 |
| ctggtggtccctgggtaca | 4643 | tgtacccagggaccaccag | 6034 | tctccaaatcccttccaca | 7425 | tgtggaagggatttggaga | 8815 |
| tggtggtccctgggtacag | 4644 | ctgtacccagggaccacca | 6035 | ctccaaatcccttccacac | 7426 | gtgtggaagggatttggag | 8816 |
| ggtggtccctgggtacagg | 4645 | cctgtacccagggaccacc | 6036 | tccaaatcccttccacact | 7427 | agtgtggaagggatttgga | 8817 |
| gtggtccctgggtacagga | 4646 | tcctgtacccagggaccac | 6037 | ccaaatcccttccacactc | 7428 | gagtgtggaagggatttgg | 8818 |
| tggtccctgggtacaggac | 4647 | gtcctgtacccagggacca | 6038 | caaatcccttccacactcg | 7429 | cgagtgtggaagggatttg | 8819 |
| ggtccctgggtacaggacg | 4648 | cgtcctgtacccagggacc | 6039 | aaatcccttccacactcga | 7430 | tcgagtgtggaagggattt | 8820 |
| gtccctgggtacaggacgt | 4649 | acgtcctgtacccagggac | 6040 | aatcccttccacactcgag | 7431 | ctcgagtgtggaagggatt | 8821 |
| tccctgggtacaggacgta | 4650 | tacgtcctgtacccaggga | 6041 | atcccttccacactcgagg | 7432 | cctcgagtgtggaagggat | 8822 |
| ccctgggtacaggacgtag | 4651 | ctacgtcctgtacccaggg | 6042 | tcccttccacactcgaggt | 7433 | acctcgagtgtggaaggga | 8823 |
| cctgggtacaggacgtagc | 4652 | gctacgtcctgtacccagg | 6043 | cccttccacactcgaggtt | 7434 | aacctcgagtgtggaaggg | 8824 |
| ctgggtacaggacgtagcc | 4653 | ggctacgtcctgtacccag | 6044 | ccttccacactcgaggttt | 7435 | aaacctcgagtgtggaagg | 8825 |
| tgggtacaggacgtagcct | 4654 | aggctacgtcctgtaccca | 6045 | cttccacactcgaggtttt | 7436 | aaaacctcgagtgtggaag | 8826 |
| gggtacaggacgtagccta | 4655 | taggctacgtcctgtaccc | 6046 | ttccacactcgaggttttc | 7437 | gaaaacctcgagtgtggaa | 8827 |
| ggtacaggacgtagcctat | 4656 | ataggctacgtcctgtacc | 6047 | tccacactcgaggttttcg | 7438 | cgaaaacctcgagtgtgga | 8828 |
| gtacaggacgtagcctatg | 4657 | cataggctacgtcctgtac | 6048 | ccacactcgaggttttcga | 7439 | tcgaaaacctcgagtgtgg | 8829 |
| tacaggacgtagcctatga | 4658 | tcataggctacgtcctgta | 6049 | cacactcgaggttttcgac | 7440 | gtcgaaaacctcgagtgtg | 8830 |
| acaggacgtagcctatgac | 4659 | gtcataggctacgtcctgt | 6050 | acactcgaggttttcgaca | 7441 | tgtcgaaaacctcgagtgt | 8831 |
| caggacgtagcctatgacc | 4660 | ggtcataggctacgtcctg | 6051 | cactcgaggttttcgacac | 7442 | gtgtcgaaaacctcgagtg | 8832 |
| aggacgtagcctatgacct | 4661 | aggtcataggctacgtcct | 6052 | actcgaggttttcgacact | 7443 | agtgtcgaaaacctcgagt | 8833 |
| ggacgtagcctatgacctg | 4662 | caggtcataggctacgtcc | 6053 | ctcgaggttttcgacactg | 7444 | cagtgtcgaaaacctcgag | 8834 |
| gacgtagcctatgacctgt | 4663 | acaggtcataggctacgtc | 6054 | tcgaggttttcgacactgg | 7445 | ccagtgtcgaaaacctcga | 8835 |
| acgtagcctatgacctgtg | 4664 | cacaggtcataggctacgt | 6055 | cgaggttttcgacactgga | 7446 | tccagtgtcgaaaacctcg | 8836 |
| cgtagcctatgacctgtgg | 4665 | ccacaggtcataggctacg | 6056 | gaggttttcgacactggaa | 7447 | ttccagtgtcgaaaacctc | 8837 |
| gtagcctatgacctgtggc | 4666 | gccacaggtcataggctac | 6057 | aggttttcgacactggaac | 7448 | gttccagtgtcgaaaacct | 8838 |
| tagcctatgacctgtggca | 4667 | tgccacaggtcataggcta | 6058 | ggttttcgacactggaacc | 7449 | ggttccagtgtcgaaaacc | 8839 |
| agcctatgacctgtggcag | 4668 | ctgccacaggtcataggct | 6059 | gttttcgacactggaaccc | 7450 | gggttccagtgtcgaaaac | 8840 |
| gcctatgacctgtggcagg | 4669 | cctgccacaggtcataggc | 6060 | ttttcgacactggaaccca | 7451 | tgggttccagtgtcgaaaa | 8841 |
| cctatgacctgtggcagga | 4670 | tcctgccacaggtcatagg | 6061 | tttcgacactggaacccag | 7452 | ctgggttccagtgtcgaaa | 8842 |
| ctatgacctgtggcaggag | 4671 | ctcctgccacaggtcatag | 6062 | ttcgacactggaacccagt | 7453 | actgggttccagtgtcgaa | 8843 |
| tatgacctgtggcaggagg | 4672 | cctcctgccacaggtcata | 6063 | tcgacactggaacccagtg | 7454 | cactgggttccagtgtcga | 8844 |
| atgacctgtggcaggagga | 4673 | tcctcctgccacaggtcat | 6064 | cgacactggaacccagtgc | 7455 | gcactgggttccagtgtcg | 8845 |
| tgacctgtggcaggaggag | 4674 | ctcctcctgccacaggtca | 6065 | gacactggaacccagtgca | 7456 | tgcactgggttccagtgtc | 8846 |
| gacctgtggcaggaggaga | 4675 | tctcctcctgccacaggtc | 6066 | acactggaacccagtgcag | 7457 | ctgcactgggttccagtgt | 8847 |
| acctgtggcaggaggagag | 4676 | ctctcctcctgccacaggt | 6067 | cactggaacccagtgcagc | 7458 | gctgcactgggttccagtg | 8848 |
| cctgtggcaggaggagagt | 4677 | actctcctcctgccacagg | 6068 | actggaacccagtgcagcg | 7459 | cgctgcactgggttccagt | 8849 |
| ctgtggcaggaggagagta | 4678 | tactctcctcctgccacag | 6069 | ctggaacccagtgcagcgt | 7460 | acgctgcactgggttccag | 8850 |
| tgtggcaggaggagagtaa | 4679 | ttactctcctcctgccaca | 6070 | tggaacccagtgcagcgtt | 7461 | aacgctgcactgggttcca | 8851 |
| gtggcaggaggagagtaac | 4680 | gttactctcctcctgccac | 6071 | ggaacccagtgcagcgttg | 7462 | caacgctgcactgggttcc | 8852 |
| tggcaggaggagagtaacc | 4681 | ggttactctcctcctgcca | 6072 | gaacccagtgcagcgttgg | 7463 | ccaacgctgcactgggttc | 8853 |
| ggcaggaggagagtaacca | 4682 | tggttactctcctcctgcc | 6073 | aacccagtgcagcgttggc | 7464 | gccaacgctgcactgggtt | 8854 |
| gcaggaggagagtaaccac | 4683 | gtggttactctcctcctgc | 6074 | acccagtgcagcgttggct | 7465 | agccaacgctgcactgggt | 8855 |
| caggaggagagtaaccacg | 4684 | cgtggttactctcctcctg | 6075 | cccagtgcagcgttggctt | 7466 | aagccaacgctgcactggg | 8856 |
| aggaggagagtaaccacga | 4685 | tcgtggttactctcctcct | 6076 | ccagtgcagcgttggcttg | 7467 | caagccaacgctgcactgg | 8857 |
| ggaggagagtaaccacgag | 4686 | ctcgtggttactctcctcc | 6077 | cagtgcagcgttggcttgg | 7468 | ccaagccaacgctgcactg | 8858 |
| gaggagagtaaccacgagt | 4687 | actcgtggttactctcctc | 6078 | agtgcagcgttggcttggg | 7469 | cccaagccaacgctgcact | 8859 |
| aggagagtaaccacgagtg | 4688 | cactcgtggttactctcct | 6079 | gtgcagcgttggcttggga | 7470 | tcccaagccaacgctgcac | 8860 |
| ggagagtaaccacgagtgc | 4689 | gcactcgtggttactctcc | 6080 | tgcagcgttggcttgggac | 7471 | gtcccaagccaacgctgca | 8861 |
| gagagtaaccacgagtgca | 4690 | tgcactcgtggttactctc | 6081 | gcagcgttggcttgggacc | 7472 | ggtcccaagccaacgctgc | 8862 |
| agagtaaccacgagtgcac | 4691 | gtgcactcgtggttactct | 6082 | cagcgttggcttgggaccg | 7473 | cggtcccaagccaacgctg | 8863 |
| gagtaaccacgagtgcacc | 4692 | ggtgcactcgtggttactc | 6083 | agcgttggcttgggaccgc | 7474 | gcggtcccaagccaacgct | 8864 |
| agtaaccacgagtgcacca | 4693 | tggtgcactcgtggttact | 6084 | gcgttggcttgggaccgcc | 7475 | ggcggtcccaagccaacgc | 8865 |
| gtaaccacgagtgcaccaa | 4694 | ttggtgcactcgtggttac | 6085 | cgttggcttgggaccgcca | 7476 | tggcggtcccaagccaacg | 8866 |
| taaccacgagtgcaccaag | 4695 | cttggtgcactcgtggtta | 6086 | gttggcttgggaccgccat | 7477 | atggcggtcccaagccaac | 8867 |
| aaccacgagtgcaccaagg | 4696 | ccttggtgcactcgtggtt | 6087 | ttggcttgggaccgccatg | 7478 | catggcggtcccaagccaa | 8868 |
| accacgagtgcaccaaggc | 4697 | gccttggtgcactcgtggt | 6088 | tggcttgggaccgccatga | 7479 | tcatggcggtcccaagcca | 8869 |
| ccacgagtgcaccaaggct | 4698 | agccttggtgcactcgtgg | 6089 | ggcttgggaccgccatgaa | 7480 | ttcatggcggtcccaagcc | 8870 |
| cacgagtgcaccaaggctg | 4699 | cagccttggtgcactcgtg | 6090 | gcttgggaccgccatgaag | 7481 | cttcatggcggtcccaagc | 8871 |
| acgagtgcaccaaggctgt | 4700 | acagccttggtgcactcgt | 6091 | cttgggaccgccatgaaga | 7482 | tcttcatggcggtcccaag | 8872 |
| cgagtgcaccaaggctgtg | 4701 | cacagccttggtgcactcg | 6092 | ttgggaccgccatgaagac | 7483 | gtcttcatggcggtcccaa | 8873 |
| gagtgcaccaaggctgtga | 4702 | tcacagccttggtgcactc | 6093 | tgggaccgccatgaagacc | 7484 | ggtcttcatggcggtccca | 8874 |
| agtgcaccaaggctgtgaa | 4703 | ttcacagccttggtgcact | 6094 | gggaccgccatgaagaccc | 7485 | gggtcttcatggcggtccc | 8875 |
| gtgcaccaaggctgtgaac | 4704 | gttcacagccttggtgcac | 6095 | ggaccgccatgaagacccc | 7486 | ggggtcttcatggcggtcc | 8876 |
| tgcaccaaggctgtgaact | 4705 | agttcacagccttggtgca | 6096 | gaccgccatgaagacccca | 7487 | tggggtcttcatggcggtc | 8877 |
| gcaccaaggctgtgaactt | 4706 | aagttcacagccttggtgc | 6097 | accgccatgaagaccccat | 7488 | atggggtcttcatggcggt | 8878 |
| caccaaggctgtgaacttt | 4707 | aaagttcacagccttggtg | 6098 | ccgccatgaagaccccatg | 7489 | catggggtcttcatggcgg | 8879 |
| accaaggctgtgaactttg | 4708 | caaagttcacagccttggt | 6099 | cgccatgaagaccccatgt | 7490 | acatggggtcttcatggcg | 8880 |
| ccaaggctgtgaactttgc | 4709 | gcaaagttcacagccttgg | 6100 | gccatgaagaccccatgtt | 7491 | aacatggggtcttcatggc | 8881 |
| caaggctgtgaactttgcc | 4710 | ggcaaagttcacagccttg | 6101 | ccatgaagaccccatgttt | 7492 | aaacatggggtcttcatgg | 8882 |
| aaggctgtgaactttgcca | 4711 | tggcaaagttcacagcctt | 6102 | catgaagaccccatgtttg | 7493 | caaacatggggtcttcatg | 8883 |
| aggctgtgaactttgccat | 4712 | atggcaaagttcacagcct | 6103 | atgaagaccccatgtttgc | 7494 | gcaaacatggggtcttcat | 8884 |
| ggctgtgaactttgccatg | 4713 | catggcaaagttcacagcc | 6104 | tgaagaccccatgtttgcg | 7495 | cgcaaacatggggtcttca | 8885 |
| gctgtgaactttgccatgc | 4714 | gcatggcaaagttcacagc | 6105 | gaagaccccatgtttgcga | 7496 | tcgcaaacatggggtcttc | 8886 |
| ctgtgaactttgccatgca | 4715 | tgcatggcaaagttcacag | 6106 | aagaccccatgtttgcgac | 7497 | gtcgcaaacatggggtctt | 8887 |
| tgtgaactttgccatgcac | 4716 | gtgcatggcaaagttcaca | 6107 | agaccccatgtttgcgaca | 7498 | tgtcgcaaacatggggtct | 8888 |
| gtgaactttgccatgcacg | 4717 | cgtgcatggcaaagttcac | 6108 | gaccccatgtttgcgacag | 7499 | ctgtcgcaaacatggggtc | 8889 |
| tgaactttgccatgcacga | 4718 | tcgtgcatggcaaagttca | 6109 | accccatgtttgcgacagg | 7500 | cctgtcgcaaacatggggt | 8890 |
| gaactttgccatgcacgag | 4719 | ctcgtgcatggcaaagttc | 6110 | ccccatgtttgcgacagga | 7501 | tcctgtcgcaaacatgggg | 8891 |
| aactttgccatgcacgagc | 4720 | gctcgtgcatggcaaagtt | 6111 | cccatgtttgcgacaggag | 7502 | ctcctgtcgcaaacatggg | 8892 |
| actttgccatgcacgagct | 4721 | agctcgtgcatggcaaagt | 6112 | ccatgtttgcgacaggaga | 7503 | tctcctgtcgcaaacatgg | 8893 |
| ctttgccatgcacgagctg | 4722 | cagctcgtgcatggcaaag | 6113 | catgtttgcgacaggagag | 7504 | ctctcctgtcgcaaacatg | 8894 |
| tttgccatgcacgagctgc | 4723 | gcagctcgtgcatggcaaa | 6114 | atgtttgcgacaggagagc | 7505 | gctctcctgtcgcaaacat | 8895 |
| ttgccatgcacgagctgca | 4724 | tgcagctcgtgcatggcaa | 6115 | tgtttgcgacaggagagcc | 7506 | ggctctcctgtcgcaaaca | 8896 |
| tgccatgcacgagctgcag | 4725 | ctgcagctcgtgcatggca | 6116 | gtttgcgacaggagagcct | 7507 | aggctctcctgtcgcaaac | 8897 |
| gccatgcacgagctgcagc | 4726 | gctgcagctcgtgcatggc | 6117 | tttgcgacaggagagcctg | 7508 | caggctctcctgtcgcaaa | 8898 |
| ccatgcacgagctgcagct | 4727 | agctgcagctcgtgcatgg | 6118 | ttgcgacaggagagcctgg | 7509 | ccaggctctcctgtcgcaa | 8899 |
| catgcacgagctgcagctc | 4728 | gagctgcagctcgtgcatg | 6119 | tgcgacaggagagcctggg | 7510 | cccaggctctcctgtcgca | 8900 |
| atgcacgagctgcagctca | 4729 | tgagctgcagctcgtgcat | 6120 | gcgacaggagagcctgggt | 7511 | acccaggctctcctgtcgc | 8901 |
| tgcacgagctgcagctcat | 4730 | atgagctgcagctcgtgca | 6121 | cgacaggagagcctgggtt | 7512 | aacccaggctctcctgtcg | 8902 |
| gcacgagctgcagctcatc | 4731 | gatgagctgcagctcgtgc | 6122 | gacaggagagcctgggttg | 7513 | caacccaggctctcctgtc | 8903 |
| cacgagctgcagctcatcc | 4732 | ggatgagctgcagctcgtg | 6123 | acaggagagcctgggttgg | 7514 | ccaacccaggctctcctgt | 8904 |
| acgagctgcagctcatccg | 4733 | cggatgagctgcagctcgt | 6124 | caggagagcctgggttggt | 7515 | accaacccaggctctcctg | 8905 |
| cgagctgcagctcatccgt | 4734 | acggatgagctgcagctcg | 6125 | aggagagcctgggttggtg | 7516 | caccaacccaggctctcct | 8906 |
| gagctgcagctcatccgtg | 4735 | cacggatgagctgcagctc | 6126 | ggagagcctgggttggtgt | 7517 | acaccaacccaggctctcc | 8907 |
| agctgcagctcatccgtgt | 4736 | acacggatgagctgcagct | 6127 | gagagcctgggttggtgtt | 7518 | aacaccaacccaggctctc | 8908 |
| gctgcagctcatccgtgtg | 4737 | cacacggatgagctgcagc | 6128 | agagcctgggttggtgttg | 7519 | caacaccaacccaggctct | 8909 |
| ctgcagctcatccgtglgg | 4738 | ccacacggatgagctgcag | 6129 | gagcctgggttggtgttga | 7520 | tcaacaccaacccaggctc | 8910 |
| tgcagctcatccgtgtgga | 4739 | tccacacggatgagctgca | 6130 | agcctgggttggtgttgag | 7521 | ctcaacaccaacccaggct | 8911 |
| gcagctcatccgtgtggag | 4740 | ctccacacggatgagctgc | 6131 | gcctgggttggtgttgagt | 7522 | actcaacaccaacccaggc | 8912 |
| cagctcatccgtgtggaga | 4741 | tctccacacggatgagctg | 6132 | cctgggttggtgttgagta | 7523 | tactcaacaccaacccagg | 8913 |
| agctcatccgtgtggagaa | 4742 | ttctccacacggatgagct | 6133 | ctgggttggtgttgagtac | 7524 | gtactcaacaccaacccag | 8914 |
| gctcatccgtgtggagaag | 4743 | cttctccacacggatgagc | 6134 | tgggttggtgttgagtaca | 7525 | tgtactcaacaccaaccca | 8915 |
| ctcatccgtgtggagaagc | 4744 | gcttctccacacggatgag | 6135 | gggttggtgttgagtacat | 7526 | atgtactcaacaccaaccc | 8916 |
| tcatccgtgtggagaagca | 4745 | tgcttctccacacggatga | 6136 | ggttggtgttgagtacata | 7527 | tatgtactcaacaccaacc | 8917 |
| catccgtgtggagaagcag | 4746 | ctgcttctccacacggatg | 6137 | gttggtgttgagtacataa | 7528 | ttatgtactcaacaccaac | 8918 |
| atccgtgtggagaagcagt | 4747 | actgcttctccacacggat | 6138 | ttggtgttgagtacataag | 7529 | cttatgtactcaacaccaa | 8919 |
| tccgtgtggagaagcagta | 4748 | tactgcttctccacacgga | 6139 | tggtgttgagtacataaga | 7530 | tcttatgtactcaacacca | 8920 |
| ccgtgtggagaagcagtat | 4749 | atactgcttctccacacgg | 6140 | ggtgttgagtacataagag | 7531 | ctcttatgtactcaacacc | 8921 |
| cgtgtggagaagcagtatc | 4750 | gatactgcttctccacacg | 6141 | gtgttgagtacataagaga | 7532 | tctcttatgtactcaacac | 8922 |
| gtgtggagaagcagtatcc | 4751 | ggatactgcttctccacac | 6142 | tgttgagtacataagagac | 7533 | gtctcttatgtactcaaca | 8923 |
| tgtggagaagcagtatccc | 4752 | gggatactgcttctccaca | 6143 | gttgagtacataagagacg | 7534 | cgtctcttatgtactcaac | 8924 |
| gtggagaagcagtatcccc | 4753 | ggggatactgcttctccac | 6144 | ttgagtacataagagacga | 7535 | tcgtctcttatgtactcaa | 8925 |
| tggagaagcagtatcccca | 4754 | tggggatactgcttctcca | 6145 | tgagtacataagagacgag | 7536 | ctcgtctcttatgtactca | 8926 |
| ggagaagcagtatccccac | 4755 | gtggggatactgcttctcc | 6146 | gagtacataagagacgagg | 7537 | cctcgtctcttatgtactc | 8927 |
| gagaagcagtatccccacc | 4756 | ggtggggatactgcttctc | 6147 | agtacataagagacgaggc | 7538 | gcctcgtctcttatgtact | 8928 |
| agaagcagtatccccacca | 4757 | tggtggggatactgcttct | 6148 | gtacataagagacgaggca | 7539 | tgcctcgtctcttatgtac | 8929 |
| gaagcagtatccccaccac | 4758 | gtggtggggatactgcttc | 6149 | tacataagagacgaggcaa | 7540 | ttgcctcgtctcttatgta | 8930 |
| aagcagtatccccaccaca | 4759 | tgtggtggggatactgctt | 6150 | acataagagacgaggcaag | 7541 | cttgcctcgtctcttatgt | 8931 |
| agcagtatccccaccacag | 4760 | ctgtggtggggatactgct | 6151 | cataagagacgaggcaagg | 7542 | ccttgcctcgtctcttatg | 8932 |
| gcagtatccccaccacagc | 4761 | gctgtggtggggatactgc | 6152 | ataagagacgaggcaaggt | 7543 | accttgcctcgtctcttat | 8933 |
| cagtatccccaccacagcc | 4762 | ggctgtggtggggatactg | 6153 | taagagacgaggcaaggtt | 7544 | aaccttgcctcgtctctta | 8934 |
| agtatccccaccacagcct | 4763 | aggctgtggtggggatact | 6154 | aagagacgaggcaaggttc | 7545 | gaaccttgcctcgtctctt | 8935 |
| gtatccccaccacagcctg | 4764 | caggctgtggtggggatac | 6155 | agagacgaggcaaggttca | 7546 | tgaaccttgcctcgtctct | 8936 |
| tatccccaccacagcctgg | 4765 | ccaggctgtggtggggata | 6156 | gagacgaggcaaggttcag | 7547 | ctgaaccttgcctcgtctc | 8937 |
| atccccaccacagcctgga | 4766 | tccaggctgtggtggggat | 6157 | agacgaggcaaggttcagc | 7548 | gctgaaccttgcctcgtct | 8938 |
| tccccaccacagcctggac | 4767 | gtccaggctgtggtgggga | 6158 | gacgaggcaaggttcagct | 7549 | agctgaaccttgcctcgtc | 8939 |
| ccccaccacagcctggacc | 4768 | ggtccaggctgtggtgggg | 6159 | acgaggcaaggttcagcta | 7550 | tagctgaaccttgcctcgt | 8940 |
| cccaccacagcctggacca | 4769 | tggtccaggctgtggtggg | 6160 | cgaggcaaggttcagctaa | 7551 | ttagctgaaccttgcctcg | 8941 |
| ccaccacagcctggaccac | 4770 | gtggtccaggctgtggtgg | 6161 | gaggcaaggttcagctaag | 7552 | cttagctgaaccttgcctc | 8942 |
| caccacagcctggaccacc | 4771 | ggtggtccaggctgtggtg | 6162 | aggcaaggttcagctaagt | 7553 | acttagctgaaccttgcct | 8943 |
| accacagcctggaccacct | 4772 | aggtggtccaggctgtggt | 6163 | ggcaaggttcagctaagtc | 7554 | gacttagctgaaccttgcc | 8944 |
| ccacagcctggaccacctg | 4773 | caggtggtccaggctgtgg | 6164 | gcaaggttcagctaagtct | 7555 | agacttagctgaaccttgc | 8945 |
| cacagcctggaccacctgg | 4774 | ccaggtggtccaggctgtg | 6165 | caaggttcagctaagtctt | 7556 | aagacttagctgaaccttg | 8946 |
| acagcctggaccacctggt | 4775 | accaggtggtccaggctgt | 6166 | aaggttcagctaagtcttt | 7557 | aaagacttagctgaacctt | 8947 |
| cagcctggaccacctggtg | 4776 | caccaggtggtccaggctg | 6167 | aggttcagctaagtctttg | 7558 | caaagacttagctgaacct | 8948 |
| agcctggaccacctggtgg | 4777 | ccaccaggtggtccaggct | 6168 | ggttcagctaagtctttgt | 7559 | acaaagacttagctgaacc | 8949 |
| gcctggaccacctggtgga | 4778 | tccaccaggtggtccaggc | 6169 | gttcagctaagtctttgtc | 7560 | gacaaagacttagctgaac | 8950 |
| cctggaccacctggtggag | 4779 | ctccaccaggtggtccagg | 6170 | ttcagctaagtctttgtcc | 7561 | ggacaaagacttagctgaa | 8951 |
| ctggaccacctggtggagg | 4780 | cctccaccaggtggtccag | 6171 | tcagctaagtctttgtccc | 7562 | gggacaaagacttagctga | 8952 |
| tggaccacctggtggagga | 4781 | tcctccaccaggtggtcca | 6172 | cagctaagtctttgtccca | 7563 | tgggacaaagacttagctg | 8953 |
| ggaccacctggtggaggag | 4782 | ctcctccaccaggtggtcc | 6173 | agctaagtctttgtcccag | 7564 | ctgggacaaagacttagct | 8954 |
| gaccacctggtggaggagc | 4783 | gctcctccaccaggtggtc | 6174 | gctaagtctttgtcccagc | 7565 | gctgggacaaagacttagc | 8955 |
| accacctggtggaggagct | 4784 | agctcctccaccaggtggt | 6175 | ctaagtctttgtcccagcc | 7566 | ggctgggacaaagacttag | 8956 |
| ccacctggtggaggagctc | 4785 | gagctcctccaccaggtgg | 6176 | taagtctttgtcccagcct | 7567 | aggctgggacaaagactta | 8957 |
| cacctggtggaggagctct | 4786 | agagctcctccaccaggtg | 6177 | aagtctttgtcccagcctt | 7568 | aaggctgggacaaagactt | 8958 |
| acctggtggaggagctctt | 4787 | aagagctcctccaccaggt | 6178 | agtctttgtcccagcctta | 7569 | taaggctgggacaaagact | 8959 |
| cctggtggaggagctcttc | 4788 | gaagagctcctccaccagg | 6179 | gtctttgtcccagccttaa | 7570 | ttaaggctgggacaaagac | 8960 |
| ctggtggaggagctcttcc | 4789 | ggaagagctcctccaccag | 6180 | tctttgtcccagccttaat | 7571 | attaaggctgggacaaaga | 8961 |
| tggtggaggagctcttcct | 4790 | aggaagagctcctccacca | 6181 | ctttgtcccagccttaatg | 7572 | cattaaggctgggacaaag | 8962 |
| ggtggaggagctcttcctg | 4791 | caggaagagctcctccacc | 6182 | tttgtcccagccttaatgc | 7573 | gcattaaggctgggacaaa | 8963 |
| gtggaggagctcttcctgg | 4792 | ccaggaagagctcctccac | 6183 | ttgtcccagccttaatgct | 7574 | agcattaaggctgggacaa | 8964 |
| tggaggagctcttcctggg | 4793 | cccaggaagagctcctcca | 6184 | tgtcccagccttaatgcta | 7575 | tagcattaaggctgggaca | 8965 |
| ggaggagctcttcctgggc | 4794 | gcccaggaagagctcctcc | 6185 | gtcccagccttaatgctaa | 7576 | ttagcattaaggctgggac | 8966 |
| gaggagctcttcctgggcg | 4795 | cgcccaggaagagctcctc | 6186 | tcccagccttaatgctaac | 7577 | gttagcattaaggctggga | 8967 |
| aggagctcttcctgggcga | 4796 | tcgcccaggaagagctcct | 6187 | cccagccttaatgctaacc | 7578 | ggttagcattaaggctggg | 8968 |
| ggagctcttcctgggcgac | 4797 | gtcgcccaggaagagctcc | 6188 | ccagccttaatgctaacca | 7579 | tggttagcattaaggctgg | 8969 |
| gagctcttcctgggcgaca | 4798 | tgtcgcccaggaagagctc | 6189 | cagccttaatgctaaccaa | 7580 | ttggttagcattaaggctg | 8970 |
| agctcttcctgggcgacat | 4799 | atgtcgcccaggaagagct | 6190 | agccttaatgctaaccaac | 7581 | gttggttagcattaaggct | 8971 |
| gctcftcctgggcgacatc | 4800 | gatgtcgcccaggaagagc | 6191 | gccttaatgctaaccaaca | 7582 | tgttggttagcattaaggc | 8972 |
| ctcttcctgggcgacatcc | 4801 | ggatgtcgcccaggaagag | 6192 | ccttaatgctaaccaacac | 7583 | gtgttggttagcattaagg | 8973 |
| tcttcctgggcgacatcca | 4802 | tggatgtcgcccaggaaga | 6193 | cttaatgctaaccaacact | 7584 | agtgttggttagcattaag | 8974 |
| cttcctgggcgacatccac | 4803 | gtggatgtcgcccaggaag | 6194 | ttaatgctaaccaacactt | 7585 | aagtgttggttagcattaa | 8975 |
| ttcctgggcgacatccaca | 4804 | tgtggatgtcgcccaggaa | 6195 | taatgctaaccaacacttt | 7586 | aaagtgttggttagcatta | 8976 |
| tcctgggcgacatccacac | 4805 | gtgtggatgtcgcccagga | 6196 | aatgctaaccaacactttc | 7587 | gaaagtgttggttagcatt | 8977 |
| cctgggcgacatccacacg | 4806 | cgtgtggatgtcgcccagg | 6197 | atgctaaccaacactttca | 7588 | tgaaagtgttggttagcat | 8978 |
| ctgggcgacatccacacgg | 4807 | ccgtgtggatgtcgcccag | 6198 | tgctaaccaacactttcag | 7589 | ctgaaagtgttggttagca | 8979 |
| tgggcgacatccacacgga | 4808 | tccgtgtggatgtcgccca | 6199 | gctaaccaacactttcaga | 7590 | tctgaaagtgttggttagc | 8980 |
| gggcgacatccacacggac | 4809 | gtccgtgtggatgtcgccc | 6200 | ctaaccaacactttcagaa | 7591 | ttctgaaagtgttggttag | 8981 |
| ggcgacatccacacggacg | 4810 | cgtccgtgtggatgtcgcc | 6201 | taaccaacactttcagaaa | 7592 | tttctgaaagtgttggtta | 8982 |
| gcgacatccacacggacgc | 4811 | gcgtccgtgtggatgtcgc | 6202 | aaccaacactttcagaaat | 7593 | atttctgaaagtgttggtt | 8983 |
| cgacatccacacggacgct | 4812 | agcgtccgtgtggatgtcg | 6203 | accaacactttcagaaatg | 7594 | catttctgaaagtgttggt | 8984 |
| gacatccacacggacgcta | 4813 | tagcgtccgtgtggatgtc | 6204 | ccaacactttcagaaatgt | 7595 | acatttctgaaagtgttgg | 8985 |
| acatccacacggacgctac | 4814 | gtagcgtccgtgtggatgt | 6205 | caacactttcagaaatgtt | 7596 | aacatttctgaaagtgttg | 8986 |
| catccacacggacgctacc | 4815 | ggtagcgtccgtgtggatg | 6206 | aacactttcagaaatgttg | 7597 | caacatttctgaaagtgtt | 8987 |
| atccacacggacgctaccc | 4816 | gggtagcgtccgtgtggat | 6207 | acactttcagaaatgttga | 7598 | tcaacatttctgaaagtgt | 8988 |
| tccacacggacgctaccca | 4817 | tgggtagcgtccgtgtgga | 6208 | cactttcagaaatgttgag | 7599 | ctcaacatttctgaaagtg | 8989 |
| ccacacggacgctacccag | 4818 | ctgggtagcgtccgtgtgg | 6209 | actttcagaaatgttgagt | 7600 | actcaacatttctgaaagt | 8990 |
| cacacggacgctacccaga | 4819 | tctgggtagcgtccgtgtg | 6210 | ctttcagaaatgttgagta | 7601 | tactcaacatttctgaaag | 8991 |
| acacggacgctacccagag | 4820 | ctctgggtagcgtccgtgt | 6211 | tttcagaaatgttgagtag | 7602 | ctactcaacatttctgaaa | 8992 |
| cacggacgctacccagagg | 4821 | cctctgggtagcgtccgtg | 6212 | ttcagaaatgttgagtaga | 7603 | tctactcaacatttctgaa | 8993 |
| acggacgctacccagaggg | 4822 | ccctctgggtagcgtccgt | 6213 | tcagaaatgttgagtagag | 7604 | ctctactcaacatttctga | 8994 |
| cggacgctacccagagggt | 4823 | accctctgggtagcgtccg | 6214 | cagaaatgttgagtagaga | 7605 | tctctactcaacatttctg | 8995 |
| ggacgctacccagagggtg | 4824 | caccctctgggtagcgtcc | 6215 | agaaatgttgagtagagag | 7606 | ctctctactcaacatttct | 8996 |
| gacgctacccagagggtgt | 4825 | acaccctctgggtagcgtc | 6216 | gaaatgttgagtagagagg | 7607 | cctctctactcaacatttc | 8997 |
| acgctacccagagggtgtt | 4826 | aacaccctctgggtagcgt | 6217 | aaatgttgagtagagaggt | 7608 | acctctctactcaacattt | 8998 |
| cgctacccagagggtgttc | 4827 | gaacaccctctgggtagcg | 6218 | aatgttgagtagagaggtg | 7609 | cacctctctactcaacatt | 8999 |
| gctacccagagggtgttct | 4828 | agaacaccctctgggtagc | 6219 | atgttgagtagagaggtgt | 7610 | acacctctctactcaacat | 9000 |
| ctacccagagggtgttcta | 4829 | tagaacaccctctgggtag | 6220 | tgttgagtagagaggtgtc | 7611 | gacacctctctactcaaca | 9001 |
| tacccagagggtgttctac | 4830 | gtagaacaccctctgggta | 6221 | gttgagtagagaggtgtcc | 7612 | ggacacctctctactcaac | 9002 |
| acccagagggtgttctacc | 4831 | ggtagaacaccctctgggt | 6222 | ttgagtagagaggtgtccc | 7613 | gggacacctctctactcaa | 9003 |
| cccagagggtgttctaccg | 4832 | cggtagaacaccctctggg | 6223 | tgagtagagaggtgtccct | 7614 | agggacacctctctactca | 9004 |
| ccagagggtgttctaccgg | 4833 | ccggtagaacaccctctgg | 6224 | gagtagagaggtgtcccta | 7615 | tagggacacctctctactc | 9005 |
| cagagggtgttctaccggc | 4834 | gccggtagaacaccctctg | 6225 | agtagagaggtgtccctaa | 7616 | ttagggacacctctctact | 9006 |
| agagggtgttctaccggcc | 4835 | ggccggtagaacaccctct | 6226 | gtagagaggtgtccctaaa | 7617 | tttagggacacctctctac | 9007 |
| gagggtgttctaccggccg | 4836 | cggccggtagaacaccctc | 6227 | tagagaggtgtccctaaag | 7618 | ctttagggacacctctcta | 9008 |
| agggtgttctaccggccgt | 4837 | acggccggtagaacaccct | 6228 | agagaggtgtccctaaagc | 7619 | gctttagggacacctctct | 9009 |
| gggtgttctaccggccgtc | 4838 | gacggccggtagaacaccc | 6229 | gagaggtgtccctaaagct | 7620 | agctttagggacacctctc | 9010 |
| ggtgttctaccggccgtcc | 4839 | ggacggccggtagaacacc | 6230 | agaggtgtccctaaagctt | 7621 | aagctttagggacacctct | 9011 |
| gtgttctaccggccgtcca | 4840 | tggacggccggtagaacac | 6231 | gaggtgtccctaaagcttc | 7622 | gaagctttagggacacctc | 9012 |
| tgttctaccggccgtccag | 4841 | ctggacggccggtagaaca | 6232 | aggtgtccctaaagcttca | 7623 | tgaagctttagggacacct | 9013 |
| gttctaccggccgtccagt | 4842 | actggacggccggtagaac | 6233 | ggtgtccctaaagcttcaa | 7624 | ttgaagctttagggacacc | 9014 |
| ttctaccggccgtccagtt | 4843 | aactggacggccggtagaa | 6234 | gtgtccctaaagcttcaat | 7625 | attgaagctttagggacac | 9015 |
| tctaccggccgtccagtta | 4844 | taactggacggccggtaga | 6235 | tgtccctaaagcttcaatg | 7626 | cattgaagctttagggaca | 9016 |
| ctaccggccgtccagttac | 4845 | gtaactggacggccggtag | 6236 | gtccctaaagcttcaatgg | 7627 | ccattgaagctttagggac | 9017 |
| taccggccgtccagttacc | 4846 | ggtaactggacggccggta | 6237 | tccctaaagcttcaatgga | 7628 | tccattgaagctttaggga | 9018 |
| accggccgtccagttacca | 4847 | tggtaactggacggccggt | 6238 | ccctaaagcttcaatggaa | 7629 | ttccattgaagctttaggg | 9019 |
| ccggccgtccagttaccag | 4848 | ctggtaactggacggccgg | 6239 | cctaaagcttcaatggaac | 7630 | gttccattgaagctttagg | 9020 |
| cggccgtccagttaccagc | 4849 | gctggtaactggacggccg | 6240 | ctaaagcttcaatggaact | 7631 | agttccattgaagctttag | 9021 |
| ggccgtccagttaccagcc | 4850 | ggctggtaactggacggcc | 6241 | taaagcttcaatggaactt | 7632 | aagttccattgaagcttta | 9022 |
| gccgtccagttaccagccg | 4851 | cggctggtaactggacggc | 6242 | aaagcttcaatggaactta | 7633 | taagttccattgaagcttt | 9023 |
| ccgtccagttaccagccgc | 4852 | gcggctggtaactggacgg | 6243 | aagcttcaatggaacttaa | 7634 | ttaagttccattgaagctt | 9024 |
| cgtccagttaccagccgcc | 4853 | ggcggctggtaactggacg | 6244 | agcttcaatggaacttaaa | 7635 | tttaagttccattgaagct | 9025 |
| gtccagttaccagccgccc | 4854 | gggcggctggtaactggac | 6245 | gcttcaatggaacttaaac | 7636 | gtttaagttccattgaagc | 9026 |
| tccagttaccagccgcccc | 4855 | ggggcggctggtaactgga | 6246 | cttcaatggaacttaaact | 7637 | agtttaagttccattgaag | 9027 |
| ccagttaccagccgcccct | 4856 | aggggcggctggtaactgg | 6247 | ttcaatggaacttaaactc | 7638 | gagtttaagttccattgaa | 9028 |
| cagttaccagccgcccctg | 4857 | caggggcggctggtaactg | 6248 | tcaatggaacttaaactct | 7639 | agagtttaagttccattga | 9029 |
| agttaccagccgcccctgc | 4858 | gcaggggcggctggtaact | 6249 | caatggaacttaaactctg | 7640 | cagagtttaagttccattg | 9030 |
| gttaccagccgcccctgca | 4859 | tgcaggggcggctggtaac | 6250 | aatggaacttaaactctgt | 7641 | acagagtttaagttccatt | 9031 |
| ttaccagccgcccctgcag | 4860 | ctgcaggggcggctggtaa | 6251 | atggaacttaaactctgtt | 7642 | aacagagtttaagttccat | 9032 |
| taccagccgcccctgcaga | 4861 | tctgcaggggcggctggta | 6252 | tggaacttaaactctgttg | 7643 | caacagagtttaagttcca | 9033 |
| accagccgcccctgcagaa | 4862 | ttctgcaggggcggctggt | 6253 | ggaacttaaactctgttga | 7644 | tcaacagagtttaagttcc | 9034 |
| ccagccgcccctgcagaat | 4863 | attctgcaggggcggctgg | 6254 | gaacttaaactctgttgac | 7645 | gtcaacagagtttaagttc | 9035 |
| cagccgcccctgcagaatg | 4864 | cattctgcaggggcggctg | 6255 | aacttaaactctgttgaca | 7646 | tgtcaacagagtttaagtt | 9036 |
| agccgcccctgcagaatgc | 4865 | gcattctgcaggggcggct | 6256 | acttaaactctgttgacaa | 7647 | ttgtcaacagagtttaagt | 9037 |
| gccgcccctgcagaatgcc | 4866 | ggcattctgcaggggcggc | 6257 | cttaaactctgttgacaag | 7648 | cttgtcaacagagtttaag | 9038 |
| ccgcccctgcagaatgcca | 4867 | tggcattctgcaggggcgg | 6258 | ttaaactctgttgacaagc | 7649 | gcttgtcaacagagtttaa | 9039 |
| cgcccctgcagaatgccaa | 4868 | ttggcattctgcaggggcg | 6259 | taaactctgttgacaagcg | 7650 | cgcttgtcaacagagttta | 9040 |
| gcccctgcagaatgccaag | 4869 | cttggcattctgcaggggc | 6260 | aaactctgttgacaagcga | 7651 | tcgcttgtcaacagagttt | 9041 |
| cccctgcagaatgccaaga | 4870 | tcttggcattctgcagggg | 6261 | aactctgttgacaagcgag | 7652 | ctcgcttgtcaacagagtt | 9042 |
| ccctgcagaatgccaagaa | 4871 | ttcttggcattctgcaggg | 6262 | actctgttgacaagcgagt | 7653 | actcgcttgtcaacagagt | 9043 |
| cctgcagaatgccaagaac | 4872 | gttcttggcattctgcagg | 6263 | ctctgttgacaagcgagtg | 7654 | cactcgcttgtcaacagag | 9044 |
| ctgcagaatgccaagaacc | 4873 | ggttcttggcattctgcag | 6264 | tctgttgacaagcgagtgc | 7655 | gcactcgcttgtcaacaga | 9045 |
| tgcagaatgccaagaacca | 4874 | tggttcttggcattctgca | 6265 | ctgttgacaagcgagtgcc | 7656 | ggcactcgcttgtcaacag | 9046 |
| gcagaatgccaagaaccac | 4875 | gtggttcttggcattctgc | 6266 | tgttgacaagcgagtgccg | 7657 | cggcactcgcttgtcaaca | 9047 |
| cagaatgccaagaaccaca | 4876 | tgtggttcttggcattctg | 6267 | gttgacaagcgagtgccgg | 7658 | ccggcactcgcttgtcaac | 9048 |
| agaatgccaagaaccacaa | 4877 | ttgtggttcttggcattct | 6268 | ttgacaagcgagtgccggt | 7659 | accggcactcgcttgtcaa | 9049 |
| gaatgccaagaaccacaac | 4878 | gttgtggttcttggcattc | 6269 | tgacaagcgagtgccggtt | 7660 | aaccggcactcgcttgtca | 9050 |
| aatgccaagaaccacaacc | 4879 | ggttgtggttcttggcatt | 6270 | gacaagcgagtgccggttt | 7661 | aaaccggcactcgcttgtc | 9051 |
| atgccaagaaccacaacca | 4880 | tggttgtggttcttggcat | 6271 | acaagcgagtgccggtttt | 7662 | aaaaccggcactcgcttgt | 9052 |
| tgccaagaaccacaaccat | 4881 | atggttgtggttcttggca | 6272 | caagcgagtgccggttttc | 7663 | gaaaaccggcactcgcttg | 9053 |
| gccaagaaccacaaccatg | 4882 | catggttgtggttcttggc | 6273 | aagcgagtgccggttttca | 7664 | tgaaaaccggcactcgctt | 9054 |
| ccaagaaccacaaccatgc | 4883 | gcatggttgtggttcttgg | 6274 | agcgagtgccggttttcac | 7665 | gtgaaaaccggcactcgct | 9055 |
| caagaaccacaaccatgcg | 4884 | cgcatggttgtggttcttg | 6275 | gcgagtgccggttttcact | 7666 | agtgaaaaccggcactcgc | 9056 |
| aagaaccacaaccatgcgt | 4885 | acgcatggttgtggttctt | 6276 | cgagtgccggttttcactt | 7667 | aagtgaaaaccggcactcg | 9057 |
| agaaccacaaccatgcgtg | 4886 | cacgcatggttgtggttct | 6277 | gagtgccggttttcacttg | 7668 | caagtgaaaaccggcactc | 9058 |
| gaaccacaaccatgcgtgc | 4887 | gcacgcatggttgtggttc | 6278 | agtgccggttttcacttgt | 7669 | acaagtgaaaaccggcact | 9059 |
| aaccacaaccatgcgtgca | 4888 | tgcacgcatggttgtggtt | 6279 | gtgccggttttcacttgtt | 7670 | aacaagtgaaaaccggcac | 9060 |
| accacaaccatgcgtgcat | 4889 | atgcacgcatggttgtggt | 6280 | tgccggttttcacttgttg | 7671 | caacaagtgaaaaccggca | 9061 |
| ccacaaccatgcgtgcata | 4890 | tatgcacgcatggttgtgg | 6281 | gccggttttcacttgttga | 7672 | tcaacaagtgaaaaccggc | 9062 |
| cacaaccatgcgtgcatag | 4891 | ctatgcacgcatggttgtg | 6282 | ccggttttcacttgttgag | 7673 | ctcaacaagtgaaaaccgg | 9063 |
| acaaccatgcgtgcatagc | 4892 | gctatgcacgcatggttgt | 6283 | cggttttcacttgttgaga | 7674 | tctcaacaagtgaaaaccg | 9064 |
| caaccatgcgtgcatagcc | 4893 | ggctatgcacgcatggttg | 6284 | ggttttcacttgttgagaa | 7675 | ttctcaacaagtgaaaacc | 9065 |
| aaccatgcgtgcatagcct | 4894 | aggctatgcacgcatggtt | 6285 | gttttcacttgttgagaag | 7676 | cttctcaacaagtgaaaac | 9066 |
| accatgcgtgcatagcctg | 4895 | caggctatgcacgcatggt | 6286 | ttttcacttgttgagaaga | 7677 | tcttctcaacaagtgaaaa | 9067 |
| ccatgcgtgcatagcctgc | 4896 | gcaggctatgcacgcatgg | 6287 | tttcacttgttgagaagag | 7678 | ctcttctcaacaagtgaaa | 9068 |
| catgcgtgcatagcctgcc | 4897 | ggcaggctatgcacgcatg | 6288 | ttcacttgttgagaagaga | 7679 | tctcttctcaacaagtgaa | 9069 |
| atgcgtgcatagcctgccg | 4898 | cggcaggctatgcacgcat | 6289 | tcacttgttgagaagagat | 7680 | atctcttctcaacaagtga | 9070 |
| tgcgtgcatagcctgccgc | 4899 | gcggcaggctatgcacgca | 6290 | cacttgttgagaagagatg | 7681 | catctcttctcaacaagtg | 9071 |
| gcgtgcatagcctgccgca | 4900 | tgcggcaggctatgcacgc | 6291 | acttgttgagaagagatgt | 7682 | acatctcttctcaacaagt | 9072 |
| cgtgcatagcctgccgcat | 4901 | atgcggcaggctatgcacg | 6292 | cttgttgagaagagatgtg | 7683 | cacatctcttctcaacaag | 9073 |
| gtgcatagcctgccgcatc | 4902 | gatgcggcaggctatgcac | 6293 | ttgttgagaagagatgtgt | 7684 | acacatctcttctcaacaa | 9074 |
| tgcatagcctgccgcatca | 4903 | tgatgcggcaggctatgca | 6294 | tgttgagaagagatgtgtg | 7685 | cacacatctcttctcaaca | 9075 |
| gcatagcctgccgcatcat | 4904 | atgatgcggcaggctatgc | 6295 | gttgagaagagatgtgtgc | 7686 | gcacacatctcttctcaac | 9076 |
| catagcctgccgcatcatt | 4905 | aatgatgcggcaggctatg | 6296 | ttgagaagagatgtgtgcc | 7687 | ggcacacatctcttctcaa | 9077 |
| atagcctgccgcatcattt | 4906 | aaatgatgcggcaggctat | 6297 | tgagaagagatgtgtgcca | 7688 | tggcacacatctcttctca | 9078 |
| tagcctgccgcatcatttt | 4907 | aaaatgatgcggcaggcta | 6298 | gagaagagatgtgtgccat | 7689 | atggcacacatctcttctc | 9079 |
| agcctgccgcatcattttc | 4908 | gaaaatgatgcggcaggct | 6299 | agaagagatgtgtgccata | 7690 | tatggcacacatctcttct | 9080 |
| gcctgccgcatcattttcc | 4909 | ggaaaatgatgcggcaggc | 6300 | gaagagatgtgtgccatat | 7691 | atatggcacacatctcttc | 9081 |
| cctgccgcatcattttccg | 4910 | cggaaaatgatgcggcagg | 6301 | aagagatgtgtgccatata | 7692 | tatatggcacacatctctt | 9082 |
| ctgccgcatcattttccgg | 4911 | ccggaaaatgatgcggcag | 6302 | agagatgtgtgccatatac | 7693 | gtatatggcacacatctct | 9083 |
| tgccgcatcattttccggt | 4912 | accggaaaatgatgcggca | 6303 | gagatgtgtgccatatact | 7694 | agtatatggcacacatctc | 9084 |
| gccgcatcattttccggtc | 4913 | gaccggaaaatgatgcggc | 6304 | agatgtgtgccatatactt | 7695 | aagtatatggcacacatct | 9085 |
| ccgcatcattttccggtca | 4914 | tgaccggaaaatgatgcgg | 6305 | gatgtgtgccatatacttg | 7696 | caagtatatggcacacatc | 9086 |
| cgcatcattttccggtcag | 4915 | ctgaccggaaaatgatgcg | 6306 | atgtgtgccatatacttgg | 7697 | ccaagtatatggcacacat | 9087 |
| gcatcattttccggtcaga | 4916 | tctgaccggaaaatgatgc | 6307 | tgtgtgccatatacttggt | 7698 | accaagtatatggcacaca | 9088 |
| catcattttccggtcagat | 4917 | atctgaccggaaaatgatg | 6308 | gtgtgccatatacttggtt | 7699 | aaccaagtatatggcacac | 9089 |
| atcattttccggtcagatg | 4918 | catctgaccggaaaatgat | 6309 | tgtgccatatacttggttt | 7700 | aaaccaagtatatggcaca | 9090 |
| tcattttccggtcagatga | 4919 | tcatctgaccggaaaatga | 6310 | gtgccatatacttggtttg | 7701 | caaaccaagtatatggcac | 9091 |
| cattttccggtcagatgaa | 4920 | ttcatctgaccggaaaatg | 6311 | tgccatatacttggtttgg | 7702 | ccaaaccaagtatatggca | 9092 |
| attttccggtcagatgaac | 4921 | gttcatctgaccggaaaat | 6312 | gccatatacttggtttggt | 7703 | accaaaccaagtatatggc | 9093 |
| ttttccggtcagatgaaca | 4922 | tgttcatctgaccggaaaa | 6313 | ccatatacttggtttggtg | 7704 | caccaaaccaagtatatgg | 9094 |
| tttccggtcagatgaacac | 4923 | gtgttcatctgaccggaaa | 6314 | catatacttggtttggtgg | 7705 | ccaccaaaccaagtatatg | 9095 |
| ttccggtcagatgaacacc | 4924 | ggtgttcatctgaccggaa | 6315 | atatacttggtttggtggc | 7706 | gccaccaaaccaagtatat | 9096 |
| tccggtcagatgaacacca | 4925 | tggtgttcatctgaccgga | 6316 | tatacttggtttggtggct | 7707 | agccaccaaaccaagtata | 9097 |
| ccggtcagatgaacaccac | 4926 | gtggtgttcatctgaccgg | 6317 | atacttggtttggtggcta | 7708 | tagccaccaaaccaagtat | 9098 |
| cggtcagatgaacaccacc | 4927 | ggtggtgttcatctgaccg | 6318 | tacttggtttggtggctac | 7709 | gtagccaccaaaccaagta | 9099 |
| ggtcagatgaacaccaccc | 4928 | gggtggtgttcatctgacc | 6319 | acttggtttggtggctaca | 7710 | tgtagccaccaaaccaagt | 9100 |
| gtcagatgaacaccaccct | 4929 | agggtggtgttcatctgac | 6320 | cttggtttggtggctacag | 7711 | ctgtagccaccaaaccaag | 9101 |
| tcagatgaacaccaccctc | 4930 | gagggtggtgttcatctga | 6321 | ttggtttggtggctacaga | 7712 | tctgtagccaccaaaccaa | 9102 |
| cagatgaacaccaccctcc | 4931 | ggagggtggtgttcatctg | 6322 | tggtttggtggctacagag | 7713 | ctctgtagccaccaaacca | 9103 |
| agatgaacaccaccctccc | 4932 | gggagggtggtgttcatct | 6323 | ggtttggtggctacagagt | 7714 | actctgtagccaccaaacc | 9104 |
| gatgaacaccaccctccca | 4933 | tgggagggtggtgttcatc | 6324 | gtttggtggctacagagta | 7715 | tactctgtagccaccaaac | 9105 |
| atgaacaccaccctcccat | 4934 | atgggagggtggtgttcat | 6325 | tttggtggctacagagtac | 7716 | gtactctgtagccaccaaa | 9106 |
| tgaacaccaccctcccata | 4935 | tatgggagggtggtgttca | 6326 | ttggtggctacagagtaca | 7717 | tgtactctgtagccaccaa | 9107 |
| gaacaccaccctcccatac | 4936 | gtatgggagggtggtgttc | 6327 | tggtggctacagagtacag | 7718 | ctgtactctgtagccacca | 9108 |
| aacaccaccctcccatact | 4937 | agtatgggagggtggtgtt | 6328 | ggtggctacagagtacagc | 7719 | gctgtactctgtagccacc | 9109 |
| acaccaccctcccatactg | 4938 | cagtatgggagggtggtgt | 6329 | gtggctacagagtacagcc | 7720 | ggctgtactctgtagccac | 9110 |
| caccaccctcccatactgc | 4939 | gcagtatgggagggtggtg | 6330 | tggctacagagtacagcct | 7721 | aggctgtactctgtagcca | 9111 |
| accaccctcccatactgcc | 4940 | ggcagtatgggagggtggt | 6331 | ggctacagagtacagcctg | 7722 | caggctgtactctgtagcc | 9112 |
| ccaccctcccatactgccc | 4941 | gggcagtatgggagggtgg | 6332 | gctacagagtacagcctgc | 7723 | gcaggctgtactctgtagc | 9113 |
| caccctcccatactgcccc | 4942 | ggggcagtatgggagggtg | 6333 | ctacagagtacagcctgct | 7724 | agcaggctgtactctgtag | 9114 |
| accctcccatactgccccc | 4943 | gggggcagtatgggagggt | 6334 | tacagagtacagcctgctg | 7725 | cagcaggctgtactctgta | 9115 |
| ccctcccatactgcccccc | 4944 | ggggggcagtatgggaggg | 6335 | acagagtacagcctgctgc | 7726 | gcagcaggctgtactctgt | 9116 |
| cctcccatactgcccccca | 4945 | tggggggcagtatgggagg | 6336 | cagagtacagcctgctgct | 7727 | agcagcaggctgtactctg | 9117 |
| ctcccatactgccccccaa | 4946 | ttggggggcagtatgggag | 6337 | agagtacagcctgctgctt | 7728 | aagcagcaggctgtactct | 9118 |
| tcccatactgccccccaag | 4947 | cttggggggcagtatggga | 6338 | gagtacagcctgctgctta | 7729 | taagcagcaggctgtactc | 9119 |
| cccatactgccccccaagg | 4948 | ccttggggggcagtatggg | 6339 | agtacagcctgctgcttaa | 7730 | ttaagcagcaggctgtact | 9120 |
| ccatactgccccccaaggc | 4949 | gccttggggggcagtatgg | 6340 | gtacagcctgctgcttaac | 7731 | gttaagcagcaggctgtac | 9121 |
| catactgccccccaaggct | 4950 | agccttggggggcagtatg | 6341 | tacagcctgctgcttaacc | 7732 | ggttaagcagcaggctgta | 9122 |
| atactgccccccaaggctg | 4951 | cagccttggggggcagtat | 6342 | acagcctgctgcttaaccc | 7733 | gggttaagcagcaggctgt | 9123 |
| tactgccccccaaggctga | 4952 | tcagccttggggggcagta | 6343 | cagcctgctgcttaaccct | 7734 | agggttaagcagcaggctg | 9124 |
| actgccccccaaggctgac | 4953 | gtcagccttggggggcagt | 6344 | agcctgctgcttaaccctc | 7735 | gagggttaagcagcaggct | 9125 |
| ctgccccccaaggctgacc | 4954 | ggtcagccttggggggcag | 6345 | gcctgctgcttaaccctca | 7736 | tgagggttaagcagcaggc | 9126 |
| tgccccccaaggctgacct | 4955 | aggtcagccttggggggca | 6346 | cctgctgcttaaccctcag | 7737 | ctgagggttaagcagcagg | 9127 |
| gccccccaaggctgacctg | 4956 | caggtcagccttggggggc | 6347 | ctgctgcttaaccctcaga | 7738 | tctgagggttaagcagcag | 9128 |
| ccccccaaggctgacctga | 4957 | tcaggtcagccttgggggg | 6348 | tgctgcttaaccctcagag | 7739 | ctctgagggttaagcagca | 9129 |
| cccccaaggctgacctgac | 4958 | gtcaggtcagccttggggg | 6349 | gctgcttaaccctcagagg | 7740 | cctctgagggttaagcagc | 9130 |
| ccccaaggctgacctgacc | 4959 | ggtcaggtcagccttgggg | 6350 | ctgcttaaccctcagagga | 7741 | tcctctgagggttaagcag | 9131 |
| cccaaggctgacctgacca | 4960 | tggtcaggtcagccttggg | 6351 | tgcttaaccctcagaggag | 7742 | ctcctctgagggttaagca | 9132 |
| ccaaggctgacctgaccat | 4961 | atggtcaggtcagccttgg | 6352 | gcttaaccctcagaggaga | 7743 | tctcctctgagggttaagc | 9133 |
| caaggctgacctgaccatt | 4962 | aatggtcaggtcagccttg | 6353 | cttaaccctcagaggagac | 7744 | gtctcctctgagggttaag | 9134 |
| aaggctgacctgaccattg | 4963 | caatggtcaggtcagcctt | 6354 | ttaaccctcagaggagact | 7745 | agtctcctctgagggttaa | 9135 |
| aggctgacctgaccattgg | 4964 | ccaatggtcaggtcagcct | 6355 | taaccctcagaggagactg | 7746 | cagtctcctctgagggtta | 9136 |
| ggctgacctgaccattggc | 4965 | gccaatggtcaggtcagcc | 6356 | aaccctcagaggagactga | 7747 | tcagtctcctctgagggtt | 9137 |
| gctgacctgaccattggcc | 4966 | ggccaatggtcaggtcagc | 6357 | accctcagaggagactgat | 7748 | atcagtctcctctgagggt | 9138 |
| ctgacctgaccattggcct | 4967 | aggccaatggtcaggtcag | 6358 | ccctcagaggagactgatc | 7749 | gatcagtctcctctgaggg | 9139 |
| tgacctgaccattggcctc | 4968 | gaggccaatggtcaggtca | 6359 | cctcagaggagactgatcc | 7750 | ggatcagtctcctctgagg | 9140 |
| gacctgaccattggcctcc | 4969 | ggaggccaatggtcaggtc | 6360 | ctcagaggagactgatcca | 7751 | tggatcagtctcctctgag | 9141 |
| acctgaccattggcctcca | 4970 | tggaggccaatggtcaggt | 6361 | tcagaggagactgatccag | 7752 | ctggatcagtctcctctga | 9142 |
| cctgaccattggcctccac | 4971 | gtggaggccaatggtcagg | 6362 | cagaggagactgatccagt | 7753 | actggatcagtctcctctg | 9143 |
| ctgaccattggcctccacg | 4972 | cgtggaggccaatggtcag | 6363 | agaggagactgatccagtt | 7754 | aactggatcagtctcctct | 9144 |
| tgaccattggcctccacgg | 4973 | ccgtggaggccaatggtca | 6364 | gaggagactgatccagttg | 7755 | caactggatcagtctcctc | 9145 |
| gaccattggcctccacggg | 4974 | cccgtggaggccaatggtc | 6365 | aggagactgatccagttgg | 7756 | ccaactggatcagtctcct | 9146 |
| accattggcctccacgggg | 4975 | ccccgtggaggccaatggt | 6366 | ggagactgatccagttggg | 7757 | cccaactggatcagtctcc | 9147 |
| ccattggcctccacgggga | 4976 | tccccgtggaggccaatgg | 6367 | gagactgatccagttggga | 7758 | tcccaactggatcagtctc | 9148 |
| cattggcctccacggggaa | 4977 | ttccccgtggaggccaatg | 6368 | agactgatccagttgggaa | 7759 | ttcccaactggatcagtct | 9149 |
| attggcctccacggggaat | 4978 | attccccgtggaggccaat | 6369 | gactgatccagttgggaaa | 7760 | tttcccaactggatcagtc | 9150 |
| ttggcctccacggggaatg | 4979 | cattccccgtggaggccaa | 6370 | actgatccagttgggaaat | 7761 | atttcccaactggatcagt | 9151 |
| tggcctccacggggaatgg | 4980 | ccattccccgtggaggcca | 6371 | ctgatccagttgggaaatt | 7762 | aatttcccaactggatcag | 9152 |
| ggcctccacggggaatggg | 4981 | cccattccccgtggaggcc | 6372 | tgatccagttgggaaattc | 7763 | gaatttcccaactggatca | 9153 |
| gcctccacggggaatgggt | 4982 | acccattccccgtggaggc | 6373 | gatccagttgggaaattca | 7764 | tgaatttcccaactggatc | 9154 |
| cctccacggggaatgggtg | 4983 | cacccattccccgtggagg | 6374 | atccagttgggaaattcag | 7765 | ctgaatttcccaactggat | 9155 |
| ctccacggggaatgggtga | 4984 | tcacccattccccgtggag | 6375 | tccagttgggaaattcaga | 7766 | tctgaatttcccaactgga | 9156 |
| tccacggggaatgggtgag | 4985 | ctcacccattccccgtgga | 6376 | ccagttgggaaattcagag | 7767 | ctctgaatttcccaactgg | 9157 |
| ccacggggaatgggtgagc | 4986 | gctcacccattccccgtgg | 6377 | cagttgggaaattcagagc | 7768 | gctctgaatttcccaactg | 9158 |
| cacggggaatgggtgagcc | 4987 | ggctcacccattccccgtg | 6378 | agttgggaaattcagagca | 7769 | tgctctgaatttcccaact | 9159 |
| acggggaatgggtgagcca | 4988 | tggctcacccattccccgt | 6379 | gttgggaaattcagagcag | 7770 | ctgctctgaatttcccaac | 9160 |
| cggggaatgggtgagccag | 4989 | ctggctcacccattccccg | 6380 | ttgggaaattcagagcagc | 7771 | gctgctctgaatttcccaa | 9161 |
| ggggaatgggtgagccagc | 4990 | gctggctcacccattcccc | 6381 | tgggaaattcagagcagct | 7772 | agctgctctgaatttccca | 9162 |
| gggaatgggtgagccagcg | 4991 | cgctggctcacccattccc | 6382 | gggaaattcagagcagctc | 7773 | gagctgctctgaatttccc | 9163 |
| ggaatgggtgagccagcgc | 4992 | gcgctggctcacccattcc | 6383 | ggaaattcagagcagctcc | 7774 | ggagctgctctgaatttcc | 9164 |
| gaatgggtgagccagcgct | 4993 | agcgctggctcacccattc | 6384 | gaaattcagagcagctcct | 7775 | aggagctgctctgaatttc | 9165 |
| aatgggtgagccagcgctg | 4994 | cagcgctggctcacccatt | 6385 | aaattcagagcagctcctg | 7776 | caggagctgctctgaattt | 9166 |
| atgggtgagccagcgctgc | 4995 | gcagcgctggctcacccat | 6386 | aattcagagcagctcctgc | 7777 | gcaggagctgctctgaatt | 9167 |
| tgggtgagccagcgctgcg | 4996 | cgcagcgctggctcaccca | 6387 | attcagagcagctcctgct | 7778 | agcaggagctgctctgaat | 9168 |
| gggtgagccagcgctgcga | 4997 | tcgcagcgctggctcaccc | 6388 | ttcagagcagctcctgctc | 7779 | gagcaggagctgctctgaa | 9169 |
| ggtgagccagcgctgcgag | 4998 | ctcgcagcgctggctcacc | 6389 | tcagagcagctcctgctcc | 7780 | ggagcaggagctgctctga | 9170 |
| gtgagccagcgctgcgagg | 4999 | cctcgcagcgctggctcac | 6390 | cagagcagctcctgctcca | 7781 | tggagcaggagctgctctg | 9171 |
| tgagccagcgctgcgaggt | 5000 | acctcgcagcgctggctca | 6391 | agagcagctcctgctccag | 7782 | ctggagcaggagctgctct | 9172 |
| gagccagcgctgcgaggta | 5001 | tacctcgcagcgctggctc | 6392 | gagcagctcctgctccagg | 7783 | cctggagcaggagctgctc | 9173 |
| agccagcgctgcgaggtac | 5002 | gtacctcgcagcgctggct | 6393 | agcagctcctgctccaggc | 7784 | gcctggagcaggagctgct | 9174 |
| gccagcgctgcgaggtacg | 5003 | cgtacctcgcagcgctggc | 6394 | gcagctcctgctccaggca | 7785 | tgcctggagcaggagctgc | 9175 |
| ccagcgctgcgaggtacgc | 5004 | gcgtacctcgcagcgctgg | 6395 | cagctcctgctccaggcag | 7786 | ctgcctggagcaggagctg | 9176 |
| cagcgctgcgaggtacgcc | 5005 | ggcgtacctcgcagcgctg | 6396 | agctcctgctccaggcagc | 7787 | gctgcctggagcaggagct | 9177 |
| agcgctgcgaggtacgccc | 5006 | gggcgtacctcgcagcgct | 6397 | gctcctgctccaggcagcc | 7788 | ggctgcctggagcaggagc | 9178 |
| gcgctgcgaggtacgcccc | 5007 | ggggcgtacctcgcagcgc | 6398 | ctcctgctccaggcagcca | 7789 | tggctgcctggagcaggag | 9179 |
| cgctgcgaggtacgccccg | 5008 | cggggcgtacctcgcagcg | 6399 | tcctgctccaggcagccag | 7790 | ctggctgcctggagcagga | 9180 |
| gctgcgaggtacgccccga | 5009 | tcggggcgtacctcgcagc | 6400 | cctgctccaggcagccaga | 7791 | tctggctgcctggagcagg | 9181 |
| ctgcgaggtacgccccgag | 5010 | ctcggggcgtacctcgcag | 6401 | ctgctccaggcagccagaa | 7792 | ttctggctgcctggagcag | 9182 |
| tgcgaggtacgccccgagg | 5011 | cctcggggcgtacctcgca | 6402 | tgctccaggcagccagaag | 7793 | cttctggctgcctggagca | 9183 |
| gcgaggtacgccccgaggt | 5012 | acctcggggcgtacctcgc | 6403 | gctccaggcagccagaagc | 7794 | gcttctggctgcctggagc | 9184 |
| cgaggtacgccccgaggtc | 5013 | gacctcggggcgtacctcg | 6404 | ctccaggcagccagaagca | 7795 | tgcttctggctgcctggag | 9185 |
| gaggtacgccccgaggtcc | 5014 | ggacctcggggcgtacctc | 6405 | tccaggcagccagaagcag | 7796 | ctgcttctggctgcctgga | 9186 |
| aggtacgccccgaggtcct | 5015 | aggacctcggggcgtacct | 6406 | ccaggcagccagaagcagc | 7797 | gctgcttctggctgcctgg | 9187 |
| ggtacgccccgaggtcctc | 5016 | gaggacctcggggcgtacc | 6407 | caggcagccagaagcagca | 7798 | tgctgcttctggctgcctg | 9188 |
| gtacgccccgaggtcctct | 5017 | agaggacctcggggcgtac | 6408 | aggcagccagaagcagcag | 7799 | ctgctgcttctggctgcct | 9189 |
| tacgccccgaggtcctctt | 5018 | aagaggacctcggggcgta | 6409 | ggcagccagaagcagcagt | 7800 | actgctgcttctggctgcc | 9190 |
| acgccccgaggtcctcttc | 5019 | gaagaggacctcggggcgt | 6410 | gcagccagaagcagcagtg | 7801 | cactgctgcttctggctgc | 9191 |
| cgccccgaggtcctcttcc | 5020 | ggaagaggacctcggggcg | 6411 | cagccagaagcagcagtgg | 7802 | ccactgctgcttctggctg | 9192 |
| gccccgaggtcctcttcct | 5021 | aggaagaggacctcggggc | 6412 | agccagaagcagcagtggg | 7803 | cccactgctgcttctggct | 9193 |
| ccccgaggtcctcttcctc | 5022 | gaggaagaggacctcgggg | 6413 | gccagaagcagcagtgggg | 7804 | ccccactgctgcttctggc | 9194 |
| cccgaggtcctcttcctca | 5023 | tgaggaagaggacctcggg | 6414 | ccagaagcagcagtggggg | 7805 | cccccactgctgcttctgg | 9195 |
| ccgaggtcctcttcctcac | 5024 | gtgaggaagaggacctcgg | 6415 | cataagcagcagtgggggg | 7806 | ccccccactgctgcttctg | 9196 |
| cgaggtcctcttcctcacc | 5025 | ggtgaggaagaggacctcg | 6416 | agaagcagcagtggggggt | 7807 | accccccactgctgcttct | 9197 |
| gaggtcctcttcctcaccc | 5026 | gggtgaggaagaggacctc | 6417 | gaagcagcagtggggggtg | 7808 | caccccccactgctgcttc | 9198 |
| aggtcctcttcctcacccg | 5027 | cgggtgaggaagaggacct | 6418 | aagcagcagtggggggtgg | 7809 | ccaccccccactgctgctt | 9199 |
| ggtcctcttcctcacccgc | 5028 | gcgggtgaggaagaggacc | 6419 | agcagcagtggggggtggg | 7810 | cccaccccccactgctgct | 9200 |
| gtcctcttcctcacccgcc | 5029 | ggcgggtgaggaagaggac | 6420 | gcagcagtggggggtgggg | 7811 | ccccaccccccactgctgc | 9201 |
| tcctcttcctcacccgcca | 5030 | tggcgggtgaggaagagga | 6421 | cagcagtggggggtggggg | 7812 | cccccaccccccactgctg | 9202 |
| cctcttcctcacccgccac | 5031 | gtggcgggtgaggaagagg | 6422 | agcagtggggggtgggggt | 7813 | acccccaccccccactgct | 9203 |
| ctcttcctcacccgccact | 5032 | agtggcgggtgaggaagag | 6423 | gcagtggggggtgggggtg | 7814 | cacccccaccccccactgc | 9204 |
| tcttcctcacccgccactt | 5033 | aagtggcgggtgaggaaga | 6424 | cagtggggggtgggggtgg | 7815 | ccacccccaccccccactg | 9205 |
| cttcctcacccgccacttc | 5034 | gaagtggcgggtgaggaag | 6425 | agtggggggtgggggtggg | 7816 | cccacccccaccccccact | 9206 |
| ttcctcacccgccacttca | 5035 | tgaagtggcgggtgaggaa | 6426 | gtggggggtgggggtgggg | 7817 | ccccacccccaccccccac | 9207 |
| tcctcacccgccacttcat | 5036 | atgaagtggcgggtgagga | 6427 | tggggggtgggggtgggga | 7818 | tccccacccccacccccca | 9208 |
| cctcacccgccacttcatc | 5037 | gatgaagtggcgggtgagg | 6428 | ggggggtgggggtggggat | 7819 | atccccacccccacccccc | 9209 |
| ctcacccgccacttcatct | 5038 | agatgaagtggcgggtgag | 6429 | gggggtgggggtggggatt | 7820 | aatccccacccccaccccc | 9210 |
| tcacccgccacttcatctt | 5039 | aagatgaagtggcgggtga | 6430 | ggggtgggggtggggattc | 7821 | gaatccccacccccacccc | 9211 |
| cacccgccacttcatcttc | 5040 | gaagatgaagtggcgggtg | 6431 | gggtgggggtggggattcc | 7822 | ggaatccccacccccaccc | 9212 |
| acccgccacttcatcttcc | 5041 | ggaagatgaagtggcgggt | 6432 | ggtgggggtggggattcct | 7823 | aggaatccccacccccacc | 9213 |
| cccgccacttcatcttcca | 5042 | tggaagatgaagtggcggg | 6433 | gtgggggtggggattcctt | 7824 | aaggaatccccacccccac | 9214 |
| ccgccacttcatcttccac | 5043 | gtggaagatgaagtggcgg | 6434 | tgggggtggggattccttg | 7825 | caaggaatccccaccccca | 9215 |
| cgccacttcatcttccacg | 5044 | cgtggaagatgaagtggcg | 6435 | gggggtggggattccttgt | 7826 | acaaggaatccccaccccc | 9216 |
| gccacttcatcttccacga | 5045 | tcgtggaagatgaagtggc | 6436 | ggggtggggattccttgtg | 7827 | cacaaggaatccccacccc | 9217 |
| ccacttcatcttccacgac | 5046 | gtcgtggaagatgaagtgg | 6437 | gggtggggattccttgtgt | 7828 | acacaaggaatccccaccc | 9218 |
| cacttcatcttccacgaca | 5047 | tgtcgtggaagatgaagtg | 6438 | ggtggggattccttgtgtt | 7829 | aacacaaggaatccccacc | 9219 |
| acttcatcttccacgacaa | 5048 | ttgtcgtggaagatgaagt | 6439 | gtggggattccttgtgtta | 7830 | taacacaaggaatccccac | 9220 |
| cttcatcttccacgacaac | 5049 | gttgtcgtggaagatgaag | 6440 | tggggattccttgtgttaa | 7831 | ttaacacaaggaatcccca | 9221 |
| ttcatcttccacgacaaca | 5050 | tgttgtcgtggaagatgaa | 6441 | ggggattccttgtgttaag | 7832 | cttaacacaaggaatcccc | 9222 |
| tcatcttccacgacaacaa | 5051 | ttgttgtcgtggaagatga | 6442 | gggattccttgtgttaagt | 7833 | acttaacacaaggaatccc | 9223 |
| catcttccacgacaacaac | 5052 | gttgttgtcgtggaagatg | 6443 | ggattccttgtgttaagtg | 7834 | cacttaacacaaggaatcc | 9224 |
| atcttccacgacaacaaca | 5053 | tgttgttgtcgtggaagat | 6444 | gattccttgtgttaagtgc | 7835 | gcacttaacacaaggaatc | 9225 |
| tcttccacgacaacaacaa | 5054 | ttgttgttgtcgtggaaga | 6445 | attccttgtgttaagtgcc | 7836 | ggcacttaacacaaggaat | 9226 |
| cttccacgacaacaacaac | 5055 | gttgttgttgtcgtggaag | 6446 | ttccttgtgttaagtgcca | 7837 | tggcacttaacacaaggaa | 9227 |
| ttccacgacaacaacaaca | 5056 | tgttgttgttgtcgtggaa | 6447 | tccttgtgttaagtgccac | 7838 | gtggcacttaacacaagga | 9228 |
| tccacgacaacaacaacac | 5057 | gtgttgttgttgtcgtgga | 6448 | ccttgtgttaagtgccaca | 7839 | tgtggcacttaacacaagg | 9229 |
| ccacgacaacaacaacacc | 5058 | ggtgttgttgttgtcgtgg | 6449 | cttgtgttaagtgccacac | 7840 | gtgtggcacttaacacaag | 9230 |
| cacgacaacaacaacacct | 5059 | aggtgttgttgttgtcgtg | 6450 | ttgtgttaagtgccacaca | 7841 | tgtgtggcacttaacacaa | 9231 |
| acgacaacaacaacacctg | 5060 | caggtgttgttgttgtcgt | 6451 | tgtgttaagtgccacacaa | 7842 | ttgtgtggcacttaacaca | 9232 |
| cgacaacaacaacacctgg | 5061 | ccaggtgttgttgttgtcg | 6452 | gtgttaagtgccacacaac | 7843 | gttgtgtggcacttaacac | 9233 |
| gacaacaacaacacctggg | 5062 | cccaggtgttgttgttgtc | 6453 | tgttaagtgccacacaact | 7844 | agttgtgtggcacttaaca | 9234 |
| acaacaacaacacctggga | 5063 | tcccaggtgttgttgttgt | 6454 | gttaagtgccacacaactg | 7845 | cagttgtgtggcacttaac | 9235 |
| caacaacaacacctgggaa | 5064 | ttcccaggtgttgttgttg | 6455 | ttaagtgccacacaactga | 7846 | tcagttgtgtggcacttaa | 9236 |
| aacaacaacacctgggaag | 5065 | cttcccaggtgttgttgtt | 6456 | taagtgccacacaactgac | 7847 | gtcagttgtgtggcactta | 9237 |
| acaacaacacctgggaagg | 5066 | ccttcccaggtgttgttgt | 6457 | aagtgccacacaactgaca | 7848 | tgtcagttgtgtggcactt | 9238 |
| caacaacacctgggaaggg | 5067 | cccttcccaggtgttgttg | 6458 | agtgccacacaactgacaa | 7849 | ttgtcagttgtgtggcact | 9239 |
| aacaacacctgggaagggc | 5068 | gcccttcccaggtgttgtt | 6459 | gtgccacacaactgacaag | 7850 | cttgtcagttgtgtggcac | 9240 |
| acaacacctgggaagggca | 5069 | tgcccttcccaggtgttgt | 6460 | tgccacacaactgacaagg | 7851 | ccttgtcagttgtgtggca | 9241 |
| caacacctgggaagggcat | 5070 | atgcccttcccaggtgttg | 6461 | gccacacaactgacaagga | 7852 | tccttgtcagttgtgtggc | 9242 |
| aacacctgggaagggcatt | 5071 | aatgcccttcccaggtgtt | 6462 | ccacacaactgacaaggag | 7853 | ctccttgtcagttgtgtgg | 9243 |
| acacctgggaagggcatta | 5072 | taatgcccttcccaggtgt | 6463 | cacacaactgacaaggaga | 7854 | tctccttgtcagttgtgtg | 9244 |
| cacctgggaagggcattac | 5073 | gtaatgcccttcccaggtg | 6464 | acacaactgacaaggagat | 7855 | atctccttgtcagttgtgt | 9245 |
| acctgggaagggcattact | 5074 | agtaatgcccttcccaggt | 6465 | cacaactgacaaggagatc | 7856 | gatctccttgtcagttgtg | 9246 |
| cctgggaagggcattacta | 5075 | tagtaatgcccttcccagg | 6466 | acaactgacaaggagatct | 7857 | agatctccttgtcagttgt | 9247 |
| ctgggaagggcattactac | 5076 | gtagtaatgcccttcccag | 6467 | caactgacaaggagatctg | 7858 | cagatctccttgtcagttg | 9248 |
| tgggaagggcattactacc | 5077 | ggtagtaatgcccttccca | 6468 | aactgacaaggagatctgt | 7859 | acagatctccttgtcagtt | 9249 |
| gggaagggcattactacca | 5078 | tggtagtaatgcccttccc | 6469 | actgacaaggagatctgtg | 7860 | cacagatctccttgtcagt | 9250 |
| ggaagggcattactaccac | 5079 | gtggtagtaatgcccttcc | 6470 | ctgacaaggagatctgtgg | 7861 | ccacagatctccttgtcag | 9251 |
| gaagggcattactaccact | 5080 | agtggtagtaatgcccttc | 6471 | tgacaaggagatctgtgga | 7862 | tccacagatctccttgtca | 9252 |
| aagggcattactaccacta | 5081 | tagtggtagtaatgccctt | 6472 | gacaaggagatctgtggag | 7863 | ctccacagatctccttgtc | 9253 |
| agggcattactaccactac | 5082 | gtagtggtagtaatgccct | 6473 | acaaggagatctgtggagt | 7864 | actccacagatctccttgt | 9254 |
| gggcattactaccactact | 5083 | agtagtggtagtaatgccc | 6474 | caaggagatctgtggagtt | 7865 | aactccacagatctccttg | 9255 |
| ggcattactaccactactc | 5084 | gagtagtggtagtaatgcc | 6475 | aaggagatctgtggagttt | 7866 | aaactccacagatctcctt | 9256 |
| gcattactaccactactca | 5085 | tgagtagtggtagtaatgc | 6476 | aggagatctgtggagtttt | 7867 | aaaactccacagatctcct | 9257 |
| cattactaccactactcag | 5086 | ctgagtagtggtagtaatg | 6477 | ggagatctgtggagttttt | 7868 | aaaaactccacagatctcc | 9258 |
| attactaccactactcaga | 5087 | tctgagtagtggtagtaat | 6478 | gagatctgtggagtttttc | 7869 | gaaaaactccacagatctc | 9259 |
| ttactaccactactcagac | 5088 | gtctgagtagtggtagtaa | 6479 | agatctgtggagtttttct | 7870 | agaaaaactccacagatct | 9260 |
| tactaccactactcagacc | 5089 | ggtctgagtagtggtagta | 6480 | gatctgtggagtttttctc | 7871 | gagaaaaactccacagatc | 9261 |
| actaccactactcagaccc | 5090 | gggtctgagtagtggtagt | 6481 | atctgtggagtttttctcc | 7872 | ggagaaaaactccacagat | 9262 |
| ctaccactactcagaccct | 5091 | agggtctgagtagtggtag | 6482 | tctgtggagtttttctcca | 7873 | tggagaaaaactccacaga | 9263 |
| taccactactcagaccctg | 5092 | cagggtctgagtagtggta | 6483 | ctgtggagtttttctccaa | 7874 | ttggagaaaaactccacag | 9264 |
| accactactcagaccctgt | 5093 | acagggtctgagtagtggt | 6484 | tgtggagtttttctccaag | 7875 | cttggagaaaaactccaca | 9265 |
| ccactactcagaccctgtc | 5094 | gacagggtctgagtagtgg | 6485 | gtggagtttttctccaagt | 7876 | acttggagaaaaactccac | 9266 |
| cactactcagaccctgtct | 5095 | agacagggtctgagtagtg | 6486 | tggagtttttctccaagtg | 7877 | cacttggagaaaaactcca | 9267 |
| actactcagaccctgtctg | 5096 | cagacagggtctgagtagt | 6487 | ggagtttttctccaagtga | 7878 | tcacttggagaaaaactcc | 9268 |
| ctactcagaccctgtctgc | 5097 | gcagacagggtctgagtag | 6488 | gagtttttctccaagtgaa | 7879 | ttcacttggagaaaaactc | 9269 |
| tactcagaccctgtctgca | 5098 | tgcagacagggtctgagta | 6489 | agtttttctccaagtgaac | 7880 | gttcacttggagaaaaact | 9270 |
| actcagaccctgtctgcaa | 5099 | ttgcagacagggtctgagt | 6490 | gtttttctccaagtgaacc | 7881 | ggttcacttggagaaaaac | 9271 |
| ctcagaccctgtctgcaag | 5100 | cttgcagacagggtctgag | 6491 | tttttctccaagtgaacca | 7882 | tggttcacttggagaaaaa | 9272 |
| tcagaccctgtctgcaagc | 5101 | gcttgcagacagggtctga | 6492 | ttttctccaagtgaaccaa | 7883 | ttggttcacttggagaaaa | 9273 |
| cagaccctgtctgcaagca | 5102 | tgcttgcagacagggtctg | 6493 | tttctccaagtgaaccaat | 7884 | attggttcacttggagaaa | 9274 |
| agaccctgtctgcaagcac | 5103 | gtgcttgcagacagggtct | 6494 | ttctccaagtgaaccaatc | 7885 | gattggttcacttggagaa | 9275 |
| gaccctgtctgcaagcacc | 5104 | ggtgcttgcagacagggtc | 6495 | tctccaagtgaaccaatcc | 7886 | ggattggttcacttggaga | 9276 |
| accctgtctgcaagcaccc | 5105 | gggtgcttgcagacagggt | 6496 | ctccaagtgaaccaatccc | 7887 | gggattggttcacttggag | 9277 |
| ccctgtctgcaagcacccc | 5106 | ggggtgcttgcagacaggg | 6497 | tccaagtgaaccaatcccc | 7888 | ggggattggttcacttgga | 9278 |
| cctgtctgcaagcacccca | 5107 | tggggtgcttgcagacagg | 6498 | ccaagtgaaccaatcccct | 7889 | aggggattggttcacttgg | 9279 |
| ctgtctgcaagcaccccac | 5108 | gtggggtgcttgcagacag | 6499 | caagtgaaccaatcccctg | 7890 | caggggattggttcacttg | 9280 |
| tgtctgcaagcaccccaca | 5109 | tgtggggtgcttgcagaca | 6500 | aagtgaaccaatcccctgt | 7891 | acaggggattggttcactt | 9281 |
| gtctgcaagcaccccacat | 5110 | atgtggggtgcttgcagac | 6501 | agtgaaccaatcccctgtg | 7892 | cacaggggattggttcact | 9282 |
| tctgcaagcaccccacatt | 5111 | aatgtggggtgcttgcaga | 6502 | gtgaaccaatcccctgtgt | 7893 | acacaggggattggttcac | 9283 |
| ctgcaagcaccccacattc | 5112 | gaatgtggggtgcttgcag | 6503 | tgaaccaatcccctgtgtc | 7894 | gacacaggggattggttca | 9284 |
| tgcaagcaccccacattca | 5113 | tgaatgtggggtgcttgca | 6504 | gaaccaatcccctgtgtcc | 7895 | ggacacaggggattggttc | 9285 |
| gcaagcaccccacattcac | 5114 | gtgaatgtggggtgcttgc | 6505 | aaccaatcccctgtgtcct | 7896 | aggacacaggggattggtt | 9286 |
| caagcaccccacattcacc | 5115 | ggtgaatgtggggtgcttg | 6506 | accaatcccctgtgtcctg | 7897 | caggacacaggggattggt | 9287 |
| aagcaccccacattcacca | 5116 | tggtgaatgtggggtgctt | 6507 | ccaatcccctgtgtcctgg | 7898 | ccaggacacaggggattgg | 9288 |
| agcaccccacattcaccat | 5117 | atggtgaatgtggggtgct | 6508 | caatcccctgtgtcctggc | 7899 | gccaggacacaggggattg | 9289 |
| gcaccccacattcaccatc | 5118 | gatggtgaatgtggggtgc | 6509 | aatcccctgtgtcctggct | 7900 | agccaggacacaggggatt | 9290 |
| caccccacattcaccatct | 5119 | agatggtgaatgtggggtg | 6510 | atcccctgtgtcctggctc | 7901 | gagccaggacacaggggat | 9291 |
| accccacattcaccatcta | 5120 | tagatggtgaatgtggggt | 6511 | tcccctgtgtcctggctca | 7902 | tgagccaggacacagggga | 9292 |
| ccccacattcaccatctac | 5121 | gtagatggtgaatgtgggg | 6512 | cccctgtgtcctggctcac | 7903 | gtgagccaggacacagggg | 9293 |
| cccacattcaccatctacg | 5122 | cgtagatggtgaatgtggg | 6513 | ccctgtgtcctggctcaca | 7904 | tgtgagccaggacacaggg | 9294 |
| ccacattcaccatctacgc | 5123 | gcgtagatggtgaatgtgg | 6514 | cctgtgtcctggctcacac | 7905 | gtgtgagccaggacacagg | 9295 |
| cacattcaccatctacgct | 5124 | agcgtagatggtgaatgtg | 6515 | ctgtgtcctggctcacact | 7906 | agtgtgagccaggacacag | 9296 |
| acattcaccatctacgctc | 5125 | gagcgtagatggtgaatgt | 6516 | tgtgtcctggctcacactg | 7907 | cagtgtgagccaggacaca | 9297 |
| cattcaccatctacgctcg | 5126 | cgagcgtagatggtgaatg | 6517 | gtgtcctggctcacactgt | 7908 | acagtgtgagccaggacac | 9298 |
| attcaccatctacgctcga | 5127 | tcgagcgtagatggtgaat | 6518 | tgtcctggctcacactgtg | 7909 | cacagtgtgagccaggaca | 9299 |
| ttcaccatctacgctcgag | 5128 | ctcgagcgtagatggtgaa | 6519 | gtcctggctcacactgtgg | 7910 | ccacagtgtgagccaggac | 9300 |
| tcaccatctacgctcgagg | 5129 | cctcgagcgtagatggtga | 6520 | tcctggctcacactgtggt | 7911 | accacagtgtgagccagga | 9301 |
| caccatctacgctcgaggc | 5130 | gcctcgagcgtagatggtg | 6521 | cctggctcacactgtggtt | 7912 | aaccacagtgtgagccagg | 9302 |
| accatctacgctcgaggcc | 5131 | ggcctcgagcgtagatggt | 6522 | ctggctcacactgtggtta | 7913 | taaccacagtgtgagccag | 9303 |
| ccatctacgctcgaggccg | 5132 | cggcctcgagcgtagatgg | 6523 | tggctcacactgtggttag | 7914 | ctaaccacagtgtgagcca | 9304 |
| catctacgctcgaggccgc | 5133 | gcggcctcgagcgtagatg | 6524 | ggctcacactgtggttagg | 7915 | cctaaccacagtgtgagcc | 9305 |
| atctacgctcgaggccgct | 5134 | agcggcctcgagcgtagat | 6525 | gctcacactgtggttaggg | 7916 | ccctaaccacagtgtgagc | 9306 |
| tctacgctcgaggccgcta | 5135 | tagcggcctcgagcgtaga | 6526 | ctcacactgtggttagggt | 7917 | accctaaccacagtgtgag | 9307 |
| ctacgctcgaggccgctac | 5136 | gtagcggcctcgagcgtag | 6527 | tcacactgtggttagggtg | 7918 | caccctaaccacagtgtga | 9308 |
| tacgctcgaggccgctaca | 5137 | tgtagcggcctcgagcgta | 6528 | cacactgtggttagggtgg | 7919 | ccaccctaaccacagtgtg | 9309 |
| acgctcgaggccgctacag | 5138 | ctgtagcggcctcgagcgt | 6529 | acactgtggttagggtggg | 7920 | cccaccctaaccacagtgt | 9310 |
| cgctcgaggccgctacagc | 5139 | gctgtagcggcctcgagcg | 6530 | cactgtggttagggtgggc | 7921 | gcccaccctaaccacagtg | 9311 |
| gctcgaggccgctacagcc | 5140 | ggctgtagcggcctcgagc | 6531 | actgtggttagggtgggca | 7922 | tgcccaccctaaccacagt | 9312 |
| ctcgaggccgctacagccg | 5141 | cggctgtagcggcctcgag | 6532 | ctgtggttagggtgggcac | 7923 | gtgcccaccctaaccacag | 9313 |
| tcgaggccgctacagccgc | 5142 | gcggctgtagcggcctcga | 6533 | tgtggttagggtgggcaca | 7924 | tgtgcccaccctaaccaca | 9314 |
| cgaggccgctacagccgcg | 5143 | cgcggctgtagcggcctcg | 6534 | gtggttagggtgggcacat | 7925 | atgtgcccaccctaaccac | 9315 |
| gaggccgctacagccgcgg | 5144 | ccgcggctgtagcggcctc | 6535 | tggttagggtgggcacatc | 7926 | gatgtgcccaccctaacca | 9316 |
| aggccgctacagccgcggt | 5145 | accgcggctgtagcggcct | 6536 | ggttagggtgggcacatcc | 7927 | ggatgtgcccaccctaacc | 9317 |
| ggccgctacagccgcggtg | 5146 | caccgcggctgtagcggcc | 6537 | gttagggtgggcacatcca | 7928 | tggatgtgcccaccctaac | 9318 |
| gccgctacagccgcggtgt | 5147 | acaccgcggctgtagcggc | 6538 | ttagggtgggcacatccac | 7929 | gtggatgtgcccaccctaa | 9319 |
| ccgctacagccgcggtgtg | 5148 | cacaccgcggctgtagcgg | 6539 | tagggtgggcacatccact | 7930 | agtggatgtgcccacccta | 9320 |
| cgctacagccgcggtgtgc | 5149 | gcacaccgcggctgtagcg | 6540 | agggtgggcacatccactc | 7931 | gagtggatgtgcccaccct | 9321 |
| gctacagccgcggtgtgct | 5150 | agcacaccgcggctgtagc | 6541 | gggtgggcacatccactct | 7932 | agagtggatgtgcccaccc | 9322 |
| ctacagccgcggtgtgctc | 5151 | gagcacaccgcggctgtag | 6542 | ggtgggcacatccactctg | 7933 | cagagtggatgtgcccacc | 9323 |
| tacagccgcggtgtgctct | 5152 | agagcacaccgcggctgta | 6543 | gtgggcacatccactctgc | 7934 | gcagagtggatgtgcccac | 9324 |
| acagccgcggtgtgctctc | 5153 | gagagcacaccgcggctgt | 6544 | tgggcacatccactctgcc | 7935 | ggcagagtggatgtgccca | 9325 |
| cagccgcggtgtgctctca | 5154 | tgagagcacaccgcggctg | 6545 | gggcacatccactctgcca | 7936 | tggcagagtggatgtgccc | 9326 |
| agccgcggtgtgctctcat | 5155 | atgagagcacaccgcggct | 6546 | ggcacatccactctgccat | 7937 | atggcagagtggatgtgcc | 9327 |
| gccgcggtgtgctctcatc | 5156 | gatgagagcacaccgcggc | 6547 | gcacatccactctgccatc | 7938 | gatggcagagtggatgtgc | 9328 |
| ccgcggtgtgctctcatct | 5157 | agatgagagcacaccgcgg | 6548 | cacatccactctgccatct | 7939 | agatggcagagtggatgtg | 9329 |
| cgcggtgtgctctcatcta | 5158 | tagatgagagcacaccgcg | 6549 | acatccactctgccatctt | 7940 | aagatggcagagtggatgt | 9330 |
| gcggtgtgctctcatctaa | 5159 | ttagatgagagcacaccgc | 6550 | catccactctgccatcttt | 7941 | aaagatggcagagtggatg | 9331 |
| cggtgtgctctcatctaag | 5160 | cttagatgagagcacaccg | 6551 | atccactctgccatcttta | 7942 | taaagatggcagagtggat | 9332 |
| ggtgtgctctcatctaagg | 5161 | ccttagatgagagcacacc | 6552 | tccactctgccatctttaa | 7943 | ttaaagatggcagagtgga | 9333 |
| gtgtgctctcatctaaggt | 5162 | accttagatgagagcacac | 6553 | ccactctgccatctttaac | 7944 | gttaaagatggcagagtgg | 9334 |
| tgtgctctcatctaaggtc | 5163 | gaccttagatgagagcaca | 6554 | cactctgccatctttaaca | 7945 | tgttaaagatggcagagtg | 9335 |
| gtgctctcatctaaggtca | 5164 | tgaccttagatgagagcac | 6555 | actctgccatctttaacac | 7946 | gtgttaaagatggcagagt | 9336 |
| tgctctcatctaaggtcat | 5165 | atgaccttagatgagagca | 6556 | ctctgccatctttaacaca | 7947 | tgtgttaaagatggcagag | 9337 |
| gctctcatctaaggtcatg | 5166 | catgaccttagatgagagc | 6557 | tctgccatctttaacacac | 7948 | gtgtgttaaagatggcaga | 9338 |
| ctctcatctaaggtcatgg | 5167 | ccatgaccttagatgagag | 6558 | | | | |

In a further embodiment, an siRNA directed to human APCDD1L can comprise any one of SEQ ID NOS: 9339-9716. Table 3 lists siRNA sequences comprising SEQ ID NOS: 9339-9716.

TABLE 3
List of siRNAs directed to human APCDD1L.

| | SEQ | | |
|---|---|---|---|
| | ID | | SEQ |
| Sense (5′-3′) | NO: | Anti-sense (5′-3′) | ID NO: |
| acaactatcaacagccggg | 9339 | cccggctgttgatagttgt | 9528 |
| caactatcaacagccggga | 9340 | tcccggctgttgatagttg | 9529 |
| aactatcaacagccgggaa | 9341 | ttcccggctgttgatagtt | 9530 |
| actatcaacagccgggaag | 9342 | cttcccggctgttgatagt | 9531 |
| ctatcaacagccgggaagg | 9343 | ccttcccggctgttgatag | 9532 |
| tatcaacagccgggaaggc | 9344 | gccttcccggctgttgata | 9533 |
| atcaacagccgggaaggct | 9345 | agccttcccggctgttgat | 9534 |
| tcaacagccgggaaggctg | 9346 | cagccttcccggctgttga | 9535 |
| caacagccgggaaggctga | 9347 | tcagccttcccggctgttg | 9536 |
| aacagccgggaaggctgag | 9348 | ctcagccttcccggctgtt | 9537 |
| acagccgggaaggctgagc | 9349 | gctcagccttcccggctgt | 9538 |
| cagccgggaaggctgagcg | 9350 | cgctcagccttcccggctg | 9539 |
| agccgggaaggctgagcgc | 9351 | gcgctcagccttcccggct | 9540 |
| gccgggaaggctgagcgcg | 9352 | cgcgctcagccttcccggc | 9541 |
| ccgggaaggctgagcgcgt | 9353 | acgcgctcagccttcccgg | 9542 |
| cgggaaggctgagcgcgtg | 9354 | cacgcgctcagccttcccg | 9543 |
| gggaaggctgagcgcgtgt | 9355 | acacgcgctcagccttccc | 9544 |
| ggaaggctgagcgcgtgtg | 9356 | cacacgcgctcagccttcc | 9545 |
| gaaggctgagcgcgtgtga | 9357 | tcacacgcgctcagccttc | 9546 |
| aaggctgagcgcgtgtgag | 9358 | ctcacacgcgctcagcctt | 9547 |
| aggctgagcgcgtgtgagc | 9359 | gctcacacgcgctcagcct | 9548 |
| ggctgagcgcgtgtgagcg | 9360 | cgctcacacgcgctcagcc | 9549 |
| gctgagcgcgtgtgagcgc | 9361 | gcgctcacacgcgctcagc | 9550 |
| ctgagcgcgtgtgagcgcc | 9362 | ggcgctcacacgcgctcag | 9551 |
| tgagcgcgtgtgagcgccg | 9363 | cggcgctcacacgcgctca | 9552 |
| gagcgcgtgtgagcgccga | 9364 | tcggcgctcacacgcgctc | 9553 |
| agcgcgtgtgagcgccgag | 9365 | ctcggcgctcacacgcgct | 9554 |
| gcgcgtgtgagcgccgagg | 9366 | cctcggcgctcacacgcgc | 9555 |
| cgcgtgtgagcgccgaggg | 9367 | ccctcggcgctcacacgcg | 9556 |
| gcgtgtgagcgccgagggg | 9368 | cccctcggcgctcacacgc | 9557 |
| cgtgtgagcgccgaggggg | 9369 | ccccctcggcgctcacacg | 9558 |
| gtgtgagcgccgagggggg | 9370 | cccccctcggcgctcacac | 9559 |
| tgtgagcgccgaggggggc | 9371 | gcccccctcggcgctcaca | 9560 |
| gtgagcgccgaggggggcg | 9372 | cgcccccctcggcgctcac | 9561 |
| tgagcgccgaggggggcgc | 9373 | gcgcccccctcggcgctca | 9562 |
| gagcgccgaggggggcgca | 9374 | tgcgcccccctcggcgctc | 9563 |
| agcgccgaggggggcgcag | 9375 | ctgcgcccccctcggcgct | 9564 |
| gcgccgaggggggcgcagg | 9376 | cctgcgcccccctcggcgc | 9565 |
| cgccgaggggggcgcagga | 9377 | tcctgcgcccccctcggcg | 9566 |
| gccgaggggggcgcaggac | 9378 | gtcctgcgcccccctcggc | 9567 |
| ccgaggggggcgcaggacc | 9379 | ggtcctgcgcccccctcgg | 9568 |
| cgaggggggcgcaggaccc | 9380 | gggtcctgcgcccccctcg | 9569 |
| gaggggggcgcaggaccct | 9381 | agggtcctgcgcccccctc | 9570 |
| aggggggcgcaggaccctc | 9382 | gagggtcctgcgcccccct | 9571 |
| ggggggcgcaggaccctcg | 9383 | cgagggtcctgcgcccccc | 9572 |
| gggggcgcaggaccctcgc | 9384 | gcgagggtcctgcgccccc | 9573 |
| ggggcgcaggaccctcgca | 9385 | tgcgagggtcctgcgcccc | 9574 |
| gggcgcaggaccctcgcaa | 9386 | ttgcgagggtcctgcgccc | 9575 |
| ggcgcaggaccctcgcaac | 9387 | gttgcgagggtcctgcgcc | 9576 |
| gcgcaggaccctcgcaact | 9388 | agttgcgagggtcctgcgc | 9577 |
| cgcaggaccctcgcaactt | 9389 | aagttgcgagggtcctgcg | 9578 |
| gcaggaccctcgcaacttc | 9390 | gaagttgcgagggtcctgc | 9579 |
| caggaccctcgcaacttct | 9391 | agaagttgcgagggtcctg | 9580 |
| aggaccctcgcaacttctt | 9392 | aagaagttgcgagggtcct | 9581 |
| ggaccctcgcaacttcttc | 9393 | gaagaagttgcgagggtcc | 9582 |
| gaccctcgcaacttcttcg | 9394 | cgaagaagttgcgagggtc | 9583 |
| accctcgcaacttcttcgc | 9395 | gcgaagaagttgcgagggt | 9584 |
| ccctcgcaacttcttcgca | 9396 | tgcgaagaagttgcgaggg | 9585 |
| cctcgcaacttcttcgcag | 9397 | ctgcgaagaagttgcgagg | 9586 |
| ctcgcaacttcttcgcagg | 9398 | cctgcgaagaagttgcgag | 9587 |
| tcgcaacttcttcgcagga | 9399 | tcctgcgaagaagttgcga | 9588 |
| cgcaacttcttcgcaggac | 9400 | gtcctgcgaagaagttgcg | 9589 |
| gcaacttcttcgcaggact | 9401 | agtcctgcgaagaagttgc | 9590 |
| caacttcttcgcaggactc | 9402 | gagtcctgcgaagaagttg | 9591 |
| aacttcttcgcaggactcc | 9403 | ggagtcctgcgaagaagtt | 9592 |
| acttcttcgcaggactcca | 9404 | tggagtcctgcgaagaagt | 9593 |
| cttcttcgcaggactccag | 9405 | ctggagtcctgcgaagaag | 9594 |
| ttcttcgcaggactccagc | 9406 | gctggagtcctgcgaagaa | 9595 |
| tcttcgcaggactccagcc | 9407 | ggctggagtcctgcgaaga | 9596 |
| cttcgcaggactccagcct | 9408 | aggctggagtcctgcgaag | 9597 |
| ttcgcaggactccagcctg | 9409 | caggctggagtcctgcgaa | 9598 |
| tcgcaggactccagcctgg | 9410 | ccaggctggagtcctgcga | 9599 |
| cgcaggactccagcctggc | 9411 | gccaggctggagtcctgcg | 9600 |
| gcaggactccagcctggcc | 9412 | ggccaggctggagtcctgc | 9601 |
| caggactccagcctggccg | 9413 | cggccaggctggagtcctg | 9602 |
| aggactccagcctggccgc | 9414 | gcggccaggctggagtcct | 9603 |
| ggactccagcctggccgcc | 9415 | ggcggccaggctggagtcc | 9604 |
| gactccagcctggccgccg | 9416 | cggcggccaggctggagtc | 9605 |
| actccagcctggccgccgg | 9417 | ccggcggccaggctggagt | 9606 |
| ctccagcctggccgccggc | 9418 | gccggcggccaggctggag | 9607 |
| tccagcctggccgccggcg | 9419 | cgccggcggccaggctgga | 9608 |
| ccagcctggccgccggcgc | 9420 | gcgccggcggccaggctgg | 9609 |
| cagcctggccgccggcgcc | 9421 | ggcgccggcggccaggctg | 9610 |
| agcctggccgccggcgccc | 9422 | gggcgccggcggccaggct | 9611 |
| gcctggccgccggcgcccg | 9423 | cgggcgccggcggccaggc | 9612 |
| cctggccgccggcgcccgc | 9424 | gcgggcgccggcggccagg | 9613 |
| ctggccgccggcgcccgca | 9425 | tgcgggcgccggcggccag | 9614 |
| tggccgccggcgcccgcag | 9426 | ctgcgggcgccggcggcca | 9615 |
| ggccgccggcgcccgcagc | 9427 | gctgcgggcgccggcggcc | 9616 |
| gccgccggcgcccgcagcc | 9428 | ggctgcgggcgccggcggc | 9617 |
| ccgccggcgcccgcagccg | 9429 | cggctgcgggcgccggcgg | 9618 |
| cgccggcgcccgcagccgt | 9430 | acggctgcgggcgccggcg | 9619 |
| gccggcgcccgcagccgtc | 9431 | gacggctgcgggcgccggc | 9620 |
| ccggcgcccgcagccgtcc | 9432 | ggacggctgcgggcgccgg | 9621 |
| cggcgcccgcagccgtccg | 9433 | cggacggctgcgggcgccg | 9622 |
| ggcgcccgcagccgtccga | 9434 | tcggacggctgcgggcgcc | 9623 |
| gcgcccgcagccgtccgag | 9435 | ctcggacggctgcgggcgc | 9624 |
| cgcccgcagccgtccgaga | 9436 | tctcggacggctgcgggcg | 9625 |
| gcccgcagccgtccgagag | 9437 | ctctcggacggctgcgggc | 9626 |
| cccgcagccgtccgagagc | 9438 | gctctcggacggctgcggg | 9627 |
| ccgcagccgtccgagagcc | 9439 | ggctctcggacggctgcgg | 9628 |
| cgcagccgtccgagagccc | 9440 | gggctctcggacggctgcg | 9629 |
| gcagccgtccgagagccct | 9441 | agggctctcggacggctgc | 9630 |
| cagccgtccgagagccctg | 9442 | cagggctctcggacggctg | 9631 |
| agccgtccgagagccctgc | 9443 | gcagggctctcggacggct | 9632 |
| gccgtccgagagccctgcg | 9444 | cgcagggctctcggacggc | 9633 |
| ccgtccgagagccctgcgc | 9445 | gcgcagggctctcggacgg | 9634 |
| cgtccgagagccctgcgcc | 9446 | ggcgcagggctctcggacg | 9635 |
| gtccgagagccctgcgccc | 9447 | gggcgcagggctctcggac | 9636 |
| tccgagagccctgcgcccg | 9448 | cgggcgcagggctctcgga | 9637 |
| ccgagagccctgcgcccgc | 9449 | gcgggcgcagggctctcgg | 9638 |
| cgagagccctgcgcccgcg | 9450 | cgcgggcgcagggctctcg | 9639 |
| gagagccctgcgcccgcgc | 9451 | gcgcgggcgcagggctctc | 9640 |
| agagccctgcgcccgcgcc | 9452 | ggcgcgggcgcagggctct | 9641 |
| gagccctgcgcccgcgcct | 9453 | aggcgcgggcgcagggctc | 9642 |
| agccctgcgcccgcgcctc | 9454 | gaggcgcgggcgcagggct | 9643 |
| gccctgcgcccgcgcctcc | 9455 | ggaggcgcgggcgcagggc | 9644 |
| ccctgcgcccgcgcctccc | 9456 | gggaggcgcgggcgcaggg | 9645 |
| cctgcgcccgcgcctcccc | 9457 | ggggaggcgcgggcgcagg | 9646 |
| ctgcgcccgcgcctcccct | 9458 | aggggaggcgcgggcgcag | 9647 |
| tgcgcccgcgcctcccctt | 9459 | aaggggaggcgcgggcgca | 9648 |
| gcgcccgcgcctccccttg | 9460 | caaggggaggcgcgggcgc | 9649 |
| cgcccgcgcctccccttgc | 9461 | gcaaggggaggcgcgggcg | 9650 |
| gcccgcgcctccccttgcg | 9462 | cgcaaggggaggcgcgggc | 9651 |
| cccgcgcctccccttgcgc | 9463 | gcgcaaggggaggcgcggg | 9652 |
| ccgcgcctccccttgcgca | 9464 | tgcgcaaggggaggcgcgg | 9653 |
| cgcgcctccccttgcgcac | 9465 | gtgcgcaaggggaggcgcg | 9654 |
| gcgcctccccttgcgcacc | 9466 | ggtgcgcaaggggaggcgc | 9655 |
| cgcctccccttgcgcaccg | 9467 | cggtgcgcaaggggaggcg | 9656 |
| gcctccccttgcgcaccgt | 9468 | acggtgcgcaaggggaggc | 9657 |
| cctccccttgcgcaccgtg | 9469 | cacggtgcgcaaggggagg | 9658 |
| ctccccttgcgcaccgtgg | 9470 | ccacggtgcgcaaggggag | 9659 |
| tccccttgcgcaccgtggc | 9471 | gccacggtgcgcaagggga | 9660 |
| ccccttgcgcaccgtggca | 9472 | tgccacggtgcgcaagggg | 9661 |
| cccttgcgcaccgtggcag | 9473 | ctgccacggtgcgcaaggg | 9662 |
| ccttgcgcaccgtggcagc | 9474 | gctgccacggtgcgcaagg | 9663 |
| cttgcgcaccgtggcagcg | 9475 | cgctgccacggtgcgcaag | 9664 |
| ttgcgcaccgtggcagcgc | 9476 | gcgctgccacggtgcgcaa | 9665 |
| tgcgcaccgtggcagcgcc | 9477 | ggcgctgccacggtgcgca | 9666 |
| gcgcaccgtggcagcgccc | 9478 | gggcgctgccacggtgcgc | 9667 |
| cgcaccgtggcagcgcccg | 9479 | cgggcgctgccacggtgcg | 9668 |
| gcaccgtggcagcgcccgg | 9480 | ccgggcgctgccacggtgc | 9669 |
| caccgtggcagcgcccggc | 9481 | gccgggcgctgccacggtg | 9670 |
| accgtggcagcgcccggcg | 9482 | cgccgggcgctgccacggt | 9671 |
| ccgtggcagcgcccggcgg | 9483 | ccgccgggcgctgccacgg | 9672 |
| cgtggcagcgcccggcggg | 9484 | cccgccgggcgctgccacg | 9673 |
| gtggcagcgcccggcgggc | 9485 | gcccgccgggcgctgccac | 9674 |
| tggcagcgcccggcgggcg | 9486 | cgcccgccgggcgctgcca | 9675 |
| ggcagcgcccggcgggcgg | 9487 | ccgcccgccgggcgctgcc | 9676 |
| gcagcgcccggcgggcggt | 9488 | accgcccgccgggcgctgc | 9677 |
| cagcgcccggcgggcggtc | 9489 | gaccgcccgccgggcgctg | 9678 |
| agcgcccggcgggcggtcc | 9490 | ggaccgcccgccgggcgct | 9679 |
| gcgcccggcgggcggtcct | 9491 | aggaccgcccgccgggcgc | 9680 |
| cgcccggcgggcggtcctg | 9492 | caggaccgcccgccgggcg | 9681 |
| gcccggcgggcggtcctgc | 9493 | gcaggaccgcccgccgggc | 9682 |
| cccggcgggcggtcctgcc | 9494 | ggcaggaccgcccgccggg | 9683 |
| ccggcgggcggtcctgcca | 9495 | tggcaggaccgcccgccgg | 9684 |
| cggcgggcggtcctgccag | 9496 | ctggcaggaccgcccgccg | 9685 |
| ggcgggcggtcctgccagc | 9497 | gctggcaggaccgcccgcc | 9686 |
| gcgggcggtcctgccagcc | 9498 | ggctggcaggaccgcccgc | 9687 |
| cgggcggtcctgccagccc | 9499 | gggctggcaggaccgcccg | 9688 |
| gggcggtcctgccagcccc | 9500 | ggggctggcaggaccgccc | 9689 |
| ggcggtcctgccagccccg | 9501 | cggggctggcaggaccgcc | 9690 |
| gcggtcctgccagccccga | 9502 | tcggggctggcaggaccgc | 9691 |
| cggtcctgccagccccgac | 9503 | gtcggggctggcaggaccg | 9692 |
| ggtcctgccagccccgacg | 9504 | cgtcggggctggcaggacc | 9693 |
| gtcctgccagccccgacgg | 9505 | ccgtcggggctggcaggac | 9694 |
| tcctgccagccccgacggg | 9506 | cccgtcggggctggcagga | 9695 |
| cctgccagccccgacggga | 9507 | tcccgtcggggctggcagg | 9696 |
| ctgccagccccgacgggat | 9508 | atcccgtcggggctggcag | 9697 |
| tgccagccccgacgggatg | 9509 | catcccgtcggggctggca | 9698 |
| gccagccccgacgggatgc | 9510 | gcatcccgtcggggctggc | 9699 |
| ccagccccgacgggatgcc | 9511 | ggcatcccgtcggggctgg | 9700 |
| cagccccgacgggatgccc | 9512 | gggcatcccgtcggggctg | 9701 |
| agccccgacgggatgcccg | 9513 | cgggcatcccgtcggggct | 9702 |
| gccccgacgggatgcccgc | 9514 | gcgggcatcccgtcggggc | 9703 |
| ccccgacgggatgcccgca | 9515 | tgcgggcatcccgtcgggg | 9704 |
| cccgacgggatgcccgcag | 9516 | ctgcgggcatcccgtcggg | 9705 |
| ccgacgggatgcccgcagc | 9517 | gctgcgggcatcccgtcgg | 9706 |
| cgacgggatgcccgcagcc | 9518 | ggctgcgggcatcccgtcg | 9707 |
| gacgggatgcccgcagcca | 9519 | tggctgcgggcatcccgtc | 9708 |
| acgggatgcccgcagccat | 9520 | atggctgcgggcatcccgt | 9709 |
| cgggatgcccgcagccatg | 9521 | catggctgcgggcatcccg | 9710 |
| gggatgcccgcagccatgc | 9522 | gcatggctgcgggcatccc | 9711 |
| ggatgcccgcagccatgct | 9523 | agcatggctgcgggcatcc | 9712 |
| gatgcccgcagccatgctc | 9524 | gagcatggctgcgggcatc | 9713 |
| atgcccgcagccatgctcc | 9525 | ggagcatggctgcgggcat | 9714 |
| tgcccgcagccatgctccc | 9526 | gggagcatggctgcgggca | 9715 |
| gcccgcagccatgctcccc | 9527 | gggg | 9716 |

RNA polymerase III transcribed DNAs contain promoters, such as the U6 promoter. These DNAs can be transcribed to produce small hairpin RNAs in the cell that can function as siRNA or linear RNAs that can function as antisense RNA. The APCDD1 modulating compound can contain ribonucleotides, deoxyribonucleotides, synthetic nucleotides, or any suitable combination such that the target RNA and/or gene is inhibited. In addition, these forms of nucleic acid can be single, double, triple, or quadruple stranded. (see for example Bass (2001) Nature, 411, 428 429; Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914).

An APCDD1 modulating compound can also be a small molecule that binds to APCDD1 and disrupts its function, or conversely, enhances its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate APCDD1 can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described below (Werner et al., (2006) Brief Funct. Genomic Proteomic 5(1):32-6).

Knowledge of the primary sequence of a molecule of interest, such as an APCDD1 polypeptide, and the similarity of that sequence with proteins of known function, can provide information as to the inhibitors or antagonists of the protein of interest in addition to agonists. Identification and screening of agonists and antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Test compounds, such as APCDD1 modulating compounds, can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76). Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. For example, libraries can also include, but are not limited to, peptide-on-plasmid libraries, synthetic small molecule libraries, aptamer libraries, in vitro translation-based libraries, polysome libraries, synthetic peptide libraries, neurotransmitter libraries, and chemical libraries.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383.

Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/18318.

In vitro translation-based libraries include but are not limited to those described in PCT Publication No. WO 91/05058; and Mattheakis et al., (1994) Proc. Natl. Acad. Sci. USA 91:9022-9026.

As used herein, the term “ligand source” can be any compound library described herein, a library of neurotransmitters, or tissue extract prepared from various organs in an organism's system, that can be used to screen for compounds that would act as an agonist or antagonist of APCDD1. Screening compound libraries listed herein [also see U.S. Patent Application Publication No. 2005/0009163, which is hereby incorporated by reference in its entirety], in combination with in vivo animal studies, functional and signaling assays described below can be used to identify APCDD1 modulating compounds that regulate hair growth or treat hair loss disorders.

For example, functional assays for compound screening can involve axis duplication assays in xenopus embryos (Liao et al. (2006) PNAS, 103(44):1613-18; Fahnert et al., (2004) J Biol Chem, 279(46): 47520-27; Funayama, N. et al., (1995) J. Cell Biol. 128:959-968; and Moser et al., (2003) Mol Cell Biol, 23(16): 5664-79, each of which are incorporated by reference in their entireties). For example, if APCDD1 acts as an inhibitor of wnt signaling, then it should show this effect in the xenopus assay referenced above. This assay can then be used to identify APCDD1 modulating compounds, and later show that they regulate hair growth or treat hair loss disorders using mouse models

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318.

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

In one non-limiting example, non-peptide libraries, such as a benzodiazepine library (see e.g., Bunin et al., (1994) Proc. Natl. Acad. Sci. USA 91:4708-4712), can be screened. Peptoid libraries, such as that described by Simon et al., (1992) Proc. Natl. Acad. Sci. USA 89:9367-9371, can also be used. Another example of a library that can be used, in which the amide functionalities in peptides have been permethylated to generate a chemically transformed combinatorial library, is described by Ostresh et al. (1994), Proc. Natl. Acad. Sci. USA 91:11138-11142.

Computer modeling and searching technologies permit the identification of compounds, or the improvement of already identified compounds, that can modulate APCDD1 expression or activity. Having identified such a compound or composition, the active sites or regions of an APCDD1 molecule can be subsequently identified via examining the sites to which the compounds bind. These sites can be ligand binding sites and can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

The three dimensional geometric structure of a site, for example that of an APCDD1 polypeptide, can be determined by known methods in the art, such as X-ray crystallography, which can determine a complete molecular structure. Solid or liquid phase NMR can be used to determine certain intramolecular distances. Any other experimental method of structure determination can be used to obtain partial or complete geometric structures. The geometric structures can be measured with a complexed ligand, natural or artificial, which can increase the accuracy of the active site structure determined.

Other methods for preparing or identifying peptides that bind to a target are known in the art. Molecular imprinting, for instance, can be used for the de novo construction of macromolecular structures such as peptides that bind to a molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Mosbach, (1994) Trends in Biochem. Sci., 19(9); and Wulff, G., in Polymeric Reagents and Catalysts (Ford, W. T., Ed.) ACS Symposium Series No. 308, pp 186-230, American Chemical Society (1986). One method for preparing mimics of an APCDD1 modulating compound involves the steps of: (i) polymerization of functional monomers around a known substrate (the template) that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are prepared by the free radical polymerization of functional monomers, resulting in a compound with a nonbiodegradable backbone. Other methods for designing such molecules include for example drug design based on structure activity relationships, which require the synthesis and evaluation of a number of compounds and molecular modeling.

Screening Assays

APCDD1 Modulating Compounds

An APCDD1 modulating compound can be a compound that affects the activity and/or expression of an APCDD1 molecule in vivo and/or in vitro. APCDD1 modulating compounds can be agonists and antagonists of an APCDD1 molecule, and can be compounds that exert their effect on the activity of APCDD1 via the expression, via post-translational modifications, or by other means.

Test compounds or agents which bind to an APCDD1 molecule, and/or have a stimulatory or inhibitory effect on the activity or the expression of an APCDD1 molecule, can be identified by two types of assays: (a) cell-based assays which utilize cells expressing an APCDD1 molecule or a variant thereof on the cell surface; or (b) cell-free assays, which can make use of isolated APCDD1 molecules or APCDD1 mutants described herein. These assays can employ various APCDD1 molecules (e.g., a biologically active fragment of APCDD1, full-length APCDD1, a fusion protein which includes all or a portion of APCDD1, or an APCDD1 mutant previously presented—having the biochemical variations just described, i.e., a fusion protein or fragments thereof). An APCDD1 molecule can be obtained from any suitable mammalian species (e.g., human APCDD1, rat APCDD1, chick APCDD1, or murine APCDD1). The assay can be a binding assay comprising direct or indirect measurement of the binding of a test compound or a known APCDD1 ligand. The assay can also be an activity assay comprising direct or indirect measurement of the activity of an APCDD1 molecule, for example measuring the activation of downstream Wnt signaling targets such as by examining Lef/TCF transcription by way of luciferase assays. The assay can also be an expression assay comprising direct or indirect measurement of the expression of APCDD1 mRNA or protein. The various screening assays can be combined with an in vivo assay comprising measuring the effect of the test compound on the symptoms of a hair loss disorder or disease in a subject (for example, androgenetic alopecia, Alopecia greata, Alopecia greata, alopecia totalis, or alopecia universalis), loss of hair pigmentation in a subject, or even hypertrichosis.

An in vivo assay can also comprise assessing the effect of a test compound on regulating hair growth in known mammalian models that display defective or aberrant hair growth phenotypes (such as mouse models having mutations in the APCDD1 protein) or mammals that contain a mutation in the APCDD1 open reading frame (ORF) that affects hair growth regulation or hair density, or hair pigmentation (Konyukhov et al., (2004) Russian J Gen 40(7): 968-74; Peters et al., (2003) J Invest Dermatol 121(4): 674-680; Green (1974) Mouse News Lett 51:1-23). In one embodiment, controlling hair growth can comprise an induction of hair growth or density in the subject. In another embodiment, controlling hair growth can comprise promoting hair loss in a subject. Here, the compound's effect in regulating hair growth can be observed either visually via examining the organism's physical hair growth or loss, or by assessing protein or mRNA expression using methods known in the art.

Assays for screening test compounds that bind to or modulate the activity of an APCDD1 molecule can also be carried out. The test compound can be obtained by any suitable means, such as from conventional compound libraries. Determining the ability of the test compound to bind to a membrane-bound form of the APCDD1 molecule can be accomplished via coupling the test compound with a radioisotope or enzymatic label such that binding of the test compound to the APCDD1-expressing cell can be measured by detecting the labeled compound in a complex. For example, the test compound can be labeled with ³H, ¹⁴C, ³⁵S, or ¹²⁵I, either directly or indirectly, and the radioisotope can be subsequently detected by direct counting of radioemmission or by scintillation counting. Alternatively, the test compound can be enzymatically labeled with, for example, horseradish peroxidase, alkaline phosphatase, or luciferase, and the enzymatic label detected by determination of conversion of an appropriate substrate to product.

A cell-based assay can comprise contacting a cell expressing a membrane-bound form of an APCDD1 molecule (for example, a biologically active fragment of APCDD1 or a variant thereof; full-length APCDD1 or a variant thereof; or a fusion protein which includes all or a portion of APCDD1 or a variant thereof) expressed on the cell surface with a test compound and determining the ability of the test compound to modulate (such as increase or decrease) the activity of the membrane-bound form of an APCDD1 molecule. Determining the ability of the test compound to modulate the activity of the membrane-bound APCDD1 molecule can be accomplished by any method suitable for measuring the activity of a protein involved in the Wnt/β-catenin signaling pathway. The activity of such a protein can be measured in various ways, such as activation of glycogen synthase kinase 3β (GSK3β), β-catenin phosphorylation, alteration in intracellular adenomatous polyposis coli (APC) protein concentration, alteration in intracellular axin concentration, β-catenin nuclear translocation, LEF/TCF transcription, or a combination thereof. For examples of assays, see also Cignal™ TCF/LEF Reporter Assay (luc; Kit: CCS-018L; SABiosciences, Frederick, Md.); Tao et al. (2005) Cell 120(6): 857-71; Labbe et al. (2000) PNAS 97(15): 8358-63; Labbe et al., (2007) Cancer Res 67(1): 75-84; and Letamendia et al. (2001) J Bone Joint Surg 83A(1): S31-39, which are all hereby incorporated by reference in their entireties.

The ability of a test compound to modulate the activity of an APCDD1 molecule or a variant thereof can be accomplished via determining the ability of the molecule to bind to or interact with a target molecule. The target molecule can be a molecule that binds or interacts with APCDD1 or an APCDD1 mutant in nature. Non-limiting examples include: a molecule on the surface of a cell which expresses APCDD1 or a variant thereof, a molecule in the extracellular milieu, a molecule on the surface of a second cell, a cytoplasmic molecule, or a molecule associated with the internal surface of a cell membrane. The target molecule can be a component of a signal transduction pathway which transduces an extracellular signal.

The cell-free assays of the present invention entail use of either a membrane-bound form of an APCDD1 molecule or an APCDD1 mutant described herein, or a soluble fragment thereof. In the case of cell-free assays comprising the membrane-bound form of the polypeptide, a solubilizing agent can be used in order for the membrane-bound form of the polypeptide to be maintained in solution. Examples of such solubilizing agents include but are not limited to non-ionic detergents such as Triton X-100, Triton X-114, n-octylglucoside, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), n-dodecylglucoside, n-dodecylmaltoside, octanoyl-N-methylglucamide, decanoyl-N-methylglucamide, Thesit, or N-dodecyl=N,N-dimethyl-3-ammonio-1-propane sulfonate.

An APCDD1 molecule or an APCDD1-target molecule can be immobilized to facilitate the separation of complexed from uncomplexed forms of one or both of the proteins. Binding of a test compound to an APCDD1 molecule or a variant thereof, or interaction of APCDD1 with a target molecule in the presence and absence of a test compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix (for example, glutathione-S-transferase (GST) fusion proteins or glutathione-S-transferase fusion proteins can be adsorbed onto glutathione sepharose beads (Sigma Chemical; St. Louis, Mo.) or glutathione derivatized microtiter plates).

An APCDD1 molecule, or a variant thereof, can also be immobilized via being bound to a solid support. Non-limiting examples of suitable solid supports include glass or plastic slides, tissue culture plates, microtiter wells, tubes, silicon chips, or particles such as beads (including, but not limited to, latex, polystyrene, or glass beads). Any method known in the art can be used to attach a polypeptide (or polynucleotide) corresponding to APCDD1 or a variant thereof, or test compound to a solid support, including use of covalent and non-covalent linkages, or passive absorption.

The diagnostic assay of the screening methods of the invention can also involve monitoring the expression of an APCDD1 molecule. For example, regulators of the expression of an APCDD1 molecule can be identified via contacting a cell with a test compound and determining the expression of APCDD1 protein or APCDD1 mRNA in the cell. The protein or mRNA expression level of APCDD1 in the presence of the test compound is compared to the protein or mRNA expression level of APCDD1 in the absence of the test compound. The test compound can then be identified as a regulator of APCDD1 expression based on this comparison. For example, when expression of APCDD1 protein or mRNA is statistically or significantly greater in the presence of the test compound than in its absence, the test compound is identified as a stimulator/enhancer of expression of APCDD1 protein or mRNA. In other words, the test compound can be said to be an APCDD1 modulating compound (such as an agonist). Alternatively, when expression of APCDD1 protein or mRNA is statistically or significantly less in the presence of the test compound than in its absence, the compound is identified as an inhibitor of the expression of APCDD1 protein or mRNA. In other words, the test compound can also be said to be an APCDD1 modulating compound (such as an antagonist). The expression level of APCDD1 protein or mRNA in cells can be determined by methods previously described.

For binding assays, the test compound can be a small molecule which binds to and occupies the binding site of an APCDD1 polypeptide, or a variant thereof. This can make the ligand binding site inaccessible to substrate such that normal biological activity is prevented. Examples of such small molecules include, but are not limited to, small peptides or peptide-like molecules. In binding assays, either the test compound or the APCDD1 polypeptide can comprise a detectable label, such as a fluorescent, radioisotopic, chemiluminescent, or enzymatic label (for example, alkaline phosphatase, horseradish peroxidase, or luciferase). Detection of a test compound which is bound to a polypeptide of APCDD1 or an APCDD1 mutant described herein can then be determined via direct counting of radioemmission, by scintillation counting, or by determining conversion of an appropriate substrate to a detectable product.

Determining the ability of a test compound to bind to an APCDD1 molecule or a variant thereof, such as an APCDD1 mutant described herein, also can be accomplished using real-time Biamolecular Interaction Analysis (BIA) [McConnell, (1992); Sjolander, (1991)]. BIA is a technology for studying biospecific interactions in real time, without labeling any of the interactants (for example, BIA-core™). Changes in the optical phenomenon surface plasmon resonance (SPR) can be used as an indication of real-time reactions between biological molecules.

To identify other proteins which bind to or interact with an APCDD1 molecule and modulate its activity, an APCDD1 polypeptide can be used as a bait protein in a two-hybrid assay or three-hybrid assay (Szabo, (1995); U.S. Pat. No. 5,283,317), according to methods practiced in the art. The two-hybrid system is based on the modular nature of most transcription factors, which consist of separable DNA-binding and activation domains.

Functional Assays

Test compounds can be tested for the ability to increase or decrease the activity of an APCDD1 molecule, or a variant thereof. Activity can be measured after contacting a purified APCDD1 molecule, a cell membrane preparation, or an intact cell with a test compound. A test compound that decreases the activity of an APCDD1 molecule by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for decreasing the activity of an APCDD1 molecule, for example an antagonist. A test compound that increases the activity of an APCDD1 molecule by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for increasing the activity of an APCDD1 molecule, for example an agonist.

Diagnosis

The invention provides diagnosis methods based on monitoring the APCDD1 gene in a subject. As used herein, the term “diagnosis” includes the detection, typing, monitoring, dosing, comparison, at various stages, including early, pre-symptomatic stages, and late stages, in adults and children. Diagnosis can include the assessment of a predisposition or risk of development, the prognosis, or the characterization of a subject to define most appropriate treatment (pharmacogenetics).

The invention provides diagnostic methods to determine whether an individual is at risk of developing a hair-loss disorder, or suffers from a hair-loss disorder, wherein the disease results from an alteration in the expression of the APCDD1 gene. In one embodiment, a method of detecting the presence of or a predisposition to a hair-loss disorder in a subject is provided. The subject can be a human or a child thereof. The method can comprise detecting in a sample from the subject the presence of an alteration in the expression of the APCDD1 gene in said sample. In one embodiment, the detecting comprises detecting whether there is an alteration in the APCDD1 gene locus, while in a further embodiment the detecting comprises detecting whether a small nuclear polymorphism (SNP) is present in the APCDD1 gene locus. The SNP can comprise a single nucleotide change, or a cluster of SNPs in and around the APCDD1 gene, or other SNPS that are in linkage disequilibrium (LD) with APCDD1 and could be used as sentinel SNPS for the APCDD1 haplotype. In another embodiment, the detecting comprises detecting whether at least a portion of the APCDD1 gene is deleted. In a further embodiment, the detecting comprises detecting whether expression of APCDD1 is reduced. In some embodiments, the detecting comprises detecting in the sample whether there is a reduction in APCDD1 mRNA, APCDD1 protein, or a combination thereof. The presence of such an alteration is indicative of the presence or predisposition to a hair-loss disorder. Non-limiting examples of hair-loss disorders include androgenetic alopecia, Alopecia greata, Alopecia greata, alopecia totalis, or alopecia universalis.

The presence of an alteration in the APCDD1 gene in the sample is detected through the genotyping of a sample, for example via gene sequencing, selective hybridization, amplification, gene expression analysis, or a combination thereof. In one embodiment, the sample can comprise blood, serum, sputum, lacrimal secretions, semen, vaginal secretions, fetal tissue, skin tissue, epithelial tissue, muscle tissue, amniotic fluid, or a combination thereof. In another embodiment, a reduction in APCDD1 expression of at least 20% indicates a predisposition or presence of a hair-loss disorder in the subject.

The invention also provides a method for treating or preventing a hair-loss disorder in a subject. In one embodiment, the method comprises detecting the presence of an alteration in the APCDD1 gene in a sample from the subject, the presence of the alteration being indicative of a hair-loss disorder, or the predisposition to a hair-loss disorder, and, administering to the subject in need a therapeutic treatment against a hair-loss disorder. The therapeutic treatment can be a drug administration (for example, a pharmaceutical composition comprising a functional APCDD1 molecule). In one embodiment, the molecule comprises a APCDD1 polypeptide comprising at least about 75%, at least about 80%, at least about 85%, at least about 90%, at least about 93%, at least about 95%, at least about 97%, at least about 98%, at least about 99%, or 100% of the amino acid sequence of SEQ ID NO: 1, and exhibits the function of restoring functional APCDD1 expression in deficient individuals, thus restoring the capacity to initiate hair growth in epithelial cells derived from hair follicles or skin. In another embodiment, the molecule comprises a nucleic acid encoding a APCDD1 polypeptide comprising at least about 75%, at least about 80%, at least about 85%, at least about 90%, at least about 93%, at least about 95%, at least about 97%, at least about 98%, at least about 99%, or 100% of the nucleic acid sequence of SEQ ID NO: 2 and encodes a polypeptide with the function of restoring functional APCDD1 expression in deficient individuals, thus restoring the capacity to initiate hair growth in epithelial cells derived from hair follicles or skin.

The alteration can be determined at the level of the APCDD1 DNA, RNA, or polypeptide. Optionally, detection can be determined by performing an oligonucleotide ligation assay, a confirmation based assay, a hybridization assay, a sequencing assay, an allele-specific amplification assay, a microsequencing assay, a melting curve analysis, a denaturing high performance liquid chromatography (DHPLC) assay (for example, see Jones et al, (2000) Hum Genet., 106(6):663-8), or a combination thereof. In another embodiment, the detection is performed by sequencing all or part of the APCDD1 gene or by selective hybridization or amplification of all or part of the APCDD1 gene. An APCDD1 gene specific amplification can be carried out before the alteration identification step.

An alteration in the APCDD1 gene locus can be any form of mutation(s), deletion(s), rearrangement(s) and/or insertions in the coding and/or non-coding region of the locus, alone or in various combination(s). Mutations can include point mutations. Insertions can encompass the addition of one or several residues in a coding or non-coding portion of the gene locus. Insertions can comprise an addition of between 1 and 50 base pairs in the gene locus. Deletions can encompass any region of one, two or more residues in a coding or non-coding portion of the gene locus, such as from two residues up to the entire gene or locus. Deletions can affect smaller regions, such as domains (introns) or repeated sequences or fragments of less than about 50 consecutive base pairs, although larger deletions can occur as well. Rearrangement includes inversion of sequences. The APCDD1 gene locus alteration can result in amino acid substitutions, RNA splicing or processing, product instability, the creation of stop codons, frame-shift mutations, and/or truncated polypeptide production. The alteration can result in the production of an APCDD1 polypeptide with altered function, stability, targeting or structure. The alteration can also cause a reduction in protein expression. In one embodiment, the alteration in the APCDD1 gene locus can comprise a point mutation, a deletion, or an insertion in the APCDD1 gene or corresponding expression product. In another embodiment, the alteration can be a deletion or partial deletion of the APCDD1 gene. The alteration can be determined at the level of the APCDD1 DNA, RNA, or polypeptide.

In another embodiment, the method can comprise detecting the presence of altered APCDD1 RNA expression. Altered RNA expression includes the presence of an altered RNA sequence, the presence of an altered RNA splicing or processing, or the presence of an altered quantity of RNA. These can be detected by various techniques known in the art, including sequencing all or part of the APCDD1 RNA or by selective hybridization or selective amplification of all or part of the RNA. In a further embodiment, the method can comprise detecting the presence of an altered APCDD1 polypeptide expression. Altered APCDD1 polypeptide expression includes the presence of an altered polypeptide sequence, the presence of an altered quantity of APCDD1 polypeptide, or the presence of an altered tissue distribution. These can be detected by various techniques known in the art, including by sequencing and/or binding to specific ligands (such as antibodies).

Various techniques known in the art can be used to detect or quantify altered APCDD1 gene or RNA expression or APCDD1 nucleic acid sequence, which include, but are not limited to, hybridization, sequencing, amplification, and/or binding to specific ligands (such as antibodies). Other suitable methods include allele-specific oligonucleotide (ASO), oligonucleotide ligation, allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, denaturing HLPC, melting curve analysis, heteroduplex analysis, RNase protection, chemical or enzymatic mismatch cleavage, ELISA, radio-immunoassays (RIA) and immuno-enzymatic assays (IEMA). Some of these approaches (such as SSCA and CGGE) are based on a change in electrophoretic mobility of the nucleic acids, as a result of the presence of an altered sequence. According to these techniques, the altered sequence is visualized by a shift in mobility on gels. The fragments can then be sequenced to confirm the alteration. Some other approaches are based on specific hybridization between nucleic acids from the subject and a probe specific for wild type or altered APCDD1 gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand specific for the polypeptide, for example, the use of a specific antibody.

Sequencing

Sequencing can be carried out using techniques well known in the art, using automatic sequencers. The sequencing can be performed on the complete APCDD1 gene or on specific domains thereof, such as those known or suspected to carry deleterious mutations or other alterations.

Amplification

Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR, allele-specific PCR, or PCR-SSCP. Amplification usually requires the use of specific nucleic acid primers, to initiate the reaction. Nucleic acid primers useful for amplifying sequences from the APCDD1 gene or locus are able to specifically hybridize with a portion of the APCDD1 gene locus that flank a target region of the locus, wherein the target region is altered in certain subjects having a hair-loss disorder. In one embodiment, amplification can comprise using forward and reverse RT-PCR primers comprising nucleotide sequences of SEQ ID NOS: 57 and 103, respectively (See Table 4).

The invention provides for a nucleic acid primer, wherein the primer can be complementary to and hybridize specifically to a portion of a APCDD1 coding sequence (e.g., gene or RNA) altered in certain subjects having a hair-loss disorder. Primers of the invention can be specific for altered sequences in a APCDD1 gene or RNA. By using such primers, the detection of an amplification product indicates the presence of an alteration in the APCDD1 gene or the absence of such gene. Primers can also be used to identify small nuclear polymorphisms (SNPs) locted in or around the APCDD1 gene locus; SNPs can comprise a single nucleotide change, or a cluster of SNPs in and around the APCDD1 gene, or other SNPS that are in linkage disequilibrium (LD) with APCDD1 and could be used as sentinel SNPS for the APCDD1 haplotype. Examples of primers of this invention can be single-stranded nucleic acid molecules of about 5 to 60 nucleotides in length, or about 8 to about 25 nucleotides in length. The sequence can be derived directly from the sequence of the APCDD1 gene. Perfect complementarity is useful to ensure high specificity; however, certain mismatch can be tolerated.

For example, a nucleic acid primer or a pair of nucleic acid primers as described above can be used in a method for detecting the presence of or a predisposition to a hair-loss disorder in a subject.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4:560, 1989; Landegren, Science 241:1077, 1988; Barringer, Gene 89:117, 1990); transcription amplification (see, e.g., Kwoh, Proc. Natl. Acad. Sci. USA 86:1173, 1989); and, self-sustained sequence replication (see, e.g., Guatelli, Proc. Natl. Acad. Sci. USA 87:1874, 1990); Q Beta replicase amplification (see, e.g., Smith, J. Clin. Microbiol. 35:1477-1491, 1997), automated Q-beta replicase amplification assay (see, e.g., Burg, Mol. Cell. Probes 10:257-271, 1996) and other RNA polymerase mediated techniques (e.g., NASBA, Cangene, Mississauga, Ontario); see also Berger, Methods Enzymol. 152:307-316, 1987; Sambrook; Ausubel; U.S. Pat. Nos. 4,683,195 and 4,683,202; Sooknanan, Biotechnology 13:563-564, 1995. All the references stated above are incorporated by reference in their entireties.

Selective Hybridization

Hybridization detection methods are based on the formation of specific hybrids between complementary nucleic acid sequences that serve to detect nucleic acid sequence alteration(s). A detection technique involves the use of a nucleic acid probe specific for wild type or altered APCDD1 gene or RNA, followed by the detection of the presence of a hybrid. The probe can be in suspension or immobilized on a substrate or support (for example, as in nucleic acid array or chips technologies). The probe can be labeled to facilitate detection of hybrids. In one embodiment, the probe according to the invention can comprise a nucleic acid sequence having SEQ ID NOS: 63 or 109. For example, a sample from the subject can be contacted with a nucleic acid probe specific for a wild type APCDD1 gene or an altered APCDD1 gene, and the formation of a hybrid can be subsequently assessed. In one embodiment, the method comprises contacting simultaneously the sample with a set of probes that are specific, respectively, for the wild type APCDD1 gene and for various altered forms thereof. Thus, it is possible to detect directly the presence of various forms of alterations in the APCDD1 gene in the sample. Also, various samples from various subjects can be treated in parallel.

According to the invention, a probe can be a polynucleotide sequence which is complementary to and capable of specific hybridization with a (target portion of a) APCDD1 gene or RNA, and that is suitable for detecting polynucleotide polymorphisms associated with APCDD1 alleles which predispose to or are associated with a hair-loss disorder. Useful probes are those that are complementary to the APCDD1 gene, RNA, or target portion thereof. Probes can comprise single-stranded nucleic acids of between 8 to 1000 nucleotides in length, for instance between 10 and 800, between 15 and 700, or between 20 and 500. Longer probes can be used as well. A useful probe of the invention is a single stranded nucleic acid molecule of between 8 to 500 nucleotides in length, which can specifically hybridize to a region of a APCDD1 gene or RNA that carries an alteration.

The sequence of the probes can be derived from the sequences of the APCDD1 gene and RNA as provided herein. Nucleotide substitutions can be performed, as well as chemical modifications of the probe. Such chemical modifications can be accomplished to increase the stability of hybrids (e.g., intercalating groups) or to label the probe. Some examples of labels include, without limitation, radioactivity, fluorescence, luminescence, and enzymatic labeling.

A guide to the hybridization of nucleic acids is found in e.g., Sambrook, ed., MOLECULAR CLONING: A LABORATORY MANUAL (2^{ND} ED.), Vols. 1-3, Cold Spring Harbor Laboratory, 1989; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, PART I. Theory and Nucleic Acid Preparation, Tijssen, ed. Elsevier, N.Y., 1993.

Specific Ligand Binding

As indicated above, alteration in the APCDD1 gene locus or APCDD1 expression can also be detected by screening for alteration(s) in APCDD1 polypeptide sequence or expression levels. Different types of ligands can be used, such as specific antibodies. In one embodiment, the sample is contacted with an antibody specific for an APCDD1 polypeptide and the formation of an immune complex is subsequently determined. Various methods for detecting an immune complex can be used, such as ELISA, radioimmunoassays (RIA) and immuno-enzymatic assays (IEMA).

For example, an antibody can be a polyclonal antibody, a monoclonal antibody, as well as fragments or derivatives thereof having substantially the same antigen specificity. Fragments include Fab, Fab′2, or CDR regions. Derivatives include single-chain antibodies, humanized antibodies, or poly-functional antibodies. An antibody specific for an APCDD1 polypeptide, can be an antibody that selectively binds an APCDD1 polypeptide, namely, an antibody raised against an APCDD1 polypeptide or an epitope-containing fragment thereof. Although non-specific binding towards other antigens can occur, binding to the target APCDD1 polypeptide occurs with a higher affinity and can be reliably discriminated from non-specific binding. In one embodiment, the method can comprise contacting a sample from the subject with an antibody specific for a wild type or an altered form of a APCDD1 polypeptide, and determining the presence of an immune complex. Optionally, the sample can be contacted to a support coated with antibody specific for the wild type or altered form of an APCDD1 polypeptide. In one embodiment, the sample can be contacted simultaneously, or in parallel, or sequentially, with various antibodies specific for different forms of an APCDD1 polypeptide, such as a wild type and various altered forms thereof.

The invention also provides for a diagnostic kit comprising products and reagents for detecting in a sample from a subject the presence of an alteration in the APCDD1 gene or polypeptide, in the APCDD1 gene or polypeptide expression, and/or in APCDD1 activity. The kit can be useful for determining whether a sample from a subject exhibits reduced APCDD1 expression or exhibits an APCDD1 gene deletion. For example, the diagnostic kit according to the present invention comprises any primer, any pair of primers, any nucleic acid probe and/or any ligand, (for example, a APCDD1 antibody), described in the present invention. The diagnostic kit according to the present invention can further comprise reagents and/or protocols for performing a hybridization, amplification or antigen-antibody immune reaction. In one embodiment, the kit can comprise nucleic acid primers that specifically hybridize to and can prime a polymerase reaction from APCDD1. In another embodiment, the primer can comprise a nucleotide sequence of SEQ ID NO: 19, 21-25, 63, 65, 67-71, or 109.

The diagnosis methods can be performed in vitro, ex vivo, or in vivo, using a sample from the subject, to assess the status of the APCDD1 gene locus. The sample can be any biological sample derived from a subject, which contains nucleic acids or polypeptides. Examples of such samples include, but are not limited to, fluids, tissues, cell samples, organs, or tissue biopsies. Non-limiting examples of samples include blood, plasma, saliva, urine, or seminal fluid. Pre-natal diagnosis can also be performed by testing fetal cells or placental cells, for instance. Screening of parental samples can also be used to determine risk/likelihood of offspring possessing the germline mutation. The sample can be collected according to conventional techniques and used directly for diagnosis or stored. The sample can be treated prior to performing the method, in order to render or improve availability of nucleic acids or polypeptides for testing. Treatments include, for instance, lysis (e.g., mechanical, physical, or chemical), centrifugation. Also, the nucleic acids and/or polypeptides can be pre-purified or enriched by conventional techniques, and/or reduced in complexity. Nucleic acids and polypeptides can also be treated with enzymes or other chemical or physical treatments to produce fragments thereof. In one embodiment, the sample is contacted with reagents such as probes, primers or ligands in order to assess the presence of an altered APCDD1 gene locus. Contacting can be performed in any suitable device, such as a plate, tube, well, or glass. In specific embodiments, the contacting is performed on a substrate coated with the reagent, such as a nucleic acid array or a specific ligand array. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids or polypeptides of the sample.

Identifying an altered APCDD1 polypeptide, RNA, or DNA in the sample is indicative of the presence of an altered APCDD1 gene in the subject, which can be correlated to the presence, predisposition or stage of progression of a hair-loss disorder. For example, an individual having a germ line APCDD1 mutation has an increased risk of developing a hair-loss disorder. The determination of the presence of an altered APCDD1 gene locus in a subject also allows the design of appropriate therapeutic intervention, which is more effective and customized. Also, this determination at the pre-symptomatic level allows a preventive regimen to be applied.

Gene Therapy and Protein Replacement Methods

Delivery of nucleic acids into viable cells can be effected ex vivo, in situ, or in vivo by use of vectors, such as viral vectors (e.g., lentivirus, adenovirus, adeno-associated virus, or a retrovirus), or ex vivo by use of physical DNA transfer methods (e.g., liposomes or chemical treatments). Non-limiting techniques suitable for the transfer of nucleic acid into mammalian cells in vitro include the use of liposomes, electroporation, microinjection, cell fusion, DEAE-dextran, and the calcium phosphate precipitation method (See, for example, Anderson, Nature, supplement to vol. 392, no. 6679, pp. 25-20 (1998)). Introduction of a nucleic acid or a gene encoding a polypeptide of the invention can also be accomplished with extrachromosomal substrates (transient expression) or artificial chromosomes (stable expression). Cells can also be cultured ex vivo in the presence of therapeutic compositions of the present invention in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., 1992), adenovirus (Berkner, 1992; Berkner et al., 1988; Gorziglia and Kapikian, 1992; Quantin et al., 1992; Rosenfeld et al., 1992; Wilkinson et al., 1992; Stratford-Perricaudet et al., 1990), vaccinia virus (Moss, 1992), adeno-associated virus (Muzyczka, 1992; Ohi et al., 1990), herpesviruses including HSV and EBV (Margolskee, 1992; Johnson et al., 1992; Fink et al., 1992; Breakfield and Geller, 1987; Freese et al., 1990), and retroviruses of avian (Biandyopadhyay and Temin, 1984; Petropoulos et al., 1992), murine (Miller, 1992; Miller et al., 1985; Sorge et al., 1984; Mann and Baltimore, 1985; Miller et al., 1988), and human origin (Shimada et al., 1991; Helseth et al., 1990; Page et al., 1990; Buchschacher and Panganiban, 1992). Non-limiting examples of in vivo gene transfer techniques include transfection with viral (typically retroviral) vectors (see U.S. Pat. No. 5,252,479, which is incorporated by reference in its entirety) and viral coat protein-liposome mediated transfection (Dzau et al., Trends in Biotechnology 11:205-210 (1993), incorporated entirely by reference). For example, naked DNA vaccines are generally known in the art; see Brower, Nature Biotechnology, 16:1304-1305 (1998), which is incorporated by reference in its entirety. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of gene therapy protocols and methods see Anderson et al., Science 256:808-813 (1992); U.S. Pat. Nos. 5,252,479, 5,747,469, 6,017,524, 6,143,290, 6,410,010 6,511,847; and U.S. Application Publication Nos. 2002/0077313 and 2002/00069, which are all hereby incorporated by reference in their entireties. For additional reviews of gene therapy technology, see Friedmann, Science, 244:1275-1281 (1989); Verma, Scientific American: 68-84 (1990); Miller, Nature, 357: 455-460 (1992); Kikuchi et al., J Dermatol Sci. 2008 May; 50(2):87-98; Isaka et al., Expert Opin Drug Deliv. 2007 September; 4(5):561-71; Jager et al., Curr Gene Ther. 2007 August; 7(4):272-83; Waehler et al., Nat Rev Genet. 2007 August; 8(8):573-87; Jensen et al., Ann Med. 2007; 39(2):108-15; Herweijer et al., Gene Ther. 2007 January; 14(2):99-107; Eliyahu et al., Molecules, 2005 Jan. 31; 10(1):34-64; and Altaras et al., Adv Biochem Eng Biotechnol. 2005; 99:193-260, all of which are hereby incorporated by reference in their entireties.

Protein replacement therapy can increase the amount of protein by exogenously introducing wild-type or biologically functional protein by way of infusion. A replacement polypeptide can be synthesized according to known chemical techniques or can be produced and purified via known molecular biological techniques. Protein replacement therapy has been developed for various disorders. For example, a wild-type protein can be purified from a recombinant cellular expression system (e.g., mammalian cells or insect cells—see U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al. and U.S. Pat. No. 5,879,680 to Ginns et al.), human placenta, or animal milk (see U.S. Pat. No. 6,188,045 to Reuser et al.), or other sources known in the art. After the infusion, the exogenous protein can be taken up by tissues through non-specific or receptor-mediated mechanism.

An APCDD1 polypeptide can also be delivered in a controlled release system. For example, the polypeptide can be administered using intravenous infusion, an implantable osmotic pump, a transdermal patch, liposomes, or other modes of administration. In one embodiment, a pump can be used (see is Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

Pharmaceutical Compositions and Administration for Therapy

APCDD1 molecules and APCDD1 modulating compounds of the invention can be incorporated into pharmaceutical compositions suitable for administration. Such compositions can comprise an APCDD1 molecule or an APCDD1 modulating compound and a pharmaceutically acceptable carrier.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

The invention also provides for a kit that comprises a pharmaceutically acceptable carrier and an APCDD1 modulating compound identified using the screening assays of the invention packaged with instructions for use. For modulators that are antagonists of the activity of an APCDD1 molecule, or which reduce the expression of an APCDD1 molecule, the instructions would specify use of the pharmaceutical composition for promoting the loss of hair on the body surface of a mammal (for example, the arms, legs, bikini area, face, and the like).

For APCDD1 modulating compounds that are agonists of the activity of an APCDD1 molecule or increase the expression of an APCDD1, the instructions would specify use of the pharmaceutical composition for regulating hair growth. In one embodiment, the instructions would specify use of the pharmaceutical composition for the treatment of hair loss disorders. In another embodiment, the instructions would specify use of the pharmaceutical composition for promoting hair growth in a subject. In a further embodiment, the instructions would specify use of the pharmaceutical composition for restoring hair pigmentation. For example, administering an APCDD1 agonist can reduce hair graying in a subject.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition containing an APCDD1 modulating compound can be administered in conjunction with a pharmaceutically acceptable carrier, for any of the therapeutic effects discussed herein. Such pharmaceutical compositions can comprise, for example antibodies directed to human APCDD1 or a variant thereof, APCDD1 agonists, APCDD1 antagonists, or APCDD1 inhibitors. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the APCDD1 modulating compound (e.g., a polypeptide or antibody) in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art

In some embodiments, the APCDD1 modulating compound can be applied via transdermal delivery systems, which slowly releases the active compound for percutaneous absorption. Permeation enhancers can be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Various routes of administration and various sites of cell implantation can be utilized, such as, subcutaneous or intramuscular, in order to introduce the aggregated population of cells into a site of preference. Once implanted in a subject (such as a mouse, rat, or human), the aggregated cells can then stimulate the formation of a hair follicle and the subsequent growth of a hair structure at the site of introduction. In another embodiment, transfected cells (for example, cells expressing APCDD1) are implanted in a subject to promote the formation of hair follicles within the subject. In further embodiments, the transfected cells are cells derived from the end bulb of a hair follicle (such as dermal papilla cells or dermal sheath cells).

Aggregated cells (for example, cells grown in a hanging drop culture) or transfected cells (for example, cells produced as described herein) maintained for 1 or more passages can be introduced (or implanted) into a subject (such as a rat, mouse, dog, cat, human, and the like).

“Subcutaneous” administration can refer to administration just beneath the skin (i.e., beneath the dermis). Generally, the subcutaneous tissue is a layer of fat and connective tissue that houses larger blood vessels and nerves. The size of this layer varies throughout the body and from person to person. The interface between the subcutaneous and muscle layers can be encompassed by subcutaneous administration.

This mode of administration can be feasible where the subcutaneous layer is sufficiently thin so that the factors present in the compositions can migrate or diffuse from the locus of administration and contact the hair follicle cells responsible for hair formation. Thus, where intradermal administration is utilized, the bolus of composition administered is localized proximate to the subcutaneous layer.

Administration of the cell aggregates (such as DP or DS aggregates) is not restricted to a single route, but can encompass administration by multiple routes. For instance, exemplary administrations by multiple routes include, among others, a combination of intradermal and intramuscular administration, or intradermal and subcutaneous administration. Multiple administrations can be sequential or concurrent. Other modes of application by multiple routes will be apparent to the skilled artisan.

In other embodiments, this implantation method will be a one-time treatment for some subjects. In further embodiments of the invention, multiple cell therapy implantations will be required. In some embodiments, the cells used for implantation will generally be subject-specific genetically engineered cells. In another embodiment, cells obtained from a different species or another individual of the same species can be used. Thus, using such cells can require administering an immunosuppressant to prevent rejection of the implanted cells. Such methods have also been described in United States Patent Application Publication 2004/0057937 and PCT application publication WO 2001/32840, and are hereby incorporated by reference.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Mutations in the Hypotrichin/APCDD1 Gene, a Target of wnt Signaling, Underlie Hereditary Hypotrichosis Simplex

Very few forms of hereditary hair loss exist that bear similarities to common hair loss such as androgenetic alopecia. Hereditary hypotrichosis simplex (HHS; OMIM 146520/605389) is one such form of hair loss that has been infrequently described in the literature^{1,2}.

To identify a gene involved in HHS, we performed genetic linkage analysis in two families of Pakistani origin with autosomal dominant HHS, characterized by progressive loss and thinning of scalp and body hairs. We identified a region of linkage on chromosome 18p11.22 in both families. Fine mapping of critical recombination events delineated a region of 1.8 Mb, containing 8 known genes, 4 pseudogenes and 3 predicted transcripts. One of the known genes, APCDD1, encodes a predicted secreted protein with no known function. Resequencing of APCDD1 revealed an identical heterozygous pathogenic mutation in the signal sequence of the protein (L9R) in both families. We confirmed the role of APCDD1 in HHS by sequencing the gene in a third family from Italy that had previously been linked to the same region of chromosome 18p³, and unexpectedly identified the identical mutation. Haplotype analysis revealed that the mutation had arisen independently in each of the three families, suggesting that this nucleotide is a hotspot for mutations in HHS. APCDD1 is intensely expressed in the dermal papilla, the matrix, and the hair shaft of human hair follicles. Expression of mutant APCDD1 demonstrates that the mutation L9R dramatically prevents the translational processing, which leads to significant reduction of the expression and secretion of APCDD1. Our findings indicate that disruption of APCDD1 underlies HHS, and uncover a gene with a critical role in human hair growth.

The hair follicle (HF) is a complex organ which periodically regenerates in the form of a hair cycle. Recent advances in molecular genetics have enabled the identification of numerous genes that are expressed in the HF⁴. Disruption of some of these genes underlies different types of hereditary hypotrichosis. Although most of them are associated with other cutaneous and/or systemic abnormalities, isolated forms of hereditary hypotrichosis also exist. Of these, Marie Unna hypotrichosis (OMIM 146550) is an autosomal dominant disorder characterized by coarse, wiry and twisted hair shaft, and has been reported to show linkage to chromosome 8p22-p21⁵, though no gene has yet been identified. In addition, monilethrix is characterized by a specific hair shaft anomaly called moniliform hair. This disease can show either autosomal dominant (OMIM 158000) or recessive (OMIM 252200) inheritance trait, and several causative genes have been identified to date⁶⁻⁸.

A rare form of hereditary hypotrichosis without any characteristic hair shaft anomalies is known as hereditary hypotrichosis simplex (HHS; OMIM 146520/605389)^{1,2}. Affected individuals with HHS typically show normal hair at birth, but hair loss and thinning of the hair shaft on the scalp start during early childhood and progress with age, frequently affecting the body hairs as well. Histologically, HHS is characterized by progressive HF miniaturization, which is a pathognomonic feature to androgenetic alopecia^{1,9}. In most cases, HHS shows an autosomal dominant inheritance pattern (ADHHS)^{1-3, 10}, but recessive HHS (ARHHS) is also known¹¹. We undertook this study to discover the genetic basis of HHS.

Results

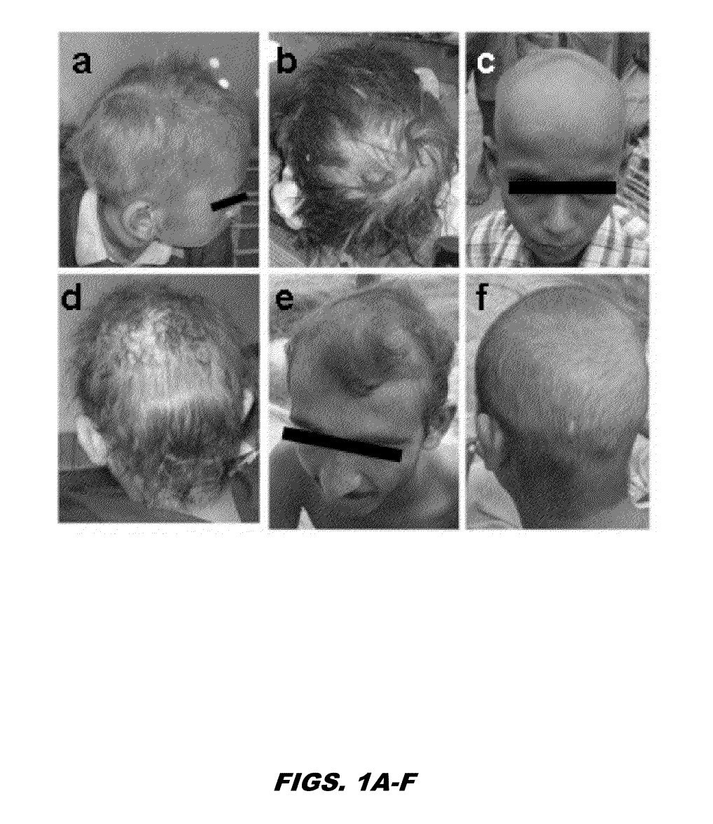

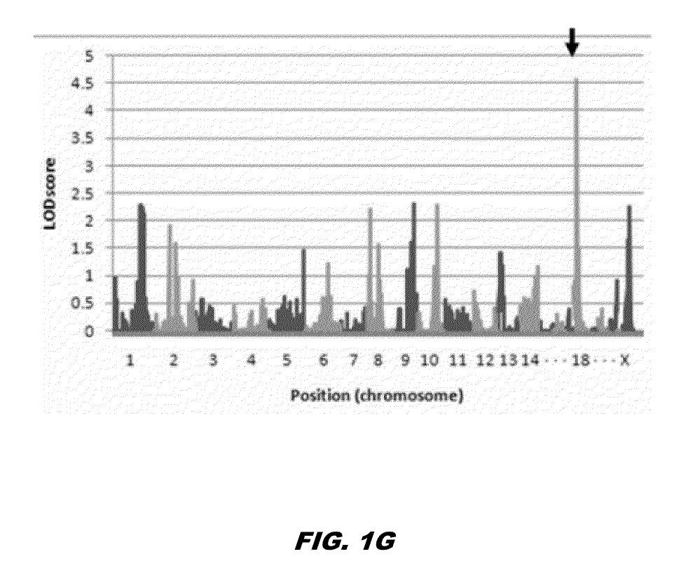

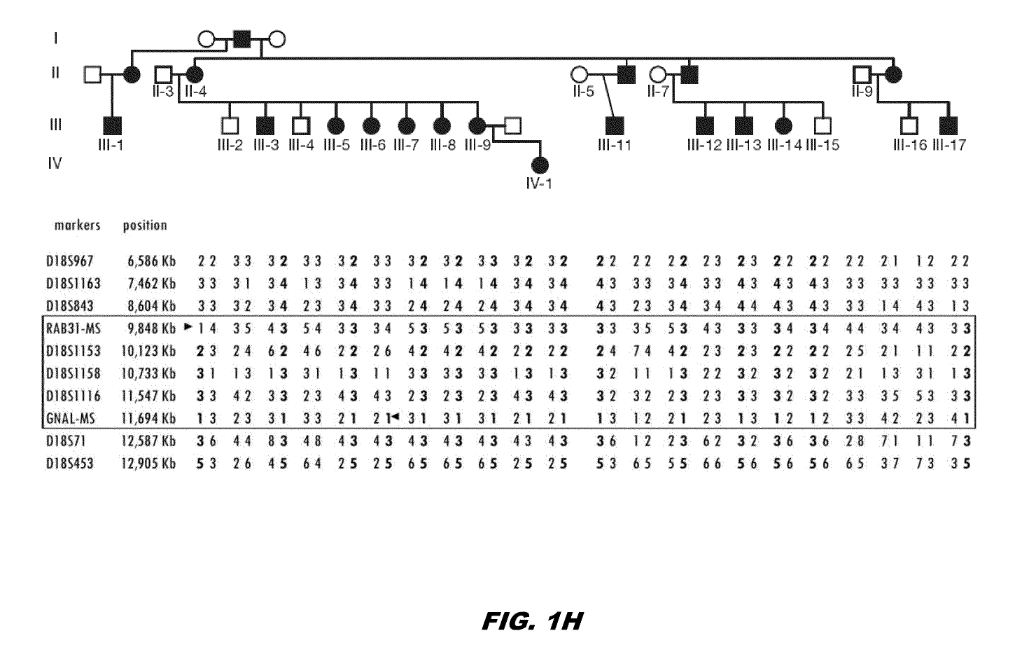

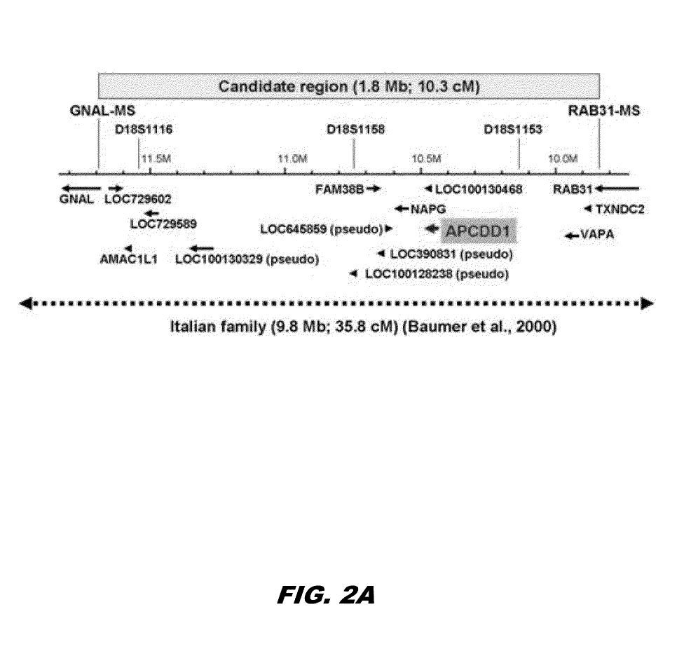

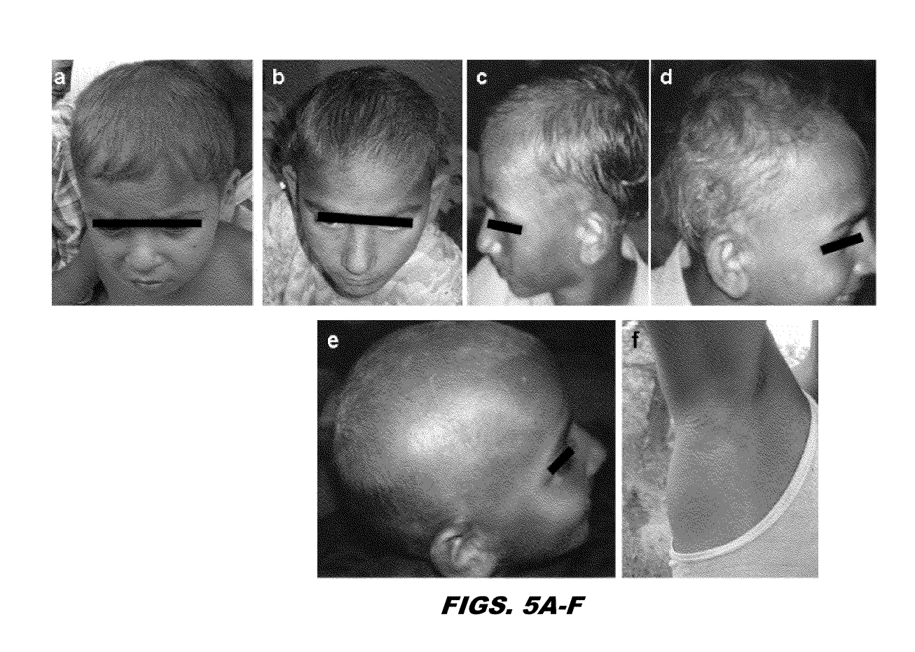

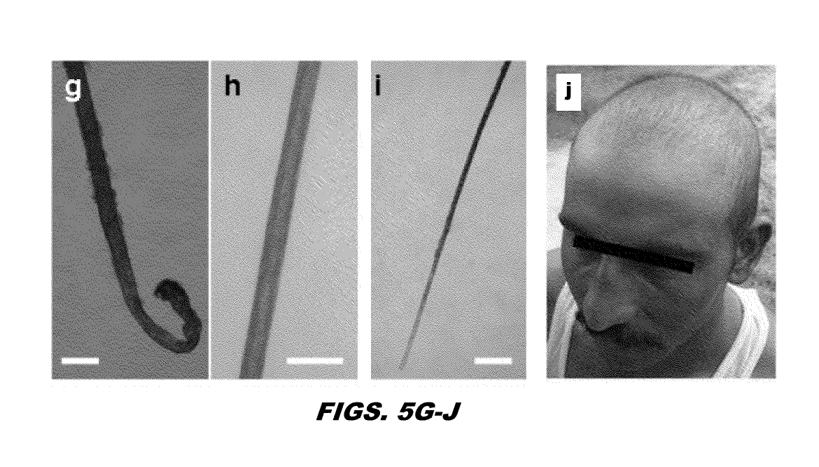

We first identified two Pakistani families, HHS1 and HHS2, with features consistent with HHS. Pedigrees of both families were consistent with autosomal dominant inheritance, and each family had multiple affected individuals. All affected individuals had normal scalp hair density at birth, and the hair loss gradually progressed with age, beginning around 2-5 years old (FIGS. 1A-F, FIG. 5). The hair grows slowly and stops growing after a few inches. Some affected individuals show light-colored or hypopigmented hair shafts (FIGS. 1A and 1C, FIG. 5A). In most cases, body hairs and sexual hairs are also sparse (FIG. 5F). Eyebrows, eyelashes, and beard hairs are not affected. Under light microscopy, the bulb portion of the plucked hair is miniaturized and shows dystrophic features (FIG. 5G). The hair shaft is thin and without any characteristic anomalies (FIG. 5H), and the distal ends appear tapered (FIG. 5I). Affected individuals in both families show normal teeth, nails, and sweating, and do not show keratosis pilaris. There was no familial history of either neurologic abnormalities or a high prevalence of cancers.

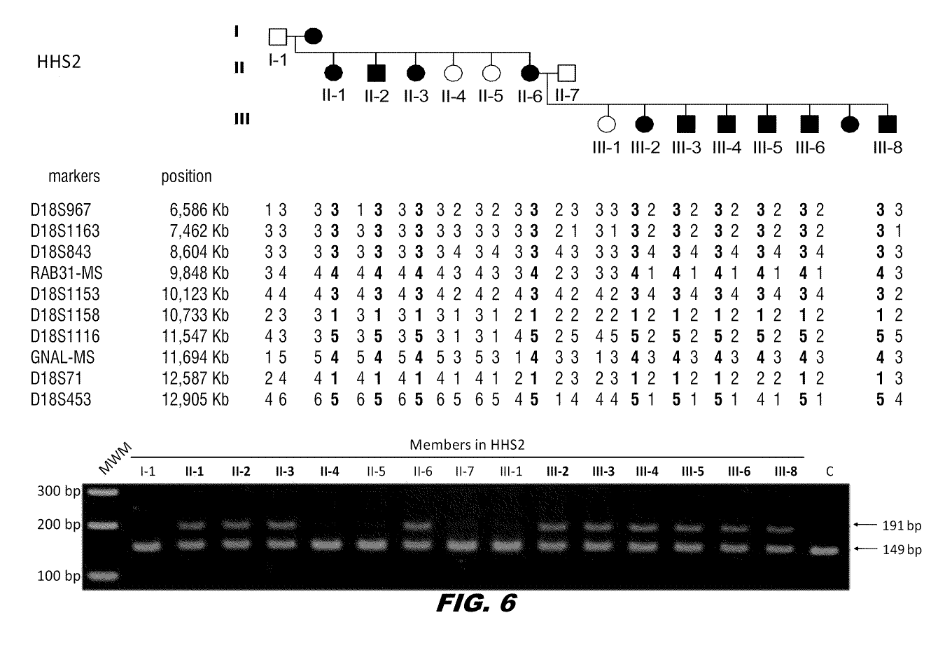

We used commercially available low density human mapping arrays (Affymetrix 10K) to genotype 16 and 12 members of each family, respectively. Parametric linkage analysis was performed on inferred haplotypes under a dominant model. A maximum LOD score of z=4.6 was obtained for a haplotype on chromosome 18p11.22. The −2LOD interval spanned from 7.4 Mb to 25 Mb (FIG. 1G). This interval was then saturated with microsatellite markers to confirm linkage and more clearly define the region (FIG. 1H, FIG. 6). Critical recombination events were detected between markers RAB31-MS and D18S1153 in the affected individual III-1 of HHS1 (FIG. 1H), as well as between markers D18S1116 and GNAS-MS in the affected individual III-4 of HHS1 (FIG. 1H), which allowed the interval of linkage to be narrowed to 1.8 Mb flanked by markers RAB31-MS and GNAL-MS.

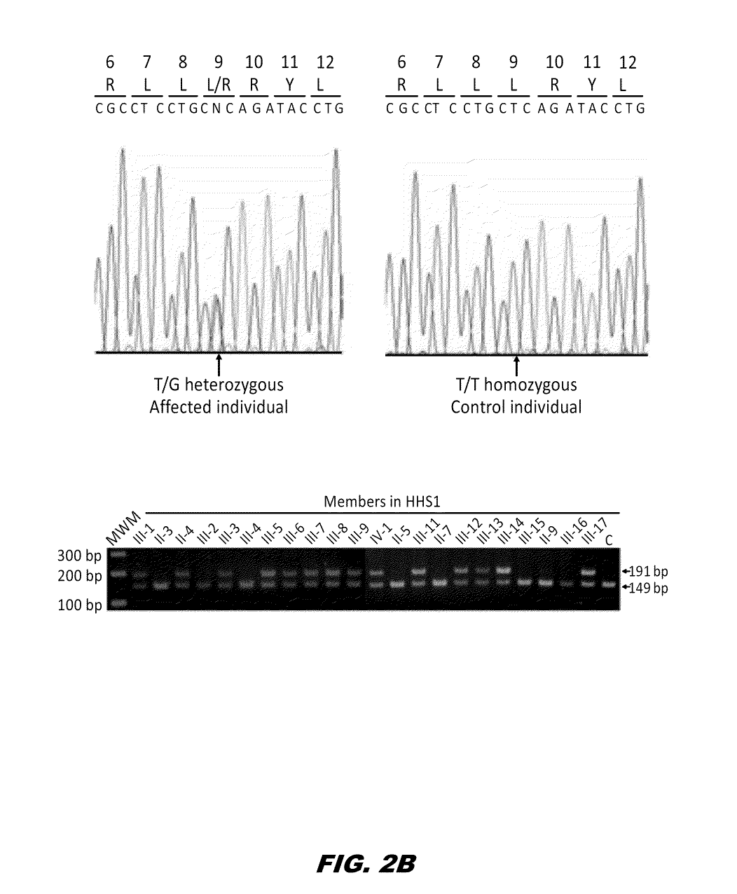

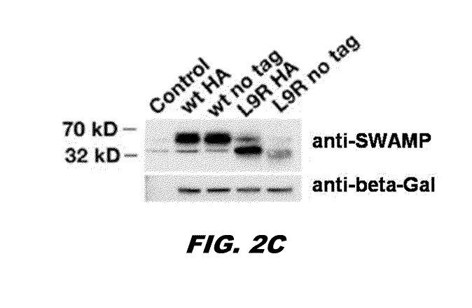

The critical region contained 8 known genes, 4 pseudogenes and 3 unknown predicted transcripts (FIG. 2A). We performed direct sequencing analysis of all known genes in the region, and identified a mutation in APCDD1 (adenomatosis polyposis coli down-regulated 1) gene¹² in both families. All affected individuals in both families carry the identical heterozygous missense mutation consisting of a T- to -G transversion at position 26 in exon 1 (26T>G), resulting in the substitution from Leucine to Arginine at codon 9, designated L9R (FIG. 2B). This nucleotide change is not reported in any of the public databases. Screening assays with the restriction enzyme DdeI show that the mutation L9R cosegregates with the disease phenotype in both families, and 100 unrelated unaffected control individuals of Pakistani origin do not carry the mutation (FIG. 2A, FIG. 6).

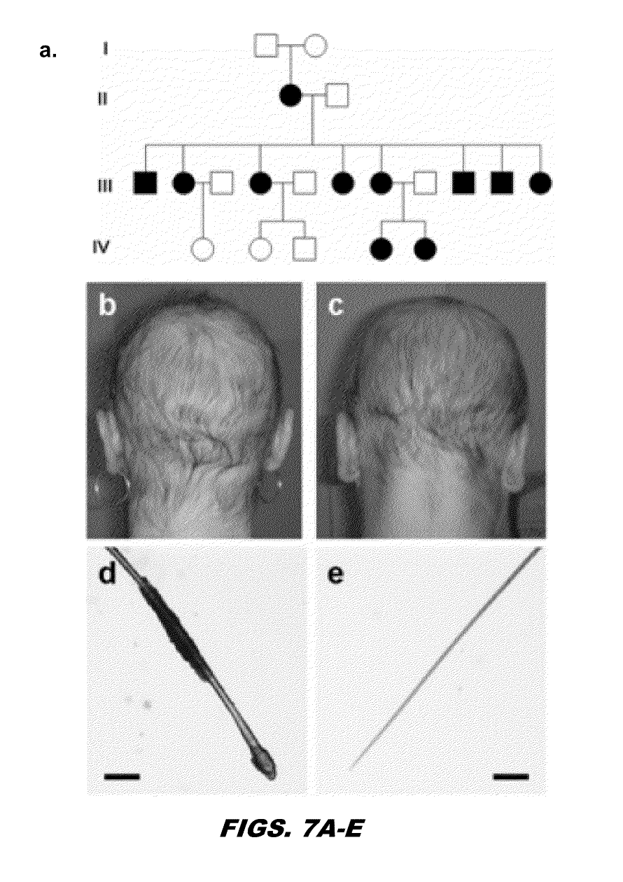

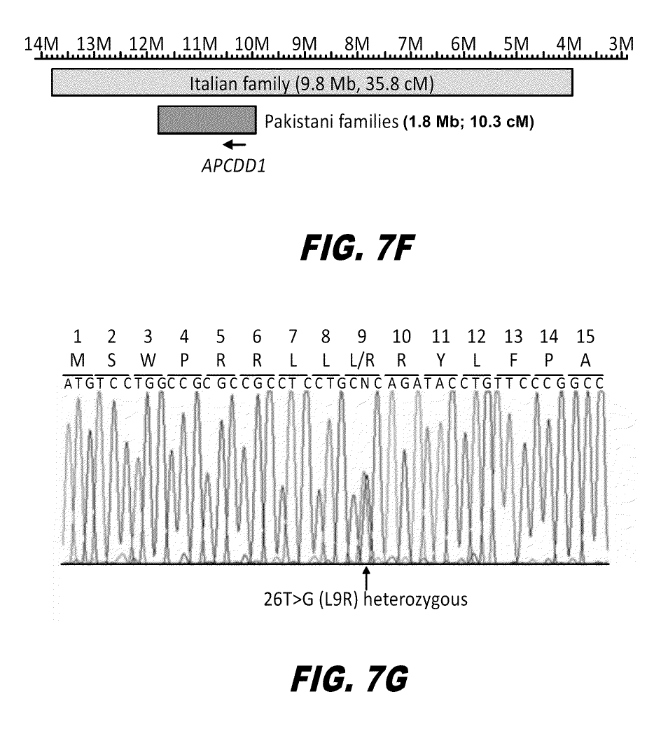

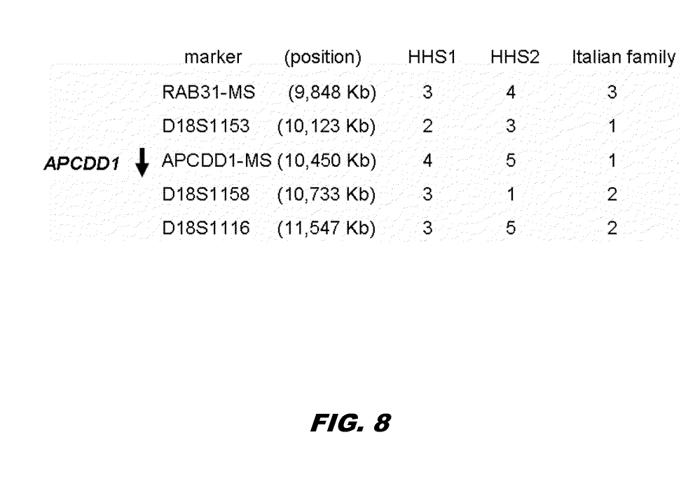

To replicate the causal role of APCDD1 in HHS, we analyzed an Italian family with autosomal dominant HHS (FIG. 7A). This family displays similar clinical features with the Pakistani families (FIG. 7B-E), and was previously reported to show linkage to a 9.8 Mb interval on chromosome 18p11.32-p11.23, in which the APCDD1 gene resides (FIG. 7F)³. Unexpectedly, direct sequencing analysis demonstrated that affected individuals in this family carry the identical heterozygous mutation 26T>G (L9R) in the APCDD1 gene (FIG. 7G). The mutation links with the disease phenotype and was excluded from 100 unrelated unaffected northern European control individuals. Haplotype analysis using microsatellite markers around and within the APCDD1 gene demonstrates that all three families show a different disease-related haplotype (FIG. 8), suggesting that the mutation arose independently in each family. Although the mutation does not exist in a CpG dinucleotide, our results strongly suggest that the nucleotide at position 26 of the APCDD1 gene is a mutational hotspot.

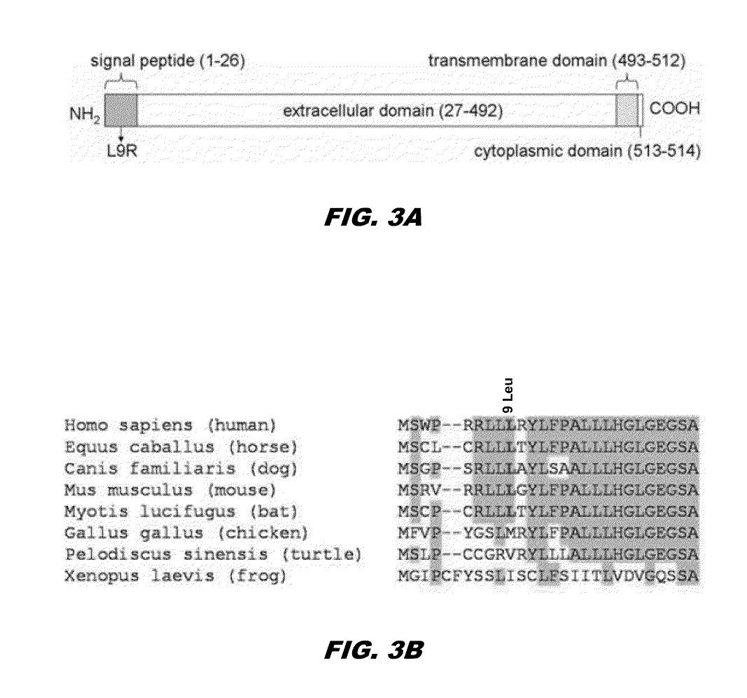

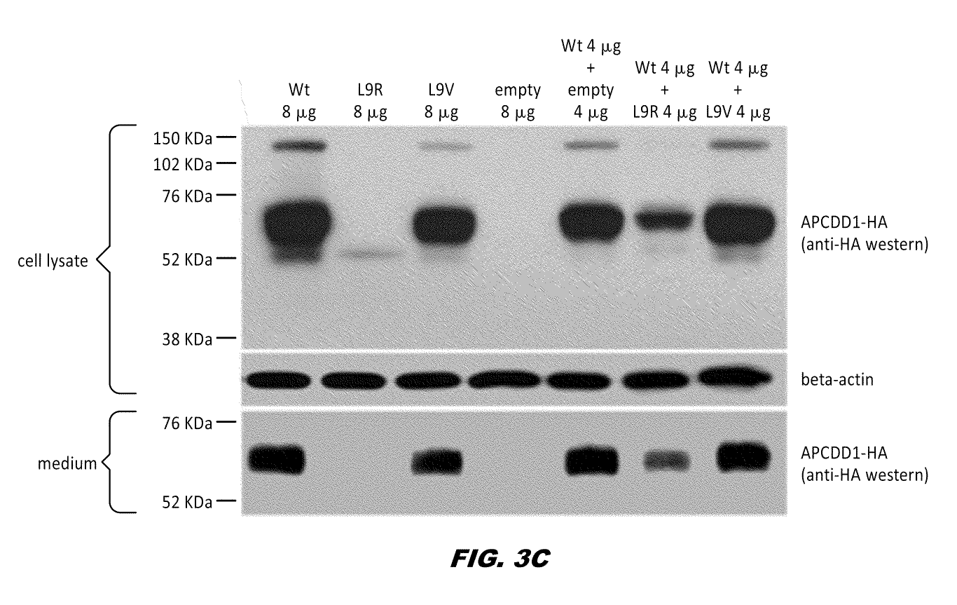

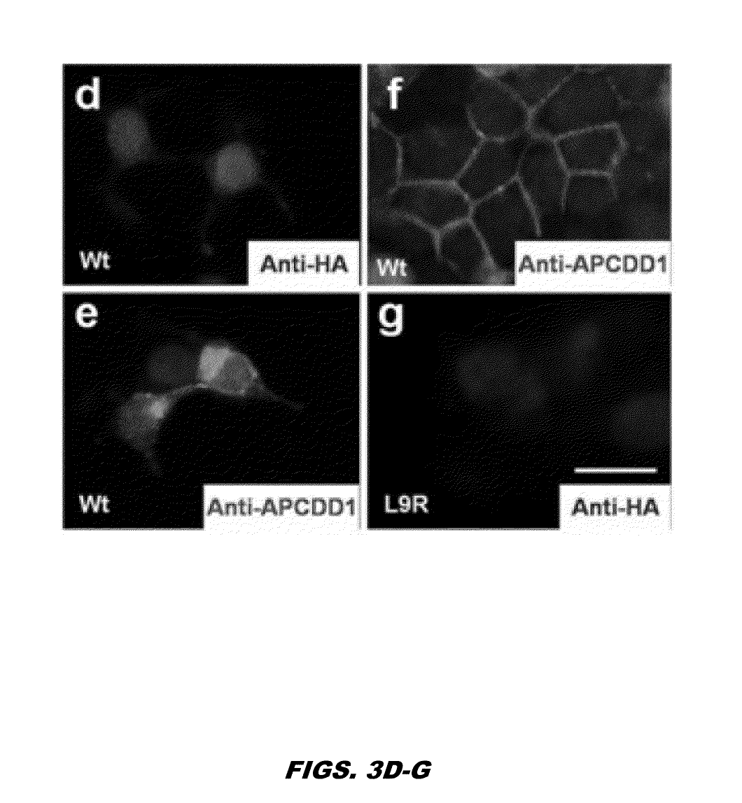

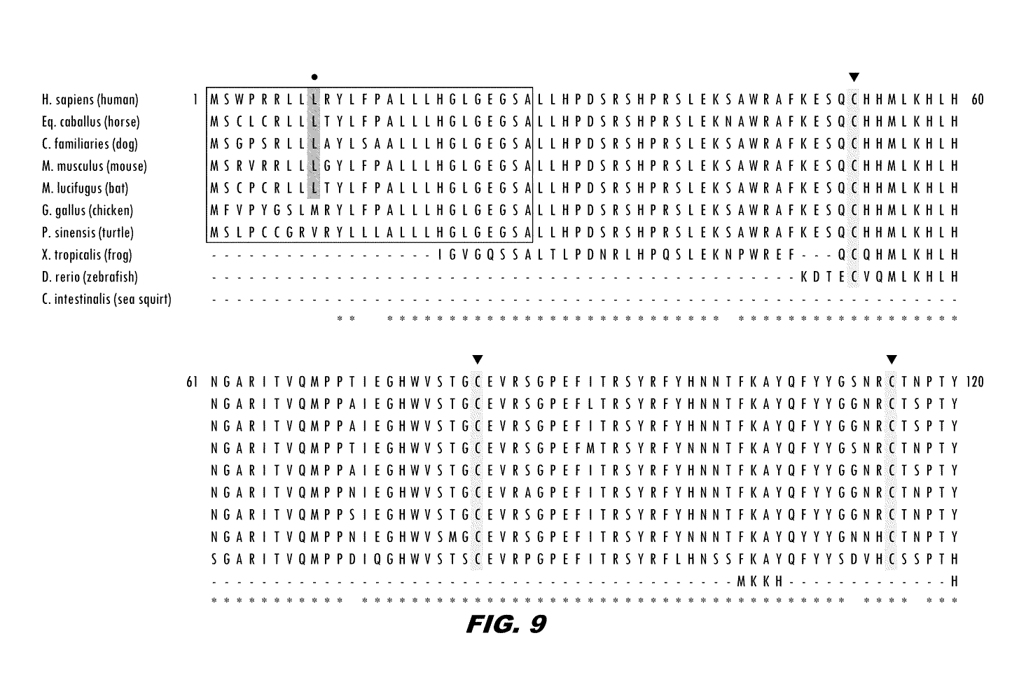

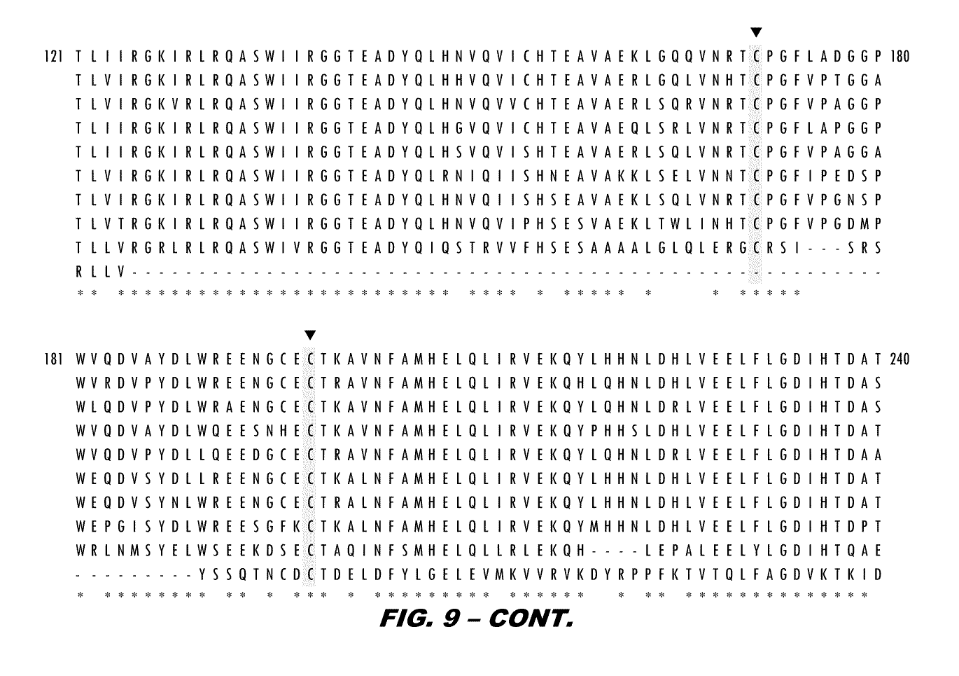

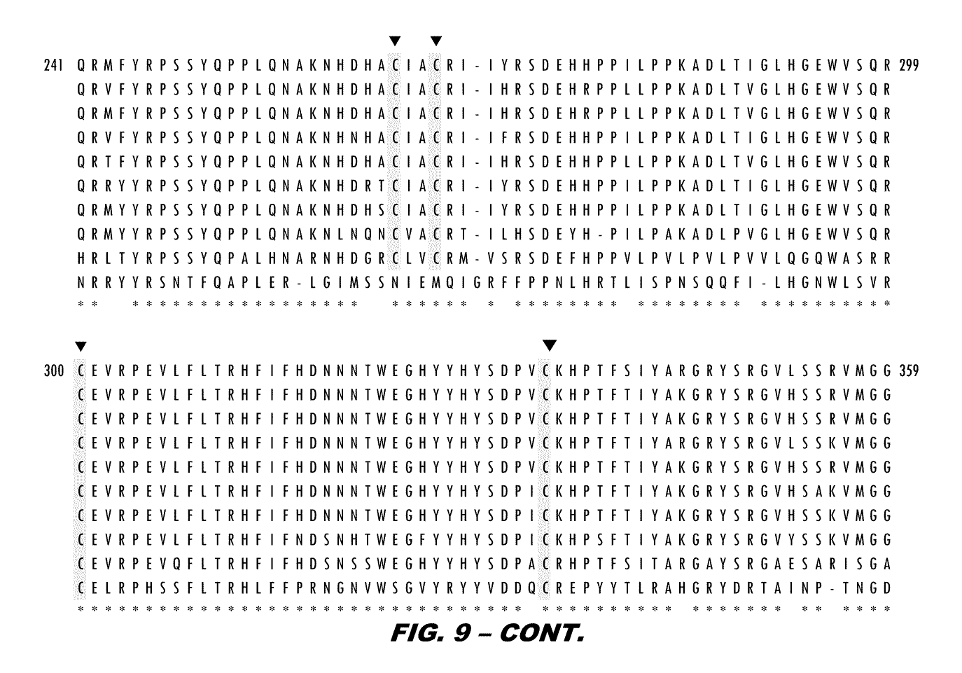

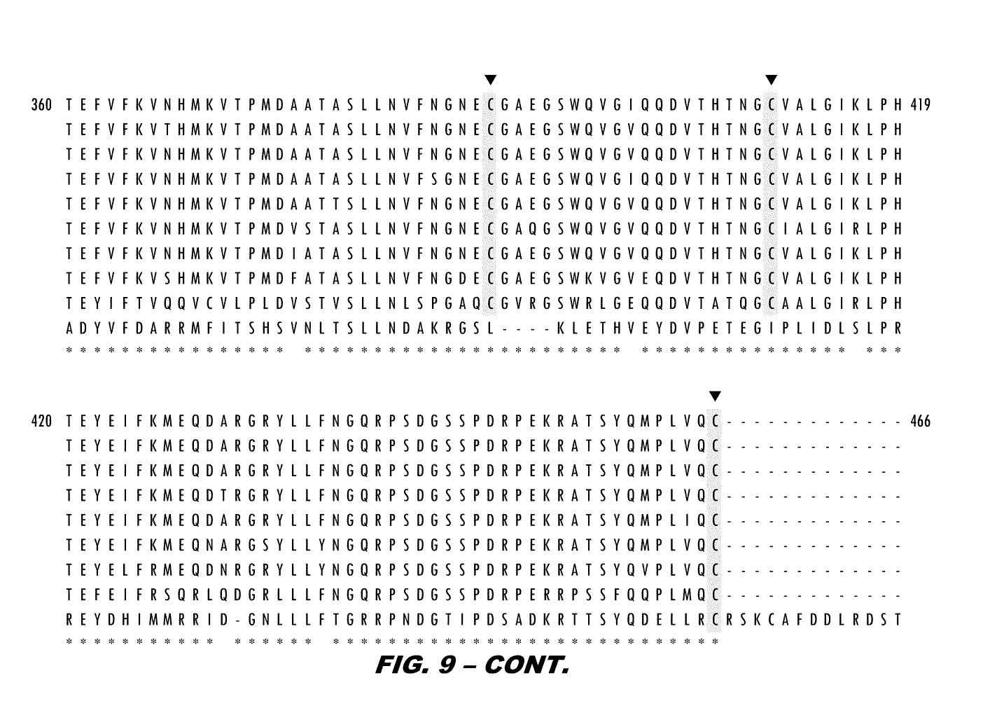

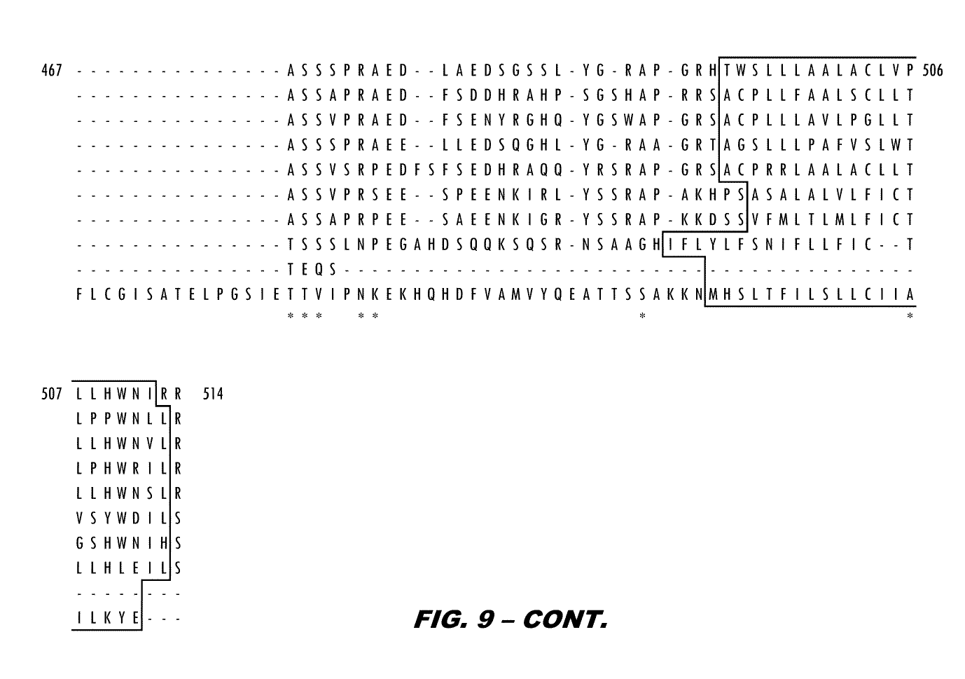

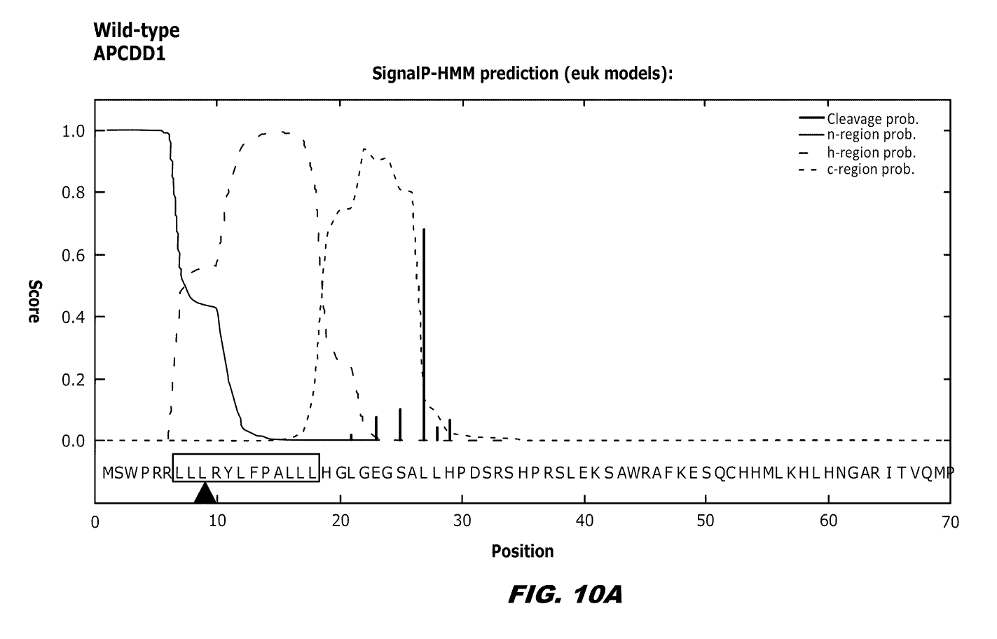

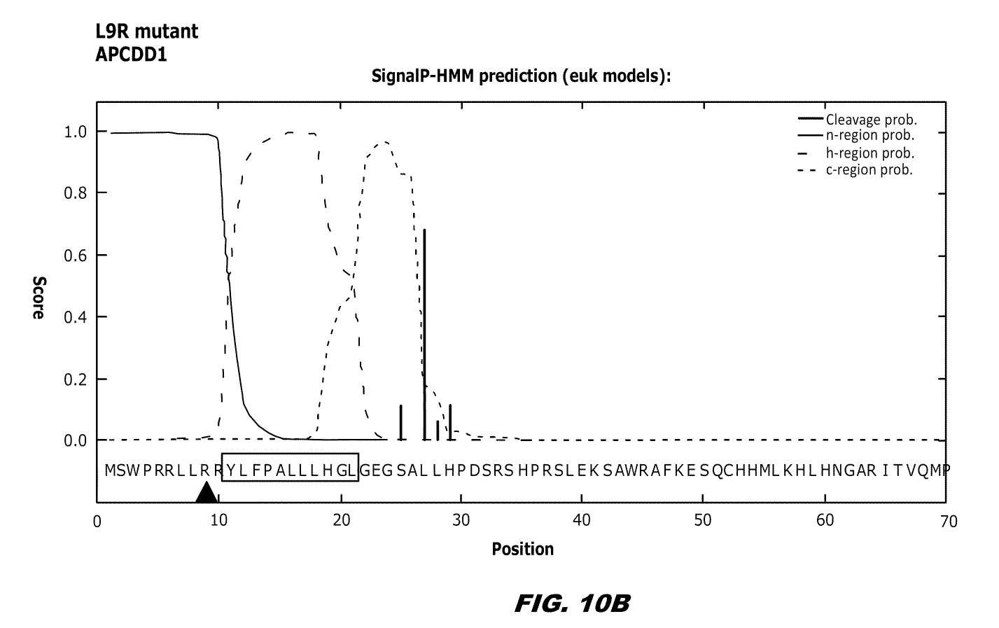

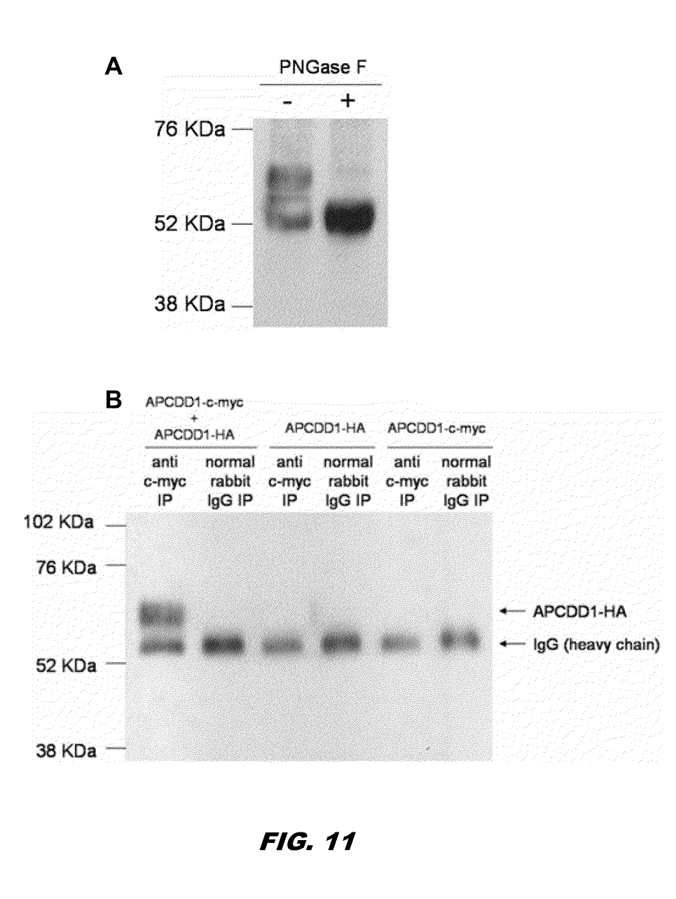

The APCDD1 gene was initially discovered in a screen for genes associated with colon cancer, and was found to be downregulated by the tumor suppressor APC¹². The amino-acid sequence of APCDD1 protein does not have any known homology domains to aid in predicting its function. In addition, there are no known family members, other than APCDD1-like gene (APCDD1L). The APCDD1 protein is 58 KDa in size and predicted to consist of the N-terminal signal peptide, followed by the large extracellular domain, the C-terminal transmembrane domain and the cytoplasmic domain (FIG. 3A). Within the extracellular domain, there is a potential N-glycosylation site at amino acid position 168. APCDD1 is highly conserved in vertebrate evolution, with homologs being present as distantly as sea squirt (FIG. 9)¹⁴. The mutation found in all three families is identical (L9R), resides in the signal peptide (FIG. 3A), and Leu9 is conserved from bat to human (FIG. 3B). The analysis of the signal peptide sequences with the SignalP-HMM program (version 3.0) shows that Leu9 is located within the hydrophobic core of the signal peptide that is critical for the cotranslational processing of the protein¹⁵. The substitution by a hydrophilic amino acid arginine is predicted to severely affect the composition of the hydrophobic core (FIGS. 10A and 10B). To test this, we transfected expression constructs of C-terminal HA-tagged wild-type and two different mutant APCDD1 into human embryonic kidney (HEK) 293 cells, and analyzed their expression patterns by western blotting with an anti-HA antibody. Three fragments around 52 KDa, 65 KDa, and 130 KDa were detected in total cell lysate of the wild-type APCDD1 construct-transfected cells (FIG. 3C; lane 1, top panel). The 65 KDa fragment was clearly digested with PNGase F, suggesting that wild-type APCDD1 is modified with N-Glycosylation (FIG. 11A).

To examine the 130 KDa fragment, we overexpressed both HA-tagged and c-myc-tagged APCDD1 in HEK293T cells, and performed immunoprecipitation using an anti-c-myc antibody, which was followed by western blot with an anti-HA antibody. The result demonstrated that HA-tagged APCDD1 was co-precipitated with c-myc-tagged APCDD1, which strongly suggests dimerization of the APCDD1 protein (FIG. 11B). A mutant APCDD1 with a conservative amino acid substitution (L9V) showed a similar expression pattern to the wild type protein (FIG. 3C; lane 3, top panel). By contrast, only a faint fragment, 52 KDa in size, was detected in total cell lysate of the L9R mutant construct-transfected cells (FIG. 3C; lane 2, top panel). In addition, a 65 KDa fragment was detected in the medium of the wild-type and the control L9V mutant APCDD1 construct-transfected cells, while no fragment in the medium of the L9R mutant construct-transfected cells (FIG. 3C; lanes 1-3, bottom panel). Furthermore, when equal amounts of the wild-type and the L9R mutant constructs are co-transfected, the expression level of the wild-type APCDD1 is markedly decreased (FIG. 3C; lane 6).

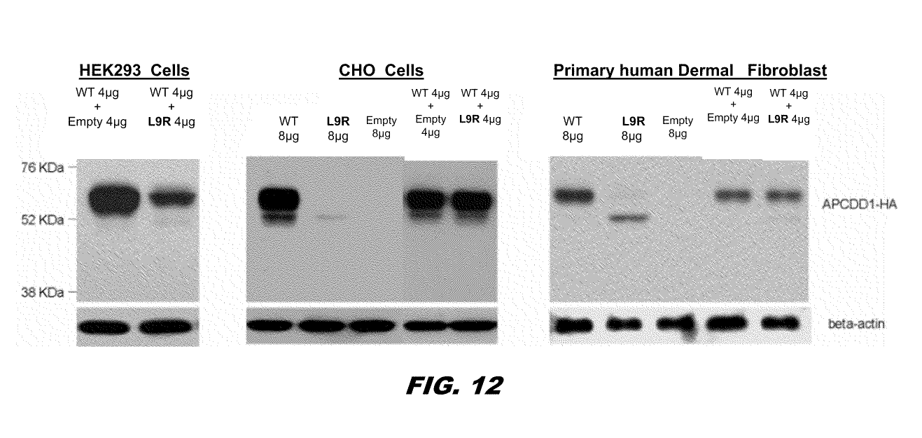

To analyze the localization of APCDD1 protein in cells, we performed immunocytostainings using the anti-HA antibody and a commercially available mouse polyclonal anti-APCDD1 antibody (from Abnova). At lower cell density, the wild-type APCDD1 is detected in the cytoplasm, as well as at the cell membrane (FIGS. 3D and 3E). Interestingly, at higher cell density, its expression is more clearly defined to the cell membrane (FIG. 3F). By contrast, the mutant L9R APCDD1 is only weakly expressed around the nucleus (FIG. 3G), suggesting that the mutant protein is retained in the endoplasmic reticulum. These results show that APCDD1 is a secreted protein which localizes at the cell membrane, and the mutation L9R in the signal peptide severely disrupts the cotranslational processing of the protein from the mutant allele. Furthermore, when equal amounts of the wild-type and the L9R mutant constructs are co-transfected, the expression level of the wild-type APCDD1 is markedly decreased (FIG. 12), suggesting that the L9R mutant APCDD1 also prevents the expression of the wild-type protein in HEK293T cells.

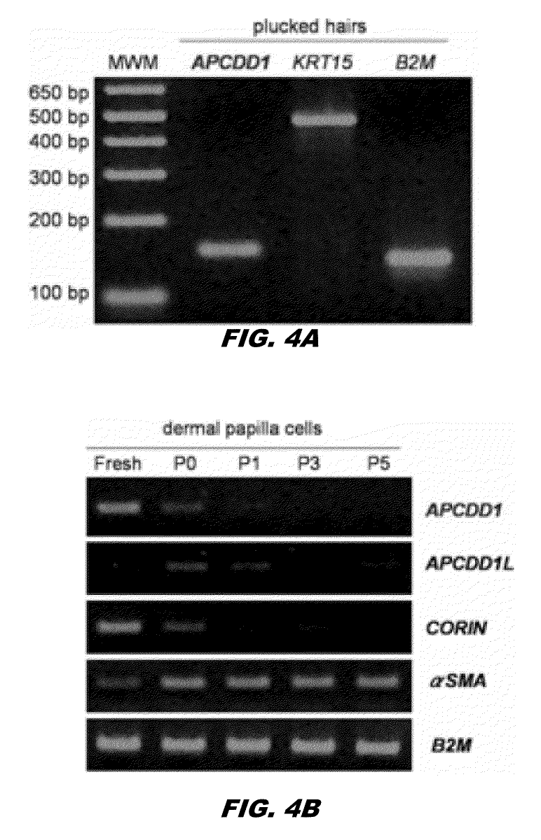

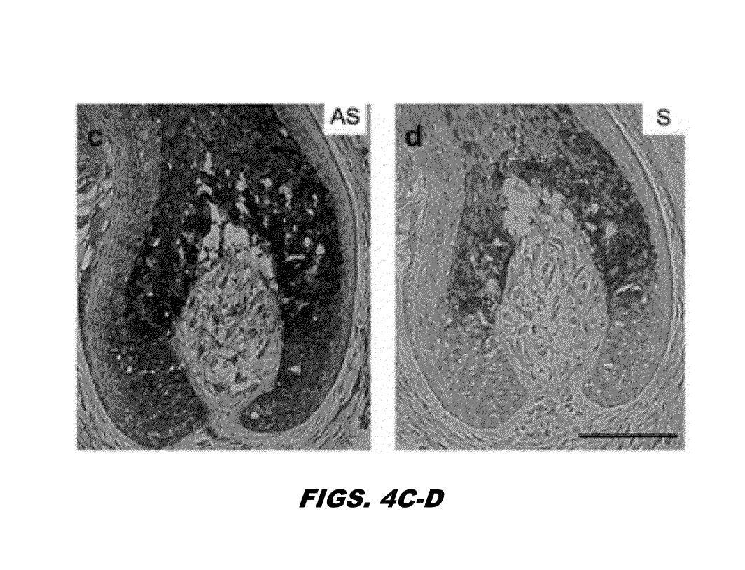

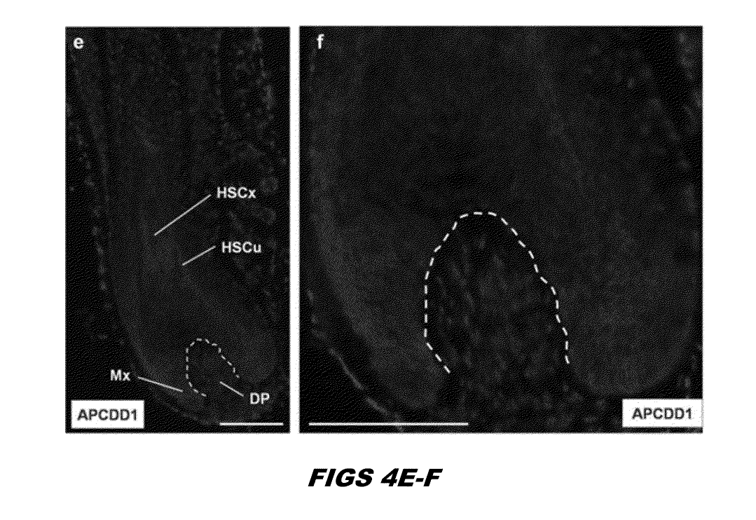

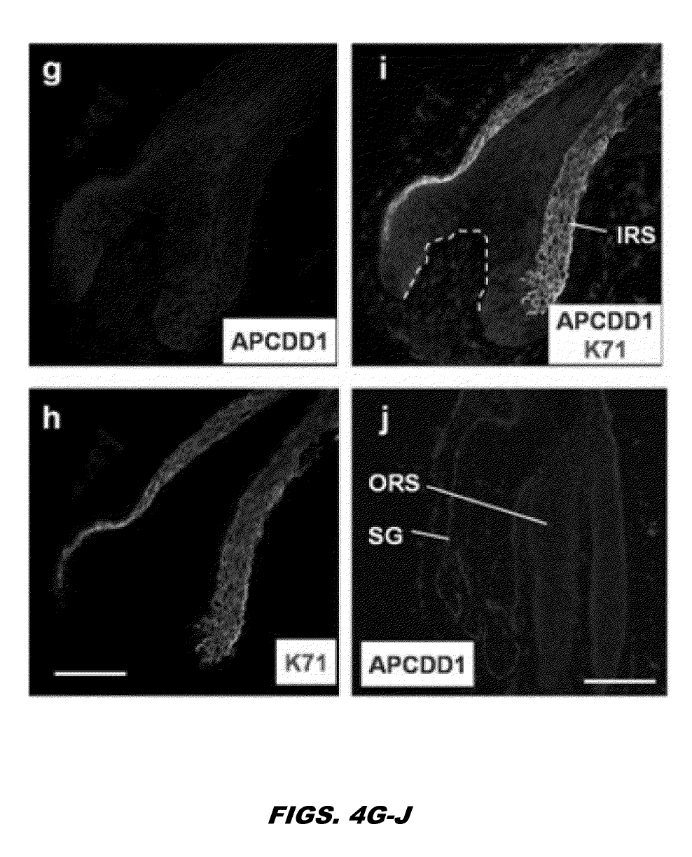

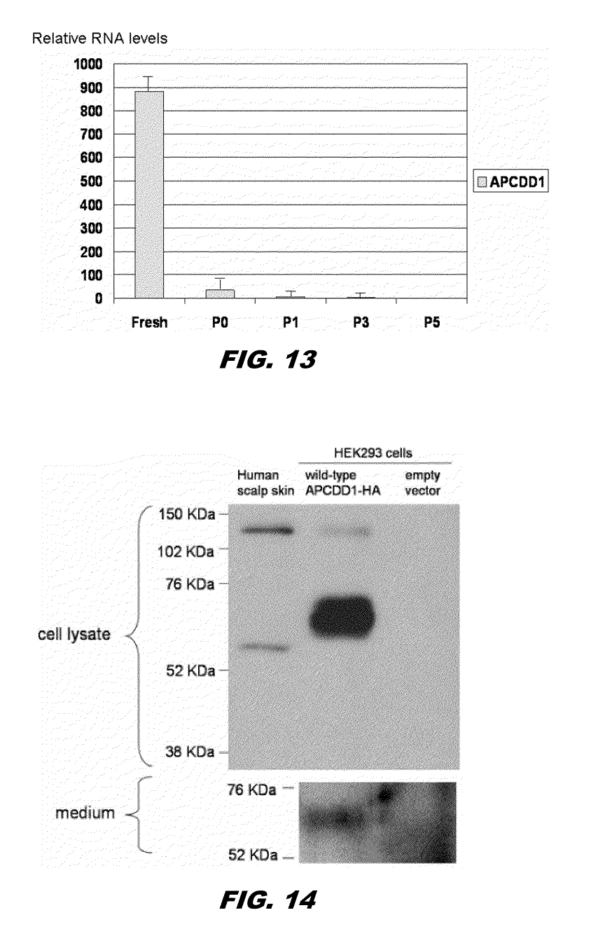

The expression pattern of Apcdd1 (also known as drapc1) in mouse was reported and shows strong expression in the dermal papilla (DP), as well as the matrix region of the adult mouse HFs¹⁴. Consistent with these data, human APCDD1-mRNA was detected in plucked HFs and DP cells by RT-PCR (FIGS. 4A and 4B). DP cells play a crucial role in dermal-epidermal interactions which produce HF, and cultured DP cells on later passages lose their capacity for HF induction¹⁶. Therefore, it is significant to look for genes that are differentially expressed in fresh DP cells. Interestingly, the expression level of the APCDD1 mRNA markedly decreases in cultured DP cells as compared with fresh DP cells (FIG. 4B, FIG. 13). By contrast, the expression of the APCDD1L (see SEQ ID NO: 110), a homologue of APCDD1, is only weakly detected in cultured DP cells, but not in fresh DP cells (FIG. 4B).

These results suggest that the APCDD1 gene could be a key for HF induction. Western blot with the anti-APCDD1 antibody showed two fragments, around 56 KDa and 130 KDa in size, in cell lysate from human scalp skin, which is likely to correspond to a monomer and a dimer of the APCDD1 protein, respectively (FIG. 14). To further implicate APCDD1 in the pathogenesis of HHS, we examined its expression in the human HFs by in situ hybridization and immunofluorescence analysis with the anti-APCDD1 antibody. These studies demonstrate that human APCDD1 is strongly expressed in the DP, the matrix region, the hair shaft, and weakly in the inner root sheath of the HFs (FIGS. 4C-I). In upper portion of the HFs, it is also expressed in the outer root sheath, as well as the sebaceous gland (FIG. 4J). Despite the widespread expression of APCDD1 in many other body sites including heart, pancreas, prostate, and colon¹², we found no evidence of other phenotypes in any affected family members from the three families. The only phenotype observed in affected individuals with the APCDD1 mutation is hypotrichosis. Therefore, we propose a new nomenclature of the APCDD1 gene as “hypotrichin”.

The polypeptide sequence of human APCDD1L is depicted in SEQ ID NO: 110. The nucleotide sequence of human APCDD1L is shown in SEQ ID NO: 111. Sequence information related to APCDD1L is accessible in public databases by GenBank Accession number NM_{—}153360.1.

SEQ ID NO: 110 is the human wild type amino acid sequence corresponding to APCDD1L (residues 1-501):

MPAAMLPYACVLVLLGAHTAPAAGEAGGSCLRWEPHCQQPLPDRVPSTAI
LPPRLNGPWISTGCEVRPGPEFLTRAYTFYPSRLFRAHQFYYEDPFCGEP
AHSLLVKGKVRLRRASWVTRGATEADYHLHKVGIVFHSRRALVDVTGRLN
QTRAGRDCARRLPPARAWLPGALYELRSARAQGDCLEALGLTMHELSLVR
VQRRLQPQPRASPRLVEELYLGDIHTDPAERRHYRPTGYQRPLQSALHHV
QPCPACGLIARSDVHHPPVLPPPLALPLHLGGWWVSSGCEVRPAVLFLTR
LFTFHGHSRSWEGYYHHFSDPACRQPTFTVYAAGRYTRGTPSTRVRGGTE
LVFEVTRAHVTPMDQVTTAMLNFSEPSSCGGAGAWSMGTERDVTATNGCL
PLGIRLPHVEYELFKMEQDPLGQSLLFIGQRPTDGSSPDTPEKRPTSYQA
PLVLCHGEAPDFSRPPQHRPSLQKHPSTGGLHIAPFPLLPLVLGLAFLHW
L

SEQ ID NO: 111 is the human wild type nucleotide sequence corresponding to APCDD1L (nucleotides 1-3112), wherein the underscored ATG denotes the beginning of the open reading frame:

acaactatcaacagccgggaaggctgagcgcgtgtgagcgccgagggggg
cgcaggaccctcgcaacttcttcgcaggactccagcctggccgccggcgc
ccgcagccgtccgagagccctgcgcccgcgcctccccttgcgcaccgtgg
cagcgcccggcgggcggtcctgccagccccgacgggatgcccgcagccat
gctcccctacgcttgcgtcctggtgcttttgggagcccacactgcaccgg
cggctggggaggccgggggcagctgcctgcgctgggaaccccactgccag
cagcccttgccagatagagtgcccagcactgcgatcctgcctccacgcct
taatggaccttggatctccacaggctgcgaggtgcgcccaggaccggagt
tcctgacccgcgcctacaccttctaccccagccggctctttcgagcccac
cagttctactacgaggaccccttctgcggggaacctgcccactcgctgct
cgtcaagggcaaagtccgcctgcgccgggcctcctgggtcacccggggag
ccaccgaggccgactaccacctgcacaaggtgggcatcgtcttccacagc
cgccgggccctggtcgacgtcaccgggcgcctcaaccagacccgcgccgg
ccgggactgcgcgcggcggctgcctccggcccgggcctggctgcctgggg
cgctgtacgagctgcggagcgcccgggctcagggggactgcctggaggcg
ctgggcctcaccatgcacgagctcagcctggtccgcgtgcagcgccgcct
gcagccgcagccccgggcgtcgccccggctggtggaggagctgtacctgg
gggacatccacaccgacccggcggagaggcggcactaccggcccacgggc
taccagcgcccgctgcagagcgcactgcaccacgtgcagccgtgcccagc
ctgtggcctcattgcccgctccgatgtgcaccacccgcccgtgctgccgc
cccctctggccctgcccctgcacctgggcggctggtgggtcagctcgggg
tgcgaggtgcgcccagcagtcctgttcctcacccggctcttcactttcca
cgggcacagccgctcctgggaagggtattaccaccacttctcagacccag
cctgccggcagcccaccttcaccgtgtatgccgccggccgctacaccagg
ggcacgccatccaccagggtccgcggcggcaccgagctggtgtttgaggt
cacacgggcccatgtgacccccatggaccaggtcaccacggccatgctca
acttctctgagccaagcagctgtgggggtgcgggggcctggtccatgggc
actgagcgggatgtcacagccaccaacggctgcctaccgctgggcatccg
gctcccgcatgtggagtacgagcttttcaagatggaacaagaccccctcg
ggcaaagcctgctcttcatcggacaaaggcccaccgatggctcaagtccc
gataccccagagaaacgtcccacctcctaccaagcacccctggtgctctg
tcatggggaggcccccgacttctccaggccaccgcagcacaggccatcgc
tgcagaagcaccccagcacagggggtcttcacatagcccccttcccactt
ctgcccctagttctagggctggccttcctccactggctatgacattggac
ttgacatcaggatggcggctctggacacccattcaacccttcagactccc
tcctggcagctgtagggaaggaaccattctcctctgctctgtcatggatg
gatgcacagccccactgcttccaaactctgcctgtgtcccatgtggctca
ggacatgagcttaacccctgcaaagcctataccacatcccacagcccggg
tccccagtcaagcacttggatgcggcagtgatgttcatcgctacgtgagt
ttctaaagatcactcccaatttttctactttcctcatccttggcagctcg
ccaacaggttcagtcagggggccacacggaacacccccatcccatgttcc
ccccagttcttcccatcctgacccttgggattccaagatgggagcaagag
gagatcctgaggctctgcctagggacgaggcctacagttctgccatgtct
gtaggttgttgtttaaagattattaattcgaatttagcaatacgatctct
aagtggtgccatgaattaaagatgccacttcgggctttcagtgcttctca
gcttttgggcaaagggcttgtgtcttcaggggcagctcagctttcctgag
tcctgactgctggcactcgtctgcatttgcctgtgcttctgcgagtcgga
cctcaagctgccaacactgcatgtggataaatccagttttcccgggccag
catgcaaaatgaagaggactccatctaagctgagaagcatggcctcccca
gagcagcctgcggcctccaagccttcctggcccaggcaaatgccagtgtg
caccaggctggctgctgggggcaggtctttggaggggagcagcatttcca
gccttctgaacatagttaatagtaatgacagccgtaacactaacgcgctc
tgcaattcgccctgcccagccatcctcggttgccaagattgcctgtgcct
gcctgacaaaggaagagaatctccgaatgtgtatctttgggcccacccta
gggagaggtcggggtcaccaggctacatggcgacatctaggcagctccgc
cttgcccagcctccttgccataatcctaatatattggtgtcctctgctca
gaggggactgtcatcatggtgggaacaggctgtgcctccccagggactct
gcccatgttcccagggcctcatctgtacactgtgaaattaactggcatcc
tggtgggcccaagggttttcaggactgggggccaatgactcaccccctcc
ttcctcctcctgatccctatctctagctcttatcacagattttgaacaat
tgtctgtgaggttaatgatggtttcagagggaagcccttttcctccctga
gactgtgtggggttcagtcagcctgctgaaattgcttccacttattaccc
atccttcctctt

In this study, we demonstrated that APCDD1 is a glycoprotein which is secreted outside of the cells (FIG. 3C, FIG. 11A). Overexpression of APCDD1 in colon cancer cells led to enhanced proliferation¹⁶. Our results further suggest a possibility that the secreted APCDD1 could bind to a certain receptor and promote cell growth in vivo. The downstream signaling and developmental pathway affected by APCDD1 remain to be determined.

The mutation L9R identified in this study is located in the signal peptide of APCDD1 protein. Substitution of a leucine residue in the signal peptide has been reported to be pathogenic in several other autosomal dominant diseases, such as familial hypocalciuric hypercalcemia (OMIM 145980)¹⁵ and antithrombin III deficiency (OMIM 107300)¹⁷. In most of these cases, mutations affected the cotranslational processing of the mutant protein^{16, 17}. Consistent with these data, the mutation L9R in APCDD1 results in a marked reduction of the expression and secretion of the mutant protein (FIG. 3C), suggesting that the mutation severely disrupts the structure and the function of the signal peptide of APCDD1. Furthermore, we have observed that the mutant protein, which is retained in ER, also blocks the processing of the wild-type protein in HEK293T cells (FIG. 3C). In addition, the mutant protein markedly represses the expression of wild-type APCDD1 in HEK293T cells (FIG. 12). Although it remains unknown whether the same phenomenon occurs in HFs, our results suggest the possibility that the expression level of APCDD1 in HFs in affected individuals with the mutation L9R can be less than 50% as compared with that in unaffected individuals. Alternatively, since several cases with 18p deletion showed some phenotypes in HFs (FIGS. 2C and 2D)^{13, 18-21}, haploinsufficiency of APCDD1 gene can be enough to affect HF development and hair growth in humans. Since APCDD1 is expressed in many critical organs¹², homozygosity for either the mutation L9R or a complete knockout APCDD1 allele could be lethal.

APCDD1 has previously been shown to be a direct target gene of WNT/β-catenin signaling, based on the evidence that β-catenin/TCF4 complexes directly binds to the APCDD1 promoter and activates its expression¹². Consistent with this data, loss of APCDD1 expression has been reported to be downregulated in Wilms tumor with inactivating mutations in the β-catenin gene²². The involvement of APCDD1 in the development of normal tissues has also been suggested, as the APCDD1 is abundantly expressed in several developing tissues, such as limb buds in mice, as well as carapacial ridge in turtles^{15, 23}. The WNT/β-catenin signaling is known to play crucial roles in HF morphogenesis and development^{24,25}. Our expression studies show that APCDD1 is expressed in the matrix, the hair shaft, and the dermal papilla cells of the human HFs, where β-catenin and the transcription factor LEF1 are abundantly expressed²⁶. It is known that the DP cells secrete a variety of proteins, such as HGF, IGF1, KGF, and α-MSA, which support proliferation and differentiation of the surrounding matrix cells and the hair follicle melanocytes²⁵. In addition to these known proteins, APCDD1 is a key regulator for hair growth which is secreted from the DP cells in vivo.

Chromosome 18p has also been implicated in the genetic etiology of two multifactorial hair diseases. Genome-wide linkage studies for Alopecia Areata²⁷ (AA) and Androgenetic Alopecia²⁸ (AGA) have suggested the presence of disease loci on chromosome 18p. AA is one of the most common causes of hair loss in humans with a lifetime risk of nearly 2%. We performed the first genome-wide linkage study performed for AA and identified several potential loci, including one located on chromosome 18p11.31²⁷. Fine-mapping was performed for this study identified significant linkage (maxLOD=3.93) at 18p11.23 and defined a −2LOD interval that spanned from 3.4 Mb to 12.9 Mb and includes the APCDD1 locus. AGA, also known as male/female pattern baldness, is another highly prevalent complex disease that causes hair loss in humans. A recent genome-wide linkage study identified several loci, one of which is located on chromosome 18 μp11-q23 (max NPL score=2.56), in a region containing APCDD1²⁸. Diminished function of the DP cells is known to play a critical role in miniaturization of the HF typical of AGA²⁹, which is also observed in affected individuals with the APCDD1 mutation. In this study, we have shown that APCDD1 is a secreted protein abundantly expressed in the DP cells in vivo, and whose expression is lost upon explant culture, when the HF inductive properties of the DP also decline. Targeting APCDD1 could represent a new therapeutic modality not only for HHS, but potentially also for more common forms of hair loss.

Methods

Clinical details and DNA extraction. Informed consent was obtained from all subjects and approval for this study was provided by the Institutional Review Board of Columbia University and. The study was conducted in adherence to the Declaration of Helsinki Principles. Peripheral blood samples were collected from the family members as well as unrelated healthy control individuals of Pakistani and European origin (100 individuals each). Genomic DNA was isolated from these samples according to standard techniques.

Linkage Analysis. Genome-wide genotyping was performed with the Affymetrix Human Mapping 10K 2.0 Array. Quality control and data analysis was performed with Genespring GT (Agilent software). Briefly, SNPs that violated Mendelian inheritance pattern were removed from the data set prior to analysis. Haplotypes were inferred from raw genotype data. By analyzing haplotypes rather than individual SNPs, Type I error introduced by linkage disequilibrium between markers is mitigated. Finally, haplotypes were analyzed for linkage under the assumption of a fully penetrant disease gene with a frequency of 0.001 transmitted by a dominant mode of inheritance.

Mutation Analysis. Using the genomic DNA of the family members, all exons and exon-intron boundaries of APCDD1 gene were amplified by PCR with the gene-specific primers (Table 4). The PCR products were directly sequenced in an ABI Prism 310 Automated Sequencer, using the ABI Prism Big Dye Terminator Cycle Sequencing Ready Reaction Kit (PE Applied Biosystems). The mutation 26T>G disrupts a DdeI restriction enzyme site, which was used to screen the family members and control individuals.

TABLE 4
Primers used in the Study

| | | | | | | | |
|---|---|---|---|---|---|---|---|
| | | | | | | Anneal- | |
| | | | | | | ing | prod- |
| | | | Seq | | Seq | temper- | uct |
| primer | | | ID | | ID | ature | size |
| name | position (bp) | forward primer (5′ to 3′) | NO | reverse primer (5′ to 3′) | NO | (C.°) | (bp) |

Primers to amplify additional microsatellite markers

| | | | | | | | |
|---|---|---|---|---|---|---|---|
| RAB31-MS | 9,847,609-9,847,756 | GCAAATGAATACACTAACTAGCCA | 18 | CACCAGGCCTAGGCTAAAATG | 64 | 55 | 148 |
| APCDD1- | 10,454,280-10,454,403 | CCAGTGAGACCTTAAAAGCTTCA | 19 | TTGCTATACTTTAGGAGCCAGAA | 65 | 55 | 124 |
| MS | | | | | | | |
| GNAL-MS | 11,693,739-11,693,854 | GGCTTGGGTACAATAATGAGCT | 20 | CAGAGCTTCCTGCACTTCAC | 66 | 55 | 116 |

Primers to amplify candidate genes from genomic DNA

| | | | | | | |
|---|---|---|---|---|---|---|
| APCDD1 | CGACGCGCCCTTTCAAGTCT | 21 | CGCCAAGGGGACAGTGTAG | 67 | 61 | 668 |
| ex1 | | | | | | |
| APCDD1 | CACTGTCTGCTGCAGAGGT | 22 | GGTCTTCCATAGTGGTGTGC | 68 | 56 | 328 |
| ex2 | | | | | | |
| APCDD1 | GAATCTCTTTCCCATACCTTCAG | 23 | TGGCAAGCCTTTAAGAAGGATC | 69 | 56 | 659 |
| ex3 | | | | | | |
| APCDD1 | CGGAAGCATGTGTGCACTG | 24 | AAGAGGTCCTTTCCCTCAGC | 70 | 56 | 481 |
| ex4 | | | | | | |
| APCDD1 | GTCTAGTTAGAGTGTGGCCAG | 25 | TTCTCTGGCATTCAAGTGCATC | 71 | 56 | 651 |
| ex5 | | | | | | |
| RAB31 | CAAAGCAGGAGTGTTGAAGCA | 26 | TGTGCTCACTCTTCTCAAAGTG | 72 | 55 | 302 |
| ex6 | | | | | | |
| TXNDC2 | GAGGGAAAACCAACTGTAACGT | 27 | CTACAACTTTCCCTTCTTGGTTC | 73 | 55 | 323 |
| ex1 | | | | | | |
| TXNDC2 | CAGACTTCATTCGTGATCTCGA | 28 | TACAAGCAGTGCCATTTGGACAT | 74 | 59 | 1,802 |
| ex2 | | | | | | |
| VAPA ex1 | AGCCTGGCCTCGTCCTAGA | 29 | AAAGCAGCCGGGAAAGGGAA | 75 | 55 | 335 |
| VAPA ex2 | GAAAGGCAGTGTTAGTAGCCAT | 30 | TATTTCCCTCCCTCCAGATG | 76 | 55 | 339 |
| VAPA ex3 | GTTTAAGGCAAATCCCAGACTT | 31 | ACTGCTAAACAACACCCACAGT | 77 | 55 | 275 |
| VAPA ex4 | GTCCCGTGAGGTGAAACTTA | 32 | GGAACTGAGAGCTCAACAGC | 78 | 55 | 203 |
| VAPA ex5 | CAGTCATTCCCAATATCATGCAG | 33 | CACAATACCACTTATCTGCTAGG | 79 | 55 | 299 |
| VAPA ex6 | ATCTGACTGCTGACATGTACTG | 34 | TGGCCTAATAACACTCACATGTC | 80 | 55 | 383 |
| VAPA ex7 | CACTAATCTACTTTCCGTCCCTA | 35 | CGTGGGCCATACTACCAATG | 81 | 55 | 350 |
| NAPG ex1 | TGTCGCGCTGCACCAGCTT | 36 | GGAGTGTGACCCAGAGGAC | 82 | 55 | 337 |
| NAPG ex2 | GACCTAGAAGGTCATTACAAGC | 37 | GCCTTATAGAAAGCATATGAGTAAGT | 83 | 55 | 249 |
| NAPG ex3 | CTGATTTGTTGCCAGTAGTCAAC | 38 | GCGAACCAGTGTGGACCTTA | 84 | 55 | 527 |
| NAPG ex4 | TGGAACCAACTGGGTCAGTG | 39 | TTACAGTTAGGAGTAAGTCTTGGT | 85 | 55 | 306 |
| NAPG ex5 | TATGTTGTGCATGAGCCCATTG | 40 | CTTGCCTTACAGTAAGGAAGCT | 86 | 55 | 261 |
| NAPG | CTGCACATGTGTCCCAGAAC | 41 | TGGTGTGTCTACAGCTTATACC | 87 | 55 | 869 |
| ex6-8 | | | | | | |
| NAPG ex9 | AAGTACATTTCACAGGGGACCT | 42 | AAGCTGTAGTGGGCTTACTCTA | 88 | 55 | 318 |
| NAPG | TCCCGTGGAAGTTACTATAGCA | 43 | GGTGAAATTCAATGCAAGTGGTC | 89 | 55 | 1,109 |
| ex10-11 | | | | | | |
| NAPG | CCACCATGGTCATGAGGCAT | 44 | GGATATCCCTAATCTATTCCCAAG | 90 | 55 | 405 |
| ex12 | | | | | | |
| FAM38B | GCTCCACCACCCATCTTATG | 45 | CTGCCTTACGCAGATAACATTTG | 91 | 55 | 299 |
| ex1 | | | | | | |
| FAM38B | GAAGGTGGGACCTGACTGGA | 46 | CGTTGACTATTGACCCAAACCT | 92 | 58 | 753 |
| ex2-3 | | | | | | |
| FAM38B | GTTGGAATGATGAAGGGGAAATTC | 47 | GTTCCACAACGATTCCCACTG | 93 | 55 | 395 |
| ex4 | | | | | | |
| FAM38B | CAGTTAAGGAACTGCTTGAGCT | 48 | TGCCTTCATTGTGAGCAGAAGT | 94 | 55 | 331 |
| ex5 | | | | | | |
| FAM38B | CTCAGGATGCAGAGAAGAGC | 49 | TTCCCACCACCTCAGGCCAT | 95 | 55 | 250 |
| ex6 | | | | | | |
| FAM38B | AGTGCAATGGAACACTCAAGAC | 50 | CCCAACTCCATGTTTAGACTGG | 96 | 55 | 389 |
| ex7 | | | | | | |
| FAM38B | TGACCGTTGTGGACCCAATG | 51 | GAAGTTGCATTCCTCATACACATG | 97 | 55 | 351 |
| ex8 | | | | | | |
| FAM38B | GAGAGTTTTGACTGTAATAGGAAC | 52 | TGTCACACAGGGAGAGATAACT | 98 | 55 | 371 |
| ex9 | | | | | | |
| FAM38B | TTAGTAACCAGATTTTGCCATCTG | 53 | CCCACTCTCAACTTTACATGATAC | 99 | 55 | 345 |
| ex10 | | | | | | |
| FAM38B | CTTCTACAGAATTTGAGGGAAAGT | 54 | TCCGTCGAACTAGAAATGCTTAG | 100 | 55 | 455 |
| ex11 | | | | | | |
| AMAC1L1 | CCACCTCAGGATAGTTCCAG | 55 | CCTCTGAATGGTTAGGTCCTTA | 101 | 55 | 1239 |
| GNAL ex1 | CTGGGCGTTAGCAAGTGATC | 56 | CCCACAGTTTAAACGCTCTGTA | 102 | 55 | 843 |

Primers used in RT-PCR experiments

| | | | | | | |
|---|---|---|---|---|---|---|
| APCDD1- | ATGCCACCCAGAGGATGTTC | 57 | GATGGTCAGGTCTGCCTTTG | 103 | 60 | 155 |
| rtpcr | | | | | | |
| KRT15- | GGGTTTTGGTGGTGGCTTTG | 58 | TCGTGGTTCTTCTTCAGGTAGGC | 104 | 60 | 474 |
| rtpcr | | | | | | |
| B2M- | CACAGCCCAAGATAGTTAAGTG | 59 | GCATAAAGTGTAAGTGTATAAGCATA | 105 | 60 | 143 |
| rtpcr | | | | | | |
| APCDD1L- | GTGGAGGAGCTGTACCTGG | 60 | GGCAATGAGGCCACAGGCT | 106 | 60 | 135 |
| rtpcr | | | | | | |
| CORIN- | GGCTGTGTCCTCATTGCCAA | 61 | GTCTTCACAAAGCGTGTCTGC | 107 | 60 | 146 |
| rtpcr | | | | | | |
| α-SMA- | CCGACCGAATGCAGAAGGA | 62 | ACAGAGTATTTGCGCTCCGAA | 108 | 60 | 88 |
| rtpcr | | | | | | |

Primers used to make the probes for in situ hybridization

| | | | | | | |
|---|---|---|---|---|---|---|
| APCDD1- | AGGACCTCGCAGAAGACAGT | 63 | GTGTCTTGATCACAGTCCCAA | 109 | 55 | 613 |
| ISH | | | | | | |

Exon 1 and adjacent boundary sequences of the APCDD1 gene were amplified using Platinum® Taq DNA Polymerase High Fidelity (Invitrogen). Due to the high G/C content, DMSO (final 5%) and MgSO₄ (final 1.6 mM) were added to the PCR reaction. Amplification conditions were 94° C. for 2 min, followed by 35 cycles of 94° C. for 30 sec, 61° C. for 30 sec, and 68° C. for 50 sec, with a final extension at 68° C. for 7 min. Other exons, as well as the exon-intron boundaries of the APCDD1 gene, were amplified using Platinum® PCR SuperMix (Invitrogen). Amplification conditions were 94° C. for 2 min, followed by 35 cycles of 94° C. for 30 sec, 56° C. for 30 sec, and 72° C. for 50 sec, with a final extension at 72° C. for 7 min.

To screen for the mutation 26T>G (L9R), a part of exon 1 and intron 1 of the APCDD1 gene was amplified by PCR using Platinum® Taq DNA Polymerase High Fidelity (Invitrogen) and the following primers: forward (5′-CCAGAGCAGGACTGGAAATG-3′; SEQ ID NO: 7), reverse (5′-CGCCAAGGGGACAGTGTAG-3′; SEQ ID NO: 8). DMSO (final 5%) and MgSO₄ (final 1.6 mM) were added to the PCR reaction. The amplification conditions were 94° C. for 2 min, followed by 35 cycles of 94° C. for 30 sec, 59° C. for 30 sec, and 68° C. for 30 sec, with a final extension at 68° C. for 7 min. The amplified PCR products, 191 bp in size, were digested with DdeI at 37° C. overnight, and run on 2.0% agarose gels.

Analysis of copy number of APCDD1 gene. Using the genomic DNA from the affected individual with 18p deletion and a control individual, copy number of APCDD1 gene was analyzed by real-time PCR on an ABI 7300 (Applied Biosystems). PCR reactions were performed using ABI SYBR Green PCR Master Mix, 300 nM primers, 50 ng genomic DNA at the following consecutive steps: (a) 50° C. for 2 min, (b) 95° C. for 10 min, (c) 40 cycles of 95° C. for 15 sec and 60° C. for 1 min. Using the accompanying software, the samples were normalized to GAPDH gene which resides on human chromosome 12p. The following primers were used: APCDD1 (forward 5′-GTCTAGTTAGAGTGTGGCCAG-3′[SEQ ID NO: 9], reverse 5′-GATGGTCAGGTCTGCCTTTG-3′ [SEQ ID NO: 10]), GAPDH (forward 5′-ATGGACA CGCTCCCCTGACT-3′[SEQ ID NO: 11], reverse 5′-GAAAGGTGGGAGC CTCAGTC-3′ [SEQ ID NO: 12]).

Cell culture. HEK293T (human embryonic kidney) cells were cultured in Dulbecco's modified Eagle's medium (DMEM; GIBCO) supplemented with 10% fetal bovine serum (FBS; GIBCO), 100 IU/ml penicillin, and 100 μg/ml streptomycin. To establish primary dermal papilla cultures intact human dermal papillae were isolated from scalp tissue and dissected using tungsten needles in sterile DMEM as previously described³⁰. Dermal papilla cells were cultured in DMEM supplemented with 10% FBS, 100 IU/ml penicillin, and 100 μg/ml streptomycin.

RT-PCR. Total RNA were isolated from 10 plucked human scalp hairs of a healthy control individual, as well as fresh and cultured dermal papilla (DP) cells (passages 0, 1, 3, and 5) using the RNeasy® Minikit (Quiagen). 1 μg of total RNA was reverse-transcribed with oligo-dT primers and SuperScript™ III (Invitrogen). The cDNAs from the plucked hairs were amplified by PCR using Platinum® PCR SuperMix and primer pairs for APCDD1, keratin 15 (KRT15), and beta-2 microgloblin (B2M) genes (Table 4). Primers for the KRT15 gene were designed as described previously¹¹. The amplification conditions were 94° C. for 2 min, followed by 35 cycles of 94° C. for 30 sec, 60° C. for 30 sec and 72° C. for 30 sec, with a final extension at 72° C. for 7 min. PCR products were run on 1.5% agarose gels.

Using the cDNAs from fresh and cultured DP cells, semiquantitative RT-PCR was performed with primers for APCDD1, APCDD1L, CORIN, alpha-smooth muscle actin (αSMA), and B2M (Table 4). Primers for the αSMA gene were designed as described previously³¹. CORIN is a DP-specific marker, and its expression has been shown to decrease in cultured DP cells³². αSMA is another control, of which expression is low in fresh DP cells and high in cultured mouse DP cells³³. The different cDNAs were equalized using B2M primers as an internal control. Aliquots of each PCR reaction were taken at 26, 29, 32, and 35 cycles, and analyzed on 1.5% agarose gels. Using the same cDNAs, the expression levels of APCDD1 gene were measured by real-time PCR on an ABI 7300 (Applied Biosystems). PCR reactions were performed using ABI SYBR Green PCR Master Mix, 300 nM primers, 20 ng cDNA at the following consecutive steps: (a) 50° C. for 2 min, (b) 95° C. for 10 min, (c) 40 cycles of 95° C. for 15 sec and 60° C. for 1 min. The samples were run in triplicate and normalized to an internal control (B2M) using the accompanying software.

APCDD1-expression vectors. To generate the expression construct for the C-terminal hemagglutin (HA)-tagged human APCDD1, the full length APCDD1 cDNA sequences were amplified by PCR using the first strand cDNA from plucked human hairs as a template and the following primers: forward (5′-AAAACTCGAGCCAGAGCAGGACTG GAAATG-3′ [SEQ ID NO: 13]), reverse (5′-AAAAGCTAGCTCAGGCGTAGTCGGGC ACGTCGTAGGGGTATCTGCGGATGTTCCAATGC-3′ [SEQ ID NO: 14]). To generate the c-myc-tagged wild-type APCDD1 expression construct, the following reverse primer was used: (5′-AAAAGCTAGCTACAGATCCTCTTCAGAGATGAGTTTCTGCTCTC TGCGGATGTTCCAATGC-3′ [SEQ ID NO: 15]). The amplified products were subcloned into the XhoI and NheI sites of the mammalian expression vector pCXN2.1³⁴, a slightly modified version of pCXN2³⁵ with multiple cloning sites. L9R and L9V mutant APCDD1 sequences were PCR-amplified using the HA-tagged-wild-type APCDD1 construct as a template and the following forward primers: L9R-F (5′-AAAACTCGAGCCAGAGCAGGA CTGGAAATGTCCTGGCCGCGCCGCCTCCTGCGCAGAT-3′ [SEQ ID NO: 16]), L9V-F (5′-AAAACTCGAGCCAGAGCAGGACTGGAAATGTCCTGGCCGCGCCGCCTCC

TGGTCAGAT-3′ [SEQ ID NO: 17]). Note that T>G and C>G substitutions were introduced into the primers, respectively (shown in bold and underlined). The reverse primer was the same as used in generating the HA-tagged-wild-type APCDD1 construct. The amplified products were subcloned into the XhoI and NheI sites of the pCXN2.1.

Transient transfections and western blots. HEK293T cells were plated in 60 mm dishes the day before transfection. Expression plasmids of APCDD1 were transfected with Lipofectamine™ 2000 (Invitrogen) at 60% confluency. Total amount of transfected plasmids were adjusted with the empty pCXN2.1 vector. The cells were cultured 24 h after transfection in DMEM with 10% FBS, and the medium was changed to DMEM without FBS. 48 h after the medium change, the cells were harvested and homogenized by sonication in 25 mM HEPES-NaOH (pH 7.4), 10 mM MgCl2, 250 mM Sucrose, and 1× Complete Mini Protease Inhibitor Cocktail (Roche Applied Science). The cell debris was removed by centrifugation at 3,000 rpm for 10 min at 4° C., and the supernatant was collected as total cell lysates. The cultured medium with 1× Complete Mini Protease Inhibitor Cocktail was centrifuged at 1,500 rpm for 5 min at 4° C. The supernatant was purified with 0.2 μm syringe filters (Thermo Fisher Science), and concentrated using Amicon Ultra-15 Centrifugal Filter Unit with Ultracel-10 Membrane (Millipore) according to the manufacturer's recommendations. To examine the N-Glycosylation of APCDD1, the total cell lysates from the wild-type APCDD1 expressing cells were treated with PNGase F (Sigma) following the manufacturer's recommendations. Total cell lysates from human scalp skin were extracted by homogenization in 50 mM Tris-HCl (pH 8.0), 150 mM NaCl, 1.0% NP40, 0.5% sodium deoxycholate, 0.1% SDS, and 1× Complete Mini Protease Inhibitor Cocktail. All samples were mixed with equal amount of Laemmli Sample Buffer (Bio-Rad Laboratories) containing 5% β-mercaptoethanol, boiled at 95° C. for 5 min, and analyzed by 10% SDS-polyacrylamide gel electrophoresis (SDS-PAGE). Western blots were performed as described previously³⁶. The primary antibodies used were rat monoclonal anti-HA 3F10 (diluted 1:1,000; Roche Applied Science), mouse polyclonal anti-APCDD1 (1:1,000; Abnova) and rabbit polyclonal anti-β-actin (1:10,000; Sigma).

Immunoprecipitation. Expression plasmids of HA-tagged APCDD1 and c-myc-tagged APCDD1 (4 μg each) were co-transfected into HEK293T cells as described above. 48 h after the transfection, the cells were harvested and homogenized in lysis buffer (50 mM Tris-HCl (pH 8.0), 150 mM NaCl, 0.5% NP40, and 1× Complete Mini Protease Inhibitor Cocktail). Total cell lysates were collected by centrifugation at 14,000 rpm for 20 min at 4° C. The samples were incubated with 0.3 μg of normal rabbit IgG (Santa Cruz Biotechnology) and 10 μl of protein A/G plus agarose (Santa Cruz) for 30 min at 4° C., and centrifuged at 10,000 rpm for 1 min at 4° C. The supernatants were incubated with 1.0 μg of either rabbit polyclonal anti-c-myc antibody (Santa Cruz) or normal rabbit IgG for overnight at 4° C. Then, 20 μl of Protein A/G Plus Agarose was added into the samples, and incubated for 2 h at 4° C. The agarose beads were washed with lysis buffer for five times. The precipitated proteins were eluted with Laemmli Sample Buffer containing 5% β-mercaptoethanol, boiled at 95° C. for 5 min, and separated by 10% SDS-PAGE. Western blots were performed using rat monoclonal anti-HA 3F10 (diluted 1:1,000).

In situ hybridization. A part of the human APCDD1 cDNA (GenBank Accession number, NM_{—}153000: nt. 1775-2387) was cloned into pCR®II-TOPO vector (Invitrogen). The antisense and sense DIG-labeled cRNA probes were synthesized from the linearized vectors with T7 and SP6 RNA polymerases (Roche Applied Science), respectively. Dissected human hair follicles were fixed with 4% paraformaldehyde-PBS at 4° C. overnight. After dehydration step with 30% sucrose-PBS, the tissues were frozen in OCT compound and sectioned on glass slides at the thickness of 7 μm. In situ hybridization was performed following the methods described previously with minor modifications³⁷. At the prehybridization steps, the sections were treated with 1 μg/ml Protease K for 10 min at 37° C. Hybridization was performed at 58° C. overnight.

Indirect immunofluorescence (IIF). IIF on cultured cells and fresh frozen sections of individually dissected hair follicles was performed as described previously³⁶. IIF on HEK293T cells were performed 48 h after the expression constructs of HA-tagged APCDD1 were transfected. The primary antibodies used were mouse polyclonal anti-APCDD1 (diluted 1:1,000; Abnova), rat monoclonal anti-HA 3F10 (1:1,000), and rabbit polyclonal anti-K71 (1:10,000)³⁷.

### REFERENCES

1. Toribio, J. & Quiñones, P. A. Hereditary hypotrichosis simplex of the scalp. Evidence for autosomal dominant inheritance. Br. J. Dermatol. 91, 687-696 (1974).
2. Bentley-Philips, B. & Grace, H. J. Hereditary hypotrichosis. A previously undescribed syndrome. Br. J. Dermatol. 101, 331-339 (1979).
3. Baumer, A., Belli, S., Trüeb, R. M. & Schinzel, A. An autosomal dominant form of hereditary hypotrichosis simplex maps to 18p11.32-p11.23 in an Italian family. Eur. J. Hum. Genet. 8, 443-448 (2000).
4. Schmidt-Ullrich, R. & Paus, R. Molecular principles of hair follicle induction and morphogenesis. Bioessays 27, 247-261 (2005).
5. van Steensel, M. et al. The gene for hypotrichosis of Marie Unna maps between D8S258 and D8S298: exclusion of the hr gene by cDNA and genomic sequencing. Am. J. Hum. Genet. 65, 413-419 (1999).
6. Winter, H. et al. Mutations in the hair cortex keratin hHb6 cause the inherited hair disease monilethrix. Nat. Genet. 16, 372-374 (1997).
7. van Steensel, M. A., Steijlen, P. M., Bladergroen, R. S., Vermeer, M. & van Geel, M. A missense mutation in the type II hair keratin hHb3 is associated with monilethrix. J. Med. Genet. 42, e19 (2005).
8. Kljuic, A. et al. Desmoglein 4 in hair follicle differentiation and epidermal adhesion: evidence from inherited hypotrichosis and acquired pemphigus vulgaris. Cell 113, 249-260 (2003).
9. LinksIbsen, H. H., Clemmensen, O. J. & Brandrup, F. Familial hypotrichosis of the scalp. Autosomal dominant inheritance in four generations. Acta. Derm. Venereol. 71, 349-351 (1991).
10. Levy-Nissenbaum, E. et al. Hypotrichosis simplex of the scalp is associated with nonsense mutations in CDSN encoding corneodesmosin. Nat. Genet. 34, 151-153 (2003).
11. Pasternack, S. M. et al. G protein-coupled receptor P2Y5 and its ligand LPA are involved in maintenance of human hair growth. Nat. Genet. 40, 329-334 (2008).
12. Takahashi, M. et al. Isolation of a novel human gene, APCDD1, as a direct target of the beta-Catenin/T-cell factor 4 complex with probable involvement in colorectal carcinogenesis. Cancer Res. 62, 5651-5656 (2002).
13. Kantaputra, P. N. et al. Contiguous gene syndrome of holoprosencephaly and hypotrichosis simplex: association with an 18p11.3 deletion. Am. J. Med. Genet. A. 140, 2598-2602 (2006).
14. Jukkola, T., Sinjushina, N. & Partanen, J. Drapc1 expression during mouse embryonic development. Gene. Expr. Patterns. 4, 755-762 (2004).
15. Pidasheva, S., Canaff, L., Simonds, W. F., Marx, S. J. & Hendy, G. N. Impaired cotranslational processing of the calcium-sensing receptor due to signal peptide missense mutations in familial hypocalciuric hypercalcemia. Hum. Mol. Genet. 14, 1679-1690 (2005).
16. Jahoda, C. A., Reynolds, A. J. & Oliver, R. F. Induction of hair growth in ear wounds by cultured dermal papilla cells. J. Invest. Dermatol. 101, 584-590 (1993).
17. Fitches, A. C. et al. Impaired cotranslational processing as a mechanism for type I antithrombin deficiency. Blood 92, 4671-4676 (1998).
18. Uchida, I. A., McRae, K. N., Wang, H. C. & Ray, M. Familial short arm deficiency of chromosome 18 concomitant with arhinencephaly and alopecia congenital. Am. J. Hum. Genet. 17, 410-419, 1965.
19. Jacobsen, P. & Mikkelsen, M. The 18p-syndrome. Report of two cases. Ann. Genet. 11, 211-216, 1968.
20. Zouboulis, C. C. et al. Ulerythema ophryogenes and keratosis pilaris in a child with monosomy 18p. Pediatr. Dermatol. 11, 172-175 (1994).
21. Nazarenko, S. A. et al. Keratosis pilaris and ulerythema ophryogenes associated with an 18p deletion caused by a Y/18 translocation. Am. J. Med. Genet. 85, 179-182 (1999).
22. Zirn, B. et al. Target genes of the WNT/beta-catenin pathway in Wilms tumors. Genes Chromosomes Cancer 45, 565-574 (2006).
23. Kuraku, S., Usuda, R. & Kuratani, S. Comprehensive survey of carapacial ridge-specific genes in turtle implies co-option of some regulatory genes in carapace evolution. Evol Dev. 7, 3-17 (2005).
24. DasGupta, R. & Fuchs E. Multiple roles for activated LEF/TCF transcription complexes during hair follicle development and differentiation. Development 126, 4557-4568 (1999).
25. Botchkarev, V. A. & Paus R. Molecular biology of hair morphogenesis: development and cycling. J. Exp. Zoolog. B Mol. Dev. Evol. 298, 164-180 (2003).
26. Cribier, B., Peltre, B., Grosshans, E., Langbein, L. & Schweizer, J. On the regulation of hair keratin expression: lessons from studies in pilomatricomas. J. Invest. Dermatol. 122, 1078-1083 (2004).
27. Martinez-Mir, A. et al., Genomewide scan for linkage reveals evidence of several susceptibility loci for alopecia greata. Am. J. Hum. Genet. 80, 316-328 (2007).
28. Hillmer, A. M. et al. Genome-wide scan and fine-mapping linkage study of androgenetic alopecia reveals a locus on chromosome 3q26. Am. J. Hum. Genet. 82, 737-743 (2008).
29. Trüleb, R. M. Molecular mechanisms of androgenic alopecia. Exp. Gerontol. 37, 981-990 (2002).
30. Messenger, A. G. The culture of dermal papilla cells from human hair follicles. Br. J. Dermatol. 110, 685-689 (1984).
31. Knerr, I. et al. Alteration of neuronal and endothelial nitric oxide synthase and neuropeptide Y in congenital ureteropelvic junction obstruction. Urol. Res. 29, 134-140 (2001).
32. Enshell-Seijffers, D., Lindon, C. & Morgan, B. A. The serine protease Corin is a novel modifier of the Agouti pathway. Development 135, 217-225 (2008).
33. Jahoda, C. A., Reynolds, A. J., Chaponnier, C., Forester, J. C. & Gabbiani, G. Smooth muscle alpha-actin is a marker for hair follicle dermis in vivo and in vitro. J. Cell Sci. 99, 627-636 (1991).
34. Noguchi, K., Ishii, S. & Shimizu, H. Identification of p2y9/GPR23 as a novel G protein-coupled receptor for lysophosphatidic acid, structurally distant from the Edg family. J. Biol. Chem. 278, 25600-25606 (2003).
35. Niwa, H., Yamamura, K. & Miyazaki, J. Efficient selection for high-expression transfectants with a novel eukaryotic vector. Gene 108, 193-199 (1991).
36. Bazzi, H. et al. Desmoglein 4 is expressed in highly differentiated keratinocytes and trichocytes in human epidermis and hair follicle. Differentiation 74, 129-140 (2006).
37. Aoki, N. et al. A novel type II cytokeratin, mK6irs, is expressed in the Huxley and Henle layers of the mouse inner root sheath. J. Invest. Dermatol. 116, 359-365 (2001).

### Example 2

### Swamp is an Inhibitor of the Wnt/β-Catenin Pathway in which Mutations Underlie Hereditary Hypotrichosis Simplex

Using genetic linkage analysis, we identified a mutation (L9R) in the APCDD1 gene, which we now rename SWAMP, on chromosome 18p11.22 of two Pakistani families with HHS. SWAMP is expressed in the dermal papilla, the matrix, and the hair shaft of human HFs. It is a membrane-bound glycoprotein that can interact with WNT3A and LRP5, two essential components of the Wnt/β-catenin signaling. Functional studies in cell lines, revealed that SWAMP inhibits Wnt signaling in a cell autonomous manner and functions upstream of β-catenin. SWAMP inhibits the activation of the Wnt/β-catenin pathway in HEK293T cells transfected with WNT3A, LRP5 and Fzd2. The mutation L9R, localized in the signal peptide of the SWAMP protein, perturbs its translational processing from ER to the plasma membrane. In addition, L9R SWAMP functions in a dominant-negative manner to inhibit the stability and membrane localization of the wild type protein, thus impairing its normal function in HHS patients. These findings uncover an inhibitor of the Wnt/β-catenin signaling pathway with an essential role in human hair growth. Since SWAMP is expressed in many other cells^{A3}, our findings suggest that SWAMP is a regulator of many biological processes controlled by the Wnt/β-catenin signaling.

We performed a genetic linkage study in two large Pakistani families (HHS1 and HHS2) with autosomal dominant HHS (FIG. 1 and FIG. 5). We used human mapping arrays with low density (Affymetrix 10K) to genotype 16 and 12 members of each family, respectively. Parametric linkage analysis performed under a dominant model yielded a maximum LOD score of z=4.6 for a haplotype on chromosome 18p11.22 (FIG. 1G). The 2LOD interval spanned from 7.4 Mb to 25 Mb. Genotyping with microsatellite markers enabled us to define the candidate region to 1.8 Mb between the markers RAB31-MS and GNAL-MS (FIG. 1H and FIG. 2B, bottom panel, and FIG. 6), which contained 8 known genes, 4 pseudogenes and 3 predicted transcripts (FIG. 2A).

Direct sequencing analysis of all known genes in the region identified a single heterozygous mutation 26T>G (L9R) in the signal peptide sequence of the Adenomatosis Polyposis Coli Down-regulated 1 (APCDD1) gene (FIG. 2B), described initially as being downregulated by the tumor suppressor APC^{A8}. Based on our functional studies, we hereafter designate the gene as SWAMP (cell Surface-localized Wnt Antagonist Mutated in hyPotrichosis). The mutation L9R cosegregates with the disease phenotype in both families, was absent in 200 unrelated healthy control Pakistani individuals and in the SNP databases, arguing against it being a polymorphism (FIG. 1H and FIG. 2B, bottom panel, and FIG. 6). In addition, surprisingly, we have also identified the identical heterozygous mutation 26T>G (L9R) in the SWAMP gene in an Italian family with autosomal dominant HHS that we reported previously (FIG. 7, FIG. 8)^{A2}. Interestingly, several other autosomal dominant diseases, such as familial hypocalciuric hypercalcemia (OMIM 145980)^{A9} and antithrombin III deficiency^{A10} are also known to be caused by substitutions in a leucine residue in the signal peptide.

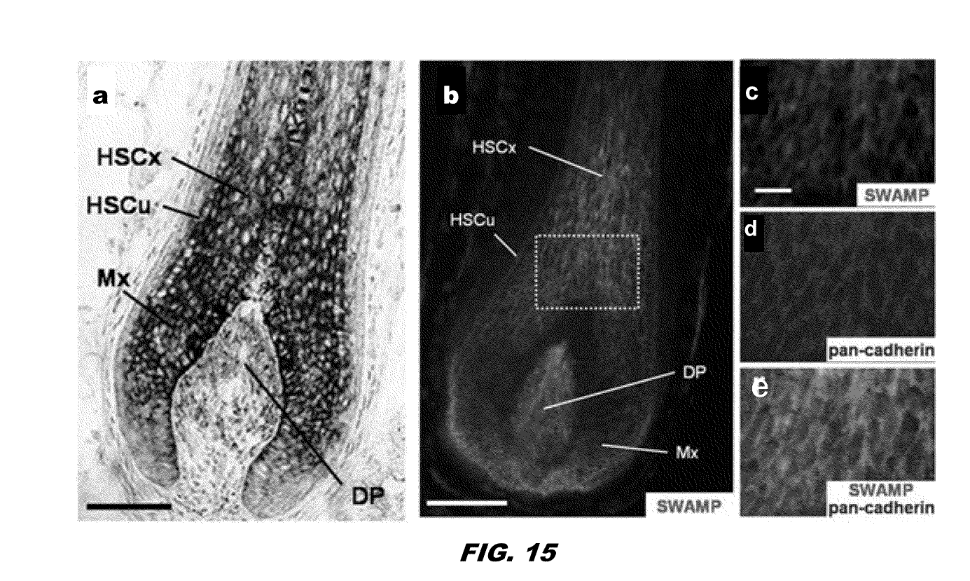

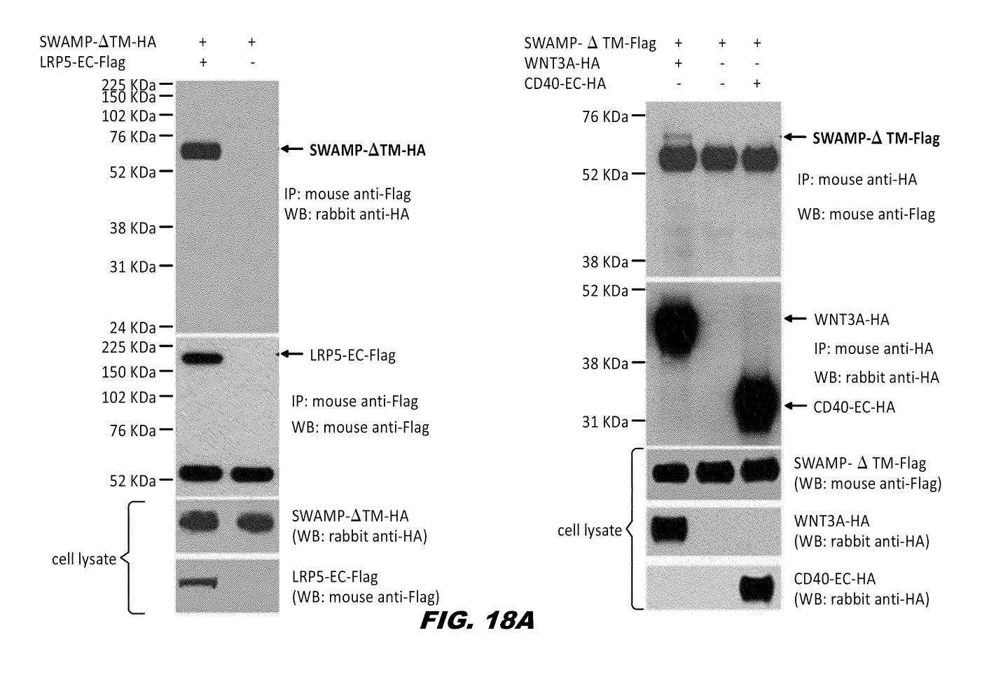

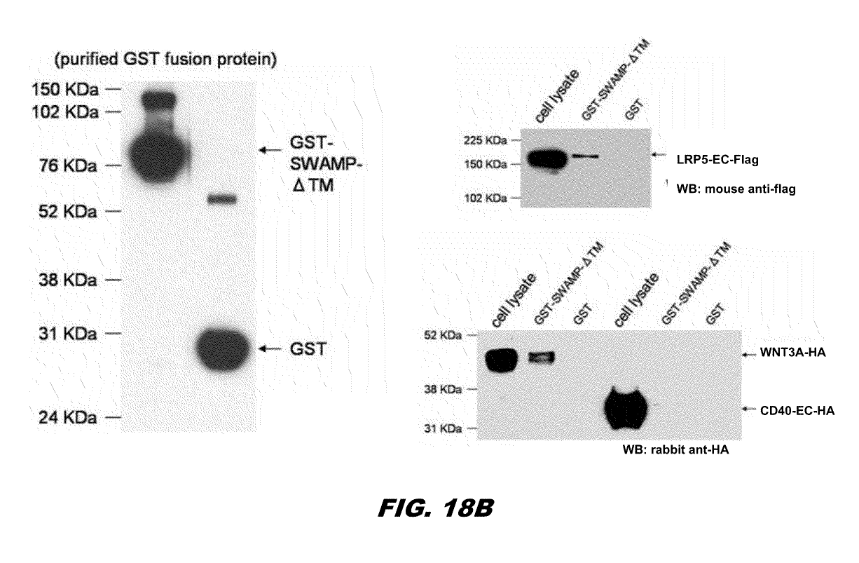

We next examined SWAMP expression in human HFs. Unlike the related APCDD1L, SWAMP was present in human scalp skin by RT-PCR (FIG. 18). In situ hybridization and immunofluorescence with an anti-SWAMP antibody (Abnova), revealed that SWAMP was expressed in the dermal papilla (DP), the matrix and differentiating cells in the hair shaft (FIG. 15A-E). Western blot from the human scalp skin with the SWAMP antibody showed two bands of 58 and 130 kDa, probably corresponding to a monomer and a dimer, respectively (FIG. 14). The mouse Swamp (also known as Drapc1, Apcdd1) mRNA is also expressed in the adult mouse HFs^{A3}, suggesting that its function can be conserved in HF development in mammals.

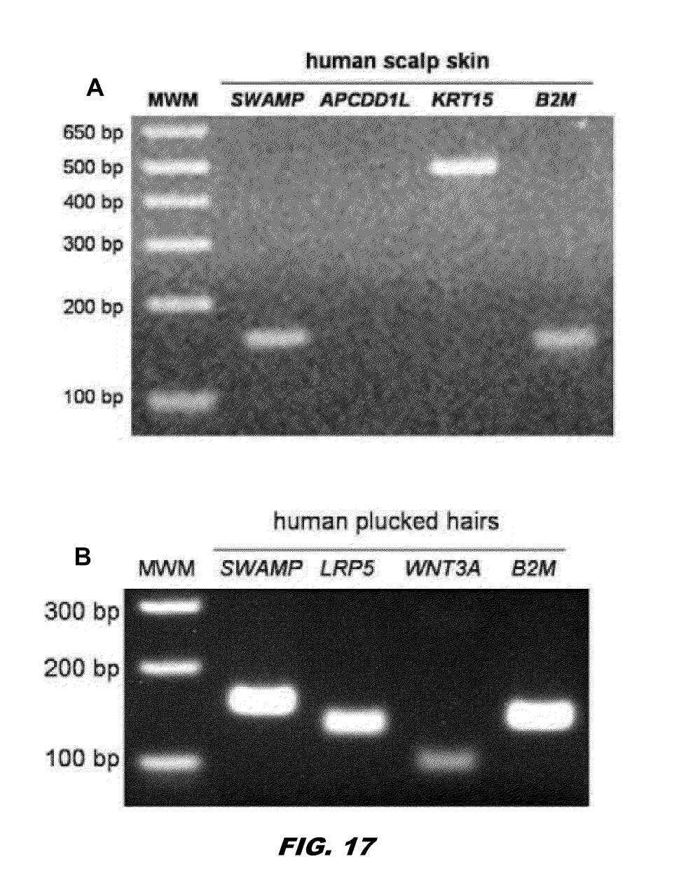

Several lines of evidence, including its activation by Wnt signaling^{A8}, the similarity in expression pattern with another Wnt inhibitor Wise^{A11}, the presence of other Wnt inhibitors in the HF (e.g. Dkk4)^{A12}, indicate that SWAMP can function as an inhibitor of Wnt signaling in a negative feedback loop^{A13}. It is noteworthy that SWAMP contains 12 highly conserved cysteine residues (FIG. 9), a structural feature present in many inhibitors of Wnt signaling and is important for interaction with Wnt ligands or their receptors^{A13, A14}._To test whether SWAMP could inhibit Wnt signaling, we first determined if it can interact with ligands and receptors of the canonical Wnt pathway. No interaction was found with Fzd2, Fzd8, and Dkk4. In contrast, Wnt3A, important for HF induction^{A15}, and LRP5, expressed in HF (FIG. 17B) coprecipitated with the extracellular domain of SWAMP (SWAMPΔTM) (FIG. 18). This suggests that SWAMP can modulate the Wnt pathway, via interaction with both WNT3A and LRP5 at the cell surface. We also investigated which domain of SWAMP mediates the Wnt inhibitory activity and the cells where SWAMP functions to inhibit the pathway and found that the Wnt inhibitory activity resides within the extracellular domain. SWAMP could affect either the signaling cell, by regulating Wnt secretion^{A25}, or the receiving cell. We conclude that SWAMP inhibits Wnt signaling in the cell autonomously, in the receiving cell.

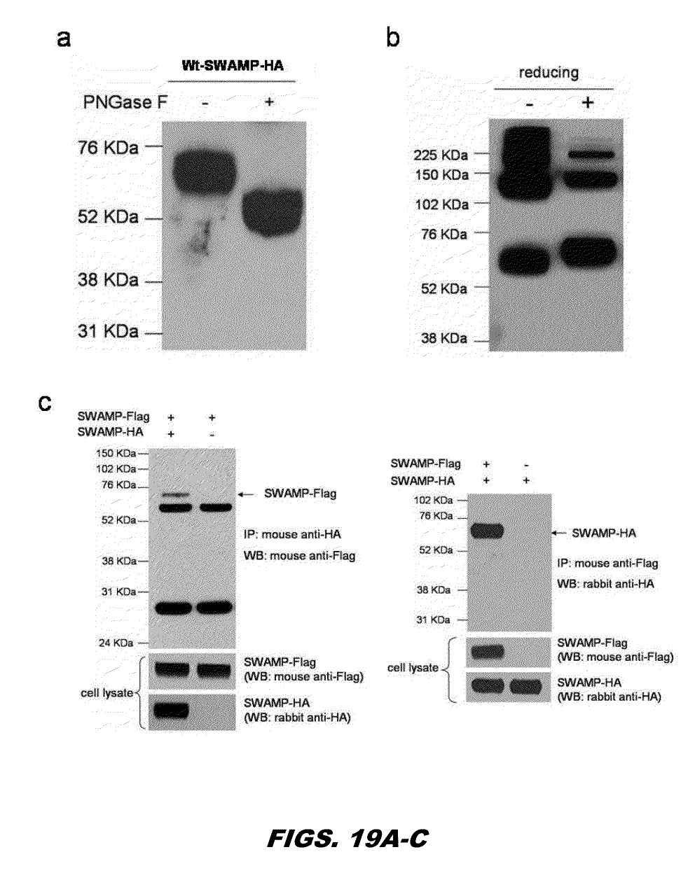

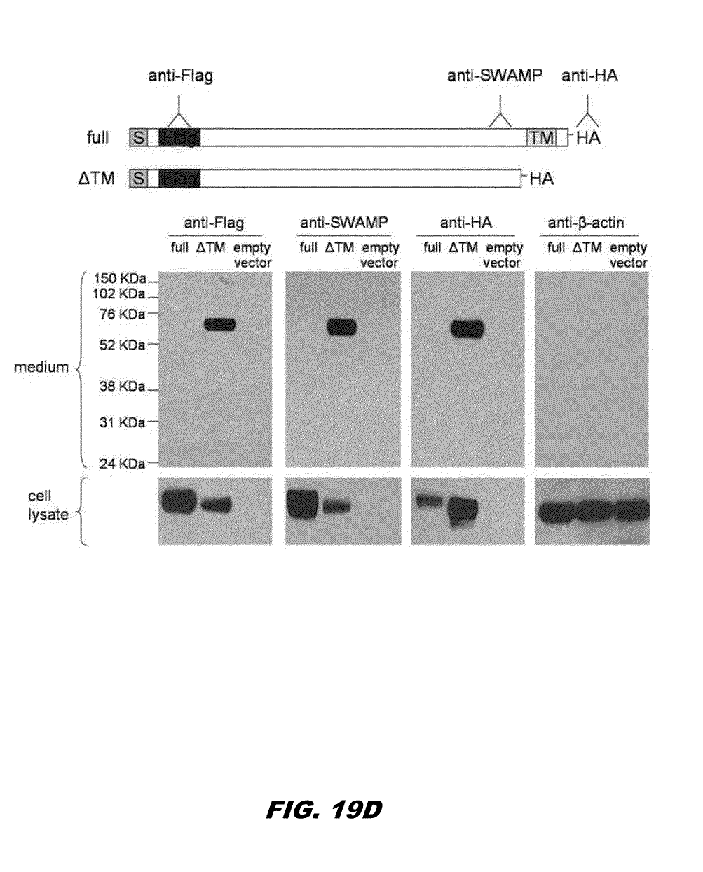

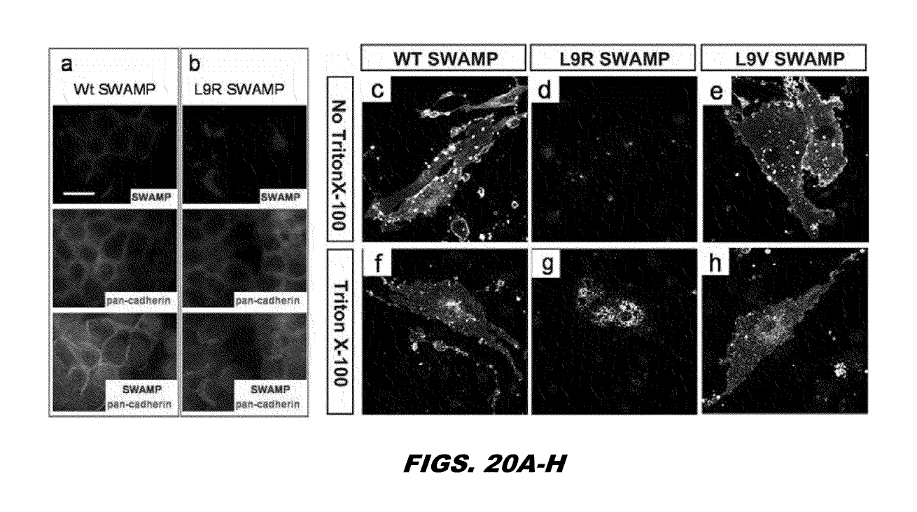

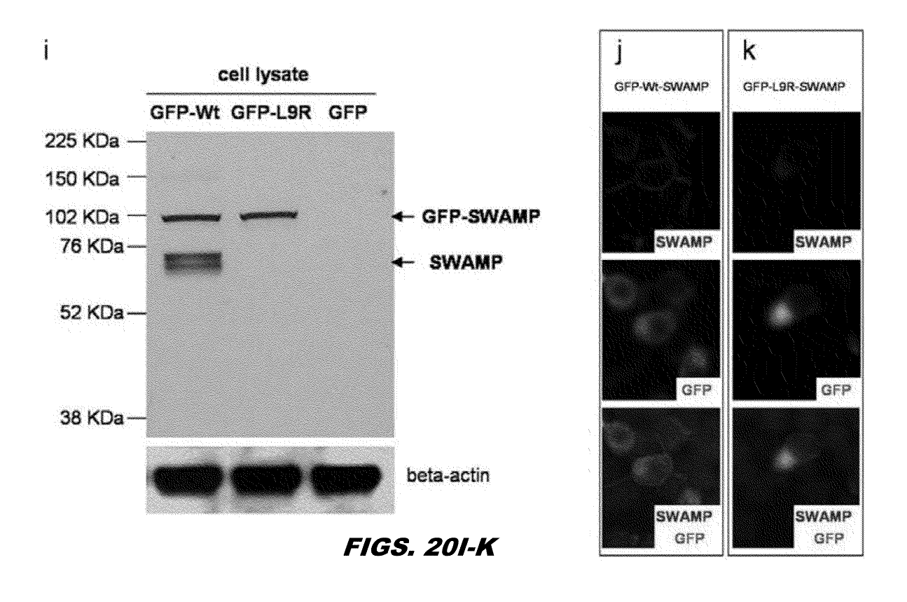

The SWAMP protein is 58 KDa in size, predicted to consist of an N-terminal signal peptide, an extracellular domain (with an N-glycosylation site at position 168), a transmembrane domain, and a C-terminal cytoplasmic domain of only two amino acids (FIG. 3A). Western blot of SWAMP expressed in HEK293T cells revealed that the protein is glycosylated and forms a dimer (FIG. 19A-C). Moreover, Wt-SWAMP is localized to the plasma membrane when transfected in a cell line (FIGS. 20A, 20C, and 20F). We tested if full length SWAMP could be cleaved to function as a diffusible Wnt inhibitor (SWAMPΔTM), but the protein was not detected in the medium of SWAMP-expressing cells, indicating that SWAMP is membrane-tethered (FIG. 19D).

The L9R mutation is predicted to disrupt the hydrophobic core of the signal peptide critical for co-translational processing of the SWAMP protein (FIG. 3B, FIG. 10)^{A9}. We analyzed protein stability and localization by western blotting and immunofluorescence, respectively, in two cell lines (HEK293T or Bend3.0) transfected with either wild type (Wt) SWAMP or two different mutations (L9R and L9V). Two fragments of 68 KDa and 130 KDa were detected in lysates of the Wt- and the conservative mutant L9V-SWAMP-transfected cells. Wt- or L9V-SWAMP protein was localized to the cell membrane, while the L9R-SWAMP was retained within the endoplasmic reticulum (ER) (FIG. 20A-H). Furthermore, overexpression of an N-terminal GFP-tagged Wt- or L9R-SWAMP protein revealed that the mutant protein was not able to undergo cleavage or localize to the membrane (FIG. 20I-K). Therefore, the L9R mutation can function in dominant-negative manner, by destabilizing the Wt protein and preventing it from reaching the plasma membrane.

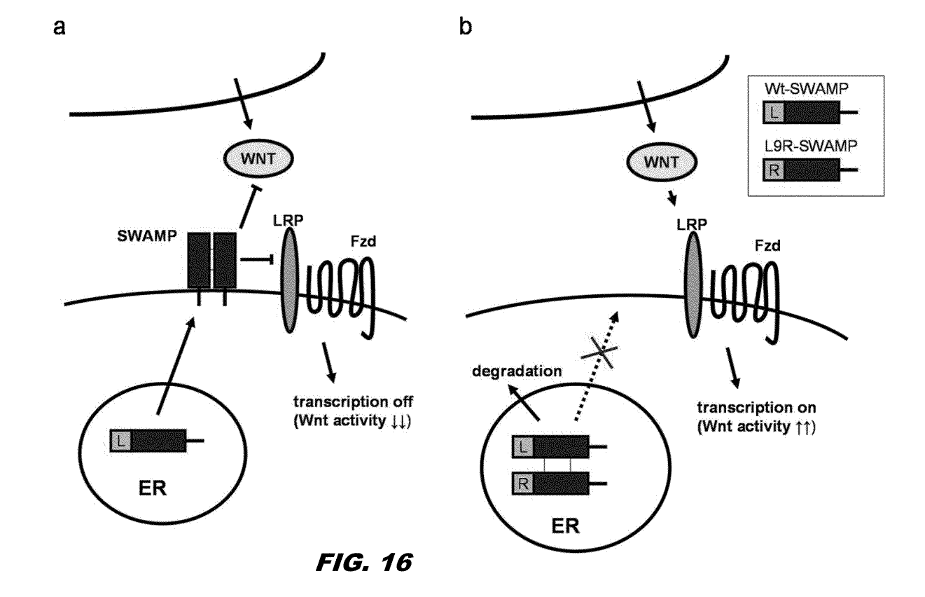

Collectively, we have shown that SWAMP is a membrane-tethered Wnt inhibitor in vivo. Since SWAMP is a direct target gene of Wnt signaling^{A8}, it can function to terminate the Wnt signal via negative feedback regulation^{A13}. The interaction of SWAMP with LRP5 and WNT3A via its extracellular domain suggests that SWAMP can prevent formation of the Wnt receptor complex (FIG. 16A). The L9R mutant is unable to repress Wnt-responsive reporters and genes, or their effect on proliferation and the generation of neurons in vivo. Moreover, our expression studies in cultured cells suggest that the L9R-SWAMP can force the Wt protein to be retained in the ER where it can undergo degradation (FIG. 16B).

Since the L9R mutation abrogates the function of the Wt protein, we predict an increase in Wnt signaling in the HF in HHS. The lack of samples from HHS patient's scalp precluded us from verifying this assumption. Nevertheless, our data in human cell lines have determined that L9R mutant SWAMP functions as a dominant-negative for the Wt protein. Studies in mice have shown that activation of Wnt signaling caused increased hair follicle density and hair follicle-derived tumors^{A27}. In contrast, mice mutant for the Wnt inhibitor Klotho show reduced hairs due to upregulation of Wnt signaling and depletion of the stem cell pool^{A28}. SWAMP is expressed in the matrix of the human HFs (FIG. 15A-E), which contains a pool of proliferating progenitors derived from the stem cell niche in the HF-bulge. We predict that upregulation of Wnt signaling in matrix cells can deplete the HF stem cell pool and cause HHS in humans. Our study provides the first genetic evidence that mutations in a Wnt inhibitor result in a hair disorder in humans. SWAMP can have a broader role in polygenic HF disorders beyond HHS, since its localization on chromosome 18 coincides with our previous linkage data in families with alopecia greata (max LOD=3.93)^{A30}, as well as linkage studies in families with AGA (max NPL score=2.56)^{A5}. Since SWAMP is widely expressed in many organs^{A3}, our findings raise the possibility that, as a Wnt inhibitor, SWAMP plays a crucial role in other processes controlled by Wnt signaling, such as early embryonic development, stem cell renewal, nervous system development, and cancer.

Methods

Linkage analysis. Genome-wide SNP-based genotyping was performed using the Affymetrix Human Mapping 10K 2.0 Array. Quality control and data analysis was performed with Genespring GT (Agilent software). Briefly, SNPs that violated a Mendelian inheritance pattern were removed from the data set prior to analysis. Haplotypes were inferred from raw genotype data. By analyzing haplotypes rather than individual SNPs, type I error introduced by linkage disequilibrium between markers is mitigated. Finally, haplotypes were analyzed for linkage under the assumption of a fully penetrant disease gene with a frequency of 0.001 transmitted by a dominant mode of inheritance.

Mutation analysis. Using the genomic DNA of the family members, all exons and exon-intron boundaries of SWAMP gene were amplified by PCR with the gene-specific primers (Table 4). In addition, the following primers in Table 5 were also used.

TABLE 5
Additional Primers Used.

| | | | | | | |
|---|---|---|---|---|---|---|
| | | | | | | Product |
| | forward primer | SEQ ID | reverse primer | SEQ ID | Annealing | size |
| Primer Name | (5′ to 3′) | NO: | (5′ to 3′) | NO: | Temp (C.°) | (bp) |

Primers used in RT-PCR experiments

| | | | | | | |
|---|---|---|---|---|---|---|
| LRP5-rtpcr | GACCCAGCCCTT | 9717 | TGTGGACGTTGAT | 9718 | 56 | 138 |
| | TGTTTTGAC | | GGTATTGGT | | | |
| WNT3A-rtpcr | GCCCCACTCGGA | 9719 | GAGGAATACTGTG | 9720 | 56 | 98 |
| | TACTTCTTACT | | GCCCAACA | | | |

Primers used to generate the probes for in situ hybridization

| | | | | | | |
|---|---|---|---|---|---|---|
| SWAMP-ISH | CCAGAGCAGGA | 9721 | CTATCTGCGGATG | 9722 | 60 | 1562 |
| | CTGGAAATG | | TTCCAATGC | | | |

The PCR products were directly sequenced in an ABI Prism 310 Automated Sequencer, using the ABI Prism Big Dye Terminator Cycle Sequencing Ready Reaction Kit (PE Applied Biosystems). The mutation 26T>G disrupts a DdeI restriction enzyme site, which was used to screen the family members and control individuals.

Clinical details and DNA extraction. Informed consent was obtained from all subjects. The study was conducted in adherence to the Declaration of Helsinki Principles. Peripheral blood samples were collected from family members as well as unrelated healthy control individuals of Pakistani and European origin (200 individuals each). Genomic DNA was isolated from these samples using the PUREGENE DNA isolation kit (Gentra System).

Genotyping. Genomic DNA from members of two Pakistani families was amplified by PCR using Platinum® PCR SuperMix (Invitrogen) and primers for microsatellite markers on chromosome 18p11. The amplification conditions for each PCR were 94° C. for 2 min, followed by 35 cycles of 94° C. for 30 sec, 55° C. for 30 sec, and 72° C. for 30 sec, with a final extension at 72° C. for 7 min. PCR products were run on 8% polyacrylamide gels and genotypes were assigned by visual inspection.

Mutation analysis of the SWAMP gene. Exon 1 and the adjacent boundary sequences of the SWAMP gene were amplified using Platinum® Taq DNA Polymerase High Fidelity (Invitrogen). Due to the high G/C content, DMSO (final 5%) and MgSO₄ (final 1.6 mM) were added to the PCR reaction. The amplification conditions were 94° C. for 2 min, followed by 35 cycles of 94° C. for 30 sec, 61° C. for 30 sec, and 68° C. for 50 sec, with, a final extension at 68° C. for 7 min. Other exons, as well as the exon-intron boundaries of the SWAMP gene, were amplified using Platinum® PCR SuperMix (Invitrogen). The amplification conditions were 94° C. for 2 min, followed by 35 cycles of 94° C. for 30 sec, 56° C. for 30 sec, and 72° C. for 50 sec, with a final extension at 72° C. for 7 min.

To screen for the mutation 26T>G (L9R), a part of exon 1 and intron 1 of the SWAMP gene was amplified by PCR using Platinum® Taq DNA Polymerase High Fidelity (Invitrogen) and the following primers: forward (5′-CCAGAGCAGGACTG GAAATG-3′) [SEQ ID NO: 9723], reverse (5′-CGCCAAGGGGACAGTGTAG-3′) [SEQ ID NO: 9724]. DMSO (final 5%) and MgSO₄ (final 1.6 mM) were added to the PCR reaction. Amplification conditions were 94° C. for 2 min, followed by 35 cycles of 94° C. for 30 sec, 59° C. for 30 sec, and 68° C. for 30 sec, with a final extension at 68° C. for 7 min. The amplified PCR products, 191 bp in size, were digested with DdeI at 37° C. overnight, and run on 2.0% agarose gels.

Cell culture. HEK293T (human embryonic kidney) cells were cultured in Dulbecco's modified Eagle's medium (DMEM; GIBCO) supplemented with 10% fetal bovine serum (FBS; GIBCO), 100 IU/ml penicillin, and 100 μg/ml streptomycin. For transfection experiments, dishes were coated with a coating medium containing 0.01 mg/ml of fibronectin (Sigma) and 0.03 mg/ml of type I collagen (Sigma) before seeding the cells so as to prevent detachment of the cells.

Anti-SWAMP antibodies. A mouse polyclonal anti-human APCDD1 (SWAMP) antibody was purchased from Abnova Corporation. This antibody was raised against the full-length human SWAMP protein. We performed epitope-mapping using three truncated GST-SWAMP proteins (amino acid (aa) residues 1-171, 166-336, and 331-514), and confirmed that the epitope of the antibody exists between aa residues 166 and 336 of the human SWAMP, which corresponds to the middle portion of the extracellular domain. This antibody recognized hair shaft and dermal papilla in human hair follicles (FIG. 15B-E), which finely overlapped with the signals detected by in situ hybridization (FIG. 15A). An affinity-purified rabbit polyclonal anti-mouse Apcdd1 (Swamp) antibody was produced by immunizing rabbits with the synthetic peptide, CQRPSDGSSPDRPEKRATSY (corresponding to the C-terminus of the extracellular domain of the mouse SWAMP protein, aa residues 441-459; SEQ ID NO: 9725) conjugated to KLH (Pierce, Rockford, Ill.). This region is completely conserved among mouse and human SWAMP proteins. The antibody was affinity-purified from the serum using the Sulfolink immobilization column (Pierce). This antibody strongly recognized human SWAMP protein in western blots and immunofluorescence.

RT-PCR in human scalp skin and plucked hairs. Total RNA were isolated from scalp skin and plucked scalp hairs of healthy control individuals using the RNeasy® Minikit (Quiagen). 2 μg of total RNA was reverse-transcribed with oligo-dT primers and Super-Script™ III (Invitrogen). The cDNAs were amplified by PCR using Platinum® PCR Super-Mix and primer pairs for SWAMP, APCDD1L, keratin 15 (KRT15), LRP5, WNT3A, and β-2 microglobulin (B2M) genes (Table 4 and Table 5). Primers for the KRT15, LRP5, and WNT3A genes were designed as described previously^{A31, A32}. Amplification conditions were 94° C. for 2 min, followed by 35 cycles of 94° C. for 30 sec, 58° C. for 30 sec and 72° C. for 30 sec, with a final extension at 72° C. for 7 min. PCR products were run on 1.5% agarose gels.

Expression vectors. To generate the expression construct for the human SWAMP (pCXN2.1-Wt-SWAMP), the full length SWAMP cDNA sequences were amplified by PCR using the first strand cDNA from human scalp skin as a template and the following primers: SWAMP-F-XhoI (5′-AAAACTCGAGCCAGAGCAGGACTGGAAATG-3′) [SEQ ID NO: 9726] and SWAMP-R-NheI (AAAAGCTAGCCTATCTGCGGATGTTCCAATGC-3′) [SEQ ID NO: 9727]. To generate the constructs for the C-terminal hemagglutin (HA)-tagged (pCXN2.1-Wt-SWAMP-HA) and the C-terminal Flag-tagged (pCXN2.1-Wt-SWAMP-Flag) SWAMP, the following reverse primers were used: SWAMP-R-HA-NheI (5′-AAAAGCT AGCTCAGGCGTAGTCGGGCACGTCGTAGGGGTATCTGCGGATGTTCCAATGC-3′) [SEQ ID NO: 9728] and SWAMP-R-Flag-NheI (5′-AAAAGCTAGCTCACTTATCGTCG TCATCCTTGTAATCTCTGCGGATGTTCCAATGC-3′) [SEQ ID NO: 9729], respectively. L9R and L9V mutant SWAMP sequences were PCR-amplified using the following forward primers: SWAMP-L9R-F-XhoI (5′-AAAACTCGAGCCAGAGCAGGACTGGAAATGTC CTGGCCGCGCCGCCTCCTGCGCAGAT-3′) [SEQ ID NO: 9730] and SWAMP-L9V-F-XhoI (5′-AAAACTCGAGCCAGAGCAGGACTGGAAATGTCCTGGCCGCGCCG CCTCCTGGTCAGAT-3′) [SEQ ID NO: 9731], respectively. Note that T>G and C>G substitutions were introduced into the primers, respectively (shown in bold and underlined).

For generating the expression constructs for truncated SWAMP (aa residues 1-486) with the C-terminal HA-tag (pCXN2.1-SWAMP-ΔTM-HA) and the C-terminal Flag-tag (pCXN2.1-SWAMP-ΔTM-Flag), the following reverse primers were used: SWAMP-ΔTM-R-HA-NheI (5′-AAAAGCTAGCTCAGGCGTAGTCGGGCACGTCGTAGGGG TAGCCATACAGGCTGCTTCCACT-3′) [SEQ ID NO: 9732] and SWAMP-ΔTM-R-Flag-NheI (5′-AAAAGCTAGCTCACTTATCGTCGTCATCCTTGTAATCGCCATACAGG CTGCTTCCACT-3′) [SEQ ID NO: 9733], respectively. The amplified products were subcloned into the XhoI and NheI sites of the mammalian expression vector pCXN2.1³³, a slightly modified version of pCXN2³⁴ with multiple cloning sites. To introduce a Flag-tag between aa residues 35 and 36 of the SWAMP protein, N-terminal region of the SWAMP was PCR-amplified using the forward primer (SWAMP-F-XhoI) and a reverse primer (SWAMP-R-Flag-AvrII: 5′-AAAACCTAGGCTTATCGTCGTCATCCTTGTAATCATGA GACCTGCTGTCTGGAT-3′) [SEQ ID NO: 9734], which was followed by digestion with restriction enzymes XhoI and AvrII. The C-terminal region of the SWAMP and the truncated SWAMP proteins with the C-terminal HA-tag was obtained through digestion of the pCXN2.1-Wt-SWAMP-HA and the pCXN2.1-SWAMP-ΔTM-HA constructs with restriction enzymes AvrII and NheI. These two fragments were ligated with AvrII site, and subsequently subcloned into the XhoI and NheI sites of the pCXN2.1 vector.

To generate expression constructs for N-terminal GFP-tagged SWAMP protein, the coding region of the SWAMP and the rabbit β-globin 3′-flanking sequences were cut out from the pCXN2.1-SWAMP constructs with restriction enzymes XhoI and BamHI, and subcloned in frame into XhoI and BamHI sites of pEGFP-C1 vector (Clontech). The templates were also subcloned into XhoI and BamHI sites of pBluescript-SK (−) vector (Stratagene). To express the GST fusion SWAMP protein in bacteria, the extracellular domain of the human SWAMP (aa residues 28-486) was PCR-amplified using the following primers: SWAMP-F-EcoRI (5′-AAAAGAATTCCCTTCATCCAGACAG CAGGTC-3′) [SEQ ID NO: 9735] and SWAMP-ΔTM-R-XhoI (5′-AAAACTCGAGTCAGCCATACA GGCTGCT TCCACT-3′) [SEQ ID NO: 9736]. The amplified fragment was subcloned in-frame into EcoRI and XhoI sites of pGEX-4T-3 vector (GE Healthcare). pGEM Wnt8, the Sia luciferase reporter gene, and pSP36 β-catenin have been previously described.

The full length mouse Swamp cDNA was amplified by RT-PCR from brain endothelial cells using the First Strand Synthesis Kit and High Fidelity Amplification Kit (Roche Applied Science) with the following primers: SwampF 5′-GGGGACAGAGAC GGACTACA-3′ [SEQ ID NO: 9739] and SwampR 5′CAAGGCATTCAAGTGCATC3′ [SEQ ID NO: 9740]. The amplified cDNA was confirmed by sequencing and subcloned into PCRII TOPO and pCAGGS vectors for in vitro transcription. The SwampΔTM isoform containing the extracellular domain of mouse Swamp (aa 1-486) was amplified by PCR from the full length cDNA using the following primers: SWAMPF 5′-GGGGACAGAGACGG ACTACA-3′ [SEQ ID NO: 9741] and SwampΔTM 5′-CTGCCCTGCCTGCCATAC AGATGACCTTGACTGTC-3′ [SEQ ID NO: 9742] and subcloned into pCAGGS vector for chick electroporation.

To generate the expression construct for the human WNT3A (pCXN2.1-WNT3A), PCR was performed using cDNA from plucked human hairs and the following primers: WNT3A-F-XhoI (5′-AAAACTCGAGCGGCGATGGCCCCACTCGGATACTT-3′) [SEQ ID NO: 9743], WNT3A-R-NheI (5′-AAAAGCTAGCCTACTTGCAGGTGTGCACG TCGT-3′) [SEQ ID NO: 9744]. For the C-terminal HA-tagged human WNT3A (pCXN2.1-WNT3A-HA), the following reverse primer was used: WNT3A-R-HA-NheI (5′-AAAAGC TAGCTAGGCGTAGTCGGGCACGTCGTAGGGGTACTTGCAGGTGTGCACGTCG-3′) [SEQ ID NO: 9745]. To generate the expression construct for the extracellular domain of the human CD40 with the C-terminal HA tag (aa residues 1-193; pCXN2.1-CD40-EC-HA), PCR was performed using human thymus cDNA as a template and the following primers: CD40-F-XhoI (5′-ATATCTCGAGCCTCGCTATGGTTCGTCTGCCT-3′) [SEQ ID NO: 9746] and CD40-R-HA-NheI (5′-ATATGCTAGCTAGGCGTAGTCGGGCACGTCGTAGGGGTAT CTCAGCCGATCCTGGGGAC-3′) [SEQ ID NO: 9747]. To generate the construct for the extracellular domain of the human LRP5 with the C-terminal Flag tag (aa residues 1-1384; pCXN2.1-LRP5-EC-Flag), the N-terminal sequences of the human LRP were PCR-amplified using the expression construct for the full-length human LRP5 as a template and the following primers: LRP5-F-EcoRI (5′-AAAAGAATTCCGGACAACATGGAGGCAG-3′) [SEQ ID NO: 9748] and LRP5-R-Flag-NheI (5′-AAAAGCTAGCTACTTATCGTCGTCA TCCTTGTAATCGCTGTGGGCCGGGCTGTCGTCTGA-3′) [SEQ ID NO: 9749]. The amplified products were subcloned into the XhoI/NheI sites (for WNT3A and CD40) or EcoRI/NheI sites (for LRP5) of the pCXN2.1 vector. To generate the expression construct for the mouse Frizzled 2 (mFzd2), the full-length open reading frame of the mFzd2 was purchased from Invitrogen (clone ID 6411627), which was subcloned into NotI sites of the pCXN2:1 vector.

Transient transfections and western blots in cultured cells and human scalp skin. HEK293T cells or Bend3.0 cells were plated in 60 mm dishes the day before transfection. Expression plasmids of SWAMP were transfected with FuGENE® 6 (Roche Applied Science) at 60% confluency for HEK293 cells or Targefect_HUVEC for Bend3.0 cells. Total amount of transfected plasmids were adjusted with the empty pCXN2.1 vector. The cells were cultured 48 h after transfection in Opti-MEM (GIBCO). The cells were harvested and homogenized by sonication in homogenization buffer (25 mM HEPES-NaOH (pH 7.4), 10 mM MgCl2, 250 mM sucrose, and 1× Complete Mini Protease Inhibitor Cocktail (Roche Applied Science)). The cell debris was removed by centrifugation at 3,000 rpm for 10 min at 4° C., and the supernatant was collected as cell lysates. To obtain membrane fraction, the cell lysates were ultracentrifuged at 100,000 g for 1 h at 4° C. The pellet was suspended with the homogenization buffer. The cultured medium with 1× Complete Mini Protease Inhibitor Cocktail was centrifuged at 1,500 rpm for 5 min at 4° C. The supernatant was purified with 0.45 μm syringe filters (Thermo Fisher Science), and concentrated using Amicon Ultra-15 Centrifugal Filter Unit with Ultracel-10 Membrane (Millipore) according to the manufacturer's recommendations. To examine the N-glycosylation of SWAMP, the cell lysates from the wild-type SWAMP expressing cells were treated with PNGase F (Sigma) following the manufacturer's recommendations. Total cell lysates from human scalp skin were extracted by homogenization in 50 mM Tris-HCl (pH 8.0), 150 mM NaCl, 1.0% NP40, 0.5% sodium deoxycholate, 0.1% SDS, and 1× Complete Mini Protease Inhibitor Cocktail. All samples were mixed with equal amount of Laemmli Sample Buffer (Bio-Rad Laboratories) containing 5% β-mercaptoethanol, boiled at 95° C. for 5 min, and analyzed by 10% SDS-polyacrylamide gel electrophoresis (SDS-PAGE). Western blots were performed as described previously^{A36}. The primary antibodies used were rabbit polyclonal anti-HA (diluted 1:4,000; Abcam), rabbit polyclonal anti-SWAMP (1:20,000), mouse polyclonal anti-APCDD1 (1:1,000; Abnova), mouse monoclonal anti-Flag M2 (1:1,000; Sigma), and rabbit polyclonal anti-β-actin (1:10,000; Sigma).

Wnt reporter assays in HEK293T cells. HEK293T cells were seeded in 12 well dishes the day before transfection. Either 100 ng of TOPFlash (active) or FOPFlash (inactive) Wnt reporter vector was transfected into each well along with constructs for WNT3A (200 ng), Fzd2 (100 ng), LRP5 (100 ng), and/or wild type SWAMP-HA (800 ng) using Lipofectamine 2000 (Invitrogen). A construct for β-galactosidase reporter (100 ng) was also transfected for normalization of transfection efficiency. The cells were lysed 36 h after transfection and the signals were assayed using the appropriate substrates for luciferase (Steady-Glo Luciferase Assay System) and β-galactosidase (Promega) on a 20/20ⁿ luminometer (Turner Biosystems) for luciferase and Model 680 microplate reader (BioRad) for β-galactosidase. The Wnt activity was measured based on the ratio of TOP/FOP luciferase activity. The results represent triplicate determination of a single experiment that is representative a total of five similar experiments.

Co-Immunoprecipitation (Co-IP) assays. Expression plasmids (total 4 μg) were transfected into HEK293T cells seeded on 60 mm dishes with FuGENE® 6 (Roche Applied Science) at 60% confluency. 24 h after the transfection, the cells were harvested and homogenized in lysis buffer (20 mM Tris-HCl (pH 7.5), 137 mM NaCl, 10% Glycerol, 2 mM EDTA, 0.5% Triton X, and 1× Complete Mini Protease Inhibitor Cocktail). Total cell lysates were collected by centrifugation at 14,000 rpm for 15 min at 4° C. The samples were incubated with either mouse monoclonal anti-Flag M2 agarose gel (Sigma) or mouse monoclonal anti-HA agarose gel (Sigma) for 3 h at 4° C. The agarose beads were washed with lysis buffer for five times. The precipitated proteins were eluted with NuPAGE® LDS Sample Buffer containing Sample Reducing Agent (Invitrogen), incubated at 75° C. for 10 min, and separated on 10% NuPAGE® gels (Invitrogen). Western blots were performed using rabbit polyclonal anti-HA (diluted 1:4,000; Abcam) or mouse monoclonal anti-Flag M2 antibody (1:1,000; Sigma).

GST pulldown assays. Expression of GST-fusion proteins was induced in DH5a (Invitrogen) by the addition of 0.1 mM isopropyl-β-D-thiogalactopyranoside at 37° C. for 3 h, and the fusion proteins were isolated from bacterial lysates by affinity chromatography with glutathione-Sepharose beads (GE Healthcare Life Sciences). HEK293T cells overexpressing LRP5-EC-Flag, WNT3A-HA, or CD40-EC-HA were dissolved in lysis buffer (20 mM Tris-HCl (pH 7.5), 137 mM NaCl, 10% Glycerol, 2 mM EDTA, 0.1% Triton X, and 1× Complete Mini Protease Inhibitor Cocktail), and centrifuged at 12,000 g at 4° C. for 30 min. Clarified supernatants were incubated in the presence of either GST alone or GST-SWAMPΔTM fusion proteins (10 μg) immobilized to glutathione beads at 4° C. for 3 h. After incubation, the beads were washed with the lysis buffer for five times, resuspended in NuPAGE® LDS Sample Buffer containing Sample Reducing Agent (Invitrogen), fractioned by 10% NuPAGE® (Invitrogen), and analyzed by western blotting. The antibodies used were: rabbit polyclonal anti-GST (1:3,000; Santa Cruz Biotechnology), rabbit polyclonal anti-HA (1:4,000; Abcam) and mouse monoclonal anti-Flag M2 (1:1,000; Sigma).

In situ hybridization. A part of the human SWAMP cDNA (GenBank Accession number, NM_{—}153000: nt. 338-1899) was cloned into pCR®II-TOPO vector (Invitrogen). The antisense and sense DIG-labelled cRNA probes were synthesized from the linearized vectors with T7 and SP6 RNA polymerases (Roche Applied Science), respectively. Dissected human hair follicles were fixed with 4% paraformaldehyde-PBS at 4° C. overnight. After dehydration step with 30% sucrose-PBS, the tissues were frozen in OCT compound and sectioned on glass slides at the thickness of 10 μm. In situ hybridization was performed following the methods described previously with minor modifications^{A37}. At the prehybridization steps, the sections were treated with 5 μg/ml Protease K for 15 min at 37° C. Hybridization was performed at 55° C. overnight. In situ hybridizations on chick spinal cord sections were performed as described^{A38}. The antisense mSwamp mRNA was generated using the In vitro transcription kit (Roche, Indianapolis, Ind.) with T7 RNA polymerase. The antisense chick Sim1 mRNA was generated using the T3 RNA polymerase.

Indirect immunofluorescence (IIF). IIF on cultured cells and fresh frozen sections of individually dissected hair follicles was performed as described previously^{A36}. IIF on HEK293T cells were performed 48 h after the SWAMP expression constructs were transfected. For some stainings, cell membrane was labeled with rhodamine-phalloidin (Invitrogen). The primary antibodies used were mouse polyclonal anti-APCDD1 (diluted 1:1,000; Abnova), rabbit polyclonal anti-SWAMP (1:4,000), rabbit polyclonal anti-pan-cadherin (1:200; Invitrogen), and goat polyclonal anti-calnexin (1:200; Santa Cruz Biotechnology). Immunofluorescence on chick spinal cord sections was performed as described^{A39}. The monoclonal antibodies against Nkx2.2, Pax6, Pax7, En-1 and Evx1 were purchased from DSHB (Iowa); rabbit anti Olig2 (Chemicon, Billerica Mass.), rabbit anti-Sox3, rabbit anti Chx10, and guinea pig anti Isl1/2, sheep anti GFP (Biogenesis) and mouse anti β3-tubulin (Tuj1; Covance) were used as described^{A39}.

Accession numbers. APCDD1 mRNA NM_{—}153000, protein NP_{—}694545.

### REFERENCES

A1. Toribio, J. & Quinones, P. A. Hereditary hypotrichosis simplex of the scalp. Evidence for autosomal dominant inheritance. Br. J. Dermatol. 91, 687-696 (1974).
A2. Baumer, A., Belli, S., Trueb, R. M. & Schinzel, A. An autosomal dominant form of hereditary hypotrichosis simplex maps to 18p11.32-p11.23 in an Italian family. Eur. J. Hum. Genet. 8, 443-448 (2000).
   A3. Jukkola, T., Sinjushina, N. & Partanen, J. Drapc1 expression during mouse embryonic development. Gene Expr. Patterns 4, 755-762 (2004).
A4. Trueb, R. M. Molecular mechanisms of androgenetic alopecia. Exp. Gerontol. 37, 981-990 (2002).
A5. Hillmer, A. M., et al. Genome-wide scan and fine-mapping linkage study of androgenetic alopecia reveals a locus on chromosome 3q26. Am. J. Hum. Genet. 82, 737-743 (2008).
A6. Hillmer, A. M., et al. Susceptibility variants for male-pattern baldness on chromosome 20p11. Nat. Genet. 40, 1279-1281 (2008).
A7. Richards, J. B., et al. Male-pattern baldness susceptibility locus at 20p11. Nat. Genet. 40, 1282-1284 (2008).
A8. Takahashi, M., et al. Isolation of a novel human gene, APCDD1, as a direct target of the beta-Catenin/T-cell factor 4 complex with probable involvement in colorectal carcinogenesis. Cancer Res. 62, 5651-5656 (2002).
A9. Pidasheva, S., Canaff, L., Simonds, W. F., Marx, S. J. & Hendy, G. N. Impaired cotranslational processing of the calcium-sensing receptor due to signal peptide missense mutations in familial hypocalciuric hypercalcemia. Hum. Mol. Genet. 14, 1679-1690 (2005).
A10. Fitches, A. C., et al. Impaired cotranslational processing as a mechanism for type I antithrombin deficiency. Blood 92, 4671-4676 (1998).
A11. O'Shaughnessy, R. F., Yeo, W., Gautier, J., Jahoda, C. A. & Christiano, A. M. The WNT signalling modulator, Wise, is expressed in an interaction-dependent manner during hair-follicle cycling. J. Invest. Dermatol. 123, 613-621 (2004).
A12. Bazzi, H., Fantauzzo, K. A., Richardson, G. D., Jahoda, C. A. & Christiano, A. M. The Wnt inhibitor, Dickkopf 4, is induced by canonical Wnt signaling during ectodermal appendage morphogenesis. Dev. Biol. 305, 498-507 (2007).
A13. Kawano, Y. & Kypta, R. Secreted antagonists of the Wnt signalling pathway. J. Cell Sci. 116, 2627-2634 (2003).
A14. Nakamura, T. & Matsumoto, K. The functions and possible significance of Kremen as the gatekeeper of Wnt signalling in development and pathology. J Cell. Mol. Med. 12, 391-408 (2008).
A15. Kishimoto, J., Burgeson, R. E. & Morgan, B. A. Wnt signaling maintains the hair-inducing activity of the dermal papilla. Genes Dev. 14, 1181-1185 (2000).
A16. Megason, S. G. & McMahon, A. P. A mitogen gradient of dorsal midline Wnts organizes growth in the CNS. Development 129, 2087-2098 (2002).
A17. Lei, Q., et al. Wnt signaling inhibitors regulate the transcriptional response to morphogenetic Shh-Gli signaling in the neural tube. Dev. Cell 11, 325-337 (2006).
A18. Yu, W., McDonnell, K., Taketo, M. M. & Bai, C. B. Wnt signaling determines ventral spinal cord cell fates in a time-dependent manner. Development 135, 3687-3696 (2008).
A19. Leyns, L., Bouwmeester, T., Kim, S. H., Piccolo, S. & De Robertis, E. M. Frzb-1 is a secreted antagonist of Wnt signaling expressed in the Spemann organizer. Cell 88, 747-756 (1997).
A20. Wang, S., Krinks, M., Lin, K., Luyten, F. P. & Moos, M., Jr. Frzb, a secreted protein expressed in the Spemann organizer, binds and inhibits Wnt-8. Cell 88, 757-766 (1997).
A21. Niehrs, C. Regionally specific induction by the Spemann-Mangold organizer. Nat. Rev. Genet. 5, 425-434 (2004).
A22. Heasman, J. Patterning the early Xenopus embryo. Development 133, 1205-1217 (2006).
A23. Brannon, M., Gomperts, M., Sumoy, L., Moon, R. T. & Kimelman, D. A beta-catenin/XTcf-3 complex binds to the siamois promoter to regulate dorsal axis specification in Xenopus. Genes Dev. 11, 2359-2370 (1997).
A24. Smith, J. C., Price, B. M., Green, J. B., Weigel, D. & Herrmann, B. G. Expression of a Xenopus homolog of Brachyury (T) is an immediate-early response to mesoderm induction. Cell 67, 79-87 (1991).
A25. Kadowaki, T., Wilder, E., Klingensmith, J., Zachary, K. & Perrimon, N. The segment polarity gene porcupine encodes a putative multitransmembrane protein involved in Wingless processing. Genes Dev. 10, 3116-3128 (1996).
A26. Hoppler, S., Brown, J. D. & Moon, R. T. Expression of a dominant-negative Wnt blocks induction of MyoD in Xenopus embryos. Genes Dev. 10, 2805-2817 (1996).
A27. Gat, U., DasGupta, R., Degenstein, L. & Fuchs, E. De Novo hair follicle morphogenesis and hair tumors in mice expressing a truncated beta-catenin in skin. Cell 95, 605-614 (1998).
A28. Liu, H., et al. Augmented Wnt signaling in a mammalian model of accelerated aging. Science 317, 803-806 (2007).
A29. Andl, T., Reddy, S. T., Gaddapara, T. & Millar, S. E. WNT signals are required for the initiation of hair follicle development. Dev. Cell 2, 643-653 (2002).
A30. Martinez-Mir, A., et al. Genomewide scan for linkage reveals evidence of several susceptibility loci for alopecia greata. Am. J. Hum. Genet. 80, 316-328 (2007).
A31. Pasternack, S. M., et al. G protein-coupled receptor P2Y5 and its ligand LPA are involved in maintenance of human hair growth. Nat. Genet. 40, 329-334 (2008).
A32. Konigshoff, M., et al. Functional Wnt signaling is increased in idiopathic pulmonary fibrosis. PLoS ONE 3, e2142 (2008).
A33. Noguchi, K., Ishii, S. & Shimizu, T. Identification of p2y9/GPR23 as a novel G protein-coupled receptor for lysophosphatidic acid, structurally distant from the Edg family. J. Biol. Chem. 278, 25600-25606 (2003).
A34. Niwa, H., Yamamura, K. & Miyazaki, J. Efficient selection for high-expression transfectants with a novel eukaryotic vector. Gene 108, 193-199 (1991).
A35. Briscoe, J., Pierani, A., Jessell, T. M. & Ericson, J. A homeodomain protein code specifies progenitor cell identity and neuronal fate in the ventral neural tube. Cell 101, 435-445 (2000).
A36. Bazzi, H., et al. Desmoglein 4 is expressed in highly differentiated keratinocytes and trichocytes in human epidermis and hair follicle. Differentiation 74, 129-140 (2006).
A37. Aoki, N., et al. A novel type II cytokeratin, mK6irs, is expressed in the Huxley and Henle layers of the mouse inner root sheath. J. Invest. Dermatol. 116, 359-365 (2001).
A38. Schaeren-Wiemers, N. & Gerfin-Moser, A. A single protocol to detect transcripts of various types and expression levels in neural tissue and cultured cells: in situ hybridization using digoxigenin-labelled cRNA probes. Histochemistry 100, 431-440 (1993).
A39. Agalliu, D. & Schieren, I. Neural Dev. 4, 2 (2009).
A40. Nieuwkoop, P. D. & Faber, J. Normal table of Xenopus laevis. North Holland Publishing Co. Amsterdam, The Netherlands (1967).
A41. Vonica, A. & Gumbiner, B. M. Zygotic Wnt activity is required for Brachyury expression in the early Xenopus laevis embryo. Dev. Biol. 250, 112-127 (2002).
A42. Taylor, M. F., Paulauskis, J. D., Weller, D. D. & Kobzik, L. In vitro efficacy of morpholino-modified antisense oligomers directed against tumor necrosis factor-alpha mRNA. J. Biol. Chem. 271, 17445-17452 (1996).
A43. Wilson, P. A. & Melton, D. A. Mesodermal patterning by an inducer gradient depends on secondary cell-cell communication. Curr. Biol. 4, 676-686 (1994).

### Example 3

### Clinical Features of Pakistani Families with HHS

We identified two Pakistani families, HHS1 and HHS2, with typical clinical features with Hereditary hypotrichosis simplex (HHS) (FIG. 1A-F, FIG. 5, FIG. 1H, bottom panel of FIG. 2B, and FIG. 6). The pedigrees of both families show clear autosomal dominant inheritance (FIG. 1H, bottom panel of FIG. 2B, and FIG. 6). All affected individuals had normal scalp hair density at birth, and the hair loss gradually progressed with age, beginning around 2-5 years old (FIG. 1A-F, FIG. 5, FIG. 1H, bottom panel of FIG. 2B, and FIG. 6). The hair grows slowly and stops growing after a few inches. Some affected individuals show light-colored or hypopigmented hair shafts (FIG. 1A, 1C and FIG. 5A). In most cases, body hairs and sexual hairs are also sparse (FIG. 5F). Eyebrows, eyelashes, and beard hairs are not affected. Under light microscopy, the bulb portion of the plucked hair shows dystrophic features (FIG. 5G) and is miniaturized (FIG. 5H). The hair shaft is thin and without any characteristic anomalies, and the distal ends appear tapered (FIG. 5I). Affected individuals in both families show normal teeth, nails, and sweating, and do not show keratosis pilaris. There was no familial history of either neurologic abnormalities or a high prevalence of cancers. We initially excluded the CDSN and HR genes from both families by linkage analysis.

### REFERENCES

S1. Schmidt-Ullrich, R. & Paus, R. Molecular principles of hair follicle induction and morphogenesis. Bioessays 27, 247-261 (2005).
S2. Sprecher, E., et al. Hypotrichosis with juvenile macular dystrophy is caused by a mutation in CDH3, encoding P-cadherin. Nat Genet 29, 134-136 (2001).
S3. Shimomura, Y., et al., P-cadherin is a p63 target gene with a crucial role in the developing human limb bud and hair follicle. Development 135, 743-753 (2008).
S4. Kjaer, K. W., et al. Distinct CDH3 mutations cause ectodermal dysplasia, ectrodactyly, macular dystrophy (EEM syndrome). J Med Genet 42, 292-298 (2005).
S5. Wen, Y., et al. Loss-of-function mutations of an inhibitory upstream ORF in the human hairless transcript cause Marie Unna hereditary hypotrichosis. Nat Genet 41, 228-233 (2009).
S6. Winter, H., et al. Mutations in the hair cortex keratin hHb6 cause the inherited hair disease monilethrix. Nat Genet 16, 372-374 (1997).
S7. van Steensel, M. A., Steijlen, P. M., Bladergroen, R. S., Vermeer, M. & van Geel, M. A missense mutation in the type II hair keratin hHb3 is associated with monilethrix. J Med Genet 42, e19 (2005).
S8. Kljuic, A., et al. Desmoglein 4 in hair follicle differentiation and epidermal adhesion. Cell 113, 249-260 (2003).
S9. Schaffer, J. V., et al. Mutations in the desmoglein 4 gene underlie localized autosomal recessive hypotrichosis with monilethrix hairs and congenital scalp erosions. J Invest Dermatol 126, 1286-1291 (2006).
S10. Shimomura, Y., Sakamoto, F., Kariya, N., Matsunaga, K. & Ito, M. Mutations in the desmoglein 4 gene are associated with monilethrix-like congenital hypotrichosis. J Invest Dermatol 126, 1281-1285 (2006).
S11. Zlotogorski, A., et al. An autosomal recessive form of monilethrix is caused by mutations in DSG4: clinical overlap with localized autosomal recessive hypotrichosis. J Invest Dermatol 126, 1292-1296 (2006).
S12. Toribio, J. & Quinones, P. A. Hereditary hypotrichosis simplex of the scalp. Evidence for autosomal dominant inheritance. Br J Dermatol 91, 687-696 (1974).
S13. Bentley-Phillips, B. & Grace, H. J. Hereditary hypotrichosis. A previously undescribed syndrome. Br J Dermatol 101, 331-339 (1979).
S14. Ibsen, H. H., et al. Acta Derm Venereol 71, 349-351 (1991).
S15. Levy-Nissenbaum, E., et al. Hypotrichosis simplex of the scalp is associated with nonsense mutations in CDSN encoding corneodesmosin. Nat Genet 34, 151-153 (2003).
S16. Baumer, A., Belli, S., Trueb, R. M. & Schinzel, A. An autosomal dominant form of hereditary hypotrichosis simplex maps to 18p11.32-p11.23 in an Italian family. Eur J Hum Genet 8, 443-448 (2000).
S17. Pasternack, S. M., et al. G protein-coupled receptor P2Y5 and its ligand LPA are involved in maintenance of human hair growth. Nat Genet 40, 329-334 (2008).
S18. Shimomura, Y., et al. Disruption of P2RY5, an orphan G protein-coupled receptor, underlies autosomal recessive woolly hair. Nat Genet 40, 335-339 (2008).
S19. Kao, K. R. & Elison, R. P. The entire mesodermal mantle behaves as Spemann's organizer in dorsoanterior enhanced Xenopus laevis embryos. Dev Biol 127, 64-77 (1988).

### Example 4

### APCDD1 Target Genes and Interaction Partners

APCDD1 is a conserved gene, not only in vertebrates but in all Deuterostomes, from sea urchin to man. As described herein, we have showed that APCDD1 acts as a Wnt inhibitor, and directly interacted with a Wnt ligand and a coreceptor. We decided to take advantage of the conserved nature of APCDD1 to use experiments in Xenopus to identify genes regulated by APCDD1 relevant for human pathology.

To identify genes regulated by APCDD1(“A1”) in early Xenopus development, we decided to deplete A1 protein on the dorsal side of pre-blastula Xenopus embryos. Antisense morpholino oligonucleotides (MO) (GeneTools; Philomath, Oreg.) were injected in the two dorsal blastomeres of 4 cell stage embryos (30 ng/injection). Control and depleted embryos were allowed to develop to stage 10.5, when the dorsal side can be easily recognized because of the presence of the dorsal lip (equivalent of the node and anterior primitive streak in amniotes). Dorsal fragments were then isolated by cutting them with a gastromaster (XenoTech; Lenexa, Kans.) and processed for RNA purification using a proteinase K buffer, Trizol reagent and RNA purification kit (Qiagen; Germantown, Md.). cRNA was produced with Affymetrix labeling kit and hybridized to Affymetrix Xenopus Laevis 2.0 arrays (Rockefeller genomic facility). Results were analyzed with the Genespring program, with a detection threshold of 20, and a minimal variation of 2 fold. Three arrays, hybridized with probes derived from different embryos, were used for both control and depleted samples. Identified genes are shown below in Table 6. The validity of microarray results was tested by qPCR on cDNA obtained from a different experiment on an ABI real-time PCR machine.

TABLE 6
Genes Increased by APCDD1 MO vs. control (dorsal side of embryos only)

| | Fold | |
|---|---|---|
| Gene | Increase | Description |
| angiotensin receptor- | 16 | Required for cardiovascular development in Xenopus (Inui |
| related protein 1b | | Dev Biol. 2006 Oct. 1; 298(1): 188-200; and D B Cox et al., |
| (apelin receptor, agrtl | | Dev Biol. 2006 Aug. 1; 296(1): 177-89) |
| 1b) | | Required for heart field formation in zebrafish (Solnica- |
| | | Krezel Dev Cell. 2007 March; 12(3): 391-402, Stainier Dev |
| | | Cell. 2007 March; 12(3): 403-13). |
| | | Apelin 13 (ligand) might be working through NF-kβ |
| | | transcription in regulating vascular tension. |
| foxi1-ema/ | 13.3 | This gene is normally expressed on the VENTRAL side of |
| Xema/Foxi1/HNF-3 | | the embryo. It is absent in the controls because only the |
| | | dorsal side was analyzed. (Wessely De Rob organizer |
| | | genes). |
| | | It is implicated in negative regulation of endodermal cell |
| | | fate specification |
| | | Positive regulation of transcription, DNA-binding factor |
| | | Negative regulation of mesodermal cell fate determination |
| | | (Suri et al. Development 2005 132: 2733-2742) |
| | | Closest homology to Foxi1/HNF-3 isoform-a in humans. |
| | | HNF-3 cooperates with NF-kβ to activate CRP (C-reactive |
| | | protein) |
| histone 3r | 10.6 | Expressed in the neurula and tadpole stages, possibly |
| | | ectoderm or neural MOD (Pollet Mech Dev. 2005 |
| | | March; 122(3): 365-439). |
| polo-like kinase 2 | 8.9 | Involved in growth. |
| (plk2) | | Also called serum-inducible kinase(SNK)/polo-like kinase |
| | | 2 PKC-like superfamily |
| | | Inhibited by drugs in multiple myeloma treatment |
| | | Plk1 cooperates with Dsh for mitotic progression; Plk2 is |
| | | an activity-inducible kinase that homeostatically decreases |
| | | excitatory synapse number and strength. (EMBO J. 2010 |
| | | Oct. 20; 29(20): 3470-83) |
| | | Phosphorylates centrosomal P4.1-associated protein |
| | | (CPAP), required for cell cycle progression through |
| | | phosphorylation of NPM/B23 (Nucleophosmin) which |
| | | leads to centriole duplication. It is a p53 target (EMBO J. |
| | | 2010 Jul. 21; 29(14): 2395-406) |
| | | Required for NGF-induced neuronal differentiation. The |
| | | gene is silenced in B-cell malignancies. (J Biol Chem. |
| | | 2009 Nov. 13; 284(46): 32053-65) |
| | | Knock-out of gene: retarded growth, slow growth of |
| | | primary fibroblasts (Mol Cell Biol. 2003 October; 23(19): |
| | | 6936-43) |
| | | Expressed in G1 hypocampal cells. |
| | | In XENOPUS, the gene is called Plx2 (Exp Cell Res. 2001 |
| | | Oct. 15; 270(1): 78-87). It accelerates progesterone-induced |
| | | oocyte maturation when overexpressed. |
| | | Present in notochord at stage 13, not detected situ before |
| | | stage 10.5. |
| cyclin G1 (ccng1) | 6.2 | Involved in Growth |
| | | cyclin G1 is repressed by p53, and increases in p53 Knock- |
| | | Out together with p21 |
| PARP3 - poly (ADP- | 4.8 | Role in ectodermal specification and neural crest |
| ribose) polymerase | | development (PLoS One. 2011 Jan. 17; 6(1): e15834). |
| family, member 3 | | Expression is shown to overlap APCDD1. |
| | | Involved in DNA repair with (ADP-ribose)-binding protein |
| | | APLF (Rulten Mol Cell. 2011 Jan. 7; 41(1): 33-45) |
| haeme peroxidase | 4.15 | |
| E3 ubiquitin-protein | 4 | Has been described in a screen for dorsal genes. |
| ligase, Ring finger | | Expressed in anterior endomesoderm. |
| | | In Arabidopsis, a Ring finger ubiquitination protein targets |
| | | Histone 2b |
| ras-like 11b (rasl11b) | 3.46 | Nodal-independent rescue of oep-dep endodermal and |
| | | prechordal plate defects of zygotic oep(−/−) mutants (Zoep). |
| | | No effect on Smad2 phosphorylation (PLoS One. 2008 Jan. |
| | | 16; 3(1): e1434) |
| | | Expressed in organizer and ventral ectoderm, tail tip (like |
| | | APCDD1), the spinal cord (sc), and in several head |
| | | structures such as the hindbrain (hb), the isthmic organizer |
| | | (i), and the otic vesicle (ov). This is a similar expression |
| | | pattern as seen for APCDD1 |
| | | oncogenic activation of ras also activates NF-kβ through |
| | | nonconventional Ikβ kinases |
| Histone 2B | 3.18 | Monoubiquitinated in active genes |
| 5′-nucleotidase, | 3 | |
| cytosolic III (cytosolic | | |
| 5′-nucleotidase III) | | |
| angiotensin receptor- | 2.8 | Decreased in retina of norrin (Wnt receptor) Knock-Out |
| related protein 1 | | mouse (Schafer, Invest Ophthalmol Vis Sci. 2009 |
| (agtrl1, XAngio1 in | | February; 50(2): 906-16. Epub 2008 Oct. 31) |
| Xenopus) | | |
| RAB40B | 2.48 | small GTPase mediated signal transduction. |
| | | May be a substrate-recognition component of a SCF-like |
| | | ECS (Elongin-Cullin-SOCS-box protein) E3 ubiquitin |
| | | ligase complex |
| Histone 2 | 2.45 | |
| FGF receptor 2 | 2.4 | Binds FGF3 (J Biol Chem. 1995 Mar. 24; 270(12): 6779- |
| | | 87.) |
| | | Mutated (activating mutation) in Cruzon syndrome (early |
| | | cranial synostosis) and Apert syndrome (skeletal, neural, |
| | | visceral anomalies). Accelerates cartilage maturation to |
| | | bone. Required for renal development |
| | | PTEN (inhibitor of PI3K) Knock-Out phenotype in bone is |
| | | partially rescued by FGFR2 Knock-Out because its effect |
| | | is due to increased FGF signaling (Dev. 2011 |
| | | April; 138(7): 1433-44.) |
| | | Knock-Out lacks epiblast and basal membrane of visceral |
| | | endoderm |
| | | Expressed in trophectoderm mouse |
| | | requires RAB14 for membrane localization through |
| | | endosomal transport (Dev Cell Volume 20, Issue 1, 18 |
| | | Jan. 2011, Pages 60-71) |
| | | inhibited by miR-125b, which is decreased psoriasis. |
| | | FGFR2 is increased, with incr. proliferation. |
| | | FGFR2(IIb) in keratinocytes; binds KGF; inhibitory for |
| | | hair follicle formation in skin (Dev. 2009 |
| | | July; 136(13): 2153-64. Epub 2009 May 27). |

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method for controlling hair growth in a subject, the method comprising:
a) administering to the subject an effective amount of an APCDD1 modulating compound,
thereby controlling hair growth in the subject.

2. The method of claim 1, wherein controlling hair growth comprises an induction of hair growth in the subject or a promotion of hair loss in the subject.

3. A method for controlling loss of hair pigmentation in a subject, the method comprising:
a) administering to the subject an effective amount of an APCDD1 modulating compound,
thereby controlling hair pigmentation in the subject.

4. The method of claim 1 or 3, wherein the compound comprises an antibody that specifically binds to an APCDD1 protein or a fragment thereof; an antisense RNA or antisense DNA that inhibits expression of an APCDD1 polypeptide; a siRNA that specifically targets an APCDD1 gene; or a combination thereof.

5. The method of claim 1 or 3, wherein the compound comprises a peptide comprising at least about 10 amino acids of SEQ ID NO: 1 or a vector comprising a nucleic acid sequence encoding a polypeptide comprising SEQ ID NO: 1.

6. The method of claim 1 or 3, wherein the subject is a human, a primate, a feline, a canine, or an equine.

7. The method of claim 1 or 3, wherein the subject is afflicted with hypotrichosis.

8. The method of claim 1 or 3, wherein the subject is afflicted with a hair-loss disorder.

9. The method of claim 8, wherein the hair-loss disorder comprises androgenetic alopecia, Telogen effluvium, Alopecia greata, telogen effluvium, Tinea capitis, alopecia totalis, hypotrichosis, hereditary hypotrichosis simplex, or alopecia universalis.

10. The method of claim 1 or 3, wherein the subject is afflicted with hypertrichosis.

11. The method of claim 1 or 3, wherein administering comprises dispersing the APCDD1 modulating compound to a subject via subcutaneous, intradermal, intramuscular, intra-peritoneal, or intravenous injection; infusion; oral, nasal, or topical delivery; or a combination thereof.

12. The method of claim 1 or 3, wherein administering comprises dispersing the APCDD1 modulating compound to an epithelial cell derived from a hair follicle or skin.

13. A composition for modulating APCDD1 protein expression or activity in a subject in need thereof, wherein the composition comprises an siRNA that specifically targets an APCDD1 gene.

14. The composition of claim 13, wherein the siRNA comprises a nucleic acid sequence comprising any one sequence of SEQ ID NO: 112-3776.

15. The composition of claim 13, wherein APCDD1 protein expression is decreased by at least about 40%, at least about 45%, at least about 50%, at least about 55%, at least about 60%, at least about 65%, at least about 70%, at least about 75%, at least about 80%, at least about 90%, at least about 95%, at least about 99%, or 100%.

16. A composition for controlling hair growth or loss of hair pigmentation in a subject, the composition in an admixture of a pharmaceutically acceptable carrier comprising an APCDD1 modulating compound.

17. The composition of claim 16, wherein the pharmaceutically acceptable carrier comprises water, a glycol, an ester, an alcohol, a lipid, or a combination thereof.

18. The composition of claim 16, wherein controlling hair growth comprises an induction of hair growth in the subject or a promotion of hair loss in the subject.

19. The composition of claim 16, wherein the compound comprises an antibody that specifically binds to an APCDD1 protein or a fragment thereof; an antisense RNA or antisense DNA that inhibits expression of an APCDD1 polypeptide; a siRNA that specifically targets an APCDD1 gene; or a combination thereof.

20. The composition of claim 16, wherein the compound comprises a peptide comprising at least about 10 amino acids of SEQ ID NO: 1 or a vector comprising a nucleic acid sequence encoding a polypeptide comprising SEQ ID NO: 1.

21. The composition of claim 13 or 16, wherein the subject is a human, a primate, a feline, a canine, or an equine.

22. The composition of claim 13 or 16, wherein the subject is afflicted with hypotrichosis.

23. The composition of claim 13 or 16, wherein the subject is afflicted with a hair-loss disorder.

24. The composition of claim 23, wherein the hair-loss disorder comprises androgenetic alopecia, Alopecia greata, telogen effluvium, hypotrichosis, alopecia totalis, or alopecia universalis.

25. The composition of claim 13 or 16, wherein the subject is afflicted with hypertrichosis.

26. A kit for controlling hair growth, the kit comprising a container having the composition of claim 16 disposed therein and instructions for use.

27. A method for identifying a compound that modulates APCDD1 protein activity, the method comprising:
a) expressing APCDD1 protein in a cell;
b) contacting a cell with a ligand source for an effective period of time;
c) measuring a secondary messenger response, wherein the response is indicative of a ligand binding to APCDD1 protein;
d) isolating the ligand from the ligand source; and
e) identifying the structure of the ligand that binds APCDD1 protein,
thereby identifying which compound would modulate the activity of APCDD1 protein.

28. The method of claim 27, further comprising:
f) obtaining or synthesizing the compound determined to bind to APCDD1 protein or to modulate APCDD1 protein activity;
g) contacting APCDD1 protein with the compound under a condition suitable for binding; and
h) determining whether the compound modulates APCDD1 protein activity using a diagnostic assay.

29. The method of claim 27, wherein the compound is a APCDD1 agonist or a APCDD1 antagonist.

30. The method of claim 29, wherein the antagonist decreases APCDD1 protein or RNA expression or APCDD1 activity by at least about 10%, at least about 20%, at least about 30%, at least about 40%, at least about 50%, at least about 60%, at least about 70%, at least about 75%, at least about 80%, at least about 90%, at least about 95%, or at least about 99%.

31. The method of claim 29, wherein the antagonist decreases APCDD1 protein or RNA expression or APCDD1 activity by 100%.

32. The method of claim 29, wherein the agonist increases APCDD1 protein or RNA expression or APCDD1 activity by at least about 10%, at least about 20%, at least about 30%, at least about 40%, at least about 50%, at least about 60%, at least about 70%, at least about 75%, at least about 80%, at least about 90%, at least about 95%, or at least about 99%.

33. The method of claim 29, wherein the agonist increases APCDD1 protein or RNA expression or APCDD1 activity by 100%.

34. The method of claim 29, wherein the compound comprises an antibody that specifically binds to an APCDD1 protein or a fragment thereof; an antisense RNA or antisense DNA that inhibits expression of an APCDD1 polypeptide; a siRNA that specifically targets an APCDD1 gene, a peptide comprising at least 10 amino acids of SEQ ID NO:1 wherein the peptide competes with endogenous APCDD1 for ligand binding; or a combination thereof.

35. The method of claim 27, wherein the cell is a bacterium, a yeast, an insect cell, or a mammalian cell.

36. The method of claim 27, wherein the ligand source is a compound library or a tissue extract.

37. The method of claim 27, wherein measuring comprises detecting an increase or decease in a secondary messenger concentration.

38. The method of claim 27, wherein the assay determines the concentration of the secondary messenger within the cell.

39. The method of claim 38, wherein the secondary messenger comprises glycogen synthase kinase 3β (GSK3β), β-catenin, adenomatous polyposis coli (APC), axin, or a combination thereof.

40. The method of claim 28, wherein contacting comprises administering the compound to a mammal in vivo or a cell in vitro.

41. The method of claim 40, wherein the mammal is a mouse.

42. The method of claim 27, wherein the compound increases or decreases downstream signaling of the APCDD1 protein.

43. The method of claim 28, wherein the assay measures an intracellular concentration of glycogen synthase kinase 3β (GSK3β), β-catenin, adenomatous polyposis coli (APC), or axin.

44. The method of claim 28, wherein the assay measures LEF/TCF transcription.

45. The method of claim 28, wherein the assay measures β-catenin phosphorylation or β-catenin nuclear translocation.

46. A method for detecting the presence of or a predisposition to a hair-loss disorder in a human subject, the method comprising:
(a) obtaining a biological sample from a human subject; and
(b) detecting whether or not there is an alteration in the expression of APCDD1 protein in the subject as compared to a subject not afflicted with a hair-loss disorder.

47. The method of claim 46, wherein the detecting comprises detecting whether there is an alteration in the APCDD1 gene locus.

48. The method of claim 47, wherein the alteration comprises a missense mutation.

49. The method of claim 48, wherein the mutation is thymine to guanine substitution at position 26 of SEQ ID NO: 2.

50. The method of claim 46, wherein the detecting comprises detecting whether a small nuclear polymorphism (SNP) is present in the APCDD1 gene locus.

51. The method of claim 50, wherein the SNP comprises a single nucleotide change, or a cluster of SNPs in and around the APCDD1 gene, or other SNPS that are in linkage disequilibrium (LD) with APCDD1.

52. The method of claim 46, wherein the detecting comprises detecting whether at least a portion of the APCDD1 gene is deleted.

53. The method of claim 46, wherein the detecting comprises detecting whether the signal peptide sequence of the APCDD1 protein is altered.

54. The method of claim 46, wherein the detecting comprises detecting whether there is an alteration in the APCDD1 protein.

55. The method of claim 54, wherein the alteration comprises a Leucine to Arginine substitution at amino acid position 9 of SEQ ID NO: 1.

56. The method of claim 46, wherein the detecting comprises detecting whether expression of APCDD1 is reduced.

57. The method of claim 46, wherein the detecting comprises detecting in the sample whether there is a reduction in APCDD1 mRNA, APCDD1 protein, or a combination thereof.

58. The method of claim 46, wherein detecting comprises gene sequencing, selective hybridization, amplification, gene expression analysis, or a combination thereof.

59. The method of claim 46, wherein amplification comprises using forward and reverse RT-PCR primers comprising nucleotide sequences of SEQ ID NOS: 9, 10, 13, 14, 57, or 103.

60. The method of claim 46, wherein the subject is a human, a dog, or a mouse.

61. The method of claim 46, wherein the sample comprises blood, serum, sputum, lacrimal secretions, semen, vaginal secretions, fetal tissue, skin tissue, epithelial tissue, muscle tissue, amniotic fluid, or a combination thereof.

62. The method of claim 46, wherein a reduction in APCDD1 expression of at least 20% indicates a predisposition to or presence of a hair-loss disorder in the subject.

63. The method of claim 46, the hair-loss disorder comprises androgenetic alopecia, Alopecia greata, telogen effluvium, alopecia totalis, hypotrichosis, hereditary hypotrichosis simplex, or alopecia universalis.

64. A diagnostic kit for determining whether a sample from a subject exhibits reduced APCDD1 expression or exhibits an APCDD1 gene mutation, the kit comprising nucleic acid primers that specifically hybridize to and are capable of priming a polymerase reaction from APCDD1.

65. The kit of claim 64, wherein the primers comprise a nucleotide sequence of SEQ ID NOS: 9, 10, 13, 14, 21, 22, 23, 24, 25, 67, 68, 69, 70, or 71.

66. The kit of claim 64, wherein the mutation comprises a Leucine to Arginine substitution at amino acid position 9 of SEQ ID NO: 1.
